(19) United States
(12) Patent Application Publication
(10) Pub. No.: US 2007/0134243 A1
(43) Pub. Date: Jun. 14, 2007

(54) Antibody drug conjugates and methods

(76) Inventors: Lewis J. Gazzard, Belmont, CA (US); Edward HyungSuk Ha, San Francisco, CA (US); David Y. Jackson, Belmont, CA (US); Joann M. Um, Los Angeles, CA (US)

Correspondence Address: GENENTECH, INC., 1 DNA WAY, SOUTH SAN FRANCISCO, CA, 94080, US

(21) Appl. No.: 11/311,591

(22) Filed: Nov. 29, 2005

Related U.S. Application Data
(60) Provisional application No. 60/632,613, filed on Dec. 1, 2004.

(51) Int. Cl. A61K 39/395; C07K 16/46; C07K 16/28; C07K 16/24

(52) U.S. Cl. 424/145.1; 424/178.1; 530/388.23; 530/391.1

(57) ABSTRACT

The invention relates to antibody drug conjugate (ADC) compounds represented by Formula I:
Ab-(L-D)ₚ I
where one or more 1,8 bis-naphthalimide drug moieties (D) having Formulas IIa and IIb are covalently linked by a linker (L) to an antibody (Ab). The invention also relates to pharmaceutical compositions comprising an effective amount of a Formula I ADC for treatment of hyperproliferative disorders and other disorders. The invention also relates to methods for killing or inhibiting the multiplication of a tumor cell or cancer cell including administering to a patient an effective amount of a Formula I ADC.

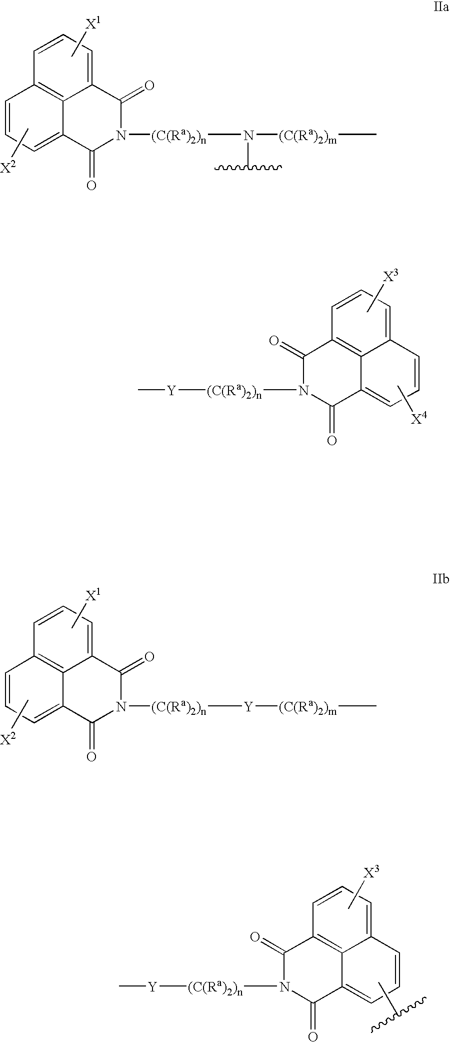

This non-provisional application filed under 37 CFR § 1.53(b), claims the benefit under 35 USC §119(e) of U.S. Provisional Application Ser. No. 60/632,613 filed on 1 Dec. 2004, which is incorporated by reference in entirety.

### FIELD OF THE INVENTION

The invention relates generally to compounds with anti-cancer activity and more specifically to antibodies conjugated with chemotherapeutic drugs or toxins. The invention also relates to methods of using antibody drug conjugate compounds for in vitro, in situ, and in vivo diagnosis or treatment of mammalian cells, or associated pathological conditions.

### BACKGROUND OF THE INVENTION

Improving the delivery of drugs and other agents to target cells, tissues and tumors to achieve maximal efficacy and minimal toxicity has been the focus of considerable research for many years. Though many attempts have been made to develop effective methods for importing biologically active molecules into cells, both in vivo and in vitro, none has proved to be entirely satisfactory. Optimizing the association of the drug with its intracellular target, while minimizing intercellular redistribution of the drug, e.g. to neighboring cells, is often difficult or inefficient.

Monoclonal antibody therapy has been established for the targeted treatment of patients with cancer, immunological and angiogenic disorders. One example, HERCEPTIN®(trastuzumab; Genentech, Inc.; South San Francisco, Calif.) is a recombinant DNA-derived humanized monoclonal antibody that selectively binds with high affinity in a cell-based assay (Kd=5 nM) to the extracellular domain of the human epidermal growth factor receptor2 protein, HER2 (ErbB2) (U.S. Pat. No. 5,821,337; U.S. Pat. No. 6,054,297; U.S. Pat. No. 6,407,213; U.S. Pat. No. 6,639,055; Coussens L, et al (1985) Science 230:1132-9; Slamon D J, et al (1989) Science 244:707-12). Trastuzumab is an IgG1 kappa antibody that contains human framework regions with the complementarity-determining regions (cdr) of a murine antibody (4D5) that binds to HER2. Trastuzumab binds to the HER2 antigen and thus inhibits the growth of cancerous cells. Because Trastuzumab is a humanized antibody, it minimizes any HAMA (Human Anti-Mouse Antibody) response in patients. Trastuzumab has been shown, in both in vitro assays and in animals, to inhibit the proliferation of human tumor cells that overexpress HER2 (Hudziak R M, et al (1989) Mol Cell Biol 9:1165-72; Lewis G D, et al (1993) Cancer Immunol Immunother; 37:255-63; Baselga J, et al (1998) Cancer Res. 58:2825-2831). Trastuzumab is a mediator of antibody-dependent cellular cytotoxicity, ADCC (Hotaling T E, et al (1996) [abstract]. Proc. Annual Meeting Am Assoc Cancer Res; 37:471; Pegram M D, et al (1997) [abstract]. Proc Am Assoc Cancer Res; 38:602). In vitro, Trastuzumab-mediated ADCC has been shown to be preferentially exerted on HER2 overexpressing cancer cells compared with cancer cells that do not overexpress HER2. HERCEPTIN® as a single agent is indicated for the treatment of patients with metastatic breast cancer whose tumors overexpress the HER2 protein and who have received one or more chemotherapy regimens for their metastatic disease. HERCEPTIN® in combination with paclitaxel is indicated for treatment of patients with metastatic breast cancer whose tumors overexpress the HER2 protein and who have not received chemotherapy for their metastatic disease. HERCEPTIN® is clinically active in patients with ErbB2-overexpressing metastatic breast cancers that have received extensive prior anti-cancer therapy (Baselga et al, (1996) J. Clin. Oncol. 14:737-744).

The use of antibody-drug conjugates (ADC), i.e. immunoconjugates, for the local delivery of cytotoxic or cytostatic agents to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Efforts to design and refine ADC have focused on the selectivity of monoclonal antibodies (mAbs) as well as drug-linking and drug-releasing properties. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19):1573-1581; Mandler et al (2000) Bioorganic & Med. Chem. Letters 10:1025-1028; Mandler et al (2002) Bioconjugate Chem. 13:786-791), maytansinoids (US 20050169933 A1; EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ZEVALIN® (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and ¹¹¹In or ⁹⁰Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a hu CD33 antibody linked to calicheamicin, was approved in 2000 for the treatment of acute myeloid leukemia by injection (Drugs of the Future (2000) 25(7):686; U.S. Pat. No. 4,970,198; U.S. Pat. No. 5,079,233; U.S. Pat. No. 5,585,089; U.S. Pat. No. 5,606,040; U.S. Pat. No. 5,693,762; U.S. Pat. No. 5,739,116; U.S. Pat. No. 5,767,285; U.S. Pat. No. 5,773,001). Cantuzumab mertansine (Immunogen, Inc.), an antibody drug conjugate composed of the huC242 antibody linked via the disulfide linker SPP to the maytansinoid drug moiety, DM1, is advancing into Phase H trials for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, is under development for the potential treatment of prostate tumors. The auristatin peptides, auristatin E (AE) and monomethylauristatin (MMAE) synthetic analogs of dolastatin, were conjugated to chimeric monoclonal antibodies including: cBR96 (specific to Lewis Y on carcinomas); cAC10 (specific to CD30 on hematological malignancies); and other antibodies (US 20050238649 A1) and are under therapeutic development (Doronina et al (2003) Nature Biotechnology 21(7):778-784).

Bis-1,8 naphthalimide compounds have been investigated for their anti-cancer properties (Brana et al (2004) Jour. Med. Chem. 47(6):1391-1399; Bailly et al (2003) Biochemistry 42:4136-4150; Carrasco et al (2003) 42:11751-11761; Brana, M. F. and Ramos, A. (2001) Current Med. Chem.—Anti-Cancer Agents 1:237-255; Mekapati et al (2001) Bioorganic & Med. Chem. 9:2757-2762). The investigational antitumor drug bis 1,8 naphthalimide mesylate (LU79553, N,N-bis[1,8-naphthalimido)ethyl]-1,3-diaminopropane bismethane sulfonate; N,N′-Bis[2-(1,3-dioxo-2,3-dihydro-1H-benz[de]isoquinolin-2-yl)ethyl]-1,3-diaminopropane dimethanesulfonate; 2,2′-Propane-1,3-diylbis(iminoethylene)bis(2,3-dihydro-1H-benz[de]isoquinoline-1,3-dione) dimethanesulfonate, Abbott Laboratories, Knoll A G, Ludwigshafen, D E), is composed of two tricyclic 1,8-naphthalimide chromophores separated by an aminoalkyl linker chain and designed to permit bisintercalation of the drug into DNA (Villalona-Calero et al (2001) Jour. Clinical Oncology 19(3):857-869; Bousquet et al (1995) Cancer Res. 55:1176-1180; U.S. Pat. No. 4,874,863; U.S. Pat. No. 5,416,089; U.S. Pat. No. 5,616,589; U.S. Pat. No. 5,789,418; WO 95/05365).

### SUMMARY OF THE INVENTION

The present invention provides novel compounds with biological activity against cancer cells. The compounds of the invention may inhibit tumor growth in mammals. The compounds of the invention may be useful for treating human cancer patients.

One aspect of the invention includes antibody drug conjugate (ADC) compounds represented by Formula I:
Ab-(L-D)ₚ I

where one or more 1,8 bis-naphthalimide drug moieties (D) are covalently linked by a linker (L) to an antibody (Ab).

In certain embodiments, Ab binds specifically to a tumor-associated antigen or cell-surface receptor.

In another aspect, the antibody of the Formula I ADC of the invention specifically binds to a receptor encoded by an ErbB gene such as, but not limited to, EGFR, HER2, HER3 and HER4. The antibody may bind specifically to an HER2 receptor.

In another aspect, the antibody of the antibody-drug conjugate is a humanized antibody selected from huMAb4D5-1, huMAb4D5-2, huMAb4D5-3, huMAb4D5-4, huMAb4D5-5, huMAb4D5-6, huMAb4D5-7 and huMAb4D5-8 (Trastuzumab).

In still another aspect, the invention provides pharmaceutical compositions comprising an effective amount of a Formula I ADC and a pharmaceutically acceptable carrier or vehicle.

In another aspect, the invention includes a method of treating cancer comprising administering to a mammal, such as a patient with a hyperproliferative disorder, a formulation of a Formula I ADC and a pharmaceutically acceptable diluent, carrier or excipient.

In another aspect, the invention provides methods for preventing the multiplication of a tumor cell or cancer cell including administering to a mammal, such as a patient with a hyperproliferative disorder, an effective amount of a Formula I ADC.

In yet another aspect, the invention provides methods for preventing cancer including administering to a patient with a hyperproliferative disorder, an effective amount of a Formula I ADC.

In another aspect, the invention includes a pharmaceutical composition comprising an effective amount of a Formula I ADC, or a pharmaceutically acceptable salt thereof, and a pharmaceutically acceptable diluent, carrier or excipient. The composition may further comprise a therapeutically effective amount of chemotherapeutic agent such as a tubulin-forming inhibitor, a topoisomerase inhibitor, or a DNA binder.

In another aspect, the invention includes a method for killing or inhibiting the proliferation of tumor cells or cancer cells comprising treating tumor cells or cancer cells with an amount of a Formula I ADC, or a pharmaceutically acceptable salt or solvate thereof, being effective to kill or inhibit the proliferation of the tumor cells or cancer cells.

In another aspect, the invention includes a method of inhibiting cellular proliferation comprising exposing mammalian cells in a cell culture medium to an ADC of the invention.

In another aspect, the invention includes a method for treating an autoimmune disease, comprising administering to a patient, for example a human with a hyperproliferative disorder, an amount of the ADC of Formula I or a pharmaceutically acceptable salt or solvate thereof, said amount being effective to treat an autoimmune disease.

In another aspect, the invention includes a method for killing or inhibiting the multiplication of a tumor cell or cancer cell comprising administering to a patient, for example a human, with a hyperproliferative disorder, an amount of the ADC of Formula I or a pharmaceutically acceptable salt or solvate thereof, said amount being effective to kill or inhibit the multiplication of a tumor cell or cancer cell.

In another aspect, the invention includes a method for treating cancer comprising administering to a patient, for example a human, with a hyperproliferative disorder, an amount of the ADC of Formula I or a pharmaceutically acceptable salt or solvate thereof, said amount being effective to treat cancer, alone or together with an effective amount of an additional anticancer agent.

In another aspect, the invention includes a method of inhibiting the growth of tumor cells that overexpress a growth factor receptor selected from the group consisting of HER2 receptor and EGF receptor comprising administering to a patient an antibody drug conjugate compound of the invention which binds specifically to said growth factor receptor and a chemotherapeutic agent wherein said antibody drug conjugate and said chemotherapeutic agent are each administered in amounts effective to inhibit growth of tumor cells in the patient.

In another aspect, the invention includes a method for the treatment of a human patient susceptible to or diagnosed with a disorder characterized by overexpression of ErbB2 receptor, comprising administering an effective amount of a combination of an ADC and a chemotherapeutic agent.

In another aspect, the invention includes an assay for detecting cancer cells comprising:

(a) exposing cells to a Formula I ADC; and

(b) determining the extent of binding of the antibody-drug conjugate compound to the cells.

In another aspect, the present invention provides assays for identifying ADC which specifically target and bind the overexpressed HER2 protein, the presence of which is correlated with abnormal cellular function, and in the pathogenesis of cellular proliferation and/or differentiation of mammary gland that is causally related to the development of breast tumors.

In another aspect, the invention includes an article of manufacture comprising an antibody-drug conjugate compound of the invention; a container; and a package insert or label indicating that the compound can be used to treat cancer characterized by the overexpression of an ErbB receptor.

In another aspect, the invention includes a method for the treatment of cancer in a mammal, wherein the cancer is characterized by the overexpression of an ErbB receptor and does not respond, or responds poorly, to treatment with an anti-ErbB antibody, comprising administering to the mammal a therapeutically effective amount of a Formula I ADC.

In another aspect, the invention includes a method of making an antibody drug conjugate compound comprising conjugating a 1,8 bis naphthalimide drug moiety and an antibody.

### BRIEF DESCRIPTION OF THE DRAWINGS

FIG. 1 shows an in vitro, cell proliferation assay with SK-BR-3 cells treated with: -o- trastuzumab and -●-trastuzumab-MC-vc-PAB-(N,N′-(bis-aminoethyl-1,3-propanediamine)-bis-(4-N-imidazolyl)-1,8 naphthalimide) 202, measured in Relative Fluorescence Units (RLU) versus μg/ml concentration of antibody or ADC. H=trastuzumab, where H is linked via a cysteine [cys].

FIG. 2 shows an in vitro, cell proliferation assay with SK-BR-3 cells treated with: -●- Trastuzumab and -Δ- trastuzumab-MC-ala-phe-PAB-(N,N′-(bis-aminoethyl-1,3-propanediamine)-bis-(4-N-imidazolyl)-1,8 naphthalimide) 203, measured in Relative Fluorescence Units (RLU) versus μg/ml concentration of antibody or ADC. H=trastuzumab, where H is linked via a cysteine [cys].

FIG. 3 shows an in vitro, cell proliferation assay with BT-574 cells treated with: -●- trastuzumab, and -o- trastuzumab-(succinate-gly-ala-phe)-(N,N′-(bis-aminoethyl-1,3-propanediamine)-bis 3-nitro-1,8 naphthalimide) 204, measured in Relative Fluorescence Units (RLU) versus μg/ml concentration of antibody or ADC. H=trastuzumab, where H is linked via an amino group.

FIG. 4 shows an in vitro, cell proliferation assay with BT-574 cells treated with: -●- trastuzumab, and -▴- trastuzumab-(MC-val-cit-PAB-(N,N′-(N,N′-(bis-aminoethyl-1,3-propanediamine)-3-nitro, 4-amino-1,8 naphthalimide) 205, measured in Relative Fluorescence Units (RLU) versus μg/ml concentration of antibody or ADC. H=trastuzumab, where H is linked via a cysteine [cys].

FIG. 5 shows an in vitro, cell proliferation assay with SK-BR-3 cells treated with: -●- trastuzumab, -♦-trastuzumab-MC-(N,N′-(bis-aminoethyl-1,3-propanediamine)-bis-(4-N-imidazolyl)-1,8 naphthalimide) 206, and -▾- trastuzumab-N¹-cyclopropylmethyl, N²-maleimidopropyl-gly-val-cit-PAB-(N,N′-(bis-aminoethyl-1,3-propanediamine)-bis 3-nitro-1,8 naphthalimide) 207, measured in Relative Fluorescence Units (RLU) versus μg/ml concentration of antibody or ADC. H=trastuzumab, where H is linked via a cysteine [cys].

FIG. 6 shows a method for preparing a valine-citrulline (val-cit or vc) dipeptide Linker having a maleimide Stretcher and optionally a p-aminobenzyloxycarbonyl (PAB) self-immolative Spacer where Q is —C₁-C₈ alkyl, —O—(C₁-C₈ alkyl), -halogen, -nitro or -cyano; and m is an integer ranging from 0-4.

FIG. 7 shows a method for preparing a phe-lys(Mtr) dipeptide linker reagent having a maleimide Stretcher unit and a p-aminobenzyl self-immolative Spacer unit, where Q is —C₁-C₈ alkyl, —O—(C₁-C₈ alkyl), -halogen, -nitro or -cyano; and m is an integer ranging from 0-4.

FIG. 8 shows three exemplary strategies for covalent attachment of the amino group of a drug moiety to a linker reagent to form a bis 1,8 naphthalimide-linker reagent.

FIG. 9 shows a method for synthesis of a bis 1,8 naphthalimide-linker reagent.

FIG. 10 shows a method for the synthesis of a branched linker reagent containing a BHMS group.

### DETAILED DESCRIPTION OF THE EXEMPLARY EMBODIMENTS

Reference will now be made in detail to certain exemplary embodiments of the invention, examples of which are illustrated in the accompanying structures, drawings, figures, formulas and Examples. While the invention will be described in conjunction with the enumerated embodiments, it will be understood that they are not intended to limit the invention to those embodiments. The discussion below is descriptive, illustrative and exemplary and is not to be taken as limiting the scope defined by any appended claims. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described.

Unless defined otherwise, technical and scientific terms used herein have the same meaning as commonly understood by one of ordinary skill in the art to which this invention belongs, and are consistent with: Singleton et al., (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

When trade names are used herein, applicants intend to independently include the trade name product formulation, the generic drug, and the active pharmaceutical ingredient(s) of the trade name product.

Definitions

Unless stated otherwise, the following terms and phrases as used herein are intended to have the following meanings:

The term “antibody” herein is used in the broadest sense and specifically covers monoclonal antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

An “intact antibody” herein is one comprising a VL and VH domains, as well as complete light and heavy chain constant domains.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The “monoclonal antibodies” may also be isolated from phage antibody libraries using the techniques described in Clackson et al (1991) Nature, 352:624-628; Marks et al (1991) J. Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

“Antibody fragments” comprise a portion of an intact antibody, e.g. comprising the antigen-binding or variable region thereof. Examples of antibody fragments include Fab, Fab′, F(ab′)2, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Useful non-immunoreactive protein, polypeptide, or peptide antibodies include, but are not limited to, transferrin, epidermal growth factors (“EGF”), bombesin, gastrin, gastrin-releasing peptide, platelet-derived growth factor, IL-2, IL-6, transforming growth factors (“TGF”), such as TGF-α and TGF-β, vaccinia growth factor (“VGF”), insulin and insulin-like growth factors I and II, lectins and apoprotein from low density lipoprotein.

Useful polyclonal antibodies are heterogeneous populations of antibody molecules derived from the sera of immunized animals. Various procedures well known in the art may be used for the production of polyclonal antibodies to an antigen-of-interest. For example, for the production of polyclonal antibodies, various host animals can be immunized by injection with an antigen of interest or derivative thereof, including but not limited to rabbits, mice, rats, and guinea pigs. Various adjuvants may be used to increase the immunological response, depending on the host species, and including but not limited to Freund's (complete and incomplete) adjuvant, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture. These include, but are not limited to, the hybridoma technique originally described by Köhler and Milstein (1975, Nature 256, 495-497), the human B cell hybridoma technique (Kozbor et al., 1983, Immunology Today 4: 72), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA, and IgD and any subclass thereof. The hybridoma producing the mAbs of use in this invention may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80, 7308-7312; Kozbor et al., 1983, Immunology Today 4, 72-79; and Olsson et al., 1982, Meth. Enzymol. 92, 3-16).

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually performed using affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, C_{H}2, and C_{H}3 regions. The first heavy-chain constant region (C_{H}1) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation (WO 94/04690; Suresh et al., Methods in Enzymology, 1986, 121:210; Rodrigues et al., 1993, J. of Immunology 151:6954-6961; Carter et al., 1992, Bio/Technology 10:163-167; Carter et al., 1995, J. of Hematotherapy 4:463-470; Merchant et al., 1998, Nature Biotechnology 16:677-681. Using such techniques, bispecific antibodies can be prepared for conjugation as ADC in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof (EP 105360; WO 83/03679; EP 217577).

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay) (See, for e.g., Kabat et al., 1991, Sequences of Proteins of Immunological Interest, Fifth Edition, National Institute of Health, Bethesda, Md.; Kabat E et al., 1980, J. of Immunology 125(3):961-969).

Other useful antibodies include fragments of antibodies such as, but not limited to, F(ab′)2 fragments, which contain the variable region, the light chain constant region and the CH1 domain of the heavy chain can be produced by pepsin digestion of the antibody molecule, and Fab fragments, which can be generated by reducing the disulfide bridges of the F(ab′)2 fragments. Other useful antibodies are heavy chain and light chain dimers of antibodies, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., (1989) Nature 334:544-54), or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in WO 87/02671; EP 184,187; EP 171496; EP 173494; WO 86/01533; U.S. Pat. No. 4,816,567; EP 12023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229: 1202-1207; Oi et al., 1986, BioTechniques 4: 214; U.S. Pat. No. 5,225,539; Jones et al., 1986, Nature 321: 552-525; Verhoeyan et al. (1988) Science 239:1534; and Beidler et al., 1988, J. Immunol. 141:4053-4060; each of which is incorporated herein by reference in its entirety.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies. See, e.g., U.S. Pat. Nos. 5,625,126; 5,633,425; 5,569,825; 5,661,016; 5,545,806; each of which is incorporated herein by reference in its entirety. Other human antibodies can be obtained commercially from, for example, Abgenix, Inc. (Freemont, Calif.) and Genpharm (San Jose, Calif.).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Biotechnology 12:899-903). Human antibodies can also be produced using various techniques known in the art, including phage display libraries (Hoogenboom and Winter, J. Mol. Biol., 227:381 (1991); Marks et al., J. Mol. Biol., 222:581 (1991)).

The antibody may be a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies in ADC include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor (see, e.g., WO 97/34631, which is incorporated herein by reference in its entirety). Antibodies immunospecific for a cancer cell antigen can be obtained commercially, for example, from Genentech (San Francisco, Calif.) or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

The antibody of the antibody-drug conjugates (ADC) of the invention may specifically bind to a receptor encoded by an ErbB gene. The antibody may bind specifically to an ErbB receptor selected from EGFR, HER2, HER3 and HER4. The ADC may specifically bind to the extracellular domain of the HER2 receptor and inhibit the growth of tumor cells which overexpress HER2 receptor. The antibody of the ADC may be a monoclonal antibody, e.g. a murine monoclonal antibody, a chimeric antibody, or a humanized antibody. A humanized antibody may be huMAb4D5-1, huMAb4D5-2, huMAb4D5-3, huMAb4D5-4, huMAb4D5-5, huMAb4D5-6, huMAb4D5-7 or huMAb4D5-8 (Trastuzumab). The antibody may be an antibody fragment, e.g. a Fab fragment.

Known antibodies for the treatment or prevention of cancer can be conjugated as ADC. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer; RITUXAN® (rituximab; Genentech) which is a chimeric anti-CD20 monoclonal antibody for the treatment of patients with non-Hodgkin's lymphoma; OvaRex (AltaRex Corporation, MA) which is a murine antibody for the treatment of ovarian cancer; Panorex (Glaxo Wellcome, NC) which is a murine IgG₂ₐ antibody for the treatment of colorectal cancer; Cetuximab Erbitux (Imclone Systems Inc., NY) which is an anti-EGFR IgG chimeric antibody for the treatment of epidermal growth factor positive cancers, such as head and neck cancer; Vitaxin (MedImmune, Inc., MD) which is a humanized antibody for the treatment of sarcoma; Campath I/H (Leukosite, MA) which is a humanized IgG, antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (Immunomedics, Inc., NJ) which is a humanized anti-CD22 IgG antibody for the treatment of non-Hodgkin's lymphoma; Smart ID10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA) which is a humanized anti-CD2 mAb for the treatment of Hodgkin's Disease or non-Hodgkin's lymphoma; Avastin (Genentech, Inc., CA) which is an anti-VEGF humanized antibody for the treatment of lung and colorectal cancers; Epratuzamab (Immunomedics, Inc., NJ and Amgen, Calif.) which is an anti-CD22 antibody for the treatment of non-Hodgkin's lymphoma; and CEAcide (Immunomedics, NJ) which is a humanized anti-CEA antibody for the treatment of colorectal cancer.

Other antibodies useful in the treatment of cancer include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE -4 (carcinomas), anti-transferrin receptor (carcinomas), p97 (melanoma), MUC1-KLH (breast cancer), CEA (colorectal), gp100 (melanoma), MARTI (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, Pa., et al Cancer Research (1997) 57, 100-105, mAbs against the CD40 antigen, such as S2C6 mAb (Francisco, J. A., et al Cancer Res. (2000) 60:3225-3231), mAbs against the CD70 antigen, such as 1F6 mAb, and mAbs against the CD30 antigen, such as AC10 (Bowen, M. A., et al (1993) J. Immunol., 151:5896-5906; Wahl et al., 2002 Cancer Res. 62(13):3736-42). Many other internalizing antibodies that bind to tumor associated antigens can be used and have been reviewed (Franke, A. E., et al Cancer Biother Radiopharm. (2000) 15:459-76; Murray, J. L., (2000) Semin Oncol., 27:64-70; Breitling, F., and Dubel, S., Recombinant Antibodies, John Wiley, and Sons, New York, 1998).

Known antibodies for the treatment or prevention of an autoimmune disorders may be conjugated as ADC. Autoimmune disorders include systemic lupus erythematosus (SLE), rheumatoid arthritis, Sjogren's syndrome, immune thromobocytopenia, and multiple sclerosis. Antibodies immunospecific for an antigen of a cell that is responsible for producing autoimmune antibodies can be obtained by any method known to one of skill in the art such as, e.g., chemical synthesis or recombinant expression techniques. SLE is marked by the overexpression of interferon-alpha (IFN-α) cytokine genes (Bennett et al (2003) Jour. Exp. Med. 197:711-723). Type-1 interferons (IFN-α/β) play a significant role in the pathogenesis of lupus (Santiago-Raber (2003) Jour. Exp. Med. 197:777-788). Knockout mice (-IFN-α/β) showed significantly reduced anti-erythrocyte autoantibodies, erythroblastosis, hemolytic anemia, anti-DNA autobodies, kidney disease, and mortality. These results suggest that Type-1 IFNs mediate murine lupus, and that reducing their activity in the human counterpart may be beneficial. Anti-IFN Ab conjugated to bis 1,8 naphthalimide drug moieties may be effective therapeutic agents against SLE and other autoimmune disorders.

In another embodiment, useful antibodies in ADC are immunospecific for the treatment of autoimmune diseases include, but are not limited to, Anti-Nuclear Antibody; Anti ds DNA; Anti ss DNA, Anti Cardiolipin Antibody IgM, IgG; Anti Phospholipid Antibody IgM, IgG; Anti SM Antibody; Anti Mitochondrial Antibody; Thyroid Antibody; Microsomal Antibody; Thyroglobulin Antibody; Anti SCL-70; Anti-Jo; Anti-U₁RNP; Anti-La/SSB; Anti SSA; Anti SSB; Anti Perital Cells Antibody; Anti Histones; Anti-RNP; C-ANCA; P-ANCA; Anti centromere; Anti-Fibrillarin, and Anti-GBM Antibody.

Antibodies of an ADC can bind to both a receptor or a receptor complex expressed on an activated lymphocyte, such as one associated with an autoimmune disease. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C-type, S-type, and I-type lectin.

As used herein, the term “viral antigen” includes, but is not limited to, any viral peptide, polypeptide protein (e.g., HIV gp120, HIV nef, RSV F glycoprotein, influenza virus neuraminidase, influenza virus hemagglutinin, HTLV tax, herpes simplex virus glycoprotein (e.g., Gb, Gc, Gd, and Ge) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacterial, fungi, pathogenic protozoa, or yeast polypeptide including, e.g., LPS and capsular polysaccharide 5/8) that is capable of eliciting an immune response.

Antibodies immunospecific for a viral or microbial antigen can be obtained commercially, for example, from BD Biosciences (San Francisco, Calif.), Chemicon International, Inc. (Temecula, Calif.), or Vector Laboratories, Inc. (Burlingame, Calif.) or produced by any method known to one of skill in the art such as, e.g., chemical synthesis or recombinant expression techniques. The nucleotide sequence encoding antibodies that are immunospecific for a viral or microbial antigen can be obtained, e.g., from the GenBank database or a database like it, the literature publications, or by routine cloning and sequencing.

In a specific embodiment, useful antibodies in ADC are those that treat or prevent viral or microbial infection in accordance with the methods disclosed herein. Examples of antibodies available useful for the treatment of viral infection or microbial infection include, but are not limited to, SYNAGIS (MedImmune, Inc., MD) which is a humanized anti-respiratory syncytial virus (RSV) monoclonal antibody useful for the treatment of patients with RSV infection; PRO542 (Progenics) which is a CD4 fusion antibody useful for the treatment of HIV infection; OSTAVIR (Protein Design Labs, Inc., CA) which is a human antibody useful for the treatment of hepatitis B virus; PROTOVTR (Protein Design Labs, Inc., CA) which is a humanized IgG₁ antibody useful for the treatment of cytomegalovirus (CMV); and anti-LPS antibodies.

Other antibodies useful in ADC for the treatment of infectious diseases include, but are not limited to, antibodies against the antigens from pathogenic strains of bacteria (Streptococcus pyogenes, Streptococcus pneumoniae, Neisseria gonorrheae, Neisseria meningitidis, Corynebacterium diphtheriae, Clostridium botulinum, Clostridium perfringens, Clostridium tetani, Hemophilus influenzae, Klebsiella pneumoniae, Klebsiella ozaenas, Klebsiella rhinoscieromotis, Staphylococcus aureus, Vibrio colerae, Escherichia coli, Pseudomonas aeruginosa, Campylobacter (Vibrio) fetus, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp.); pathogenic fungi (Coccidioides immitis, Aspergillus fumigatus, Candida albicans, Blastomyces dermatitidis, Cryptococcus neoformans, Histoplasma capsulatum); protozoa (Entomoeba histolytica, Toxoplasma gondii, Trichomonas tenas, Trichomonas hominis, Trichomonas vaginalis, Tryoanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, Plasmodium malaria); or Helminiths (Enterobius vermicularis, Trichuris trichiura, Ascaris lumbricoides, Trichinella spiralis, Strongyloides stercoralis, Schistosomajaponicum, Schistosoma mansoni, Schistosoma haematobium, and hookworms).

Other antibodies useful in ADC for treatment of viral disease include, but are not limited to, antibodies against antigens of pathogenic viruses, including as examples and not by limitation: Poxyiridae, Herpesviridae, Herpes Simplex virus 1, Herpes Simplex virus 2, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Hepatitis A virus, Hepatitis B virus, Hepatitis C virus, Hepatitis E virus, Non-A/Non-B Hepatitis virus, Rhinoviridae, Coronaviridae, Rotoviridae, and Human Immunodeficiency Virus.

An “ErbB receptor” is a receptor protein tyrosine kinase which belongs to the ErbB receptor family whose members are mediators of cell growth, differentiation and survival. The ErbB receptor family includes four distinct members including epidermal growth factor receptor (EGFR, ErbB1, HER1), HER2 (ErbB2 or p185^{neu}), HER3 (ErbB3) and HER4 (ErbB4 or tyro2). A panel of anti-ErbB2 antibodies has been characterized using the human breast tumor cell line SKBR3 (Hudziak et al., (1989) Mol. Cell. Biol. 9(3):1165-1172. Maximum inhibition was obtained with the antibody called 4D5 which inhibited cellular proliferation by 56%. Other antibodies in the panel reduced cellular proliferation to a lesser extent in this assay. The antibody 4D5 was further found to sensitize ErbB2-overexpressing breast tumor cell lines to the cytotoxic effects of TNF-α (U.S. Pat. No. 5,677,171). The anti-ErbB2 antibodies discussed in Hudziak et al. are further characterized in Fendly et al (1990) Cancer Research 50:1550-1558; Kotts et al. (1990) In Vitro 26(3):59A; Sarup et al. (1991) Growth Regulation 1:72-82; Shepard et al. J. (1991) Clin. Immunol. 1 (3):117-127; Kumar et al. (1991) Mol. Cell. Biol. 11(2):979-986; Lewis et al. (1993) Cancer Immunol. Immunother. 37:255-263; Pietras et al. (1994) Oncogene 9:1829-1838; Vitetta et al. (1994) Cancer Research 54:5301-5309; Sliwkowski et al. (1994) J. Biol. Chem. 269(20):14661-14665; Scott et al. (1991) J. Biol. Chem. 266:14300-5; D'souza et al. Proc. Natl. Acad. Sci. (1994) 91:7202-7206; Lewis et al. (1996) Cancer Research 56:1457-1465; and Schaefer et al. (1997) Oncogene 15:1385-1394.

The ErbB receptor will generally comprise an extracellular domain, which may bind an ErbB ligand; a lipophilic transmembrane domain; a conserved intracellular tyrosine kinase domain; and a carboxyl-terminal signaling domain harboring several tyrosine residues which can be phosphorylated. The ErbB receptor may be a “native sequence” ErbB receptor or an “amino acid sequence variant” thereof. The ErbB receptor may be a native sequence human ErbB receptor. Accordingly, a “member of the ErbB receptor family” is EGFR (ErbB1), ErbB2, ErbB3, ErbB4 or any other ErbB receptor currently known or to be identified in the future.

The terms “ErbB1”, “epidermal growth factor receptor”, “EGFR” and “HER1” are used interchangeably herein and refer to EGFR as disclosed, for example, in Carpenter et al (1987) Ann. Rev. Biochem., 56:881-914, including naturally occurring mutant forms thereof (e.g., a deletion mutant EGFR as in Humphrey et al., PNAS (USA), 87:4207-4211 (1990)). The term erbB1 refers to the gene encoding the EGFR protein product. Antibodies against HER1 are described, for example, in Murthy et al (1987) Arch. Biochem. Biophys., 252:549-560 and in WO 95/25167.

The term “ERRP”, “EGF-Receptor Related Protein”, “EGFR Related Protein” and “epidermal growth factor receptor related protein” are used interchangeably herein and refer to ERRP as disclosed, for example in U.S. Pat. No. 6,399,743 and US Publication No. 2003/0096373.

The expressions “ErbB2” and “HER2” are used interchangeably herein and refer to human HER2 protein described, for example, in Semba et al., PNAS (USA), 82:6497-6501 (1985) and Yamamoto et al., (1986) Nature, 319:230-234 (Genebank accession number X03363). The term “erbB2” refers to the gene encoding human ErbB2 and “neu” refers to the gene encoding rat p185neu. ErbB2 may be a native sequence human ErbB2.

“ErbB3” and “HER3” refer to the receptor polypeptide as disclosed, for example, in U.S. Pat. No. 5,183,884 and U.S. Pat. No. 5,480,968 as well as Kraus et al., PNAS (USA), 86:9193-9197 (1989). Antibodies against ErbB3 are known in the art and are described, for example, in U.S. Pat. Nos. 5,183,884, 5,480,968 and in WO 97/35885.

The terms “ErbB4” and “HER4” herein refer to the receptor polypeptide as disclosed, for example, in EP Pat Application No 599,274; Plowman et al., Proc. Natl. Acad. Sci. USA, 90:1746-1750 (1993); and Plowman et al., Nature, 366:473-475 (1993), including isoforms thereof, e.g., as disclosed in WO 99/19488. Antibodies against HER4 are described, for example, in WO 02/18444.

Antibodies to ErbB receptors are available commercially from a number of sources, including, for example, Santa Cruz Biotechnology, Inc., California, USA.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and preferably, they will be at least about 80%, more preferably, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody. An exemplary FcR is a native sequence human FcR. Moreover, a FcR may be one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See review M. in Daëron, Annu. Rev. Immunol., 15:203-234 (1997)). FcRs are reviewed in Ravetch and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol., 117:587 (1976) and Kim et al., J. Immunol., 24:249 (1994)).

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods, 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the O-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk (1987) J. Mol. Biol., 196:901-917). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding. For a review of scFv, see Plüickthun in The Pharmacology of Monoclonal Antibodies, vol. 113, Rosenburg and Moore eds., Springer-Verlag, New York, pp. 269-315 (1994). Anti-ErbB2 antibody scFv fragments are described in WO 93/16185; U.S. Pat. Nos. 5,571,894; and 5587458.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al (1993) Proc. Natl. Acad. Sci. USA 90:6444-6448.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework (Jones et al, (1986) Nature 321:522-525). These CDR-grafted antibodies generally do not retain their original affinity for antigen binding, and in fact, affinity is often severely impaired. Besides the CDRs, select non-human antibody framework residues must also be incorporated to maintain proper CDR conformation (Chothia et al (1989) Nature 342:877). The transfer of key mouse framework residues to the human acceptor in order to support the structural conformation of the grafted CDRs has been shown to restore antigen binding and affinity (Riechmann et al., (1992) J. Mol. Biol. 224, 487-499; Foote and Winter, (1992) J. Mol. Biol. 224:487-499; Presta et al., (1993) J. Immunol. 151, 2623-2632; Werther et al., (1996) J. Immunol. Methods 157:4986-4995; and Presta et al (2001) Thromb. Haemost. 85:379-389). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see U.S. Pat. No. 6,407,213; Jones et al (1986) Nature, 321:522-525; Riechmann et al (1988) Nature 332:323-329; and Presta, (1992) Curr. Op. Struct. Biol., 2:593-596.

Humanized anti-ErbB2 antibodies include huMAb4D5-1, huMAb4D5-2, huMAb4D5-3, huMAb4D5-4, huMAb4D5-5, huMAb4D5-6, huMAb4D5-7 and huMAb4D5-8 (HERCEPTIN®) as described in Table 3 of U.S. Pat. No. 5,821,337, expressly incorporated herein by reference; humanized 520C9 (WO 93/21319) and humanized 2C4 antibodies as described herein below.

A “parent antibody” is an antibody comprising an amino acid sequence from which one or more amino acid residues are replaced by one or more cysteine residues. The parent antibody may comprise a native or wild type sequence. The parent antibody may have pre-existing amino acid sequence modifications (such as additions, deletions and/or substitutions) relative to other native, wild type, or modified forms of an antibody. A parent antibody is directed against a target antigen of interest. Antibodies directed against nonpolypeptide antigens (such as tumor-associated glycolipid antigens; see U.S. Pat. No. 5,091,178) are also contemplated.

Other exemplary parent antibodies include those selected from, and without limitation, anti-estrogen receptor antibody, anti-progesterone receptor antibody, anti-p53 antibody, anti-HER-2/neu antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD10 antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCA/CD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokeratins antibody, anti-vimentins antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody and anti-Tn-antigen antibody.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

An antibody “which binds” a molecular target or an antigen of interest, e.g., ErbB2 antigen, is one capable of binding that antigen with sufficient affinity such that the antibody is useful in targeting a cell expressing the antigen. Where the antibody is one which binds ErbB2, it will usually preferentially bind ErbB2 as opposed to other ErbB receptors, and may be one which does not significantly cross-react with other proteins such as EGFR, ErbB3 or ErbB4. In such embodiments, the extent of binding of the antibody to these non-ErbB2 proteins (e.g., cell surface binding to endogenous receptor) will be less than 10% as determined by fluorescence activated cell sorting (FACS) analysis or radioimmunoprecipitation (RIA). Sometimes, the anti-ErbB2 antibody will not significantly cross-react with the rat neu protein, e.g., as described in Schecter et al., Nature 312:513 (1984) and Drebin et al., Nature 312:545-548 (1984).

Molecular targets for the antibody drug conjugates (ADC) encompassed by the present invention include: (i) tumor-associated antigens; (ii) cell surface receptors, (iii) CD proteins and their ligands, such as CD3, CD4, CD8, CD19, CD20, CD22, CD34, CD40, CD79a (CD79a), and CD79P (CD79b); (iv) members of the ErbB receptor family such as the EGF receptor, HER2, HER3 or HER4 receptor; (v) cell adhesion molecules such as LFA-1, Mac1, p150,95, VLA-4, ICAM-1, VCAM and αv/β3 integrin including either alpha or beta subunits thereof (e.g. anti-CD11a, anti-CD18 or anti-CD11b antibodies); and (vi) growth factors such as VEGF; IgE; blood group antigens; flk2/flt3 receptor; obesity (OB) receptor; mpl receptor; CTLA-4; protein C, BR3, c-met, tissue factor, β7 etc.

Unless indicated otherwise, the term “monoclonal antibody 4D5” refers to an antibody that has antigen binding residues of, or derived from, the murine 4D5 antibody (ATCC CRL 10463). For example, the monoclonal antibody 4D5 may be murine monoclonal antibody 4D5 or a variant thereof, such as a humanized 4D5. Exemplary humanized 4D5 antibodies include huMAb4D5-1, huMAb4D5-2, huMAb4D5-3, huMAb4D5-4, huMAb4D5-5, huMAb4D5-6, huMAb4D5-7 and huMAb4D5-8 (Trastuzumab, HERCEPTIN®) as in U.S. Pat. No. 5,821,337.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

An “ErbB-expressing cancer” is one comprising cells which have ErbB protein present at their cell surface. An “ErbB2-expressing cancer” is one which produces sufficient levels of ErbB2 at the surface of cells thereof, such that an anti-ErbB2 antibody can bind thereto and have a therapeutic effect with respect to the cancer.

A cancer “characterized by excessive activation” of an ErbB receptor is one in which the extent of ErbB receptor activation in cancer cells significantly exceeds the level of activation of that receptor in non-cancerous cells of the same tissue type. Such excessive activation may result from overexpression of the ErbB receptor and/or greater than normal levels of an ErbB ligand available for activating the ErbB receptor in the cancer cells. Such excessive activation may cause and/or be caused by the malignant state of a cancer cell. In some embodiments, the cancer will be subjected to a diagnostic or prognostic assay to determine whether amplification and/or overexpression of an ErbB receptor is occurring which results in such excessive activation of the ErbB receptor. Alternatively, or additionally, the cancer may be subjected to a diagnostic or prognostic assay to determine whether amplification and/or overexpression an ErbB ligand is occurring in the cancer which attributes to excessive activation of the receptor. In a subset of such cancers, excessive activation of the receptor may result from an autocrine stimulatory pathway.

A cancer which “overexpresses” an ErbB receptor is one which has significantly higher levels of an ErbB receptor, such as ErbB2, at the cell surface thereof, compared to a noncancerous cell of the same tissue type. Such overexpression may be caused by gene amplification or by increased transcription or translation. ErbB receptor overexpression may be determined in a diagnostic or prognostic assay by evaluating increased levels of the ErbB protein present on the surface of a cell (e.g., via an immunohistochemistry assay; IHC). Alternatively, or additionally, one may measure levels of ErbB-encoding nucleic acid in the cell, e.g., via fluorescent in situ hybridization (FISH; see WO 98/45479), southern blotting, or polymerase chain reaction (PCR) techniques, such as real time quantitative PCR (RT-PCR). Overexpression of the ErbB ligand, may be determined diagnostically by evaluating levels of the ligand (or nucleic acid encoding it) in the patient, e.g., in a tumor biopsy or by various diagnostic assays such as the IHC, FISH, southern blotting, PCR or in vivo assays described above. One may also study ErbB receptor overexpression by measuring shed antigen (e.g., ErbB extracellular domain) in a biological fluid such as serum (see, e.g., U.S. Pat. No. 4,933,294; WO 91/05264; U.S. Pat. No. 5,401,638; and Sias et al., (1990) J. Immunol. Methods, 132: 73-80). Aside from the above assays, various other in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody.

The tumors overexpressing ErbB2 (HER2) are rated by immunohistochemical scores corresponding to the number of copies of HER2 molecules expressed per cell, and can be determined biochemically: 0=0−10,000 copies/cell, 1+=at least about 200,000 copies/cell, 2+=at least about 500,000 copies/cell, 3+=about 1-2×10⁶ copies/cell. Overexpression of HER2 at the 3+level, which leads to ligand-independent activation of the tyrosine kinase (Hudziak et al., (1987) Proc. Natl. Acad. Sci. USA, 84:7159-7163), occurs in approximately 30% of breast cancers, and in these patients, relapse-free survival and overall survival are diminished (Slamon et al., (1989) Science, 244:707-712; Slamon et al., (1987) Science, 235:177-182).

Conversely, a cancer which is “not characterized by overexpression of the ErbB2 receptor” is one which, in a diagnostic assay, does not express higher than normal levels of ErbB2 receptor compared to a noncancerous cell of the same tissue type.

The term “cytotoxic agent” as used herein refers to a substance that inhibits or prevents the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of Lu), chemotherapeutic agents, and toxins such as small molecule toxins or enzymatically active toxins of bacterial, fungal, plant or animal origin, including synthetic analogs and derivatives thereof.

A “chemotherapeutic agent” and “anticancer agent” are terms that denote a chemical compound useful in the treatment of cancer, and which may be administered in combination therapy with the antibody drug conjugate compounds of the invention. Examples of chemotherapeutic agents include Erlotinib (TARCEVA®, Genentech/OSI Pharm.), Bortezomib (VELCADE®, Millenium Pharm.), Fulvestrant (FASLODEX®, Astrazeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, GlaxoSmithKline), Lonafarnib (SCH 66336), Sorafenib (BAY43-9006, Bayer Labs.), and Gefitinib (IRESSA®, Astrazeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX®, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

As used herein, the term “EGFR-targeted drug” refers to a therapeutic agent that binds to EGFR and, optionally, inhibits EGFR activation. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX®) and reshaped human 225 (H225) (see, WO 96/40210, Imclone Systems Inc.); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO 98/50433). The anti-EGFR antibody may be conjugated with a cytotoxic agent, thus generating an immunoconjugate (see, e.g., EP 659,439A2, Merck Patent GmbH). Examples of small molecules that bind to EGFR include ZD1839 or Gefitinib (IRESSA™; Astra Zeneca), Erlotinib HCl (CP-358774, TARCEVA™; Genentech/OSI) and AG1478, AG1571 (SU 5271; Sugen).

A “tyrosine kinase inhibitor” is a molecule which inhibits to some extent tyrosine kinase activity of a tyrosine kinase such as an ErbB receptor. Examples of such inhibitors include the EGFR-targeted drugs noted in the preceding paragraph as well as quinazolines such as PD 153035, 4-(3-chloroanilino) quinazoline, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines, curcumin (diferuloyl methane, 4,5-bis (4-fluoroanilino)phthalimide), tyrphostines containing nitrothiophene moieties; PD-0183805 (Warner-Lambert); antisense molecules (e.g., those that bind to ErbB-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-ErbB inhibitors such as CI-1033 (Pfizer); Affinitac (ISIS 3521; Isis/Lilly); Imatinib mesylate (Gleevec; Novartis); PKI 166 (Novartis); GW2016 (Glaxo SmithKline); CI-1033 (Pfizer); EKB-569 (Wyeth); Semaxanib (Sugen); ZD6474 (AstraZeneca); PTK-787 (Novartis/Schering AG); INC-1C11 (Imclone); or as described in any of the following patent publications: WO 99/09016 (American Cyanamid); WO 98/43960 (American Cyanamid); WO 97/38983 (Warner Lambert); WO 99/06378 (Warner Lambert); WO 99/06396 (Warner Lambert); WO 96/30347 (Pfizer, Inc); WO 96/33978 (Zeneca); WO 96/3397 (Zeneca); and WO 96/33980 (Zeneca).

An “anti-angiogenic agent” refers to a compound which blocks, or interferes with to some degree, the development of blood vessels. The anti-angiogenic factor may, for instance, be a small molecule or antibody that binds to a growth factor or growth factor receptor involved in promoting angiogenesis. An exemplary anti-angiogenic factor herein is an antibody that binds to Vascular Endothelial Growth Factor (VEGF).

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

A “liposome” is a small vesicle composed of various types of lipids, phospholipids and/or surfactant which is useful for delivery of a drug (such as the anti-ErbB2 antibodies disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

“Phage display” is a technique by which variant polypeptides are displayed as fusion proteins to a coat protein on the surface of phage, e.g., filamentous phage, particles. One utility of phage display lies in the fact that large libraries of randomized protein variants can be rapidly and efficiently sorted for those sequences that bind to a target molecule with high affinity. Display of peptide and protein libraries on phage has been used for screening millions of polypeptides for ones with specific binding properties. Polyvalent phage display methods have been used for displaying small random peptides and small proteins, typically through fusions to either PIII or PVIII of filamentous phage. Wells and Lowman, Curr. Opin. Struct. Biol., 3:355-362 (1992), and references cited therein. In monovalent phage display, a protein or peptide library is fused to a phage coat protein or a portion thereof, and expressed at low levels in the presence of wild type protein. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 3:205-0216 (1991). Phage display includes techniques for producing antibody-like molecules (Janeway, C., Travers, P., Walport, M., Shlomchik (2001) Immunobiology, 5th Ed., Garland Publishing, New York, p627-628).

A “phagemid” is a plasmid vector having a bacterial origin of replication, e.g., Co1E1, and a copy of an intergenic region of a bacteriophage. The phagemid may be used on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle.

“Alkyl” is a C₁-C₁₈ hydrocarbon moiety containing normal, secondary, tertiary or cyclic carbon atoms. Examples of alkyl radicals include C₁-C₈ hydrocarbon moieties such as: methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, cyclopropyl, cyclobutyl, cyclopentyl, cyclohexyl, cycloheptyl, and cyclooctyl.

“Alkenyl” is a C₂-C₁₈ hydrocarbon moiety containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), 1-cyclopent-1-enyl, 1-cyclopent-2-enyl, 1-cyclopent-3-enyl, 5-hexenyl (—CH₂ CH₂CH₂CH₂CH═CH₂), 1-cyclohex-1-enyl, 1-cyclohex-2-enyl, and 1-cyclohex-3-enyl.

“Alkynyl” is a C₂-C₁₈ hydrocarbon moiety containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH),

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl”, “substituted aryl”, and “substituted arylalkyl” mean alkyl, aryl, and arylalkyl respectively, in which one or more hydrogen atoms are each independently replaced with a substituent. Typical substituents include, but are not limited to, —X, —R, —O′, —OR, —SR, —S′, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃—, —SO₃H, —S(═O)₂R, —OS(═O)₂OR, —S(═O)₂NR, —S(═O)R, —OP(═O)(OR)₂, —P(═O)(OR)₂, —PO′₃, —PO₃H₂, —C(═O)R, —C(═O)X, —C(═S)R, —CO₂R, —CO₂′, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently H, C₁-C₁₈ alkyl, C₆-C₂₀ aryl, C₃-C₁₄ heterocycle, or protecting group. Alkylene, alkenylene, and alkynylene groups as described above may also be similarly substituted.

“Heteroaryl”, “heterocyclyl”, and “heterocycle” all refer to a ring system in which one or more ring atoms is a heteroatom, e.g. nitrogen, oxygen, and sulfur. The heterocycle radical comprises 1 to 20 carbon atoms and 1 to 5 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H, 6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4Ah-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” and “carbocyclyl” mean a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g. arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex- 1-enyl, 1-cyclohex-2-enyl, 1-cyclohex-3-enyl, cycloheptyl, and cyclooctyl.

“Linker”, “Linker Unit”, “Linker reagent” or “link” means a chemical moiety comprising a covalent bond or a chain of atoms that covalently attaches an antibody to a drug moiety. In various embodiments, a linker is specified as L. Linkers include, but are not limited to, a divalent radical such as an alkyldiyl, an aryldiyl, a heteroaryldiyl, moieties such as: —(CR₂)ₙO(CR₂)ₙ—, repeating units of alkyloxy (e.g. polyethylenoxy, PEG, polymethyleneoxy) and alkylamino (e.g. polyethyleneamino, Jeffamine™); and diacid ester and amides including maleimide, succinate, succinamide, diglycolate, malonate, and caproamide.

The term “label” means any moiety which can be covalently attached to an antibody and that functions to: (i) provide a detectable signal; (ii) interact with a second label to modify the detectable signal provided by the first or second label, e.g. FRET (fluorescence resonance energy transfer); (iii) stabilize interactions or increase affinity of binding, with antigen or ligand; (iv) affect mobility, e.g. electrophoretic mobility, or cell-permeability, by charge, hydrophobicity, shape, or other physical parameters, or (v) provide a capture moiety, to modulate ligand affinity, antibody/antigen binding, or ionic complexation.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The phrase “pharmaceutically acceptable salt,” as used herein, refers to pharmaceutically acceptable organic or inorganic salts of an ADC. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis -(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

“Pharmaceutically acceptable solvate” refers to an association of one or more solvent molecules and an ADC. Examples of solvents that form pharmaceutically acceptable solvates include, but are not limited to, water, isopropanol, ethanol, methanol, DMSO, ethyl acetate, acetic acid, and ethanolamine.

The following acronyms, terms, and abbreviations are used herein and have the indicated definitions:

Boc is N-(t-butoxycarbonyl), cit is citrulline (2-amino-5-ureido pentanoic acid), dap is dolaproine, DCC is 1,3-dicyclohexylcarbodiimide, DCM is dichloromethane, DEA is diethylamine, DEAD is diethylazodicarboxylate, DEPC is diethylphosphorylcyanidate, DIAD is diisopropylazodicarboxylate, DIEA is N,N-diisopropylethylamine, dil is dolaisoleuine, DMAP is 4-dimethylaminopyridine, DME is ethyleneglycol dimethyl ether (or 1,2-dimethoxyethane), DMF is N,N-dimethylformamide, DMSO is dimethylsulfoxide, doe is dolaphenine, dov is N,N-dimethylvaline, DTNB is 5,5′-dithiobis(2-nitrobenzoic acid), DTPA is diethylenetriaminepentaacetic acid, DTT is dithiothreitol, EDCI is 1-(3-dimethylaminopropyl)-3-ethylcarbodiimide hydrochloride, EEDQ is 2-ethoxy-1-ethoxycarbonyl-1,2-dihydroquinoline, ES-MS is electrospray mass spectrometry, EtOAc is ethyl acetate, Fmoc is N-(9-fluorenylmethoxycarbonyl), gly is glycine, HATU is O-(7-azabenzotriazol-1-yl)-N,N,N′,N′-tetramethyluronium hexafluorophosphate, HOBt is 1-hydroxybenzotriazole, HPLC is high pressure liquid chromatography, ile is isoleucine, lys is lysine, MeCN (CH₃CN) is acetonitrile, LC/MS is liquid chromatography and mass spectrometry, MeOH is methanol, Mtr is 4-anisyldiphenylmethyl (or 4-methoxytrityl), nor is (1S, 2R)-(+)-norephedrine, PBS is phosphate-buffered saline (Ph 7.4), PEG is polyethylene glycol, Ph is phenyl, Pnp is p-nitrophenyl, PyBrop is bromo tris-pyrrolidino phosphonium hexafluorophosphate, SEC is size-exclusion chromatography, Su is succinimide, TFA is trifluoroacetic acid, TLC is thin layer chromatography, UV is ultraviolet, and val is valine.

Antibodies: HERCEPTIN® (trastuzumab)=full length, humanized antiHER2 (MW 145167), trastuzumab F(ab′)2=derived from antiHER2 enzymatically (MW 100000), 4D5=full-length, murine antiHER2, from hybridoma, rhu4D5=transiently expressed, full-length humanized antibody, rhuFab4D5=recombinant humanized Fab (MW 47738), 4D5Fc8=full-length, murine antiHER2, with mutated FcRn binding domain

Linkers: MC=6-maleimidocaproyl, MP=maleimidopropanoyl, val-cit=valine-citrulline, dipeptide site in protease-cleavable linker, ala-phe=alanine-phenylalanine, dipeptide site in protease-cleavable linker, PAB=p-aminobenzyloxycarbonyl (“self immolative” portion of linker), SPP=N-Succinimidyl 4-(2-pyridylthio) pentanoate, SMCC=N-Succinimidyl 4-(N-maleimidomethyl) cyclohexane-1 carboxylate, SIAB=N-Succinimidyl (4-iodo-acetyl) aminobenzoate

Antibody Drug Conjugates

The compounds of the invention include those with potential utility for anticancer activity, treatment of hyperproliferative disorders, autoimmune disorders, and infectious disease. In particular, the compounds include an antibody conjugated, i.e. covalently attached by a linker, to a 1,8 bis-naphthalimide drug moiety where the corresponding drug when not conjugated to an antibody has a cytotoxic or cytostatic effect. The biological activity of the drug is thus modulated by conjugation to an antibody. The antibody drug conjugates (ADC) of the invention may selectively deliver an effective dose of a cytotoxic agent to tumor tissue whereby greater selectivity, i.e. a lower efficacious dose may be achieved, than upon delivery of the same dose of the 1,8 bis-naphthalimide compound not conjugated to an antibody.

In one embodiment, the bioavailability of the ADC of the invention, or an intracellular metabolite of the ADC, is improved in a mammal when compared to a 1,8 bis-naphthalimide compound comprising the 1,8 bis-naphthalimide moiety of the ADC. Also, the bioavailability of the ADC, or an intracellular metabolite of the ADC is improved in a mammal when compared to the analog of the ADC not having the 1,8 bis-naphthalimide drug moiety.

In one embodiment, the drug moiety of the ADC is not cleaved from the antibody until the antibody-drug conjugate enters a cell with a cell-surface receptor specific for the antibody of the antibody-drug conjugate, and the drug moiety is cleaved from the antibody when the antibody-drug conjugate does enter the cell. The 1,8 bis-naphthalimide drug moiety may be intracellularly cleaved in a mammal from the antibody of the compound, or an intracellular metabolite of the compound, by enzymatic action, hydrolysis, oxidation, or other mechanism.

An antibody-drug conjugate compound comprises an antibody covalently attached by a linker to one or more 1,8 bis-naphthalimide drug moieties, the compound having Formula I
Ab-(L-D)ₚ I
or a pharmaceutically acceptable salt or solvate thereof, wherein

Ab is an antibody;

L is a linker covalently attached to an Ab, and L is covalently attached to D;

D is a 1,8 bis-naphthalimide drug moiety selected from Formulas IIa and IIb:

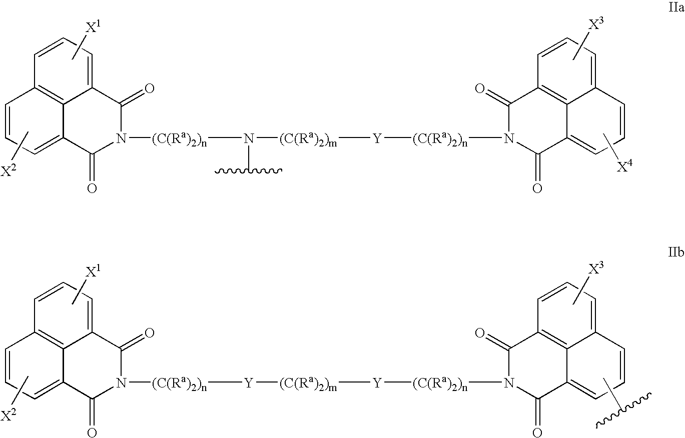

the wavy line indicates the covalent attachment to L,

Y is N(R^{b}), C(R^{a})₂, O, or S;

R^{a} is independently selected from H, F, Cl, Br, I, OH, —N(R^{b})₂, —N(R^{b})₃⁺, C₁-C₈ alkylhalide, carboxylate, sulfate, sulfamate, sulfonate, —SO₂R^{b}, —S(═O)R^{b}, —SR^{b}, —SO₂N(R^{b})₂, —C(═O)R^{b}, —CO₂R^{b}, —C(═O)N(R^{b})₂, —CN, —N₃, —NO₂, C₁-C₈ alkoxy, C₁-C₈ trifluoroalkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, two R^{a} groups on the same carbon atom form a carbonyl (═O), or on different carbon atoms form a carbocyclic, heterocyclic, or aryl ring of 3 to 7 carbon atoms;

R^{b} is independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle;

where C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from F, Cl, Br, I, OH, —N(R^{b})₂, —N(R^{b})₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R^{b}, —S(═O)R^{b}, —SR^{b}, —SO₂N(R^{b})₂, —C(═O)R^{b}, —CO₂R^{b}, —C(═O)N(R^{b})₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate;

m is 1, 2, 3, 4, 5, or 6;

n is independently selected from 1, 2, and 3;

X¹, X², X³, and X⁴ are independently selected from F, Cl, Br, I, OH, —N(R^{b})₂, —N(R^{b})₃⁺, —N(R^{b})C(═O)R^{b}, —N(R^{b})C(═O)N(R^{b})₂, —N(R^{b})SO₂N(R^{b})₂, —N(R^{b})SO₂R^{b}, OR, OC(═O)R^{b}, OC(═O)N(R^{b})₂, C₁-C₈ alkylhalide, carboxylate, sulfate, sulfamate, sulfonate, —SO₂R^{b}, —SO₂Ar, —SOAr, —SAr, SO₂N(R^{b})₂, —SOR^{b}, —CO₂R^{b}, —C(═O)N(R^{b})₂, —CN, —N₃, —NO₂, C₁-C₈ alkoxy, C₁-C₈ trifluoroalkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or

X¹ and X² together, and X³ and X⁴ together, independently form —CH₂CH₂— or —CH₂ CH₂CH₂—;

D may independently have more than one X¹, X², X³, or X⁴; and where D has more than one X¹, X², X³, or X⁴, then two X¹, X², X³, or X⁴ may form a fused C₆-C₂₀ aryl, C₆-C₂₀ substituted aryl, C₁-C₂₀ heterocycle, or C₁-C₂₀ substituted heterocycle; and

p is an integer from 1 to 20.

The drug loading is represented by p, the average number of drugs per antibody in a molecule of Formula I. Drug loading may range from 1 to 20 drugs (D) per antibody (Ab or mAb). Compositions of ADC of Formula I include collections of antibodies conjugated with a range of drugs, from 1 to 20. The average number of drugs per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis.

For some antibody drug conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody drug conjugates.

Typically, fewer than the theoretical maximum of drug moieties are conjugated to an antibody during a conjugation reaction. An antibody may contain, for example, many lysine residues that do not react with the drug-linker intermediate or linker reagent. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds of the invention exist as disulfide bridges and must be reduced with a reducing agent such as dithiothreitol (DTT), under partial or total reducing conditions. Additionally, the antibody must be subjected to denaturing conditions to reveal reactive nucleophilic groups such as lysine or cysteine. The loading (drug/antibody ratio) of an ADC may be controlled in several different manners, including: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate, or linker reagent followed by drug moiety reagent, then the resulting product is a mixture of ADC compounds with a distribution of one or more drug moieties attached to an antibody. The average number of drugs per antibody may be calculated from the mixture by dual ELISA antibody assay, specific for antibody and specific for the drug. Individual ADC molecules may be identified in the mixture by mass spectroscopy, and separated by HPLC, e.g. hydrophobic interaction chromatography (“Effect of drug loading on the pharmacology, pharmacokinetics, and toxicity of an anti-CD30 antibody-drug conjugate”, Hamblett, K. J., et al, Abstract No. 624, American Association for Cancer Research; 2004 Annual Meeting, Mar. 27-31, 2004, Proceedings of the AACR, Volume 45, March 2004; “Controlling the Location of Drug Attachment in Antibody-Drug Conjugates”, Alley, S. C., et al, Abstract No. 627, American Association for Cancer Research; 2004 Annual Meeting, Mar. 27-31, 2004, Proceedings of the AACR, Volume 45, March 2004). Thus, a homogeneous ADC with a single loading value, may be isolated from the conjugation mixture by electrophoresis or chromatography.

1,8 Bis-Naphthalimide Drug Moieties

Drug moieties (D) are the 1,8 bis-naphthalimide type and have Formulas IIa and IIb. One embodiment of a bis 1,8 naphthalimide drug moiety is the unsubstituted bis 1,8 naphthalimide, “elinafide”, drug moiety (E) having the structure:

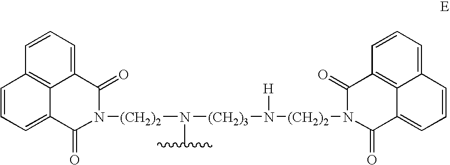

where Y is N(R^{b}), R^{b} is H, m is 3, and n is 2.

The 1,8 naphthalimide aromatic carbon atoms D moieties IIa and IIb may be independently substituted with a range of substituents (X¹-X⁴) besides H. Exemplary embodiments of IIa where the two 1,8 naphthalimide groups are the same, and where Y is N(R^{b}), n=2, m=3, R^{a} and R^{b} are H, include the exemplary structures:

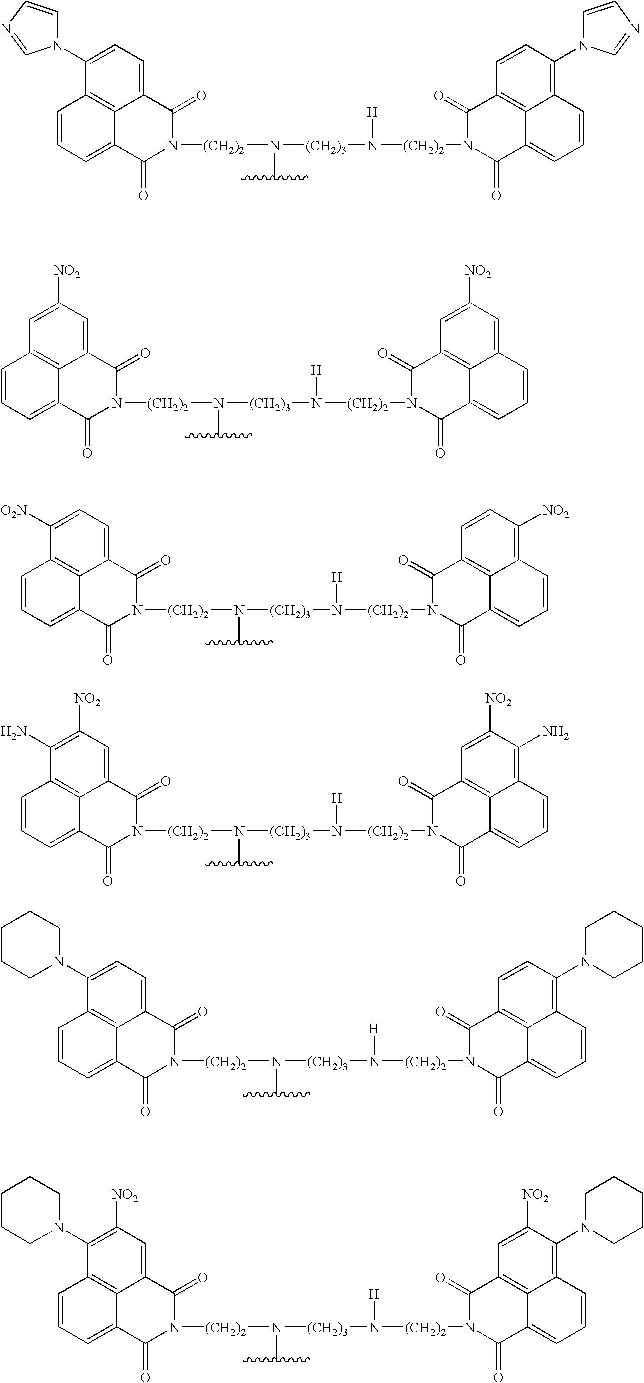

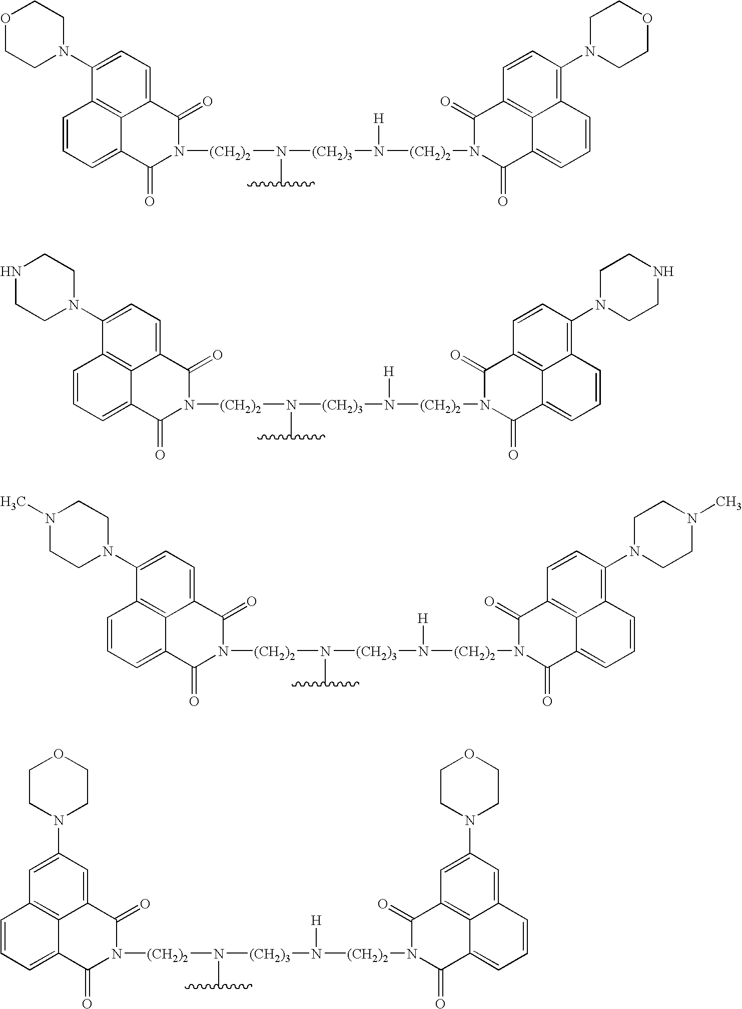

Exemplary embodiments of D moiety IIa where the two 1,8 naphthalimide groups are not the same, and where Y is N(R^{b}), n=2, m=3, R^{a} and R^{b} are H, include the structures:

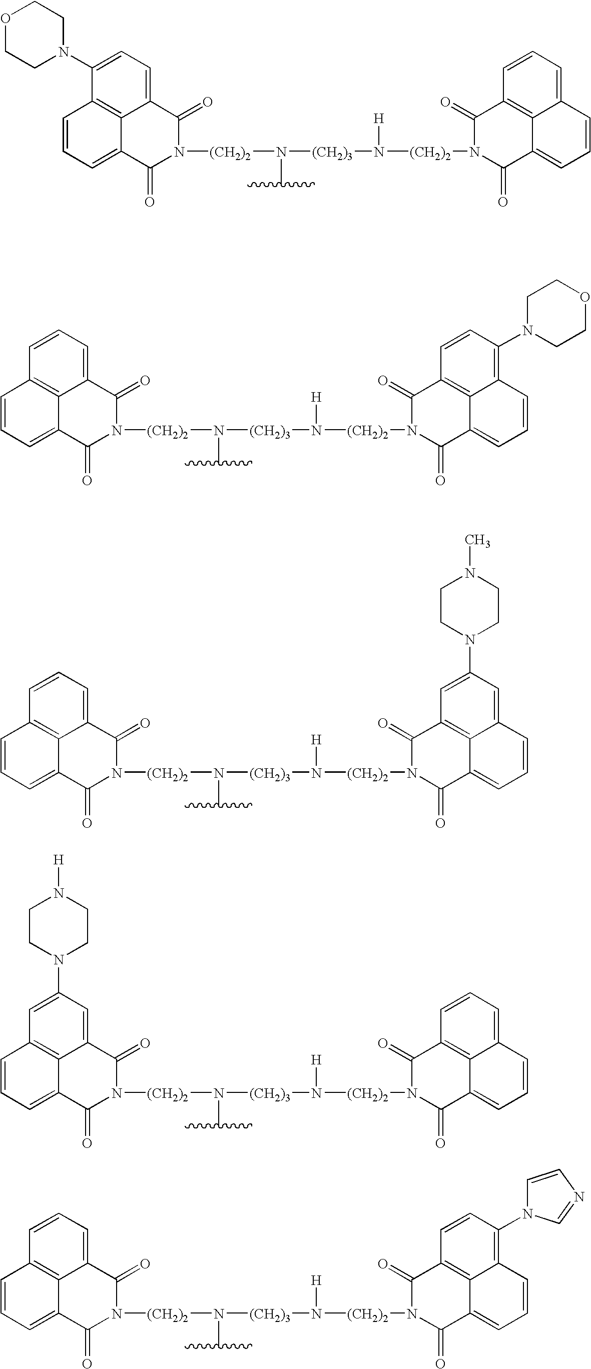

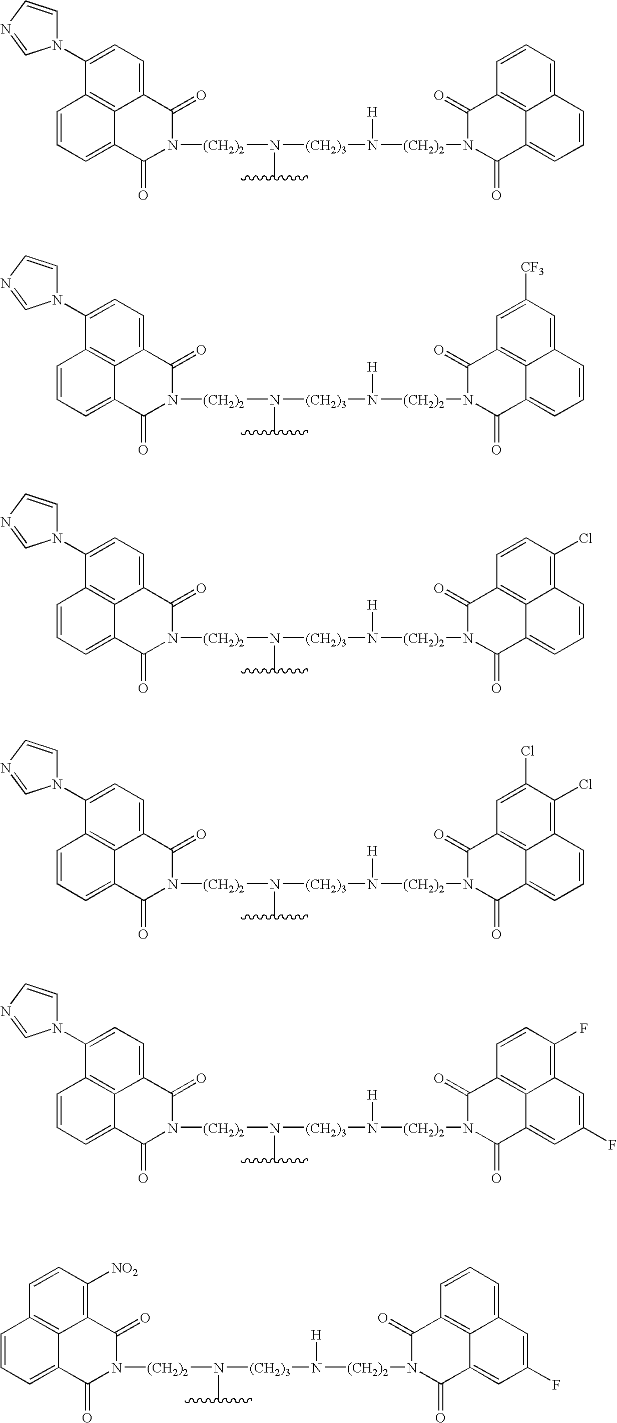

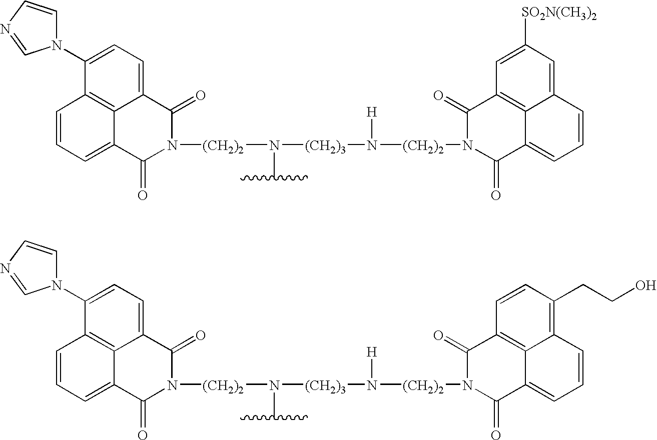

X¹ and X² together, or X³ and X⁴ together, independently may form —CR₂CH₂— or —CR₂CH₂CH₂—. Exemplary embodiments of such, and where Y is N(R^{b}), n=2, m=3, R^{a} and R^{b} are H, include the D moiety IIa structures:

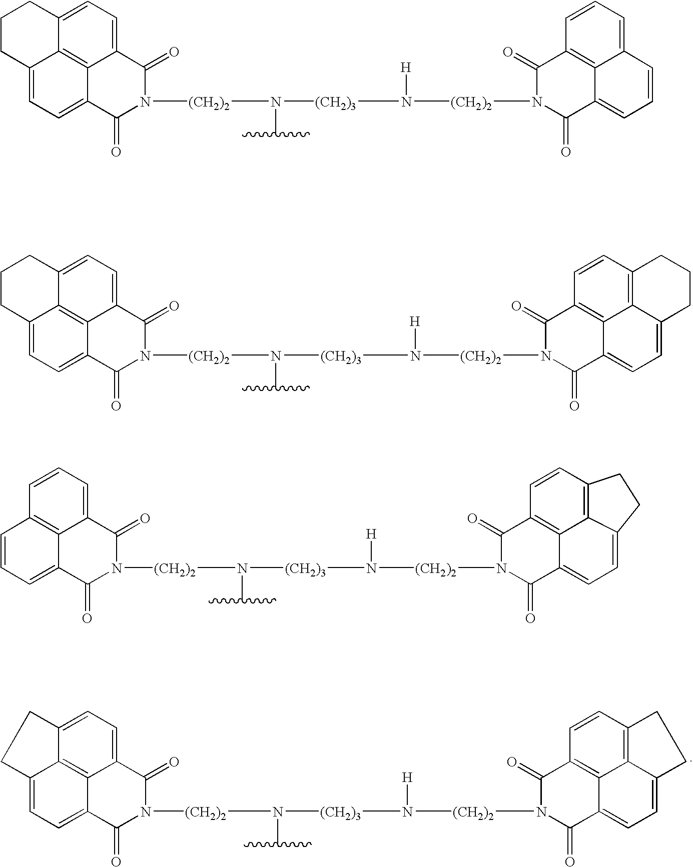

Two X¹, X², X³, or X⁴ on adjacent carbon atoms may form a fused C₆-C₂₀ aryl, C₆-C₂₀ substituted aryl, C₁-C₂₀ heterocycle, or C₁-C₂₀ substituted heterocycle. Exemplary embodiments of such, and where Y is N(R^{b}), n=2, m=3, R^{a} and R^{b} are H, include the D moiety IIa structures:

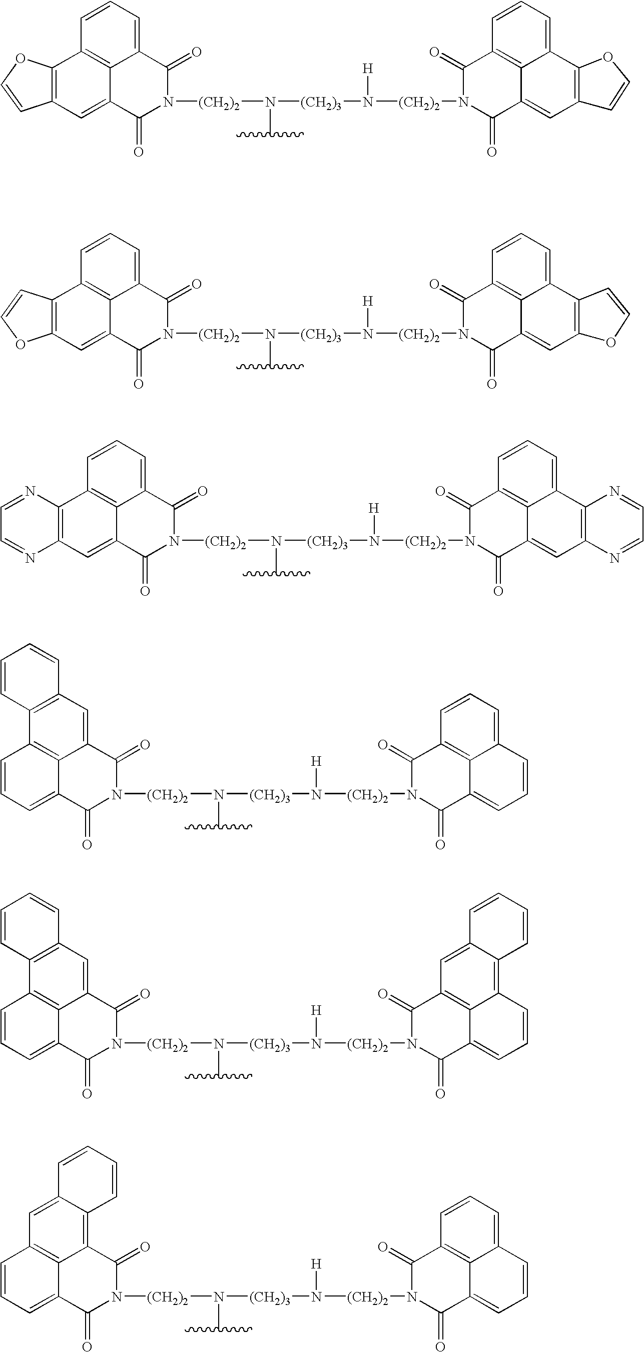

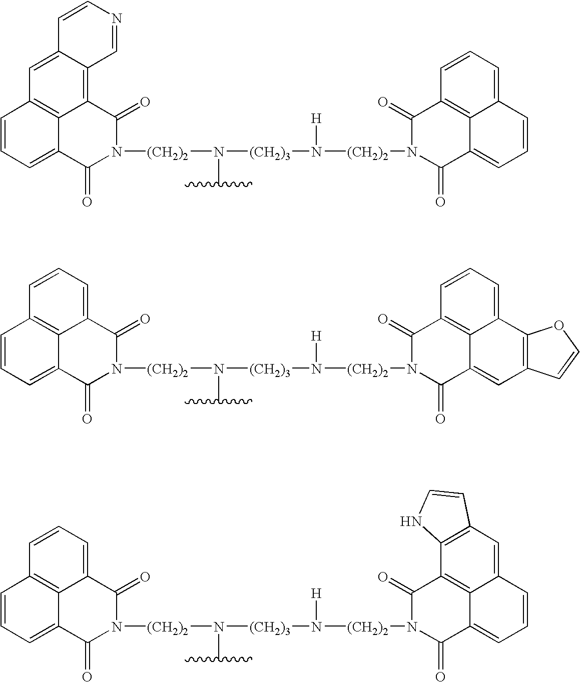

The bis-amino alkyl group that attaches the two 1,8 naphthalimide groups may bear a range of substituents besides H on the carbon atoms (R^{a}) and the nitrogen atom not linked to L (R^{b}). Exemplary embodiments of D where Y is N(R^{b}), m is 3 and n is 2 in the bis-amino alkyl group include the D moiety IIa structures:

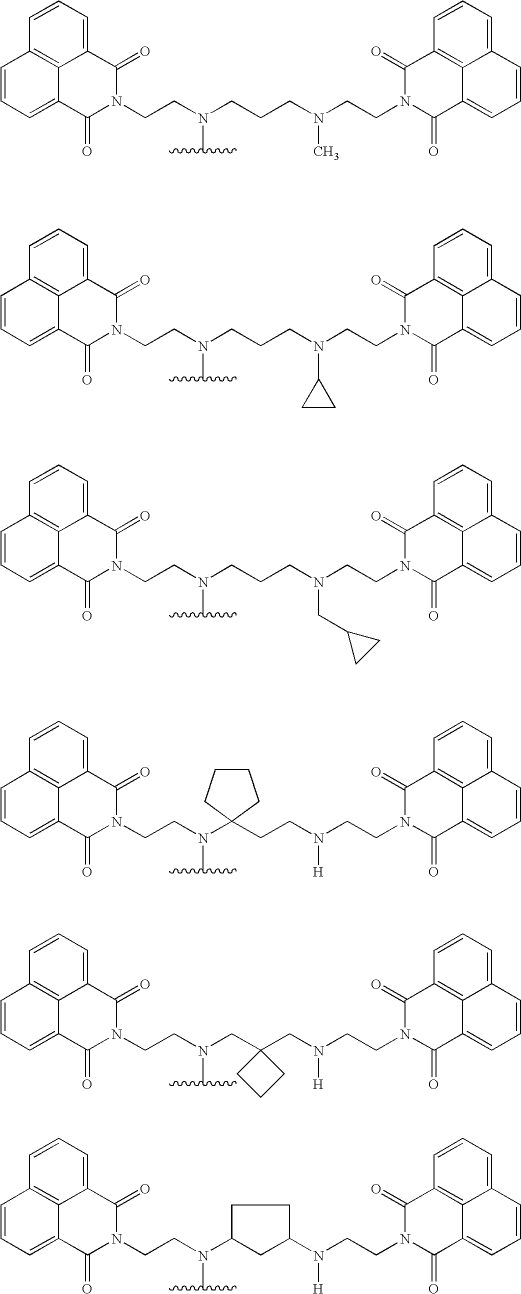

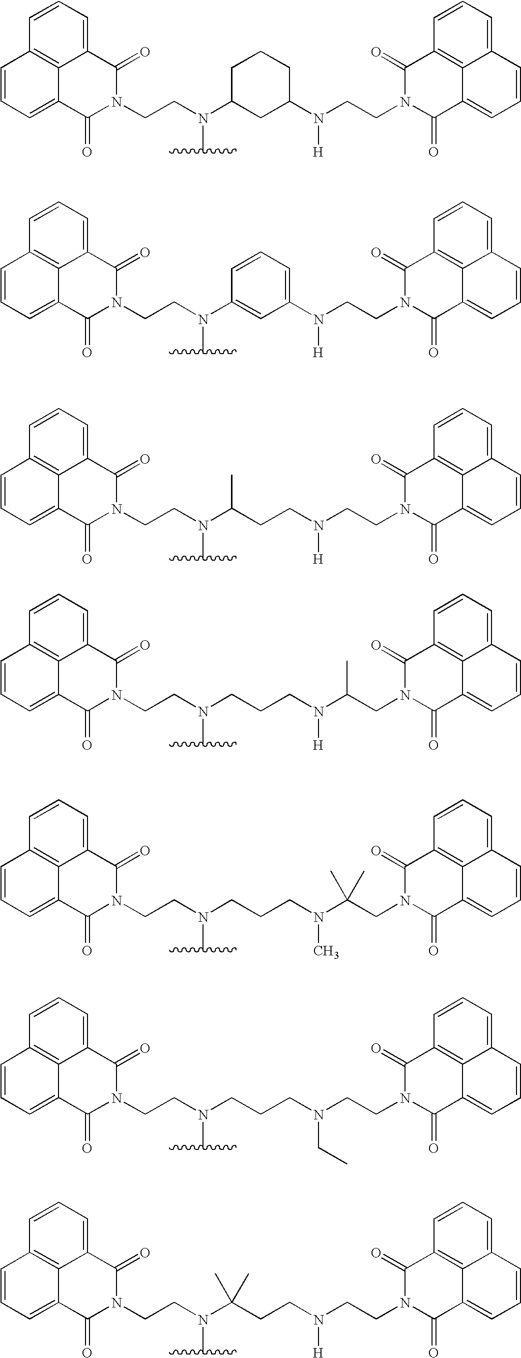

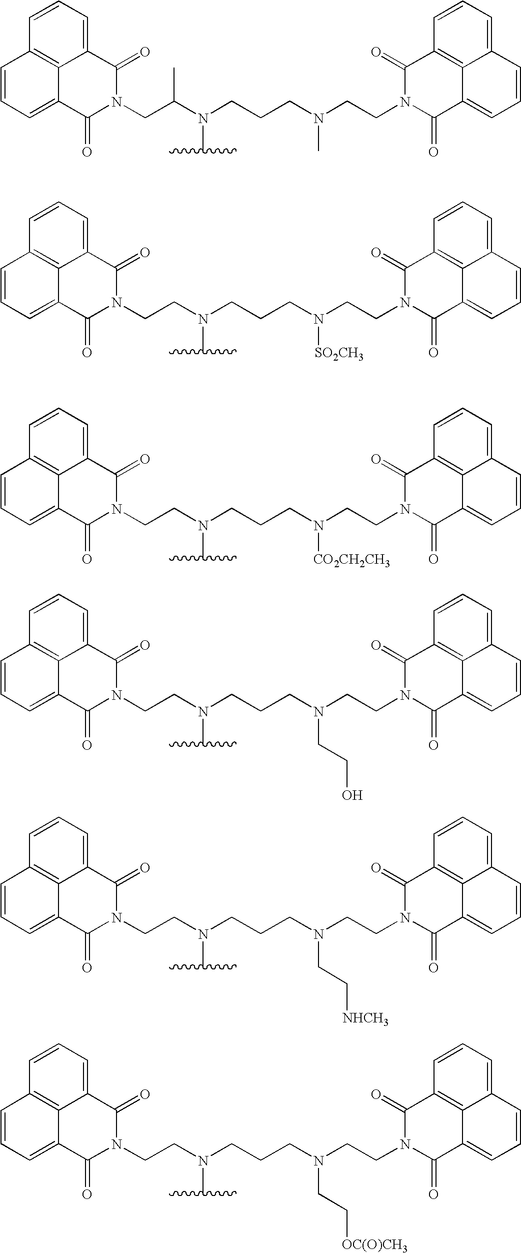

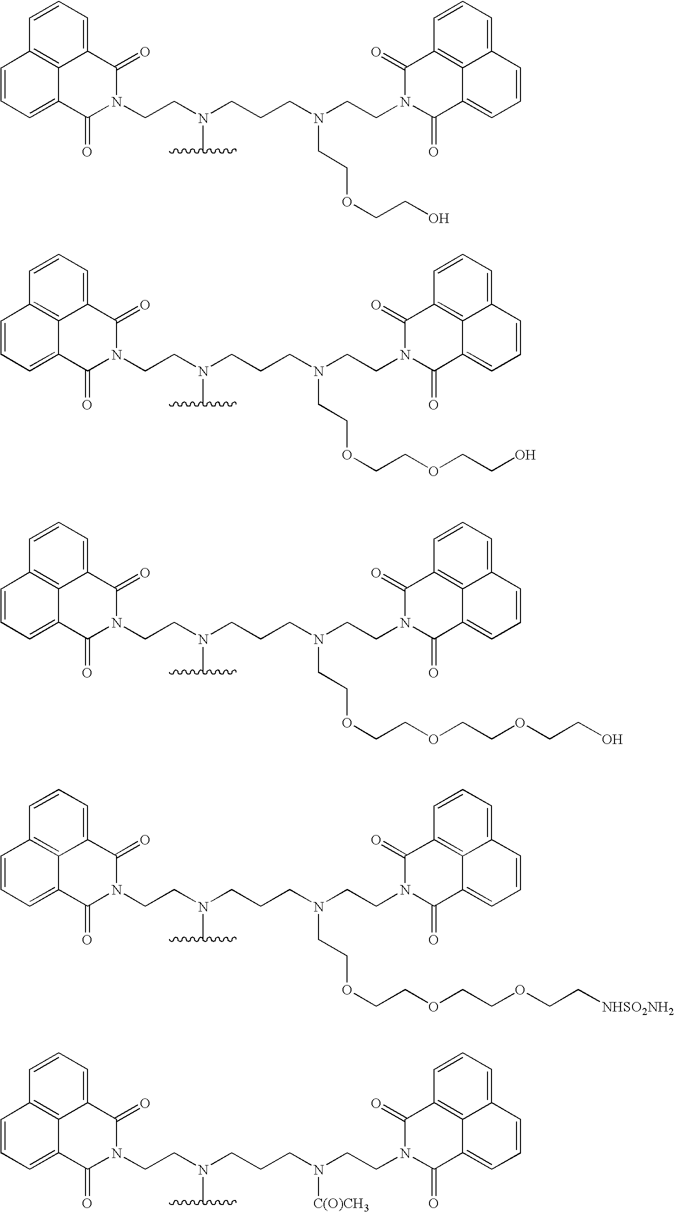

The three alkylene groups of the bis-amino alkyl group that attaches the two 1,8 naphthalimide groups may independently be of different lengths and bear a range of substituents besides H on the carbon atoms (R^{a}) and the nitrogen atom (Y═NR^{b}) not linked to L (R^{b}). The two non-equivalent alkylene groups between each 1,8 naphthalimide group and a nitrogen atom (n) are independently 1, 2, or 3 carbons in length. The alkylene group between the nitrogen atoms (m) is 1, 2, 3, 4, 5, or 6 carbons in length. The compounds of the invention thus include all 54 possible combinations of lengths of the three alkylene groups in a drug moiety (D) IIa and IIb (Y═NR^{b}). A numerical matrix designating the n and m values of the alkylene groups of the bis-amino alkyl group wherein: the length (n) of the alkylene group including the nitrogen atom bonded to the linker (N to L) is first; the length (m) of the alkylene group between the nitrogen atoms is second; and the length (n) of the alkylene group bonded to the nitrogen atom not linked to L (N not to L) is third (left to right) exemplifies the combinations in Table 1:

TABLE 1
n (1-3, N to L) · m (1-6) · n (1-3, N not to L)

| | | | | | |
|---|---|---|---|---|---|
| 1.1.1 | 1.1.2 | 1.1.3 | 1.2.1 | 1.2.2 | 1.2.3 |
| 1.3.1 | 1.3.2 | 1.3.3 | 1.4.1 | 1.4.2 | 1.4.3 |
| 1.5.1 | 1.5.2 | 1.5.3 | 1.6.1 | 1.6.2 | 1.6.3 |
| 2.1.1 | 2.1.2 | 2.1.3 | 2.2.1 | 2.2.2 | 2.2.3 |
| 2.3.1 | 2.3.2 | 2.3.3 | 2.4.1 | 2.4.2 | 2.4.3 |
| 2.5.1 | 2.5.2 | 2.5.3 | 2.6.1 | 2.6.2 | 2.6.3 |
| 3.1.1 | 3.1.2 | 3.1.3 | 3.2.1 | 3.2.2 | 3.2.3 |
| 3.3.1 | 3.3.2 | 3.3.3 | 3.4.1 | 3.4.2 | 3.4.3 |
| 3.5.1 | 3.5.2 | 3.5.3 | 3.6.1 | 3.6.2 | 3.6.3 |

The same combinatorial set of embodiments for drug moiety Iub where the linker (L) is covalently attached through an aryl carbon atom of a 1,8 naphthalimide group, are included in the compounds of the invention.

Exemplary embodiments of the bis-amino alkyl group where Y is N, and R^{a} and R^{b} are H include the drug moiety IIa structures:

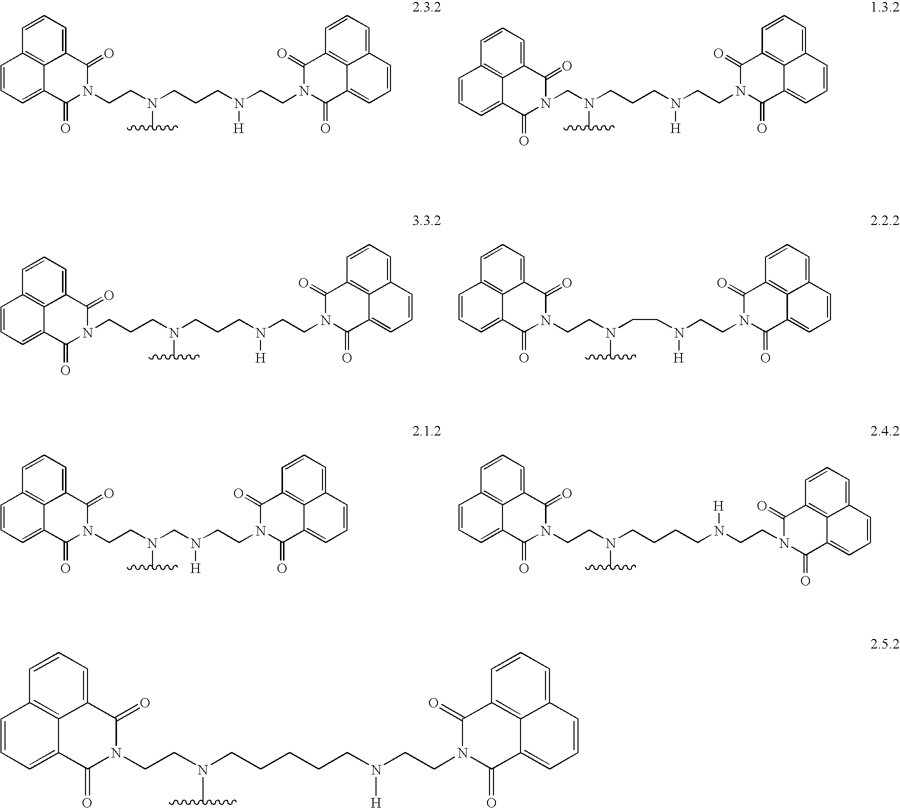

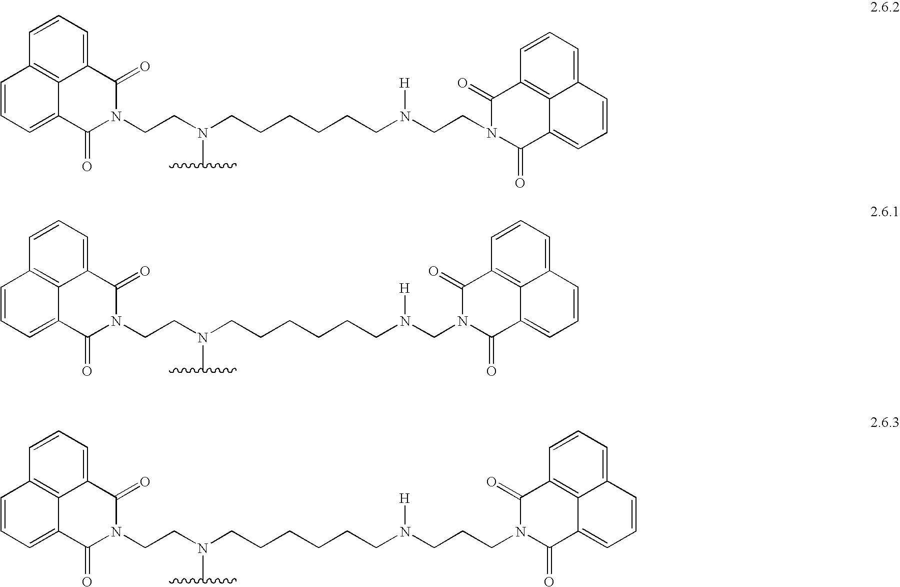

Exemplary embodiments of IIb where the two 1,8 naphthalimide groups are the same (X¹, X², X³, X⁴=H), n=2, m=3, Y is N(R^{b}), and R^{a} and R^{b} are H, include the exemplary structures:

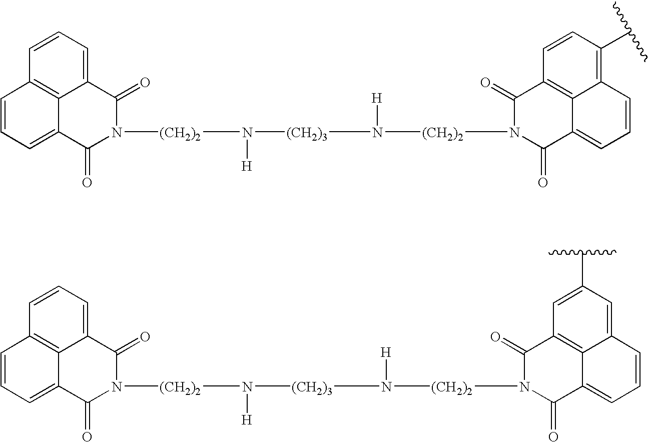

Exemplary embodiments of IIb where the linker (L) is attached through one of the 1,8 naphthalimide groups, the two 1,8 naphthalimide groups are different, n=2, m=3, and R^{a} are H, include the exemplary structures:

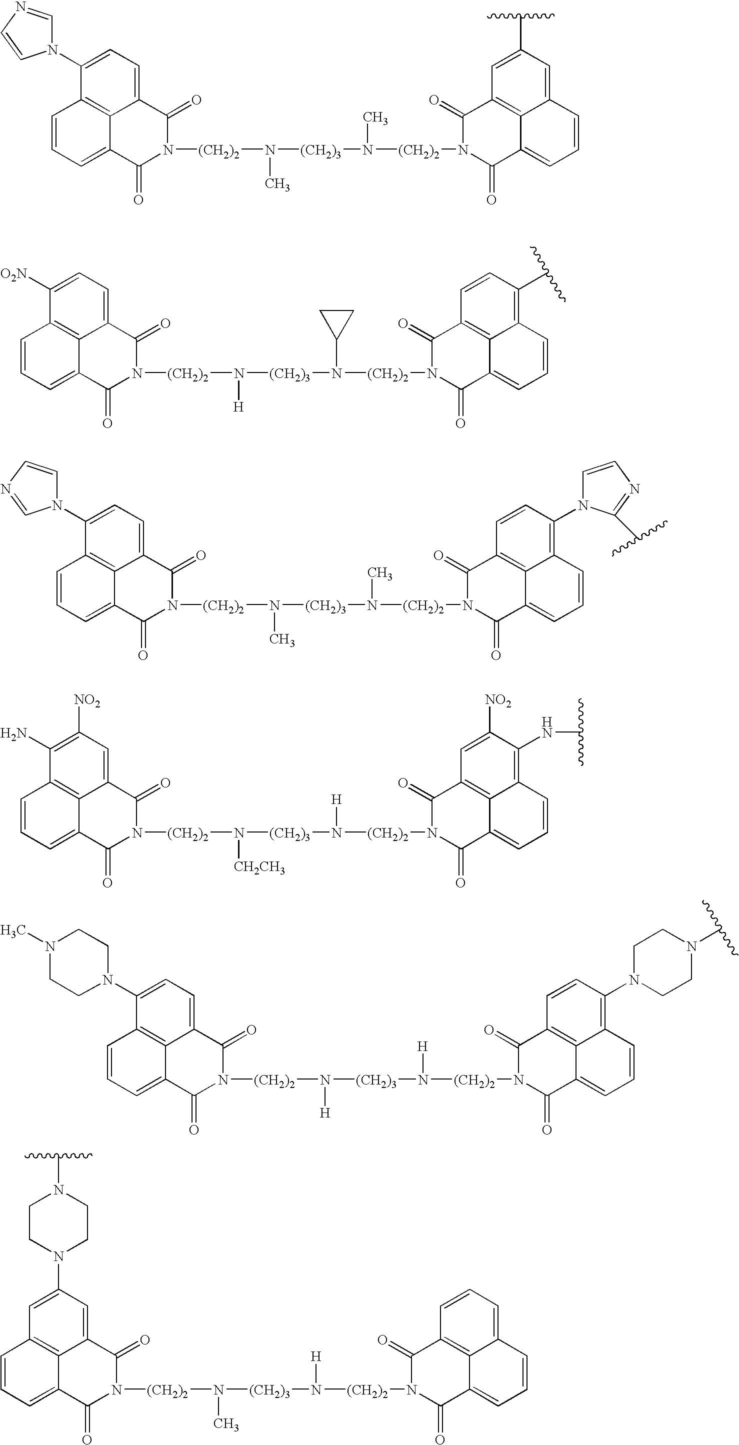

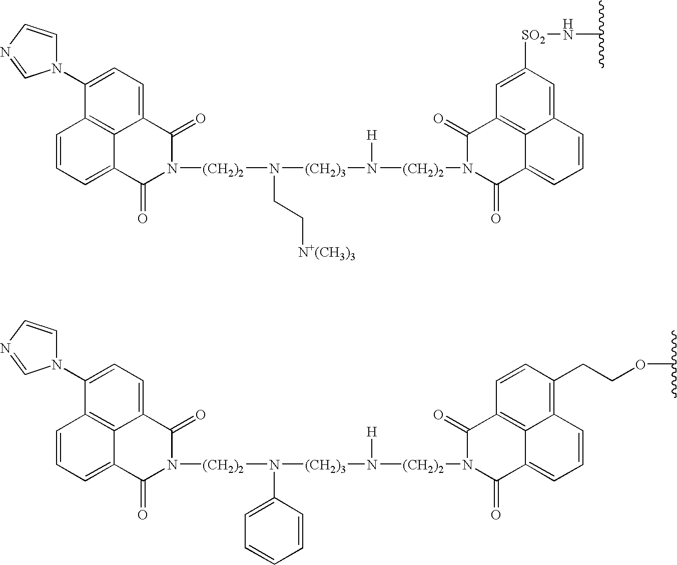

Exemplary embodiments of IIa and IIb where Y is O or S include the following structures: 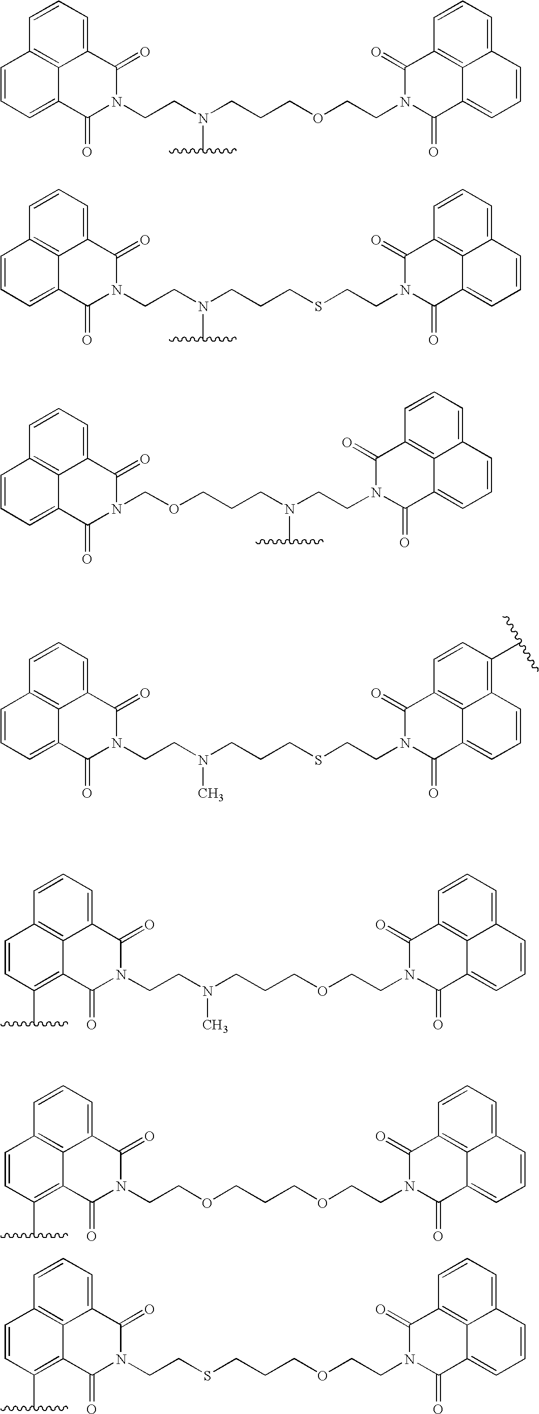
Synthesis of Bis 1,8 Naphthalimide Drug Moieties

Bis 1,8 naphthalimide drug moieties were prepared according to Brana et al (2004) J. Med. Chem. 47:1391-1399; Brana et al (2003) Org. Biomol. Chem. 1:648-654; Brana, M. F. and Ramos, A. (2001) Current Med. Chem.—Anti-Cancer Agents 1:237-255, as well as conventional organic chemistry methodology.

Generally, 1,8 naphthalimide intermediates may be prepared from 1,8-naphthalic anhydride compounds (Chem. Rev. (1970) 70:439-469; U.S. Pat. Nos. 4,146,720; 5,616,589; 5,416,089; 5,585,382; 5,552,544). Various substituted 1,8-naphthalic anhydride compounds are commercially available, such as 4-Bromo-1,8-naphthalic anhydride (Aldrich, Milwaukee, Wis.). Reaction of a 1,8-naphthalic anhydride compound with a primary amine gives the 1,8 naphthalimide. Displacement of bromine from the 4 position occurs with various nucleophilic reagents.

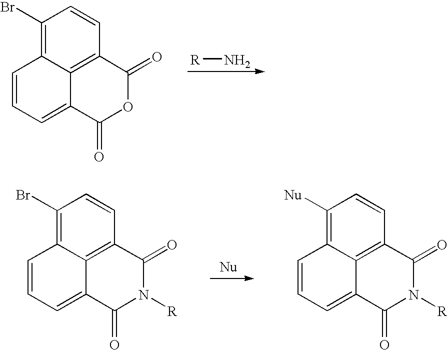

Where the amine reagent is a bis-amino compound, two 1,8-naphthalic anhydride react to form bis 1,8 naphthalimide intermediates (Brana, M. F. and Ramos, A. (2001) Current Med. Chem.—Anti-Cancer Agents 1:237-255; Brana et al (1993) Anticancer Drug Des. 8:257; Brana et al (1996) Anticancer Drug Des. 11:297; WO 94/02466; and U.S. Pat. Nos. 4,874,863; 5,206,249; 5,416,089; 5,488,110; 5,981,753; 6,177,570). For example, two equivalents of an anhydride in toluene are treated with one equivalent of the corresponding polyamine in ethanol. The mixture is heated at reflux until the reaction is complete. The bis 1,8 naphthalimide is isolated, e.g. by filtration and crystallization, as the free base and converted to a salt, such as the mesylate with methanesulfonic acid, or as the trifluoroacetate with trifluoroacetic acid (TFA), and washed with an organic solvent, according to the method of Brana et al (2004) J. Med. Chem. 47:1391-1399.

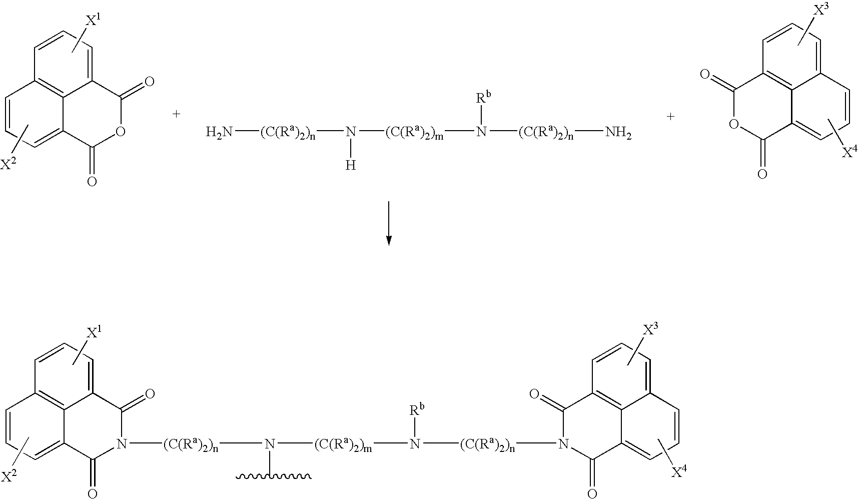

Alternatively, the 1,8 naphthalimide groups may be attached to the polyamine unit sequentially (WO 94/02466) by protecting one of the terminal amino groups of the polyamine reagent during reaction with the first 1,8 naphthalic anhydride reagent. After deprotection of the terminal amino group of the mono 1,8 naphthalimide intermediate, a second 1,8 naphthalic anhydride reagent may be reacted to form the bis 1,8 naphthalimide product. By this route, asymmetric bis 1,8 naphthalimide compounds can be prepared, i.e. where X¹ and X² are different than X³ and X⁴. Suitable amino protecting groups include mesitylenesulfonyl, dinitrobenzenesulfonyl, BOC (tert-butyloxycarbonyl), CBz (carbobenzoxy), or those detailed in Protective Groups in Organic Chemistry, Theodora W. Greene (1991) John Wiley & Sons, Inc., New York, or later editions thereto. Alternatively, the terminal amino group for coupling to the second 1,8 naphthalic anhydride reagent may be generated by reductive amination of a carbonyl group such as aldehyde or ester, or by reduction of a nitrile group.

Linker

The linker (L) is a bifunctional or multifunctional moiety which is covalently attached to one or more Drug moieties (D) and an antibody unit (Ab) to form Antibody Drug Conjugates (ADC) of the invention.

In one embodiment, the linker L of an ADC has the formula:
-Aₐ-W_{w}-SP_{y}-

wherein:

-A- is a Stretcher unit;

a is 0 or 1;

each -W- is independently an Amino Acid unit;

w is independently an integer ranging from 0 to 12;

-SP- is a Spacer unit; and

y is 0, 1 or 2.

In this embodiment, the ADC may be represented by Formula Ia:
Ab [symbol] Aₐ-W_{w}-SP_{y}-D)ₚ Ia

The linker may be a dendritic type linker for covalent attachment of more than one drug moiety through a branching, multifunctional linker moiety to an antibody (Sun et al (2002) Bioorganic & Medicinal Chemistry Letters 12:2213-2215; Sun et al (2003) Bioorganic & Medicinal Chemistry 11: 1761-1768). Dendritic linkers can increase the molar ratio of drug to antibody, i.e. loading, which is related to the potency of the ADC. Thus, where an antibody bears only one reactive group, e.g. a lysine amino or a cysteine thiol, a multitude of drug moieties may be attached through a dendritic linker.

The following exemplary embodiments of dendritic linker reagents allow up to nine nucleophilic drug moiety reagents to be conjugated by reaction with the chloroethyl nitrogen mustard functional groups: 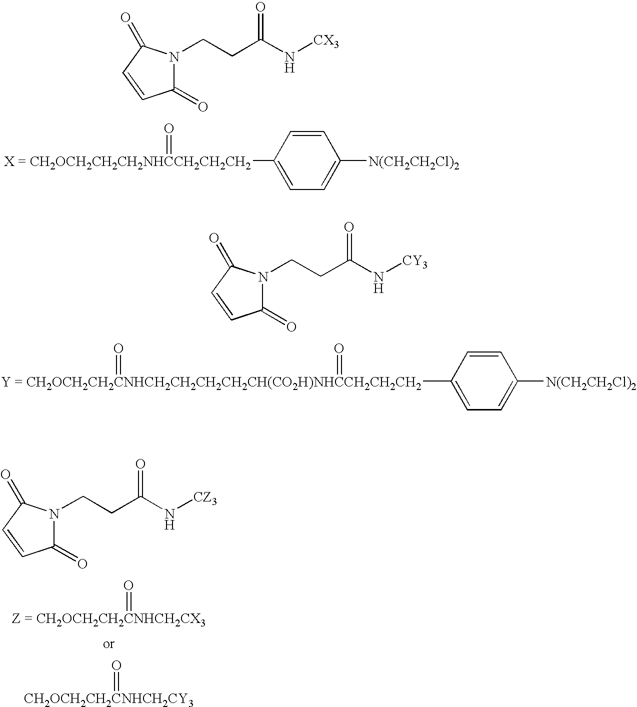
Stretcher Unit

The Stretcher unit (-A-), when present, is capable of linking an antibody (Ab) to an amino acid unit (—W—). In this regard an antibody (Ab) has a functional group that can form a bond with a functional group of a Stretcher. Useful functional groups that can be present on an antibody, either naturally or via chemical manipulation include, but are not limited to, sulfhydryl (—SH), amino, hydroxyl, carboxy, the anomeric hydroxyl group of a carbohydrate, and carboxyl. In one aspect, the reactive functional groups on the antibody are sulfhydryl and amino. Sulfhydryl groups can be generated by reduction of an intramolecular cysteine disulfide bond of an antibody. Alternatively, sulfhydryl groups can be generated by reaction of an amino group of a lysine moiety of an antibody using 2-iminothiolane (Traut's reagent) or another sulfhydryl generating reagent.

In one embodiment, the Stretcher unit forms a bond with a sulfur atom, e.g. a cysteine amino acid residue, of the Antibody unit. The sulfur atom can be derived from a sulfhydryl group of an antibody. Representative Stretcher units of this embodiment are depicted in Formulas IIIa and IIIb, wherein Ab-, -W-, -SP-, -D, w and y are as defined above and wherein R¹⁷ is selected from (CH₂)ᵣ, C₃-C₈ carbocyclyl, O—(CH₂)ᵣ, arylene, (CH₂)ᵣ-arylene, -arylene-(CH₂)ᵣ—, (CH₂)Cᵣ—(C₃-C₈ carbocyclyl), (C₃-C₈ carbocyclyl)-(CH₂)ᵣ, C₃-C₈ heterocyclyl, (CH₂)ᵣ—(C₃-C₈ heterocyclyl), —(C₃-C₈ heterocyclyl)-(CH₂)ᵣ, —(CH₂)ᵣC(O)NR^{b}(CH₂)ᵣ—, —(CH₂CH₂O)ᵣ—, -(CH₂)ᵣO(CH₂CH₂O)ᵣ(CH₂)ᵣ—, -(CH₂)ᵣC(O)NR^{b}(CH₂CH₂O)ᵣ(CH₂)ᵣ—, -(CH₂)ᵣC(O)NR^{b}(CH₂CH₂O)ᵣ—CH₂—, -(CH₂CH₂O)ᵣC(O)NR^{b}(CH₂CH₂O)ᵣ(CH₂)ᵣ—, -(CH₂CH₂O)ᵣC(O)NR^{b}(CH₂CH₂O)ᵣ—CH₂—, and -(CH₂CH₂O)ᵣC(O)NR^{b}(CH₂)ᵣ—; where r is independently an integer ranging from 1-10.

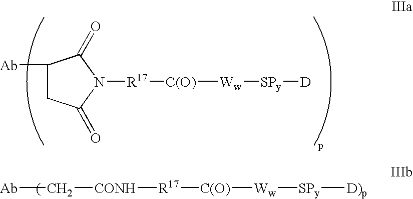

An illustrative Stretcher unit is that of Formula IIIa is derived from maleimido-caproyl (MC) wherein R¹⁷ is —(CH₂)₅—:

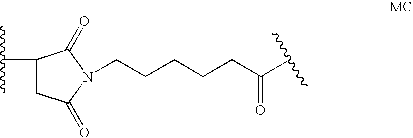

An illustrative Stretcher unit is that of Formula IIIa is derived from maleimido-propanoyl (MP) wherein R¹⁷ is —(CH₂)₂—:

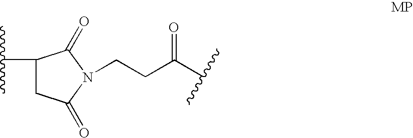

Another illustrative Stretcher unit is that of Formula IIIa wherein R¹⁷ is —(CH₂CH₂O)ᵣ—CH₂— and r is

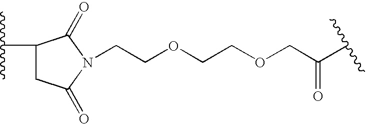

Another illustrative Stretcher unit is that of Formula IIIa wherein R¹⁷ is —(CH₂)ᵣC(O)NR^{b}(CH₂CH₂O)ᵣ—CH₂— where R^{b} is H and each r is 2:

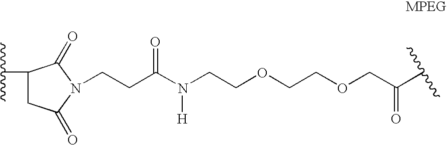

Another illustrative Stretcher unit is that of Formula IIIb wherein R¹⁷ is —(CH₂)₅—:

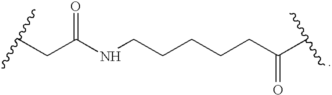

In another embodiment, the Stretcher unit is linked to the Antibody unit via a disulfide bond between a sulfur atom of the Antibody unit and a sulfur atom of the Stretcher unit. A representative Stretcher unit of this embodiment is depicted within the square brackets of Formula IV, wherein R¹⁷, Ab-, -W-, -SP-, -D, w and y are as defined above.
Ab [symbol] S-R¹⁷-C(O)-W_{w}-SP_{y}-D)ₚ IV

In yet another embodiment, the reactive group of the Stretcher contains a reactive site that can form a bond with a primary or secondary amino group of an antibody. Example of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher units of this embodiment are depicted within the square brackets of Formulas Va, Vb and Vc, wherein -R¹⁷-, Ab-, -W-, -SP-, -D, w and y are as defined above;
Ab [symbol] C(O)NH-R¹⁷-C(O)-W_{w}-SP_{y}-D)ₚ Va
Ab [symbol] C(S)NH-R¹⁷-C(O)-W_{w}-SP_{y}-D)ₚ Vb
Ab [symbol] C(O)-R¹⁷-C(O)-W_{w}-SP_{y}-D)ₚ Vc

In yet another aspect, the reactive group of the Stretcher is reactive with an aldehyde, acetal, or ketal group on a sugar (carbohydrate) of a glycosylated antibody. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko, T. et al (1991) Bioconjugate Chem 2:133-41. Representative Stretcher units of this embodiment are depicted within the square brackets of Formulas VIa, VIb, and VIc, wherein -R¹⁷—, Ab-, -W-, -SP-, -D, w and y are as defined above.
Ab [symbol] N-NH-R¹⁷-C(O)-W_{w}-SP_{y}-D)ₚ VIa
Ab [symbol] N-O-R¹⁷-C(O)-W_{w}-SP_{y}-D)ₚ VIb
Ab [symbol] N-NH-C(O)-R¹⁷-C(O)-W_{w}-SP_{y}-D)ₚ VIc
Amino Acid Unit

The Amino Acid unit (-W-), when present: (i) links the Stretcher unit to the Spacer unit if the Spacer unit is present, (ii) links the Stretcher unit to the Drug unit if the Spacer unit is absent, and (iii) links the antibody unit to the Drug unit if the Stretcher unit and Spacer unit are absent.

Amino Acid unit -W_{w}- is a dipeptide, tripeptide, tetrapeptide, pentapeptide, hexapeptide, heptapeptide, octapeptide, nonapeptide, decapeptide, undecapeptide or dodecapeptide unit. Each -W- unit independently has the formula denoted below in the square brackets, and w is an integer ranging from 0 to 12:

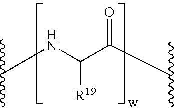

wherein R¹⁹ includes all naturally occurring amino acid side chains, and analogs thereof. R¹⁹ is selected from hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂,—(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl, and

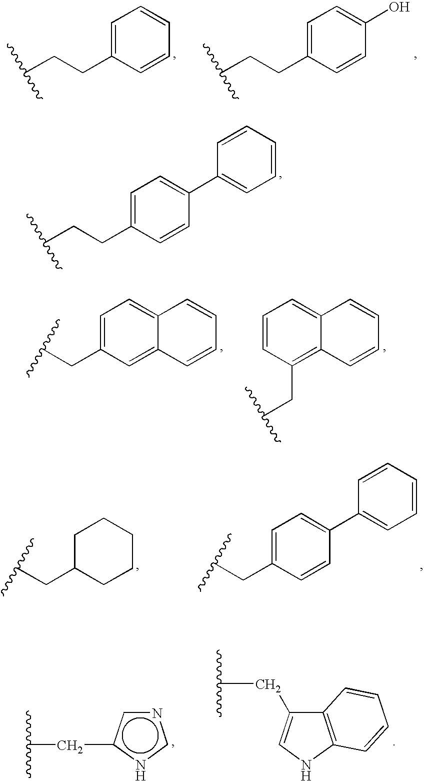

The Amino Acid unit can be enzymatically cleaved by one or more enzymes, including a tumor-associated protease or apoptotic-related enzyme such as cathepsin B, C, and D, or a plasmin protease, to liberate the drug moiety (-D).

Illustrative W_{w} units are represented by Formulas (VII)-(IX):

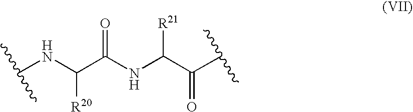

wherein R²⁰ and R²¹ are as follows:

| R²⁰ | R²¹ |
|---|---|
| benzyl | (CH₂)₄NH₂ |
| methyl | (CH₂)₄NH₂ |
| methyl | benzyl |
| isopropyl | (CH₂)₄NH₂ |
| isopropyl | (CH₂)₃NHCONH₂ |
| benzyl | (CH₂)₃NHCONH₂ |
| isobutyl | (CH₂)₃NHCONH₂ |
| sec-butyl | (CH₂)₃NHCONH₂ |
| | (CH₂)₃NHCONH₂ |

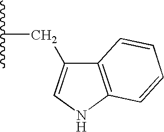

| | |
|---|---|
| benzyl | methyl |
| benzyl | (CH₂)₃NHC(═NH)NH₂ |

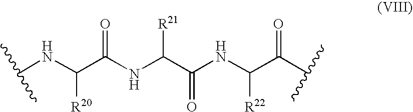

wherein R²⁰, R²¹ and R²² are as follows:

| R²⁰ | R²¹ | R²² |
|---|---|---|
| benzyl | benzyl | (CH₂)₄NH₂ |
| isopropyl | benzyl | (CH₂)₄NH₂ |
| H | benzyl | (CH₂)₄NH₂ |

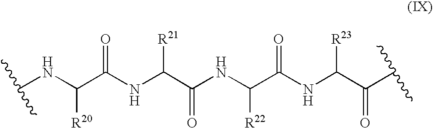

wherein R²⁰, R²¹, R²² and R²³ are as follows:

| R²⁰ | R²¹ | R²² | R²³ |
|---|---|---|---|
| H | benzyl | isobutyl | H |
| methyl | isobutyl | methyl | isobutyl |

Exemplary Amino Acid units include, but are not limited to, units of Formula (VI) where: R²⁰ is benzyl and R²¹ is —(CH₂)₄NH₂; R²⁰ isopropyl and R²¹ is —(CH₂)₄NH₂; R²⁰ isopropyl and R²¹ is —(CH₂)₃NHCONH₂. Another exemplary Amino Acid unit is a unit of Formula (VIII) wherein R²⁰ is benzyl, R²¹ is benzyl, and R²² is —(CH₂)₄NH₂.

Exemplary -W_{w}- Amino Acid units include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine- valine-citrulline and glycine-glycine-glycine.

When R¹⁹, R²⁰, R²¹, R²² or R²³ is other than hydrogen, the carbon atom to which R¹⁹, R²⁰, R²¹, R²² or R²³ is attached is chiral.

Each carbon atom to which R¹⁹, R²⁰, R²¹, R²² or R²³ is attached independently in the (S) or (R) configuration, or a racemic mixture. Amino acid units may thus be enantiomerically pure, racemic, or diastereomeric.

Spacer Unit

The Spacer unit (-SP-), when present: (i) links an Amino Acid unit to the Drug unit when an Amino Acid unit is present, (ii) links the Stretcher unit to the Drug moiety when the Amino Acid unit is absent, or (iii) links the Drug moiety to the antibody unit when both the Amino Acid unit and Stretcher unit are absent. Spacer units are of two general types: self-immolative and non self-immolative. A non self-immolative Spacer unit is one in which part or all of the Spacer unit remains bound to the Drug moiety after cleavage, particularly enzymatic, of an Amino Acid unit from the Drug-Linker-antibody Conjugate or the Drug-Linker Compound. Examples of a non self-immolative Spacer unit include, but are not limited to a (glycine-glycine) Spacer unit and a glycine Spacer unit. When an Exemplary Compound containing a glycine-glycine Spacer unit or a glycine Spacer unit undergoes enzymatic cleavage via a tumor-cell associated-protease, a cancer-cell-associated protease or a lymphocyte-associated protease, a glycine-glycine-Drug moiety or a glycine-Drug moiety is cleaved from Ab-Aₐ-W_{w}-. In one embodiment, an independent hydrolysis reaction takes place within the target cell, cleaving the glycine-Drug moiety bond and liberating the Drug.

In another embodiment, -SP_{y}- is a para-aminobenzyloxycarbonyl (PAB) unit whose phenylene portion is substituted with Qₘ wherein Q is -C₁-C₈ alkyl, -O-(C₁-C₈ alkyl), -halogen, -nitro or -cyano; and m is an integer ranging from 0-4.

Exemplary embodiments of a non self-immolative Spacer unit (-SP-) are: -Gly-Gly-; -Gly-; -Ala-Phe-; -Val-Cit-.

In one embodiment, a Drug moiety-linker or an ADC is provided in which the Spacer unit is absent (y=0), or a pharmaceutically acceptable salt or solvate thereof.

Alternatively, an ADC containing a self-immolative Spacer unit can release -D. In one embodiment, -SP- is a PAB group that is linked to -W_{w}-via the amino nitrogen atom of the PAB group, and connected directly to -D via a carbonate, carbamate or ether group, where the ADC has the exemplary structure:

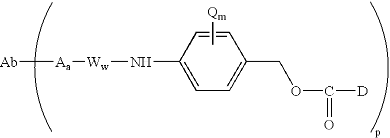

wherein Q is -C₁-C₈ alkyl, —O—(C₁-C₈ alkyl), -halogen, -nitro or -cyano; m is an integer ranging from 0-4; and p ranges from 1 to 4.

Other examples of self-immolative spacers include, but are not limited to, aromatic compounds that are electronically similar to the PAB group such as 2-aminoimidazol-5-methanol derivatives (Hay et al. (1999) Bioorg. Med. Chem. Lett. 9:2237) and ortho or para-aminobenzylacetals. Self-immolative spacers also include where the PAB group is substituted by a heterocyclic group (WO 2005/082023). Spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry et al (1990) J. Org. Chem., 55:5867). Elimination of amine-containing drugs that are substituted at glycine (Kingsbury et al (1984) J. Med. Chem., 27, 1447) are also examples of self-immolative spacer useful in ADCs.

In one embodiment, the Spacer unit is a branched bis(hydroxymethyl)styrene (BHMS), which can be used to incorporate and release multiple drugs, having the structure:

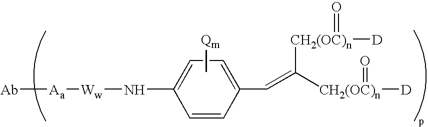

wherein Q is -C₁-C₈ alkyl, -O-(C₁-C₈ alkyl), -halogen, -nitro or -cyano; m is an integer ranging from 0-4; n is 0 or 1; and p ranges raging from 1 to 4.

In another embodiment, the -D moieties are the same.

In yet another embodiment, the -D moieties are different.

in one aspect, Spacer units (-SP_{y}-) are represented by Formulas (X)-(XII):

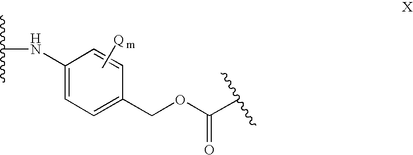

wherein Q is -C₁-C₈ alkyl, -O-(C₁-C₈ alkyl), -halogen, -nitro or -cyano; and m is an integer ranging from 0-4;

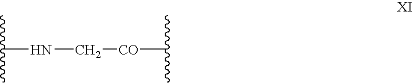

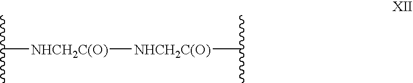

Embodiments of the Formula I antibody-drug conjugate compounds include XIIIa (val-cit), XIIIb (MC-val-cit), XIIIc (MC-val-cit-PAB):

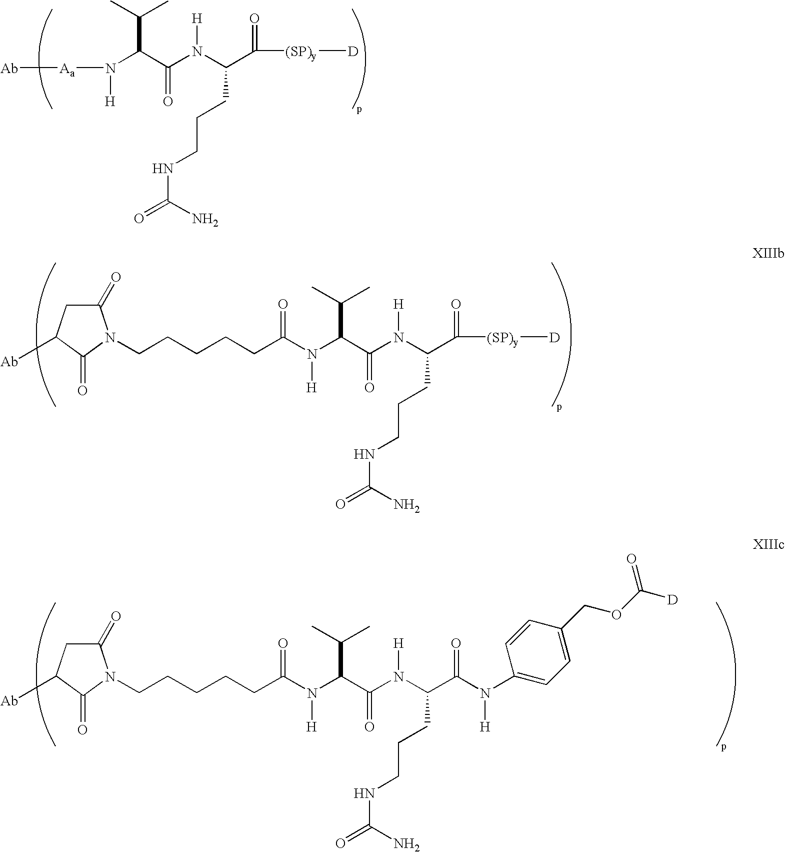

Other exemplary embodiments of the Formula Ia antibody-drug conjugate compounds include XIVa-h: 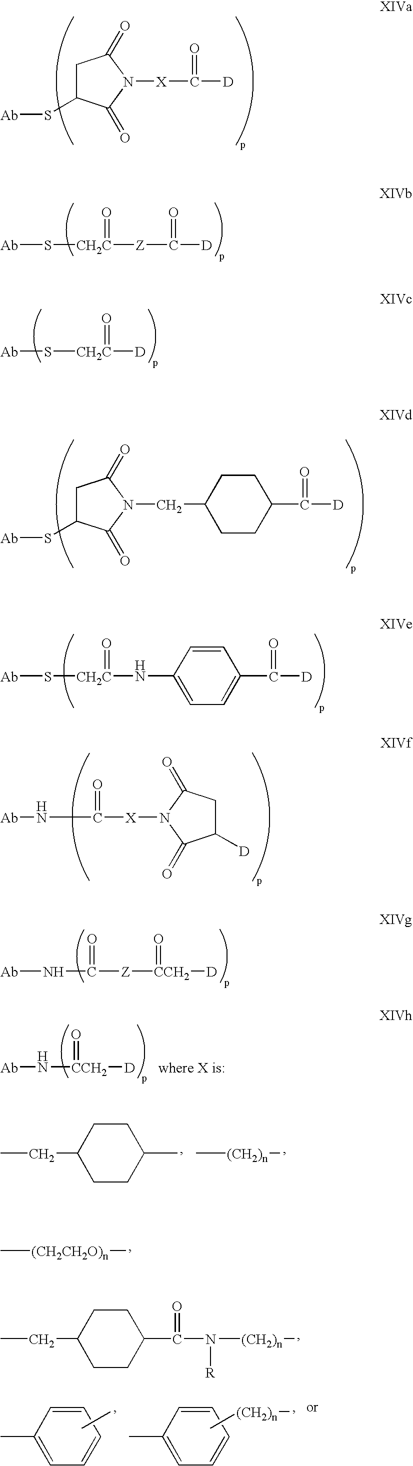 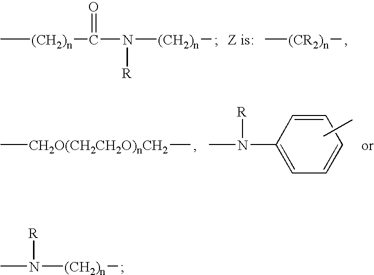
and R is independently H or C₁-C₆ alkyl; and n is 1 to 12.
BIS 1,8 Naphthalimide-Linker Reagents

Intermediates or reagents which include a bis 1,8 naphthalimide drug moiety and a reactive linker unit may comprise any combination of the bis 1,8 naphthalimide drug moieties and linker units. Bis 1,8 naphthalimide-linker reagents bear functionality which is reactive with an antibody so as to allow covalent attachment, i.e. conjugation, of the reagent to the antibody to prepare an antibody drug conjugate (ADC) of the invention. Exemplary embodiments include the following bis 1,8 naphthalimide-linker reagents: 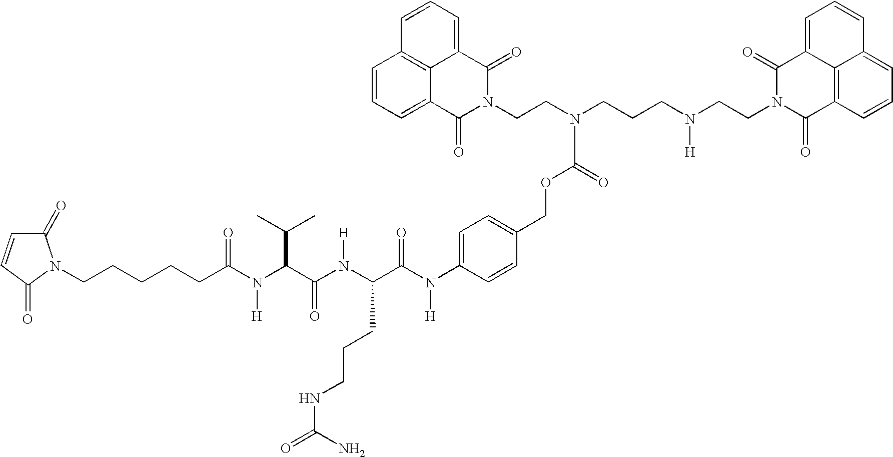
where MC is maleimido-caproyl, vc is the valine-citrulline amino acid subunit, PAB is para-aminobenzyloxycarbonyl, and E is the bis 1,8 naphthalimide drug moiety Ia where X¹, X², X³, and X⁴ are H, R^{b} is H, m is 3, and n is 2. 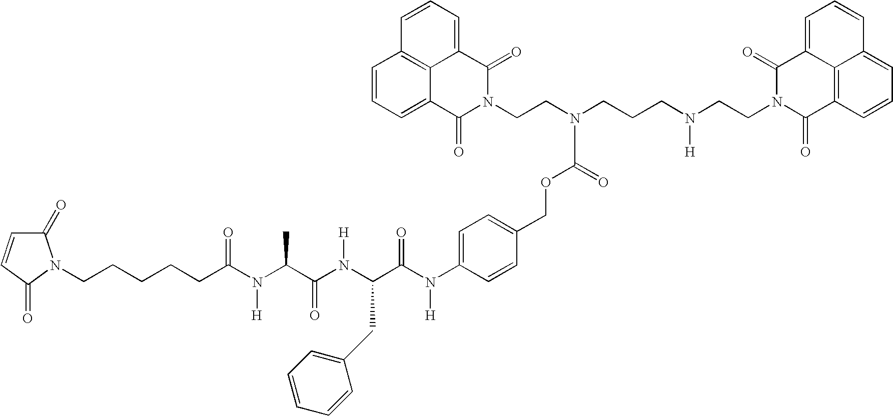
where af is the alanine-phenylalanine amino acid subunit.

Another exemplary bis 1,8 naphthalimide drug-linker reagent is MC-vc-PAB-(N,N′-(bis-aminoethyl-1,3-propanediamine)-bis-(4-N-imidazolyl)-1,8 naphthalimide) IIIa: 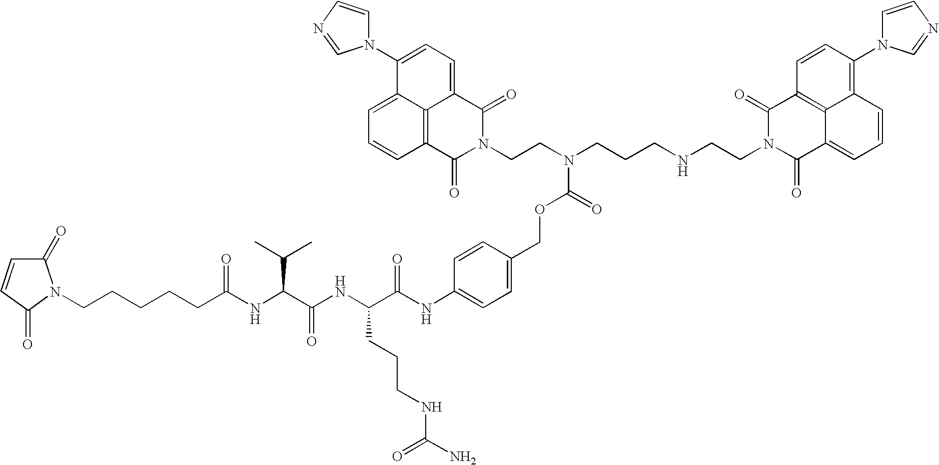
Antibodies

The antibody unit (Ab-) includes within its scope any unit of an antibody (Ab) that binds or reactively associates or complexes with a receptor, antigen or other receptive moiety associated with a given target-cell population. An antibody can be any protein or protein-like molecule that binds to, complexes with, or reacts with a moiety of a cell population sought to be therapeutically or otherwise biologically modified. In one aspect, the antibody unit acts to deliver the Drug unit to the particular target cell population with which the antibody unit reacts. Such antibodies include, but are not limited to, large molecular weight proteins such as, for example, full-length antibodies, antibody fragments.

An antibody unit can form a bond to either a linker, a Stretcher unit, an Amino Acid unit, a Spacer Unit, or a Drug moiety directly. An antibody unit can form a bond to a Linker unit via a heteroatom of the antibody. The linking heteroatoms of the antibody may be a reactive nucleophilic group on any amino acid side chain, such as a cysteine thiol, a lysine amine, an aspartic acid or glutamic acid carboxyl, a serine, threonine, or tyrosine hydroxyl, or an arginine. Heteroatoms that may be present on an antibody unit include sulfur (in one embodiment, from a sulfhydryl group of an antibody such as a cysteine thiol), oxygen (in one embodiment, from a carbonyl, carboxyl or hydroxyl group of an antibody) and nitrogen (in one embodiment, from a primary or secondary amino group of an antibody). These heteroatoms can be present on the antibody in the antibody's natural state, for example a naturally occurring antibody, or can be introduced into the antibody via chemical modification.

In another embodiment, the antibody has one or more lysine residues that can be chemically modified to introduce one or more sulfhydryl groups. The antibody unit then may bond to a linker reagent or drug-linker moiety via the sulfhydryl group's sulfur atom. The reagents that can be used to modify lysines include, but are not limited to, N-succinimidyl S-acetylthioacetate (SATA) and 2-Iminothiolane hydrochloride (Traut's Reagent).

In another embodiment, the antibody can have one or more carbohydrate groups that can be chemically modified to have one or more sulfhydryl groups. The antibody unit bonds to the linker reagent or drug-linker moiety, such as the Stretcher Unit, via the sulfhydryl group's sulfur atom.

In yet another embodiment, the antibody can have one or more carbohydrate groups that can be oxidized to provide an aldehyde (—CHO) group suitable for conjugation with a linker reagent or drug-linker moiety (see, for e.g., Laguzza, et al., J. Med. Chem. 1989, 32(3), 548-55). Suitable oxidizing reagents include periodate reagents. The corresponding aldehyde can form a bond with a Reactive Site on a Stretcher. The reaction may proceed through a Schiff's base intermediate and undergo subsequent reduction to a stable amine linkage. Reactive sites on a Stretcher that can react with a carbonyl group on an antibody include, but are not limited to, hydrazine and hydroxylamine. Other protocols for the modification of proteins for the attachment or association of Drug Units are described in Coligan et al., Current Protocols in Protein Science, vol. 2, John Wiley & Sons (2002), incorporated herein by reference.

In yet another embodiment, a tyrosine residue of the antibody may undergo diazotization by electrophilici aromatic substitution to form a diazo linkage with a linker reagent or drug-linker moiety.

In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies.

Antibodies which comprise Ab in Formula I antibody drug conjugates (ADC) and which may be useful in the treatment of cancer include, but are not limited to, antibodies against tumor-associated antigens (TAA). Such tumor-associated antigens are known in the art, and can prepared for use in generating antibodies using methods and information which are well known in the art. Examples of TAA include (1)-(35), but are not limited to TAA (1)-(36) listed below. For convenience, information relating to these antigens, all of which are known in the art, is listed below and includes names, alternative names, Genbank accession numbers and primary reference(s). Tumor-associated antigens targeted by antibodies include all amino acid sequence variants and isoforms possessing at least about 70%, 80%, 85%, 90%, or 95% sequence identity relative to the sequences identified in the cited references, or which exhibit substantially the same biological properties or characteristics as a TAA having a sequence found in the cited references. For example, a TAA having a variant sequence generally is able to bind specifically to an antibody that binds specifically to the TAA with the corresponding sequence listed. The sequences and disclosure specifically recited herein are expressly incorporated by reference.

Tumor-Associated Antigens (1)-(36):

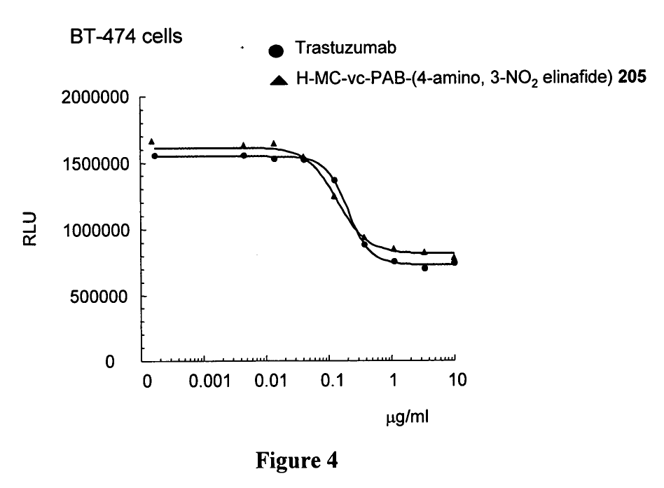

(1) BMPR1B (bone morphogenetic protein receptor-type IB, Genbank accession no. NM-001203) ten Dijke, P., et al Science 264 (5155):101-104 (1994), Oncogene 14 (11):1377-1382 (1997)); WO2004063362 (Claim 2); WO2003042661 (Claim 12); US2003134790-A1 (Page 38-39); WO2002102235 (Claim 13; Page 296); WO2003055443 (Page 91-92); WO200299122 (Example 2; Page 528-530); WO2003029421 (Claim 6); WO2003024392 (Claim 2; FIG. 112); WO200298358 (Claim 1; Page 183); WO200254940 (Page 100-101); WO200259377 (Page 349-350); WO200230268 (Claim 27; Page 376); WO200148204 (Example; FIG. 4)

NP_{—}001194 bone morphogenetic protein receptor, type IB/pid=NP_{—}001194.1—

Cross-references: MIM:603248; NP_{—}001194.1; NM_{—}001203_{—}1

(2) E16 (LAT1, SLC7A5, Genbank accession no. NM_{—}003486)

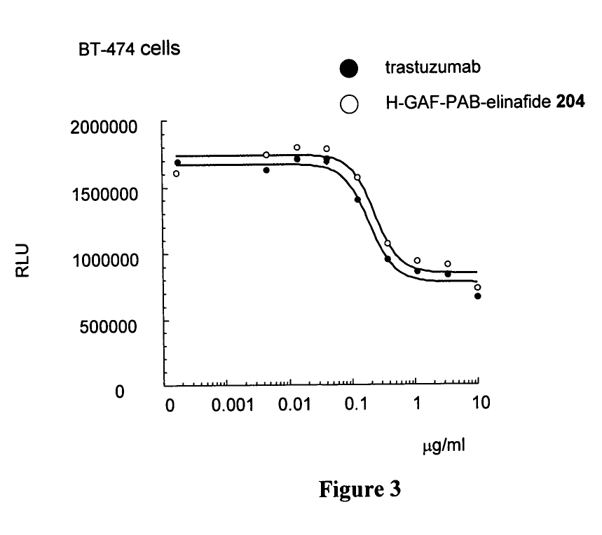

Biochem. Biophys. Res. Commun. 255 (2), 283-288 (1999), Nature 395 (6699):288-291 (1998), Gaugitsch, H. W., et al (1992) J. Biol. Chem. 267 (16):11267-11273); WO2004048938 (Example 2); WO2004032842 (Example IV); WO2003042661 (Claim 12); WO2003016475 (Claim 1); WO200278524 (Example 2); WO200299074 (Claim 19; Page 127-129); WO200286443 (Claim 27; Pages 222, 393); WO2003003906 (Claim 10; Page 293); WO200264798 (Claim 33; Page 93-95); WO200014228 (Claim 5; Page 133-136); US2003224454 (FIG. 3); WO2003025138 (Claim 12; Page 150);

NP_{—}003477 solute carrier family 7 (cationic amino acid transporter, y+system), member 5/pid=NP_{—}003477.3—Homo sapiens

Cross-references: MIM:600182; NP_{—}003477.3; NM_{—}015923; NM_{—}003486_{—}1

(3) STEAP1 (six transmembrane epithelial antigen of prostate, Genbank accession no. NM_{—}012449)

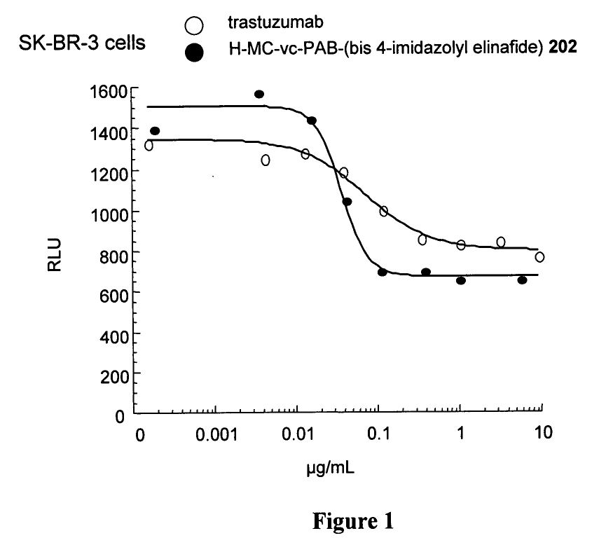

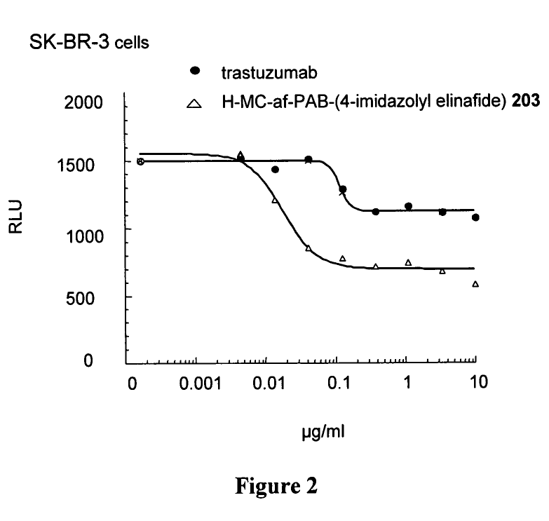

Cancer Res. 61 (15), 5857-5860 (2001), Hubert, R. S., et al (1999) Proc. Natl. Acad. Sci. U.S.A. 96 (25):14523-14528); WO2004065577 (Claim 6); WO2004027049 (FIG. 1L); EP1394274 (Example 11); WO2004016225 (Claim 2); WO2003042661 (Claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A);

NP_{—}36581 six transmembrane epithelial antigen of the prostate

Cross-references: MIM:604415; NP_{—}036581.1; NM_{—}012449_{—}1

(4) 0772P (CA125, MUC16, Genbank accession no. AF361486)

J. Biol. Chem. 276 (29):27371-27375 (2001)); WO2004045553 (Claim 14); WO200292836 (Claim 6; FIG. 12); WO200283866 (Claim 15; Page 116-121); US2003124140 (Example 16); US2003091580 (Claim 6); WO200206317 (Claim 6; Page 400-408);

Cross-references: GI:34501467; AAK74120.3; AF361486_{—}1

(5) MPF (MPF, MSLN, SMR, megakaryocyte potentiating factor, mesothelin, Genbank accession no. NM_{—}005823)

Yamaguchi, N., et al Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J. Biol. Chem. 270 (37):21984-21990 (1995)); WO2003101283 (Claim 14); (WO2002102235 (Claim 13; Page 287-288); WO2002101075 (Claim 4; Page 308-309); WO200271928 (Page 320-321); WO9410312 (Page 52-57);

Cross-references: MIM:601051; NP_{—}005814.2; NM_{—}005823_{—}1

(6) Napi3b (NAPI-3B, NPTIIb, SLC34A2, solute carrier family 34 (sodium phosphate), member 2, type II sodium-dependent phosphate transporter 3b, Genbank accession no. NM_{—}006424)

J. Biol. Chem. 277 (22):19665-19672 (2002), Genomics 62 (2):281-284 (1999), Feild, J. A., et al (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140);

Cross-references: MIM:604217; NP_{—}006415.1; NM_{—}006424_{—}1

(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878)

Nagase T., et al (2000) DNA Res. 7 (2):143-150); WO2004000997 (Claim 1); WO2003003984 (Claim 1); WO200206339 (Claim 1; Page 50); WO200188133 (Claim 1; Page 41-43, 48-58); WO2003054152 (Claim 20); WO2003101400 (Claim 11);

Accession: Q9P283; EMBL; AB040878; BAA95969.1. Genew; HGNC:10737;

(8) PSCA hlg (2700050C12Rik, C530008O16Rik, RIKEN cDNA 2700050C12, RIKEN cDNA 2700050C12 gene, Genbank accession no. AY358628);

US2003129192 (Claim 2); US2004044180 (Claim 12); US2004044179 (Claim 11); US2003096961 (Claim 11); US2003232056 (Example 5); WO2003105758 (Claim 12); US2003206918 (Example 5); EP1347046 (Claim 1); WO2003025148 (Claim 20);

Cross-references: GI:37182378; AAQ88991.1; AY358628_{—}1

(9) ETBR (Endothelin type B receptor, Genbank accession no. AY275463);

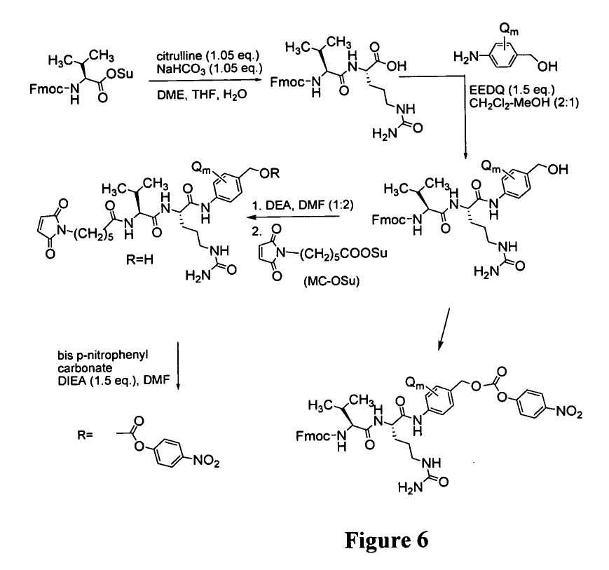

Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, s1-S4, 1992; Tsutsumi M., et al Gene 228, 43-49, 1999; Strausberg R. L., et al Proc. Natl. Acad. Sci. U.S.A. 99, 16899-16903, 2002; Bourgeois C., et al J. Clin. Endocrinol. Metab. 82, 3116-3123, 1997; Okamoto Y., et al Biol. Chem. 272, 21589-21596,1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P. J., et al Hum. Genet. 103, 145-148, 1998; Fuchs S., et al Mol. Med. 7, 115-124, 2001; Pingault V., et al (2002) Hum. Genet. 111, 198-206; WO2004045516 (Claim 1); WO2004048938 (Example 2); WO2004040000 (Claim 151); WO2003087768 (Claim 1); WO2003016475 (Claim 1); WO2003016475 (Claim 1); WO200261087 (FIG. 1); WO2003016494 (FIG. 6); WO2003025138 (Claim 12; Page 144); WO200198351 (Claim 1; Page 124-125); EP522868 (Claim 8; FIG. 2); WO200177172 (Claim 1; Page 297-299); US2003109676; U.S. Pat. No. 6,518,404 (FIG. 3); U.S. Pat. No. 5,773,223 (Claim 1a; Col 31-34); WO2004001004;

(10) MSG783 (RNF124, hypothetical protein FLJ20315, Genbank accession no. NM_{—}017763); WO2003104275 (Claim 1); WO2004046342 (Example 2); WO2003042661 (Claim 12); WO2003083074 (Claim 14; Page 61); WO2003018621 (Claim 1); WO2003024392 (Claim 2; FIG. 93); WO200166689 (Example 6);

Cross-references: LocusID:54894; NP_{—}060233.2; NM_{—}017763_{—}1

(11) STEAP2 (HGNC_{—}8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138)

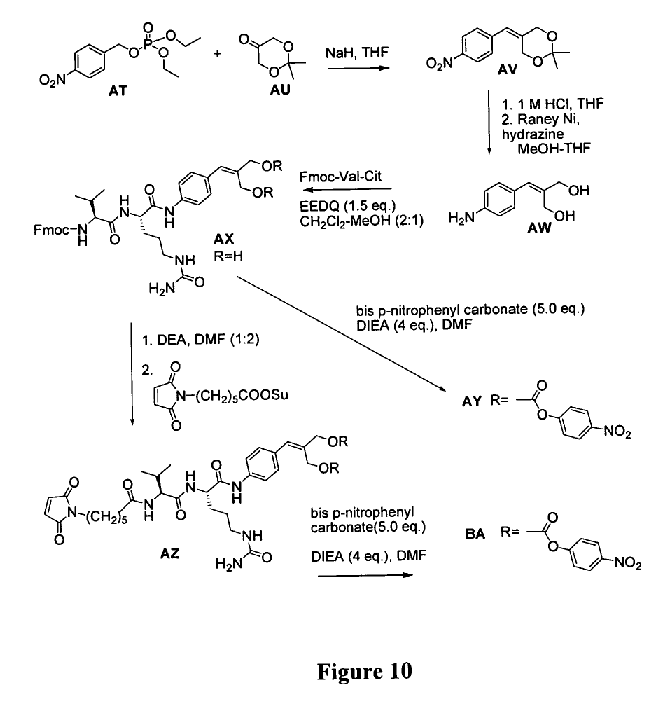

Lab. Invest. 82 (11):1573-1582 (2002)); WO2003087306; US2003064397 (Claim 1; FIG. 1); WO200272596 (Claim 13; Page 54-55); WO200172962 (Claim 1; FIG. 4B); WO2003104270 (Claim 11); WO2003104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10);

Cross-references: GI:22655488; AAN04080.1; AF455138_{—}1

(12) TrpM4 (BR22450, FLJ20041, TRPM4, TRPM4B, transient receptor potential cation channel, subfamily M, member 4, Genbank accession no. NM_{—}017636)

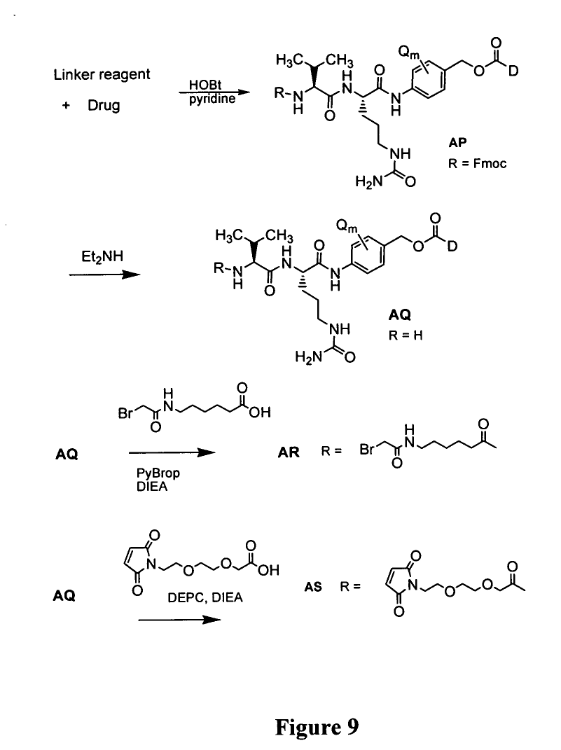

Xu, X. Z., et al Proc. Natl. Acad. Sci. U.S.A. 98 (19):10692-10697 (2001), Cell 109 (3):397-407 (2002), J. Biol. Chem. 278 (33):30813-30820 (2003)); US2003143557 (Claim 4); WO200040614 (Claim 14; Page 100-103); WO200210382 (Claim 1; FIG. 9A); WO2003042661 (Claim 12); WO200230268 (Claim 27; Page 391); US2003219806 (Claim 4); WO200162794 (Claim 14; FIG. 1A-D);

Cross-references: MIM:606936; NP_{—}060106.2; NM_{—}017636_{—}1

(13) CRIPTO (CR, CR1, CRGF, CRIPTO, TDGF1, teratocarcinoma-derived growth factor, Genbank accession no. NP_{—}003203 or NM_{—}003212)

Ciccodicola, A., et al EMBO J. 8 (7):1987-1991 (1989), Am. J. Hum. Genet. 49 (3):555-565 (1991)); US2003224411 (Claim 1); WO2003083041 (Example 1); WO2003034984 (Claim 12); WO200288170 (Claim 2; Page 52-53); WO2003024392 (Claim 2; FIG. 58); WO200216413 (Claim 1; Page 94-95, 105); WO200222808 (Claim 2; FIG. 1); U.S. Pat. No. 5,854,399 (Example 2; Col 17-18); U.S. Pat. No. 5,792,616 (FIG. 2);

Cross-references: MIM:187395; NP_{—}003203.1; NM_{—}003212_{—}1

(14) CD21 (CR2 (Complement receptor 2) or C3DR (C3d/Epstein Barr virus receptor) or Hs.73792 Genbank accession no. M26004)

Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004045520 (Example 4); US2004005538 (Example 1); WO2003062401 (Claim 9); WO2004045520 (Example 4); WO9102536 (FIG. 9. 1-9.9); WO2004020595 (Claim 1);

Accession: P20023; Q13866; Q14212; EMBL; M26004; AAA35786.1.

(15) CD79b (CD79B, CD79β, IGb (immunoglobulin-associated beta), B29, Genbank accession no. NM_{—}000626 or 11038674)

Proc. Natl. Acad. Sci. U.S.A. (2003) 100 (7):4126-4131, Blood (2002) 100 (9):3068-3076, Muller et al (1992) Eur. J. Immunol. 22 (6):1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146);

Cross-references: MIM:147245; NP_{—}000617.1; NM_{—}000626_{—}1

(16) FcRH2 (IFGP4, IRTA4, SPAP1A (SH2 domain containing phosphatase anchor protein 1a), SPAP1B, SPAP1C, Genbank accession no. NM_{—}030764)

Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004016225 (Claim 2); WO2003077836; WO200138490 (Claim 5; FIG. 18D-1-18D-2); WO2003097803 (Claim 12); WO2003089624 (Claim 25);

Cross-references: MIM:606509; NP_{—}110391.2; NM_{—}030764_{—}1

(17) HER2 (ErbB2, Genbank accession no. M11730)

Coussens L., et al Science (1985) 230(4730):1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-880, 2004; Kuhns J. J., et al J. Biol. Chem. 274, 36422-36427, 1999; Cho H.—S., et al Nature 421, 756-760, 2003; Ehsani A., et al (1993) Genomics 15, 426429; WO2004048938 (Example 2); WO2004027049 (FIG. 1I); WO2004009622; WO2003081210; WO2003089904 (Claim 9); WO2003016475 (Claim 1); US2003118592; WO2003008537 (Claim

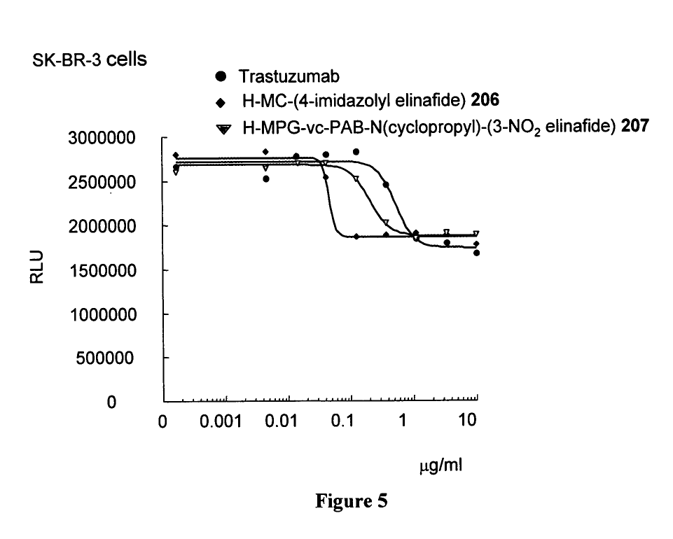

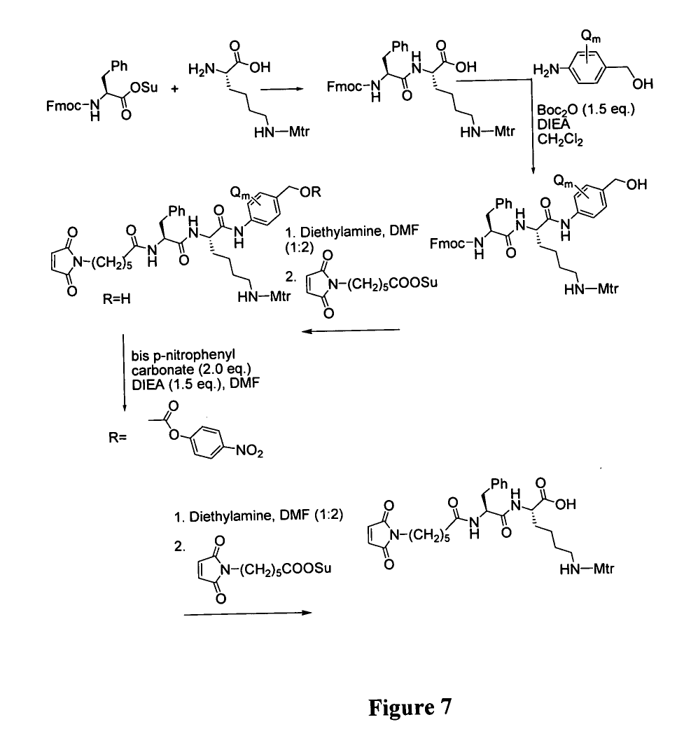

1); WO2003055439 (Claim 29; FIG. 1A-B); WO2003025228 (Claim 37; FIG. 5C); WO200222636 (Example 13; Page 95-107); WO200212341 (Claim 68; FIG. 7); WO200213847 (Page 71-74); WO200214503 (Page 114-117); WO200153463 (Claim 2; Page 41-46); WO200141787 (Page 15); WO200044899 (Claim 52; FIG. 7); WO200020579 (Claim 3; FIG. 2); U.S. Pat. No. 5,869,445 (Claim 3; Col 31-38); WO9630514 (Claim 2; Page 56-61); EP1439393 (Claim 7); WO2004043361 (Claim 7); WO2004022709; WO200100244 (Example 3; FIG. 4);

Accession: P04626; EMBL; M11767; AAA35808.1. EMBL; M11761; AAA35808.1.

(18) NCA (CEACAM6, Genbank accession no. M18728);

Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99:16899-16903, 2002; WO2004063709; EP1439393 (Claim 7); WO2004044178 (Example 4); WO2004031238; WO2003042661 (Claim 12); WO200278524 (Example 2); WO200286443 (Claim 27; Page 427); WO200260317 (Claim 2);

Accession: P40199; Q14920; EMBL; M29541; AAA59915.1. EMBL; M18728;

(19) MDP (DPEP1, Genbank accession no. BC017023)

Proc. Natl. Acad. Sci. U.S.A. 99 (26):16899-16903 (2002)); WO2003016475 (Claim 1); WO200264798 (Claim 33; Page 85-87); JP05003790 (FIG. 6-8); WO9946284 (FIG. 9);

Cross-references: MIM:179780; AAH17023.1; BC017023_{—}1

(20) IL20Rα (IL20Ra, ZCYTOR7, Genbank accession no. AF184971);

Clark H. F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) Biochemistry 42:12617-12624; Sheikh F., et al (2004) J. Immunol. 172, 2006-2010; EP1394274 (Example 11); US2004005320 (Example 5); WO2003029262 (Page 74-75); WO2003002717 (Claim 2; Page 63); WO200222153 (Page 45-47); US2002042366 (Page 20-21); WO200146261 (Page 57-59); WO200146232 (Page 63-65); WO9837193 (Claim 1; Page 55-59);

Accession: Q9UHF4; Q6UWA9; Q96SH8; EMBL; AF184971; AAF01320.1.

(21) Brevican (BCAN, BEHAB, Genbank accession no. AF229053)

Gary S. C., et al Gene 256, 139-147, 2000; Clark H. F., et al Genome Res. 13, 2265-2270, 2003; Strausberg R. L., et al Proc. Natl. Acad. Sci. U.S.A. 99, 16899-16903, 2002; US2003186372 (Claim 11); US2003186373 (Claim 11); US2003119131 (Claim 1; FIG. 52); US2003119122 (Claim 1; FIG. 52); US2003119126 (Claim 1); US2003119121 (Claim 1; FIG. 52); US2003119129 (Claim 1); US2003119130 (Claim 1); US2003119128 (Claim 1; FIG. 52); US2003119125 (Claim 1); WO2003016475 (Claim 1); WO200202634 (Claim 1);

(22) EphB2R (DRT, ERK, Hek5, EPHT3, Tyro5, Genbank accession no. NM_{—}004442)

Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42);

Cross-references: MIM:600997; NP_{—}004433.2; NM_{—}004442_{—}1

(23) ASLG659 (B7h, Genbank accession no. AX092328)

US20040101899 (Claim 2); WO2003104399 (Claim 11); WO2004000221 (FIG. 3); US2003165504 (Claim 1); US2003124140 (Example 2); US2003065143 (FIG. 60); WO2002102235 (Claim 13; Page 299); US2003091580 (Example 2); WO200210187 (Claim 6; FIG. 10); WO200194641 (Claim 12; FIG. 7b); WO200202624 (Claim 13; FIG. 1A-1B); US2002034749 (Claim 54; Page 45-46); WO200206317 (Example 2; Page 320-321, Claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (Claim 12); WO2003004989 (Claim 1); WO200271928 (Page 233-234, 452-453); WO 0116318;

(24) PSCA (Prostate stem cell antigen precursor, Genbank accession no. AJ297436)

Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004022709; EP1394274 (Example 11); US2004018553 (Claim 17); WO2003008537 (Claim 1); WO200281646 (Claim 1; Page 164); WO2003003906 (Claim 10; Page 288); WO200140309 (Example 1; FIG. 17); US2001055751 (Example 1; FIG. Ib); WO200032752 (Claim 18; FIG. 1); WO9851805 (Claim 17; Page 97); WO9851824 (Claim 10; Page 94); WO9840403 (Claim 2; FIG. 1B);

Accession: O43653; EMBL; AF043498; AAC39607.1.

(25) GEDA (Genbank accession No. AY260763);

AAP14954 lipoma HMGIC fusion-partner-like protein/pid=AAP14954.1—Homo sapiens Species: Homo sapiens (human)

WO2003054152 (Claim 20); WO2003000842 (Claim 1); WO2003023013 (Example 3, Claim 20); US2003194704 (Claim 45);

Cross-references: GI:30102449; AAP14954.1; AY260763_{—}1

(26) BAFF-R (B cell—activating factor receptor, BLyS receptor 3, BR3, Genbank accession No. NP_{—}443177.1);

NP_{—}443177 BAFF receptor/pid=NP_{—}443177.1—Homo sapiens

Thompson, J. S., et al Science 293 (5537), 2108-2111 (2001); WO2004058309; WO2004011611; WO2003045422 (Example; Page 32-33); WO2003014294 (Claim 35; FIG. 6B); WO2003035846 (Claim 70; Page 615-616); WO200294852 (Col 136-137); WO200238766 (Claim 3; Page 133); WO200224909 (Example 3; FIG. 3);

Cross-references: MIM:606269; NP_{—}443177.1; NM_{—}052945_{—}1

(27) CD22 (B-cell receptor CD22-β isoform, Genbank accession No. NP-001762.1);

Stamenkovic, I. and Seed, B., Nature 345 (6270), 74-77 (1990); US2003157113; US2003118592; WO2003062401 (Claim 9); WO2003072036 (Claim 1; FIG. 1); WO200278524 (Example 2);

Cross-references: MIM:107266; NP_{—}001762.1; NM_{—}001771_{—}1

(28) CD79a (CD79A, CD79a, immunoglobulin-associated alpha, a B cell-specific protein that covalently interacts with Ig beta (CD79B) and forms a complex on the surface with Ig M molecules, transduces a signal involved in B-cell differentiation) 226 aa, pI: 4.84, MW: 25028 TM: 2 [P] Gene Chromosome: 19q 13.2, Genbank accession No. NP-001774.10)

WO2003088808, US20030228319; WO2003062401 (Claim 9); US2002150573 (Claim 4, pages 13-14); WO9958658 (Claim 13, FIG. 16); WO9207574 (FIG. 1); U.S. Pat. No. 5,644,033; Ha et al (1992) J. Immunol. 148(5):1526-1531; Mueller et al (1992) Eur. J. Biochem. 22:1621-1625; Hashimoto et al (1994) Immunogenetics 40(4):287-295; Preud'homme et al (1992) Clin. Exp. Immunol. 90(1):141-146; Yu et al (1992) J. Immunol. 148(2) 633-637; Sakaguchi et al (1988) EMBO J. 7(11):3457-3464;

(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia) 372 aa, pI: 8.54 MW: 41959 TM: 7 [P] Gene Chromosome: 11q23.3, Genbank accession No. NP_{—}001707.1)

WO2004040000; WO2004015426; US2003105292 (Example 2); U.S. Pat. No. 6,555,339 (Example 2); WO200261087 (FIG. 1); WO200157188 (Claim 20, page 269); WO200172830 (pages 12-13); WO200022129 (Example 1, pages 152-153, Example 2, pages 254-256); WO9928468 (claim 1, page 38); U.S. Pat. No. 5,440,021 (Example 2, col 49-52); WO9428931 (pages 56-58); WO9217497 (claim 7, FIG. 5); Dobner et al (1992) Eur. J. Immunol. 22:2795-2799; Barella et al (1995) Biochem. J. 309:773-779;

(30) HLA-DOB (Beta subunit of MHC class II molecule (Ia antigen) that binds peptides and presents them to CD4+ T lymphocytes) 273 aa, pI: 6.56 MW: 30820 TM: 1 [P] Gene Chromosome: 6p21.3, Genbank accession No. NP_{—}002111.1)

Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci. USA 99:16899-16903; Servenius et al (1987) J. Biol. Chem. 262:8759-8766; Beck et al (1996) J. Mol. Biol. 255:1-13; Naruse et al (2002) Tissue Antigens 59:512-519; WO9958658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al (1989) Immunogenetics 30(1):66-68; Larhammar et al (1985) J. Biol. Chem. 260(26):14111-14119;

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability) 422 aa, pI: 7.63, MW: 47206 TM: 1 [P] Gene Chromosome: 17p13.3, Genbank accession No. NP_{—}002552.2)

Le et al (1997) FEBS Lett. 418(1-2):195-199; WO2004047749; WO2003072035 (claim 10); Touchman et al (2000) Genome Res. 10:165-173; WO200222660 (claim 20); WO2003093444 (claim 1); WO2003087768 (claim 1); WO2003029277 (page 82);

(32) CD72 (B-cell differentiation antigen CD72, Lyb-2) 359 aa, pI: 8.66, MW: 40225 TM: 1 [P] Gene Chromosome: 9p13.3, Genbank accession No. NP_{—}001773.1)

WO2004042346 (claim 65); WO2003026493 (pages 51-52, 57-58); WO200075655 (pages 105-106); Von Hoegen et al (1990) J. Immunol. 144(12):4870-4877; Strausberg et al (2002) Proc. Natl. Acad. Sci. USA 99:16899-16903;

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis) 661 aa, pI: 6.20, MW: 74147 TM: 1 [P] Gene Chromosome: 5q 12, Genbank accession No. NP_{—}005573.1)

US2002193567; WO9707198 (claim 11, pages 39-42); Miura et al (1996) Genomics 38(3):299-304; Miura et al (1998) Blood 92:2815-2822; WO2003083047; WO9744452 (claim 8, pages 57-61); WO200012130 (pages 24-26);

(34) FCRH1 (Fc receptor-like protein 1, a putative receptor for the immunoglobulin Fc domain that contains C2 type Ig-like and ITAM domains, may have a role in B-lymphocyte differentiation) 429 aa, pI: 5.28, MW: 46925 TM: 1 [P] Gene Chromosome: 1q21-1q22, Genbank accession No. NP_{—}443170.1)

WO2003077836; WO200138490 (claim 6, FIG. 18E-1-18-E-2); Davis et al (2001) Proc. Natl. Acad. Sci. USA 98(17):9772-9777; WO2003089624 (claim 8); EP1347046 (claim 1); WO2003089624 (claim 7);

(35) IRTA2 (Immunoglobulin superfamily receptor translocation associated 2, a putative immunoreceptor with possible roles in B cell development and lymphomagenesis; deregulation of the gene by translocation occurs in some B cell malignancies) 977 aa, pI: 6.88 MW: 106468 TM: 1 [P] Gene Chromosome: 1q21, Genbank accession No. NP_{—}112571.1)

WO2003024392 (claim 2, FIG. 97); Nakayama et al (2000) Biochem. Biophys. Res. Commun. 277(1):124-127; WO2003077836; WO200138490 (claim 3, FIG. 18B-1-18B-2);

(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_{—}057276; NCBI Gene: 23671; OMIM: 605734; SwissProt Q9UIK5; Genbank accession No. AF179274; AY358907, CAF85723, CQ782436

WO2004074320 (SEQ ID NO 810); JP2004113151 (SEQ ID NOS 2, 4, 8); WO2003042661 (SEQ ID NO 580); WO2003009814 (SEQ ID NO 411); EP1295944 (pages 69-70); WO200230268 (page 329); WO200190304 (SEQ ID NO 2706); US2004249130; US2004022727; WO2004063355; US2004197325; US2003232350; US2004005563; US2003124579; Horie et al (2000) Genomics 67:146-152; Uchida et al (1999) Biochem. Biophys. Res. Commun. 266:593-602; Liang et al (2000) Cancer Res. 60:4907-12; Glynne-Jones et al (2001) Int J Cancer. Oct 15;94(2):178-84.

For other tumor-associated antigens and specific antibodies thereto, see also: WO04/045516 (3 Jun. 2004); WO03/000113 (3 Jan. 2003); WO02/016429 (28 Feb. 2002); WO02/16581 (28 Feb. 2002); WO03/024392 (27 Mar. 2003); WO04/016225 (26 Feb. 2004); WO 01/40309 (7 Jun. 2001); US 20050238650 A1; all of which are incorporated herein by reference in their entirety.

Production of Recombinant Antibodies

Antibodies of the invention can be produced using any method known in the art to be useful for the synthesis of antibodies, in particular, by chemical synthesis or by recombinant expression techniques.

Recombinant expression of antibodies, or fragment, derivative or analog thereof, may be conducted by assembling a nucleic acid encoding the antibody, if the nucleotide sequence of the antibody is known, from chemically synthesized oligonucleotides (e.g., as described in Kutmeier et al., 1994, BioTechniques 17:242). This method involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody can be generated from a suitable source. If a clone containing the nucleic acid encoding the particular antibody is not available, but the sequence of the antibody is known, a nucleic acid encoding the antibody can be obtained from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

If an antibody that specifically recognizes a particular antigen is not commercially available (or a source for a cDNA library for cloning a nucleic acid encoding such an immunoglobulin), antibodies specific for a particular antigen can be generated by any method known in the art, for example, by immunizing a patient, such as a rabbit, to generate polyclonal antibodies or, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody can be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid sequence encoding at least the variable domain of the antibody is obtained, it can be introduced into a vector containing the nucleotide sequence encoding the constant regions of the antibody (see, e.g., WO 86/05807; WO 89/01036; and U.S. Pat. No. 5,122,464). Vectors containing the complete light or heavy chain that allow for the expression of a complete antibody molecule are available. Then, the nucleic acid encoding the antibody can be used to introduce the nucleotide substitutions or deletion necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis and in vitro site directed mutagenesis (Hutchinson et al., 1978, J. Biol. Chem. 253:6551).

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., 1988, Science 242:1038-1041).

Antibody fragments that recognize specific epitopes can be generated by known techniques. For example, such fragments include, but are not limited to, the F(ab′)2 fragments that can be produced by pepsin digestion of the antibody molecule and the Fab fragments that can be generated by reducing the disulfide bridges of the F(ab′)2 fragments.

Once a nucleic acid sequence encoding an antibody has been obtained, the vector for the production of the antibody can be produced by recombinant DNA technology using techniques well known in the art. Methods that are well known to those skilled in the art can be used to construct expression vectors containing the antibody coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2^{nd} Ed., Cold Spring Harbor Laboratory, Cold Spring Harbor, N.Y.) and Ausubel et al. (eds., 1998, Current Protocols in Molecular Biology, John Wiley & Sons, NY).

Polyclonal antibodies may be raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). Lymphocytes then are fused with myeloma cells using a suitable fusing agent, such as polyethylene glycol, to form a hybridoma cell (Goding, Monoclonal Antibodies: Principles and Practice, pp. 59-103, Academic Press, 1986). Review articles on recombinant expression in bacteria of DNA encoding the antibody include Skerra et al., Curr. Opinion in Immunol., 5:256-262 (1993) and Plückthun, Immunol. Revs., 130:151-188 (1992).

Monoclonal antibodies or antibody fragments can be isolated from antibody phage libraries generated using the techniques described in McCafferty et al (1990) Nature 348:552-554; Clackson et al (1991) Nature, 352:624-628; and Marks et al (1991) J. Mol. Biol. 222:581-597. Subsequent publications describe the production of high affinity (nm range) human antibodies by chain shuffling (Marks et al (1992) Bio/Technology, 10:779-783) as well as combinatorial infection and in vivo recombination as a strategy for constructing very large phage libraries (Waterhouse et al (1993) Nuc. Acids. Res., 21:2265-2266). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al (1984) Proc. Natl. Acad. Sci. USA 81:6851), or by covalently joining to the immunoglobulin coding sequence all or part of the coding sequence for a non-immunoglobulin polypeptide.

Humanization can be performed by substituting hypervariable region sequences for the corresponding sequences of a human antibody (Jones et al (1986) Nature 321:522-525; Riechmann et al (1988) Nature 332:323-327; Verhoeyen et al (1988) Science 239:1534-1536). Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies (Sims et al (1993) J. Immunol., 151:2296; Chothia et al (1987) J. Mol. Biol., 196:901; Carter et al (1992) Proc. Natl. Acad. Sci. USA, 89:4285; Presta et al., (1993) J. Immunol., 151:2623).

Various forms of the humanized antibody are contemplated. For example, the humanized antibody may be an antibody fragment, such as a Fab. Alternatively, the humanized antibody may be an intact antibody, such as an intact IgG1 antibody. The murine monoclonal antibody 4D5 which specifically binds the extracellular domain of ErbB2 is produced, as described in Fendly et al (1990) Cancer Research 50:1550-1558, from NIH 3T3/HER2-3₄₀₀ cells (expressing approximately 1×10⁵ ErbB2 molecules/cell), as described in Hudziak et al (1987) Proc. Natl. Acad. Sci. (USA) 84:7158-7163 and harvested with phosphate buffered saline (PBS) containing 25 mM EDTA and used to immunize BALB/c mice. Hybridoma supernatants were screened for ErbB2-binding by ELISA and radioimmunoprecipitation.

As an alternative to humanization, human antibodies can be generated (Jakobovits et al (1993) Proc. Natl. Acad. Sci. USA, 90:2551; Jakobovits et al (1993) Nature, 362:255-258; Bruggermann et al (1993) Year in Immuno. 7:33; and U.S. Pat. No. 5,591,669, U.S. Pat. No. 5,589,369, U.S. Pat. No. 5,545,807).

Alternatively, phage display technology (McCafferty et al., (1990) Nature 348:552-553) can be used to produce human antibodies and antibody fragments in vitro, from immunoglobulin variable (V) domain gene repertoires from unimmunized donors (Johnson, Kevin S. and Chiswell, David J., (1993) Current Opinion in Structural Biology 3:564-571; Clackson et al (1991) Nature, 352:624-628). Human antibodies may also be generated by in vitro activated B cells (see U.S. Pat. No. 5,567,610 and U.S. Pat. No. 5,229,275). Human anti-ErbB2 antibodies are described in U.S. Pat. No. 5,772,997 and WO 97/00271.

Various techniques have been developed for the production of antibody fragments (Morimoto et al (1992) Journal of Biochem. and Biophys. Methods 24:107-117; and Brennan et al (1985) Science, 229:81; Carter et al (1992) Bio/Technology 10:163-167; WO 93/16185; U.S. Pat. No. 5,571,894; U.S. Pat. No. 5,587,458; U.S. Pat. No. 5,641,870).

Amino acid sequence modification(s) of the antibodies described herein are contemplated. For example, it may be desirable to improve the binding affinity and/or other biological properties of the antibody. Amino acid sequence variants are prepared by introducing appropriate nucleotide changes into the antibody expressing nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or -position of glycosylation sites.

A useful method for identification of certain residues or regions of an antibody that are preferred locations for mutagenesis is called “alanine scanning mutagenesis” (Cunningham and Wells (1989) Science, 244:1081-1085. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the antibody fused to a cytotoxic polypeptide. Other insertional variants include the fusion to the N- or C-terminus of the antibody to an enzyme (e.g. for ADEPT) or a polypeptide which increases the serum half-life of the antibody.

Peptide sequences which specifically bind to albumin may be fused or conjugated to the antibody which comprises the antibody drug conjugates (ADC). Plasma-protein binding can be an effective means of improving the pharmacokinetic properties of short lived molecules, such as antibodies or ADC. Serum albumin binding peptides (ABP) can alter the pharmacodynamics of fused active domain proteins, including alteration of tissue uptake, clearance, penetration, and diffusion, and increase serum half life. These pharmacodynamic parameters can be modulated by specific selection of the appropriate serum albumin binding peptide sequence (US 20040001827 at [0076]). A series of albumin binding peptides were identified by phage display screening (Dennis et al. (2002) J Biol. Chem. 277:35035-35043 at Tables III and IV, page 35038; WO 01/45746); and WO 01/45746 at pages 12-13, all of which are incorporated herein by reference.

Another type of variant is an amino acid substitution variant. The sites of greatest interest for substitutional mutagenesis include the hypervariable regions, but FR alterations are also contemplated.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Some antibodies are glycosylated at conserved positions in their constant regions (Jefferis and Lund, (1997) Chem. Immunol. 65:111-128; Wright and Morrison, (1997) TibTECH 15:26-32). The oligosaccharide side chains of the immunoglobulins affect protein function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Hefferis and Lund, supra; Wyss and Wagner, (1996) Current Opin. Biotech. 7:409-416; Malhotra et al., (1995) Nature Med. 1:237-243; Hse et al., (1997) J. Biol. Chem. 272:9062-9070; U.S. Pat. No. 5,047,335; U.S. Pat. No. 5,510,261; U.S. Pat. No. 5,278,299).

In Vitro Cell Proliferation Assays

Generally, the cytotoxic or cytostatic activity of an antibody drug conjugate (ADC) is measured by: exposing mammalian cells having receptor proteins, e.g. HER2, to the antibody of the ADC in a cell culture medium; culturing the cells for a period from about 6 hours to about 5 days; and measuring cell viability. Cell-based in vitro assays were used to measure viability (proliferation), cytotoxicity, and induction of apoptosis (caspase activation) of the ADC of the invention.

The in vitro potency of antibody drug conjugates was measured by a cell proliferation assay (FIGS. 1-5). The CellTiter-Glo® Luminescent Cell Viability Assay is a commercially available (Promega Corp., Madison, Wis.), homogeneous assay method based on the recombinant expression of Coleoptera luciferase (U.S. Pat. No. 5,583,024; U.S. Pat. No. 5,674,713 and U.S. Pat. No. 5,700,670). This cell proliferation assay determines the number of viable cells in culture based on quantitation of the ATP present, an indicator of metabolically active cells (Crouch et al (1993) J. Immunol. Meth. 160:81-88, U.S. Pat. No. 6,602,677). The CellTiter-Glo® Assay was conducted in 96 well format, making it amenable to automated high-throughput screening (HTS) (Cree et al (1995) AntiCancer Drugs 6:398-404). The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells/well in a 384-well format in 10 minutes after adding reagent and mixing. The cells may be treated continuously with ADC, or they may be treated and separated from ADC. Generally, cells treated briefly, i.e. 3 hours, showed the same potency effects as continuously treated cells.

The homogeneous “add-mix-measure” format results in cell lysis and generation of a luminescent signal proportional to the amount of ATP present. The amount of ATP is directly proportional to the number of cells present in culture. The CellTiter-Glo® Assay generates a “glow-type” luminescent signal, produced by the luciferase reaction, which has a half-life generally greater than five hours, depending on cell type and medium used. Viable cells are reflected in relative luminescence units (RLU). The substrate, Beetle Luciferin, is oxidatively decarboxylated by recombinant firefly luciferase with concomitant conversion of ATP to AMP and generation of photons. The extended half-life eliminates the need to use reagent injectors and provides flexibility for continuous or batch mode processing of multiple plates. This cell proliferation assay can be used with various multiwell formats, e.g. 96 or 384 well format. Data can be recorded by luminometer or CCD camera imaging device. The luminescence output is presented as relative light units (RLU), measured over time.

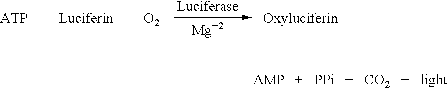

The anti-proliferative effects of antibody drug conjugates were measured by the cell proliferation, in vitro cell killing assay above against two breast tumor cell lines (FIGS. 1-5). IC₅₀ values of the ADC were established against SK-BR-3 and BT-474 (Table 2), which are known to over-express HER2 receptor protein.

FIG. 1 shows an in vitro, cell proliferation assay with SK-BR-3 cells treated with: -o-trastuzumab and -●- trastuzumab-MC-vc-PAB-(N,N′-(bis-aminoethyl-1,3-propanediamine)-bis-(4-N-imidazolyl)-1,8 naphthalimide) 202, measured in Relative Fluorescence Units (RLU) versus μg/ml concentration of antibody or ADC. H=trastuzumab, where H is linked via a cysteine [cys].

FIG. 2 shows an in vitro, cell proliferation assay with SK-BR-3 cells treated with: -●-Trastuzumab and -Δ- trastuzumab-MC-ala-phe-PAB-(N,N′-(bis-aminoethyl-1,3-propanediamine)-bis-(4-N-imidazolyl)-1,8 naphthalimide) 203, measured in Relative Fluorescence Units (RLU) versus μg/ml concentration of antibody or ADC. H=trastuzumab, where H is linked via a cysteine [cys].

FIG. 3 shows an in vitro, cell proliferation assay with BT-474 cells treated with: —O-trastuzumab, and —O— trastuzumab-(succinate-gly-ala-phe)-(N,N′-(bis-aminoethyl-1,3-propanediamine)-bis 3-nitro-1,8 naphthalimide) 204, measured in Relative Fluorescence Units (RLU) versus μg/ml concentration of antibody or ADC. H=trastuzumab, where H is linked via an amino group.

FIG. 4 shows an in vitro, cell proliferation assay with BT-474 cells treated with: -●-trastuzumab, and -▴- trastuzumab-(MC-val-cit-PAB-(N,N′-(N,N′-(bis-aminoethyl-1,3-propanediamine)-3-nitro, 4-amino-1,8 naphthalimide) 205, measured in Relative Fluorescence Units (RLU) versus μg/ml concentration of antibody or ADC. H=trastuzumab, where H is linked via a cysteine [cys].

FIG. 5 shows an in vitro, cell proliferation assay with SK-BR-3 cells treated with: -●-trastuzumab, -♦- trastuzumab-MC-(N,N′-(bis-aminoethyl-1,3-propanediamine)-bis-(4-N-imidazolyl)-1,8 naphthalimide) 206, and -▾- trastuzumab-N¹-cyclopropylmethyl, N²-maleimidopropyl-gly-val-cit-PAB-(N, N′-(bis-aminoethyl-1,3-propanediamine)-bis 3-nitro-1,8 naphthalimide) 207, measured in Relative Fluorescence Units (RLU) versus μg/ml concentration of antibody or ADC. H=trastuzumab, where H is linked via a cysteine [cys].

TABLE 2

| | IC₅₀ (μg ADC/ml) | IC₅₀ (μg ADC/ml) BT- |
|---|---|---|
| Antibody Bis 1,8 naphthalimide Drug Conjugate | SK-BR-3 cells | 474 cells |
| H-MC-vc-PAB-(bis 4-imidazolyl E) 202 | 0.04 | 0.021 |
| H-MPG-vc-PAB-(bis 4-imidazolyl E) | 0.018 | 0.012 |
| H-MPEG-vc-PAB-(bis 4-imidazolyl E) | 0.018-0.030 | 0.012-0.033 |
| H-MC-gg-PAB-(bis 4 imidazolyl E) | 0.045 | NSA |
| H-MC-af-PAB-(bis 4-imidazolyl E) 203 | 0.22 | 0.013 |
| H-MC-(bis 4-imidazolyl E) 206 | 0.045 | NSA |
| H-MC-hydrazone-E | NSA | NSA |
| H-MC-hydrazone-1-E | NSA | 0.39 |
| H-MC-hydrazone-2-E | NSA | NSA |
| H-succinate-gaf-PAB-(bis 3-nitro E) 204 | 0.18 | 0.22 |
| H is linked via an amino group | | |
| H-MC-vc-PAB-N(Me)val, N′-ethyl (bis 3-nitro E) | NSA | NSA |
| H-MPG-vc-PAB-N(Me)val-N′-ethyl (3-nitro E) | NSA | 0.25 |
| H-MC-vc-PAB-(bis 4-morpholino E) | 0.085 | 0.33 |
| H-MC-hydrazone-(bis 3-nitro E) | NSA | NSA |
| H-MPG-vc-PAB-N(cyclopropylmethyl)-(bis 3 | 0.20 | 0.12 |
| nitro E) 207 | | |
| H-MC-hydrazone-(bis 3-morpholino E) | NSA | NSA |
| H-MC-vc-PAB-(4-amino, 3-nitro E) 205 | 0.17 | 0.15 |
| H-MC-vc-PAB-(bis 4,4′-piperidine E) | NSA | NSA |
| H-MPG-vc-PAB-(bis 4,4′-piperidine E) | 0.19 | 0.45 |
| H-MC-(4′-Me-Piperidine E) | NSA | NSA |
| H-MPG-af-PAB-(bis 4-pip, 3-nitro E) | NSA | NSA |
| H-SMCC-E | NSA | NSA |
| H-SPP-E | NSA | 1.1 |
| (trastuzumab) | (0.10-0.20) | (0.10-0.30) |
| 2H9 (anti-EphB2R)-MC-vc-PAB-(bis 4- | — | 0.42 |
| imidazolyl E) 202 | | |

H = trastuzumab linked via a cysteine [cys] except where noted.
E = bis 1,8 naphthalimide
NSA = no significant activity

Antibody drug conjugates of Formula I were prepared where Ab included anti-EphB2R and anti-CD22 antibodies. These conjugates also showed in vitro cytotoxic or cytostatic activity.

In Vivo Serum Clearance and Stability in Mice

Serum clearance and stability of ADC may be investigated in nude, naive (without tumors received by exogenous grafts) mice. A difference in the amount of total antibody and ADC indicates cleavage of the linker and separation of the antibody from its drug moiety.

In Vivo Efficacy

Efficacy of the antibody-drug conjugates of the invention was measured in vivo by implanting allografts or xenografts of cancer cells in rodents and treating the tumors with ADC. Variable results are to be expected depending on the cell line, the specificity of antibody binding of the ADC to receptors present on the cancer cells, dosing regimen, and other factors. For example, the in vivo efficacy of anti-HER2 ADC was measured by a high expressing HER2 transgenic explant mouse model. An allograft may be propagated from the Fo5 MMTV transgenic mouse which does not respond to, or responds poorly to, HERCEPTIN therapy. Subjects were treated once with ADC and monitored over 3-6 weeks to measure the time to tumor doubling, log cell kill, and tumor shrinkage. The ADC of the invention showed only modest efficacy in slowing the progression of tumor growth. For example, an IV administration of 10 mg H-MC-af-PAB-(bis 4-imidazolyl E) 203 per kg animal showed only a slight increase in the time for mean MMTV-HER2 Fo5 tumor volume doubling in athymic nude mice relative to control (injection vehicle, PBS buffer). Follow up dose-response and multi-dose experiments may be conducted.

Rodent Toxicity

Antibody-drug conjugates and an ADC-minus control, “Vehicle”, may be evaluated in an acute toxicity rat model (Brown et al (2002) Cancer Chemother. Pharmacol. 50:333-340). Toxicity of ADC may be investigated by treatment of rats with the ADC and subsequent inspection and analysis of the effects on various organs. Based on gross observations (body weights), clinical pathology parameters (serum chemistry and hematology) and histopathology, the toxicity of ADC may be observed, characterized, and measured. Clinical chemistry, serum enzymes and hematology analysis may also be conducted periodically; concluding with complete necropsy with histopathological assessment. Toxicity signals included the clinical observation of weight loss, considering that weight loss, or weight change relative to animals dosed only with Vehicle in animals after dosing with ADC, is a gross and general indicator of systemic or localized toxicity. Hepatotoxicity may be measured by: (i) elevated liver enzymes such as AST (aspartate aminotransferase), ALT (alanine aminotransferase), GGT (g-glutamyl transferase); (ii) increased numbers of mitotic and apoptotic figures; and (iii) hepatocyte necrosis. Hematolymphoid toxicity is observed by depletion of leukocytes, primarily granuloctyes (neutrophils), and/or platelets, and lymphoid organ involvement, i.e. atrophy or apoptotic activity. Toxicity is also noted by gastrointestinal tract lesions such as increased numbers of mitotic and apoptotic figures and degenerative entercolitis.

Synthesis of Antibody Drug Conjugates

The Antibody Drug Conjugates (ADC) of the Invention can be made using the synthetic procedures outlined below. ADC can be conveniently prepared using a Linker having a reactive site for binding to the Drug and Antibody. In one aspect, a Linker has a reactive site which has an electrophilic group that is reactive to a nucleophilic group present on an antibody. Useful nucleophilic groups on an antibody include but are not limited to, sulfhydryl, hydroxyl and amino groups. The heteroatom of the nucleophilic group of an antibody is reactive to an electrophilic group on a Linker and forms a covalent bond to a Linker unit. Useful electrophilic groups include, but are not limited to, maleimide and haloacetamide groups. The electrophilic group provides a convenient site for Antibody attachment.

In another embodiment, a Linker has a reactive site which has a nucleophilic group that is reactive to an electrophilic group present on an antibody. Useful electrophilic groups on an antibody include, but are not limited to, aldehyde and ketone carbonyl groups. The heteroatom of a nucleophilic group of a Linker can react with an electrophilic group on an antibody and form a covalent bond to an antibody unit. Useful nucleophilic groups on a Linker include, but are not limited to, hydrazide, oxime, amino, hydrazine, thiosemicarbazone, hydrazine carboxylate, and arylhydrazide. The electrophilic group on an antibody provides a convenient site for attachment to a Linker.

Carboxylic acid functional groups and chloroformate functional groups are also useful reactive sites for a Linker because they can react with secondary amino groups of a Drug to form an amide linkage. Also useful as a reactive site is a carbonate functional group on a Linker, such as but not limited to p-nitrophenyl carbonate, which can react with an amino group of a Drug, such as but not limited to N-methyl valine, to form a carbamate linkage. Typically, peptide-based Drugs can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, 1965, Academic Press) that is well known in the field of peptide chemistry.

As described in more detail below, the ADC of the Invention are conveniently prepared using a Linker having two or more reactive functional groups for binding to the Drug and Antibody. In one aspect of the invention, a Linker has an electrophilic group that is reactive with a nucleophilic group present on an antibody. Useful nucleophilic groups on a Antibody include but are not limited to, sulfhydryl, hydroxyl and amino groups. The heteroatom of the nucleophilic group of an antibody is reactive to an electrophilic group on a Linker and forms a covalent bond to a Linker unit. Useful electrophilic groups include, but are not limited to, maleimide, carbonate, and haloacetamide groups. The electrophilic group provides a convenient site for Antibody attachment.

In another embodiment, a Linker has a reactive functional group which has a nucleophilic group that is reactive to an electrophilic group present on an antibody. Useful electrophilic groups on an Antibody include, but are not limited to, aldehyde and ketone carbonyl groups. The heteroatom of a nucleophilic group of a Linker can react with an electrophilic group on an antibody and form a covalent bond to an antibody unit. Useful nucleophilic groups on a Linker include, but are not limited to, hydrazide, oxime, amino, hydrazine, thiosemicarbazone, hydrazine carboxylate, and arylhydrazide. The electrophilic group on an antibody provides a convenient site for attachment to a Linker.

Typically, peptide-type Linkers can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, (1965), Academic Press) that is well known in the field of peptide chemistry.

Linker intermediates may be assembled with any combination or sequence of reactions including Spacer, Stretcher, and Amino Acid units. The Spacer, Stretcher, and Amino Acid units may employ reactive functional groups which are electrophilic, nucleophilic, free radical in nature. Reactive functional groups include, but are not limited to:

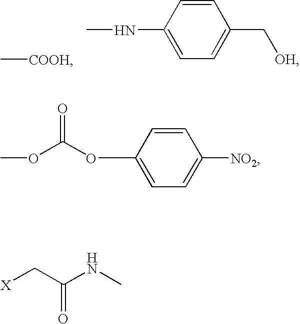

where X is a leaving group, e.g. O-mesyl, O-tosyl, —Cl, —Br, —I, an alkyldisulfide or aryidisulfide (RSS-), or a maleimide group.

In another embodiment, the Linker may be substituted with groups which modulated solubility or reactivity. For example, a sulfonate substituent may increase water solubility of the reagent and facilitate the coupling reaction of the linker reagent with the antibody or the drug moiety, or facilitate the coupling reaction of Ab-L with D, or D-L with Ab, depending on the synthetic route employed to prepare the ADC.

The compounds of the invention expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available from Pierce Biotechnology, Inc., Rockford, Il. 61105 U.S.A. See pages 467-498, 2003-2004 Applications Handbook and Catalog.

Useful Linkers can also be obtained from other commercial sources, such as Molecular Biosciences Inc. (Boulder, Colo.), or synthesized in accordance with procedures described in U.S. Pat. No. 6,214,345 to Firestone et al, J. Org. Chem. 1995, 60, 5352-5), Frisch, et al., (1996) Bioconjugate Chem., 7, 180-186.

Useful Stretchers may be incorporated into a Linker using the commercially available intermediates from Molecular Biosciences (Boulder, Colo.) described below by utilizing known techniques of organic synthesis.

Stretchers of formula (IIIa) can be introduced into a Linker by reacting the following intermediates with the N-terminus of an Amino Acid unit: 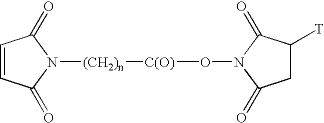
where n is an integer ranging from 1-10 and T is —H or —SO₃Na; 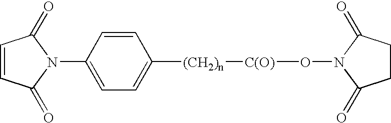
where n is an integer ranging from 0-3;

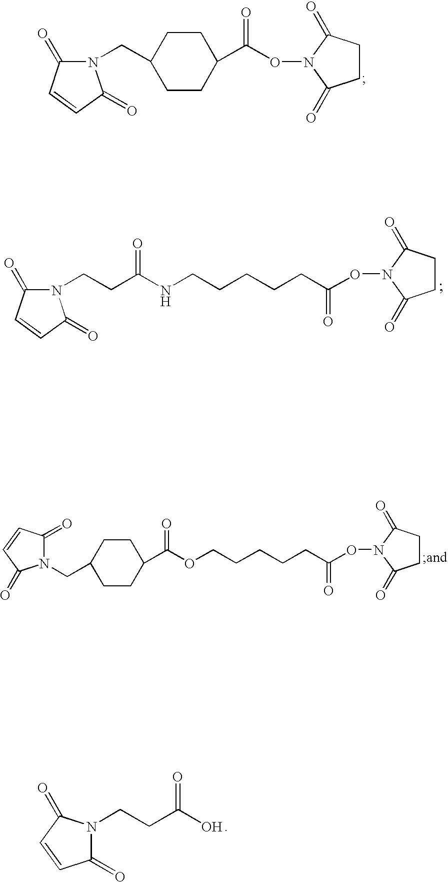

Stretcher units of can be introduced into a Linker by reacting the following bifunctional reagents with the N-terminus of an Amino Acid unit:

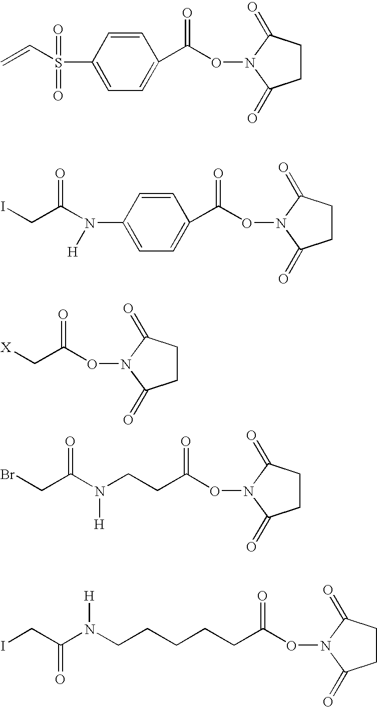

where X is Br or I. Stretcher units of Formula IIIa and IIIb can also be introduced into a Linker by reacting the following bifunctional reagents with the N-terminus of an Amino Acid unit:

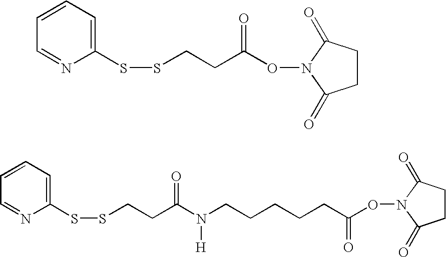

Stretcher units of formula (Va) can be introduced into a Linker by reacting the following intermediates with the N-terminus of an Amino Acid unit:

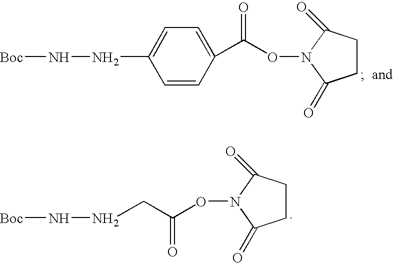

Other useful Stretchers may be synthesized according to known procedures. Aminooxy Stretchers (H₂N—O—R¹⁷—C(O)—) can be prepared by treating alkyl halides with N-Boc-hydroxylamine according to procedures described in Jones, D. S. et al., Tetrahedron Letters, 2000, 41(10), 1531-1533; and Gilon, C. et al., Tetrahedron, 1967, 23(11), 4441-4447, wherein -R¹⁷- is selected from -C₁-C₁₀ alkylene-, -C₃-C₈ carbocyclo-, —O—(C₁-C₈ alkyl)-, -arylene-, -C₁-C₁₀ alkylene-arylene-, -arylene-C₁-C₁₀ alkylene-, -C₁-C₁₀ alkylene-(C₃-C₈ carbocyclo)-, —(C₃-C₈ carbocyclo)-C₁-C₁₀ alkylene-, -C₃-C₈ heterocyclo-, -C₁-C₁₀alkylene-(C₃-C₈ heterocyclo)-, -(C₃-C₈ heterocyclo)-C₁-C₁₀alkylene-, —(CH₂CH₂O)ᵣ—, —(CH₂CH₂O), —CH₂—; and r is an integer ranging from 1-10. Isothiocyanate Stretchers (S═C═N—R¹⁷—C(O)—) may be prepared from isothiocyanatocarboxylic acid chlorides as described in Angew. Chem., 1975, 87(14), 517.

FIG. 6 shows a method for preparing a valine-citrulline (val-cit or vc) dipeptide Linker having a maleimide Stretcher and optionally a p-aminobenzyloxycarbonyl (PAB) self-immolative Spacer where Q is —C₁-C₈ alkyl, —O—(C₁-C₈ alkyl), -halogen, -nitro or -cyano; and m is an integer ranging from 0-4.

FIG. 7 illustrates the synthesis of a phe-lys(Mtr) dipeptide Linker unit having a maleimide Stretcher unit and a p-aminobenzyloxycarbonyl self-immolative Spacer unit, where Q is -C₁-C₈ alkyl, —O—(C₁-C₈ alkyl), -halogen, -nitro or -cyano; and m is an integer ranging from 0-4. Starting material, lys(Mtr), is commercially available (Bachem, Torrance, Calif.) or can be prepared according to Dubowchik, et al. (1997) Tetrahedron Letters, 38:5257-60.

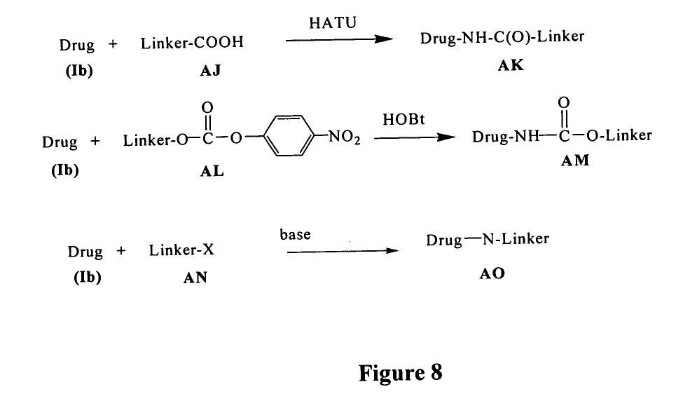

FIG. 8 shows a Linker reacted with an amino group of a Drug moiety to form an ADC that contains an amide or carbamate group, linking the Drug unit to the Linker unit. When a linker intermediate has a carboxylic acid group, as in Linker AJ, the coupling reaction can be performed using HATU or PyBrop and an appropriate amine base, resulting in a Drug-Linker Compound AK, containing an amide bond between the Drug unit and the Linker unit. When the functional group is a carbonate, as in Linker AL, the Linker can be coupled to the Drug using HOBt in a mixture of DMF/pyridine to provide a Drug-Linker Compound AM, containing a carbamate bond between the Drug unit and the Linker unit Alternately, when the reactive functional group is a good leaving group, such as halide in Linker AN, the Linker can be coupled with an amine group of a Drug via a nucleophilic substitution process to provide a Drug-Linker Compound having an amine linkage (AO) between the Drug unit and the Linker unit. Illustrative methods useful for linking a Drug to an antibody to form a Drug-Linker Compound are depicted in FIG. 8 and are outlined in General Procedures G-H.

General Procedure G: Amide formation using HATU. A Drug (Ib) (1.0 eq.) and an N-protected Linker containing a carboxylic acid group (1.0 eq.) are diluted with a suitable organic solvent, such as dichloromethane, and the resulting solution is treated with HATU (1.5 eq.) and an organic base, such as pyridine (1.5 eq.). The reaction mixture is allowed to stir under an inert atmosphere, such as argon, for 6h, during which time the reaction mixture is monitored using HPLC. The reaction mixture is concentrated and the resulting residue is purified using HPLC to yield the amide of formula AK.

General Procedure H: Carbamate formation using HOBt. A mixture of a Linker AL having a p-nitrophenyl carbonate (1.1 eq.) and Drug (Ib) (1.0 eq.) are diluted with an aprotic organic solvent, such as DMF, to provide a solution having a concentration of 50-100 mM, and the resulting solution is treated with HOBt (2.0 eq.) and placed under an inert atmosphere, such as argon. The reaction mixture is allowed to stir for 15 min, then an organic base, such as pyridine (1/4 v/v), is added and the reaction progress is monitored using HPLC. The Linker is typically consumed within 16 h. The reaction mixture is then concentrated in vacuo and the resulting residue is purified using, for example, HPLC to yield the carbamate AM.

An alternate method of preparing Drug-Linker Compounds is outlined in FIG. 8 where a drug moiety D is reacted with a Linker reagent, which does not have a Stretcher unit attached. This provides intermediate AP, which has an Amino Acid unit having an Fmoc-protected N-terminus. The Fmoc group is then removed and the resulting amine intermediate AQ is then attached to a Stretcher unit via a coupling reaction catalyzed using PyBrop or DEPC. The construction of Drug-Linker Compounds containing either a bromoacetamide Stretcher AR or a PEG maleimide Stretcher AS is illustrated in FIG. 9 where Q is -C₁-C₈ alkyl, —O—(C₁-C₈ alkyl), -halogen, -nitro or -cyano; and m is an integer ranging from 0-4.

FIG. 10 shows the preparation of a Linker unit containing a branched spacer is shown in, which illustrates the synthesis of a val-cit dipeptide linker having a maleimide Stretcher unit and a bis(4-hydroxymethyl)styrene (BHMS) unit. The synthesis of the BHMS intermediate (AW) has been improved from previous literature procedures (see WO 98/13059 and Crozet, et al (1985) Tetrahedron Lett., 26:5133-5134) and utilizes as starting materials, commercially available diethyl (4-nitrobenzyl)phosphonate (AT) and commercially available 2,2-dimethyl-1,3-dioxan-5-one (AU). Linkers AY and BA can be prepared from intermediate AW.

Conjugation of Drug Moieties to Antibodies

One exemplary method of preparing an antibody for conjugation with a bis 1,8 naphthalimide drug moiety of the invention entails treating the antibody with a reducing agent, such as dithiothreitol (DTT) to reduce some or all of the cysteine disulfide residues to form highly nucleophilic cysteine thiol groups (—CH₂SH). The partially reduced antibody thus reacts with bis 1,8 naphthalimide drug-linker compounds, or linker reagents with electrophilic functional groups such as maleimide or α-halo carbonyl, according to the conjugation method at page 766 of Klussman, et al (2004), Bioconjugate Chemistry 15(4):765-773.

For example, an antibody, e.g. trastuzumab, dissolved in 500 mM sodium borate and 500 mM sodium chloride at pH 8.0 is treated with an excess of 100 mM dithiothreitol (DTT). After incubation at 37° C. for about 30 minutes, the buffer is exchanged by elution over Sephadex G25 resin and eluted with PBS with 1 mM DTPA. The thiol/Ab value is checked by determining the reduced antibody concentration from the absorbance at 280 nm of the solution and the thiol concentration by reaction with DTNB (Aldrich, Milwaukee, Wis.) and determination of the absorbance at 412 nm. The reduced antibody dissolved in PBS is chilled on ice. The drug linker, e.g. MC-val-cit-PAB-bis 1,8 naphthalimide in DMSO, dissolved in acetonitrile and water at known concentration, is added to the chilled reduced antibody in PBS. After about one hour, an excess of maleimide is added to quench the reaction and cap any unreacted antibody thiol groups. The reaction mixture is concentrated by centrifugal ultrafiltration and the ADC, e.g. trastuzumab-MC-vc-PAB-bis 1,8 naphthalimide, is purified and desalted by elution through G25 resin in PBS, filtered through 0.2 μm filters under sterile conditions, and frozen for storage.

Administration of Antibody Drug Conjugates and Pharmaceutical Formulations

The antibody drug conjugates (ADC) of the invention may be administered by any route appropriate to the condition to be treated. The ADC will typically be administered parenterally, i.e. infusion, subcutaneous, intramuscular, intravenous, intradermal, intrathecal and epidural.

Pharmaceutical formulations of therapeutic antibody drug conjugates (ADC) of the invention are typically prepared for parenteral administration, i.e. bolus, intravenous, intratumor injection with a pharmaceutically acceptable parenteral vehicle and in a unit dosage injectable form. An antibody-drug conjugate (ADC) having the desired degree of purity is optionally mixed with pharmaceutically acceptable diluents, carriers, excipients or stabilizers (Remington's Pharmaceutical Sciences (1980) 16th edition, Osol, A. Ed.), in the form of a lyophilized formulation or an aqueous solution.

Acceptable diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). For example, lyophilized anti-ErbB2 antibody formulations are described in WO 97/04801, expressly incorporated herein by reference.

The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the ADC, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile, which is readily accomplished by filtration through sterile filtration membranes.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Aqueous suspensions of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The pharmaceutical compositions of ADC may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Although oral administration of protein therapeutics are disfavored due to hydrolysis or denaturation in the gut, formulations of ADC suitable for oral administration may be prepared as discrete units such as capsules, cachets or tablets each containing a predetermined amount of the ADC.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Exemplary unit dosage formulations include a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

Antibody Drug Conjugate Treatments

It is contemplated that the antibody drug conjugates (ADC) of the present invention may be used to treat various diseases or disorders, e.g. characterized by the overexpression of a tumor antigen. Exemplary conditions or disorders include benign or malignant tumors; leukemia and lymphoid malignancies; other disorders such as neuronal, glial, astrocytal, hypothalamic, glandular, macrophagal, epithelial, stromal, blastocoelic, inflammatory, angiogenic and immunologic disorders.

The ADC compounds which are identified in the animal models and cell-based assays can be further tested in tumor-bearing higher primates and human clinical trials. Human clinical trials can be designed similar to the clinical trials testing the efficacy of the anti-HER2 monoclonal antibody HERCEPTIN in patients with HER2 overexpressing metastatic breast cancers that had received extensive prior anti-cancer therapy as reported by Baselga et al. (1996) J. Clin. Oncol. 14:737-744. The clinical trial may be designed to evaluate the efficacy of an ADC in combinations with known therapeutic regimens, such as radiation and/or chemotherapy involving known chemotherapeutic and/or cytotoxic agents.

Generally, the disease or disorder to be treated is cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The cancer will generally comprise HER2-expressing cells, such that the ADC of the present invention are able to bind to the cancer cells. To determine ErbB2 expression in the cancer, various diagnostic/prognostic assays are available. In one embodiment, ErbB2 overexpression may be analyzed by IHC, e.g. using the HERCEPTEST (Dako). Parrafin embedded tissue sections from a tumor biopsy may be subjected to the IHC assay and accorded a ErbB2 protein staining intensity criteria as follows: Score 0, no staining is observed or membrane staining is observed in less than 10% of tumor cells; Score 1+, a faint/barely perceptible membrane staining is detected in more than 10% of the tumor cells, the cells are only stained in part of their membrane; Score 2+, a weak to moderate complete membrane staining is observed in more than 10% of the tumor cells; Score 3+, a moderate to strong complete membrane staining is observed in more than 10% of the tumor cells.

Those tumors with 0 or 1+ scores for ErbB2 overexpression assessment may be characterized as not overexpressing ErbB2, whereas those tumors with 2+ or 3+ scores may be characterized as overexpressing ErbB2.

Alternatively, or additionally, FISH assays such as the INFORM™ (Ventana Co., Ariz.) or PATHVISION™ (Vysis, Ill.) may be carried out on formalin-fixed, paraffin-embedded tumor tissue to determine the extent (if any) of ErbB2 overexpression in the tumor.

The cancer to be treated herein may be one characterized by excessive activation of an ErbB receptor, e.g. HER2. Such excessive activation may be attributable to overexpression or increased production of the ErbB receptor or an ErbB ligand. In one embodiment of the invention, a diagnostic or prognostic assay will be performed to determine whether the patient's cancer is characterized by excessive activation of an ErbB receptor. For example, ErbB gene amplification and/or overexpression of an ErbB receptor in the cancer may be determined. Various assays for determining such amplification/overexpression are available in the art and include the IHC, FISH and shed antigen assays described above. Alternatively, or additionally, levels of an ErbB ligand, such as TGF-alpha., in or associated with the tumor may be determined according to known procedures. Such assays may detect protein and/or nucleic acid encoding it in the sample to be tested. In one embodiment, ErbB ligand levels in the tumor may be determined using immunohistochemistry (IHC); see, for example, Scher et al. (1995) Clin. Cancer Research 1:545-550. Alternatively, or additionally, one may evaluate levels of ErbB ligand-encoding nucleic acid in the sample to be tested; e.g. via FISH, southern blotting, or PCR techniques.

Moreover, ErbB receptor or ErbB ligand overexpression or amplification may be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

For the prevention or treatment of disease, the appropriate dosage of an ADC will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of ADC to be administered to a patient is in the range of about 0.1 to about 10 mg/kg of patient weight.

For repeated administrations over several days or longer, depending on the condition, the treatment is sustained until a desired suppression of disease symptoms occurs. An exemplary dosing regimen comprises administering an initial loading dose of about 4 mg/kg, followed by a weekly maintenance dose of about 2 mg/kg of the anti-ErbB2 antibody. Other dosage regimens may be useful. The progress of this therapy is easily monitored by conventional techniques and assays.

Combination Therapy

An antibody drug conjugate (ADC) of the invention may be combined in a pharmaceutical combination formulation, or dosing regimen as combination therapy, with a second compound having anti-cancer properties. The second compound of the pharmaceutical combination formulation or dosing regimen may have complementary activities to the ADC of the combination such that they do not adversely affect each other.

The second compound may be a chemotherapeutic agent, cytotoxic agent, cytokine, growth inhibitory agent, anti-hormonal agent, and/or cardioprotectant. Such molecules are suitably present in combination in amounts that are effective for the purpose intended. A pharmaceutical composition containing an ADC of the invention may also have a therapeutically effective amount of a chemotherapeutic agent such as a tubulin-forming inhibitor, a topoisomerase inhibitor, or a DNA binder.

Alternatively, or additionally, the second compound may be an antibody which binds ErbB2 and blocks ligand activation of an ErbB receptor. The second antibody may be monoclonal antibody 2C4 or humanized 2C4. The second antibody may be conjugated with a cytotoxic or chemotherapeutic agent, e.g., a 1,8 bis-naphthalimide moiety. For example, it may be desirable to further provide antibodies which bind to EGFR, ErbB2, ErbB3, ErbB4, or vascular endothelial factor (VEGF) in the one formulation or dosing regimen. An exemplary combination therapy of the invention is a Formula I ADC and bevacizumab (Avastin™, Genentech, South San Francisco, Calif.).

Other therapeutic regimens may be combined with the administration of an anticancer agent identified in accordance with this invention. The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein there is a time period while both (or all) active agents simultaneously exert their biological activities.

In one embodiment, treatment with an ADC of the present invention involves the combined administration of an anticancer agent identified herein, and one or more chemotherapeutic agents or growth inhibitory agents, including coadministration of cocktails of different chemotherapeutic agents, optionally along with treatment with an anti-ErbB2 antibody, such as trastuzumab. Chemotherapeutic agents include taxanes (such as paclitaxel and doxetaxel) and/or anthracycline antibiotics. Preparation and dosing schedules for such chemotherapeutic agents may be used according to manufacturers instructions or as determined empirically by the skilled practitioner. Preparation and dosing schedules for such chemotherapy are also described in Chemotherapy Service Ed., M. C. Perry, Williams & Wilkins, Baltimore, Md. (1992).

The anticancer agent may be combined with an anti-hormonal compound; e.g., an anti-estrogen compound such as tamoxifen; an anti-progesterone such as onapristone (EP 616812); or an anti-androgen such as flutamide, in dosages known for such molecules. Where the cancer to be treated is hormone independent cancer, the patient may previously have been subjected to anti-hormonal therapy and, after the cancer becomes hormone independent, the anti-ErbB2 antibody (and optionally other agents as described herein) may be administered to the patient. It may be beneficial to also coadminister a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

Metabolites of the Antibody Drug Conjugates

Also falling within the scope of this invention are the in vivo metabolic products of the ADC compounds described herein, to the extent such products are novel and unobvious over the prior art. Such products may result for example from the oxidation, reduction, hydrolysis, amidation, esterification, enzymatic cleavage, and the like, of the administered compound. Accordingly, the invention includes novel and unobvious compounds produced by a process comprising contacting a compound of this invention with a mammal for a period of time sufficient to yield a metabolic product thereof.

Metabolite products typically may be identified by preparing a radiolabelled (e.g. C¹⁴ or H³) ADC, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS, LC/MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the ADC compounds of the invention.

Articles of Manufacture

In another embodiment of the invention, an article of manufacture, or “kit”, containing materials useful for the treatment of the disorders described above is provided. The article of manufacture comprises a container and a label or package insert on or associated with the container. Suitable containers include, for example, bottles, vials, syringes, blister pack, etc. The containers may be formed from a variety of materials such as glass or plastic. The container holds an antibody-drug conjugate (ADC) composition which is effective for treating the condition and may have a sterile access port (for example the container may be an intravenous solution bag or a vial having a stopper pierceable by a hypodermic injection needle). At least one active agent in the composition is an ADC. The label or package insert indicates that the composition is used for treating the condition of choice, such as cancer.

The article of manufacture may comprise (a) a first container with a compound contained therein, wherein the compound comprises an ADC of the present invention in which the antibody of the ADC is a first antibody inhibits growth of cancer cells; and (b) a second container with a compound contained therein, wherein the compound comprises a second compound, composition, or formulation having biological activity. The article of manufacture in this embodiment of the invention may further comprise a package insert indicating that the first and second compounds can be used to treat cancer, or other disorder. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

### Example 1

### Synthesis of 4-Morpholino-Naphthoic Anhydride 1

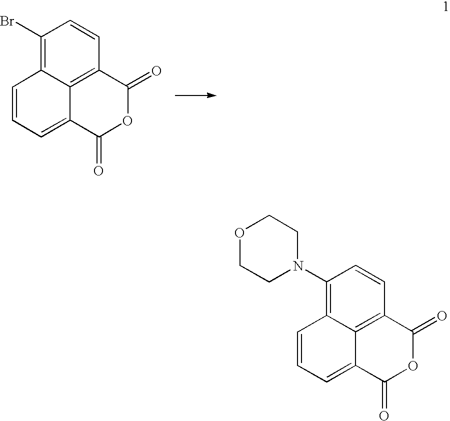

A mixture of 4-bromo, naphthalic anhydride (0.21 gm, 0.74 mmoles), morpholine 0.61 ml, 0.70 mmoles), and 5 ml ethanol was heated for 4.5 hr at 160° C. in a 15 ml sealed tube. After cooling, the mixture was concentrated under vacuum, dissolved in 30 ml dichloromethane, washed with 1 M citric acid, dried and concentrated. The orange solid was triturated with toluene to an orange solid, 4-morpholino-1,8-naphthalic anhydride 1 (0.089 gm, 51% yield). LC/MS -283 MW. ¹H NMR (300 MHz, CDCl₃): δ 8.61 (1H, d, J=7.3 Hz), 8.55 (1H, d, J=8.4 Hz), 8.48 (1H, d, J=8.5 Hz), 7.75 (1H, t, J=8.1 Hz); 7.27 (1H, dd, J=8.1 Hz); 4.31 (4H, m); 3.31 (4H, m).

### Example 2

### Synthesis of 1,3-bis glycyl-1,3 diaminopropane 2

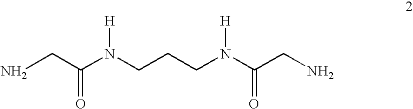

Carbonyl diimidazole (CDI, 0.71 gm, 4.40 mmoles) was added to a mixture of 10 ml dichloromethane and BOC-glycine (0.73 gm, 4.19 mmoles) at 0° C. under nitrogen. After 2 hr at 0° C., 1,3 propanediamine (0.18 ml, 2.0 mmoles) was added and the mixture was warmed to room temperature and stirred overnight. The mixture was diluted with dichloromethane and extracted with sat. NaHCO₃. The aqueous phase was extracted 2× with dichloromethane. The combined organic phases were washed with sat. NaCl, dried over MgSO₄, and concentrated under vacuum to give the 1,3 bis BOC glycyl-1,3 diaminopropane intermediate as a white sticky solid. ¹H NMR (300 MHz, CDCl₃): δ 6.76 (2H, s, br); 5.30 (2H, s, br); 3.77 (4H, d, J=5.7 Hz); 3.31 (4H, m); 1.66 (2H, m); 1.4 (9H, m). This intermediate was taken up with 16 ml 1M HCl in AcOH and stirred under nitrogen at room temperature for 2 hours. The mixture was concentrated under vacuum to a white solid which was triturated with diethyl ether to give the bis hydrochloride salt of 1,3-bis glycyl-1,3 diaminopropane 2 as a yellow oil. ¹H NMR (300 MHz, D₂O): δ 3.78 (4H, s), 3.26 (4H, t, J=5.7 Hz); 1.66 (2H, m).

### Example 3

### Synthesis of N¹,N³-bis(2-aminoethyl)malonamide 3

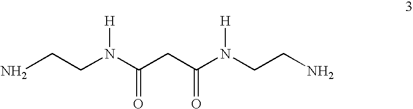

A solution of malonyl chloride (0.5 ml, 5.0 mmoles) in 4 ml dichloromethane under nitrogen was stirred at 0° C., then added dropwise over 30 minutes to a stirred solution of mono BOC-1,2-diaminoethane (1.6 ml, 10.0 mmoles) and triethylamine (1.67 ml, 12 mmoles) in 5 ml dichloromethane at 0° C. The solution was allowed to warm to room temperature and stir overnight. The cloudy orange mixture was diluted with 90 ml dichloromethane, washed with 30 ml each of 2N HCl, sat NaHCO₃, and sat. NaCl, then dried over MgSO₄ and concentrated under vacuum to give the bis BOC intermediate as a sticky orange solid ¹H NMR (300 MHz, CDCl₃): δ 7.27 (2H, m); 5.01 (4H, s, br); 3.38 (4H, m); 3.28 (4H, m); 3.16 (s, 2H). The BOC groups were removed to give N′,N³-bis(2-aminoethyl)malonamide 3.

### Example 4

### Synthesis of N-glycyl-3-nitro-1,8 naphthalimide 4

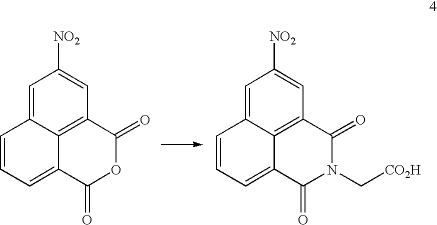

A mixture of 3-nitro-1,8-naphthalic anhydride (0.155 gm, 0.64 mmole) and glycine (0.048 gm, 0.64 mmole) in 1.5 ml dimethylformamide (DMF) was heated at 100° C. under nitrogen for about 12 hours. The mixture was diluted with ethylacetate and washed with 1.0M citric acid, dried over MgSO4, and concentrated under vacuum to give N-glycyl-3-nitro-1,8 naphthalimide 4. ¹H NMR (300 MHz, DMSO-d₆): δ 9.54 (1H, J=2.2 Hz); 8.99 (1H, d, J=2.2 Hz); 8.84 (1H, d, J=7.4 Hz); 8.72 (1H, d, J=7.3 Hz); 8.09 (1H, t, J=8.2 Hz); 4.76 (s, 2H).

### Example 5

### Synthesis of N-glycyl-4-amino-1,8 naphthalimide 5

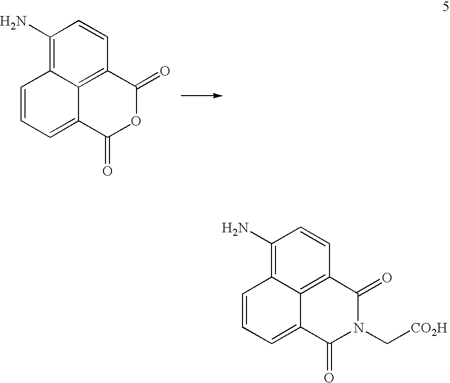

A mixture of 4-amino-1,8-naphthalic anhydride (0.230 gm, 1.03 mmole) and glycine (0.239 gm, 3.19 mmole) in 3 ml dimethylformamide (DMF) was heated by microwave treatment at 200° C. under nitrogen for 10 minutes. LC/MS analysis of the mixture showed conversion of starting anhydride to be complete. The mixture was cooled, filtered and the precipitate was dried to give N-glycyl-4-amino-1,8 naphthalimide 5. ¹H NMR (300 MHz, DMSO-d₆): δ 8.65 (1H, d, J=8.7 Hz); 8.44 (1H, d, J=6.9 Hz); 8.19 (1H, d, J=8.4 Hz); 7.67 (1H, t, J=7.5 Hz); 6.85 (1H, d, J=8.1 Hz); 4.67 (s, 2H).

### Example 6

### Synthesis of N-glycyl-4-morpholino, 1,8 naphthalimide 6

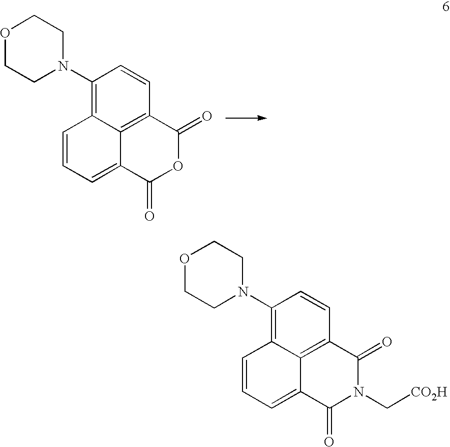

A mixture of 4-morpholino-1,8-naphthalic anhydride (0.163 gm, 0.63 mmole) and glycine (0.10 gm, 1.33 mmole) in 3 ml dimethylformamide (DMF) was heated at 200° C. with microwave treatment under nitrogen for 10 minutes. The mixture was diluted with ethylacetate and washed with 11.0M citric acid, dried over MgSO4, and concentrated under vacuum to give N-glycyl-4-morpholino-1,8 naphthalimide 6. ¹H NMR (300 MHz, DMSO-d₆): δ 8.63 (2H, m), 8.55 (1H, d, J=8.2 Hz); 7.95 (1H It, J=8.0 Hz); 7.50 (1H, d, J=8.0 Hz); 4.82 (2H, s); 4.20 (4H, m); 3.36 (4H, m).

### Example 7

### Synthesis of N-aminoethylethoxy-3-nitro-1,8 naphthalimide 7

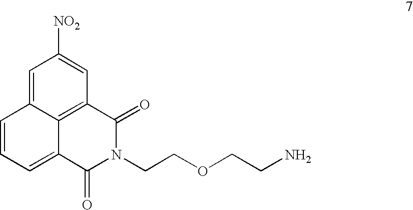

A suspension of 0.2 M 2,2′-oxydiethylamine dihydrochloride (0.247 gm, 1.35 mmole) and 0.4 M DIEA (0.47 ml, 2.7 mmole) in 4.5 DMF was added to 3-nitro-1,8-naphthalic anhydride (0.018 gm, 0.073 mmole) and heated at 150° C. with microwave treatment under nitrogen for 5 minutes. The mixture was cooled, treated with 25 ml 1.3 M aqueous TFA, and concentrated to about 2 ml. The residue was diluted with dichloromethane, washed with sat. NaCl, dried over MgSO₄, and concentrated under vacuum to give N-aminoethylethoxy-3-nitro-1,8 naphthalimide 7. MS m/z 330 (M+H)⁺.

### Example 8

### Synthesis of N-aminoethylethoxy-4-amino-1,8 naphthalimide 8

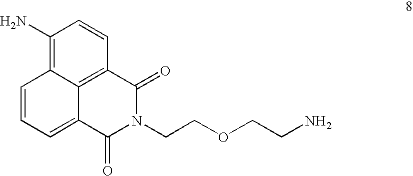

A suspension of 0.2 M 2,2′-oxydiethylamine dihydrochloride (0.167 gm, 0.92 mmole) and 0.4 M DIEA (0.40 ml, 2.3 mmole) in 2.5 DMF was added to 4-amino-1,8-naphthalic anhydride (0.067 gm, 0.30 mmole) and heated at 170° C. with microwave treatment under nitrogen for 10 minutes. The mixture was cooled, concentrated under vacuum, and purified by preparatory HPLC to give N-aminoethylethoxy-4-amino-1,8 naphthalimide 8. MS m/z 300 (M+H)⁺.

### Example 9

### Synthesis of N- aminoethylethoxy -4-morpholino-1,8 naphthalimide 9

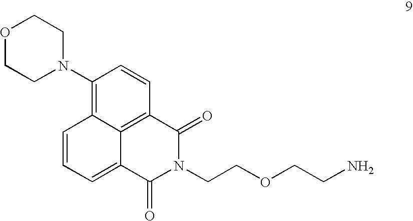

A suspension of 0.2 M 2,2′-oxydiethylamine dihydrochloride (0.135 gm, 0.74 mmole) and 0.4 M DIEA (0.32 ml, 1.85 mmole) in 2.5 DMF was added to 4-morpholino-1,8-naphthalic anhydride (0.068 gm, 0.24 mmole) and heated at 150° C. with microwave treatment under nitrogen for 12 minutes. The mixture was cooled, diluted with dichloromethane, washed with sat. NaHCO3, dried over MgSO4, and concentrated under vacuum to give N-aminoethylethoxy-4-morpholino-1,8 naphthalimide 9. ¹H NMR (300 MHz, CDCl₃): δ 8.59 (1H, d, J=7.2 Hz); 8.53 (1H, d, J=7.8 Hz); 8.42 (1H, d, J=8.4 Hz); 7.70 (1H, t, J=7.2 Hz); 7.25 (d, 1H, J=8.1 Hz); 4.43 (2H, t, J=5.8 Hz); 4.02 (4H, m); 3.80 (2H, t, J=6.0 Hz); 3.54 (2H, t, J=5.2 Hz); 3.26 (4H, m); 2.81 (2H, t, J=5.7 Hz).

### Example 10

### Synthesis of N-aminopropylethylamine-3-nitro-1,8 naphthalimide 10a and N-aminoethylpropylamine-3-nitro-1,8 naphthalimide 10b

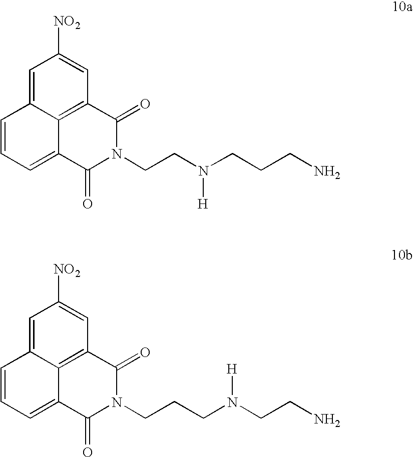

A mixture of 3-nitro-1,8-naphthalic anhydride (0.641 gm, 2.64 mmole), 2-aminoethyl-1,3-propanediamine (1 ml, 7.68 mmole) and 2.5 ml ethanol was heated from 0° C. to 100° C. over 30 minutes then at 100° C. for 2 hours. LC/MS analysis showed the reaction was complete and the products were formed in a 2:1 ratio. The mixture was cooled, concentrated under vacuum, taken up in DMF and purified by prep. HPLC which separated the products N-aminopropylethylamine-3-nitro-1,8 naphthalimide 10a ¹H NMR (300 MHz, CF₃CO₂D): 89.38 (1H, d, J=2.1 Hz); 9.32 (1H, d, J=2.1 Hz); 8.85 (1H, d, J=7.5 Hz); 8.61 (1H, d, J=8.6 Hz); 8.04 (1H, t, J=7.8 Hz); 4.75 (2H, m); 3.79 (m, 2H); 3.50 (2H, m); 3.40 (2H, m); 2.42 (m, 2H), and N-aminoethylpropylamine-3-nitro-1,8 naphthalimide 10b in about 85-91% purity.

### Example 11

### Synthesis of N-aminopropylethylamine-4-amino-1,8 naphthalimide 11a and N-aminoethylpropylamine-4-amino-1,8 naphthalimide 11b

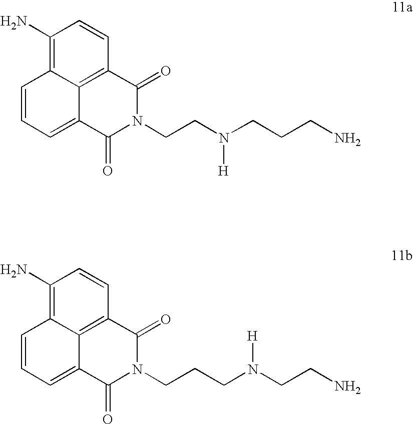

A mixture of 4-amino-1,8-naphthalic anhydride (0.477 gm, 2.13 mmole), 2-aminoethyl-1,3-propanediamine (0.83 ml, 6.38 mmole) and 2.5 ml ethanol was heated from 0° C. to 100° C. over 30 minutes then at 100° C. for 2 hours with microwave treatment. LC/MS analysis showed the reaction was complete and the products were formed in a 2:1 ratio. The mixture was cooled, concentrated under vacuum, taken up in DMF and purified by prep. HPLC which separated the products N-aminopropylethylamine-4-amino-1,8 naphthalimide 11a ¹H NMR (300 MHz, CF₃CO₂D): δ 8.82 (1H, d, J=7.5 Hz); 8.61 (1H, d, J=8.4 Hz); 8.07 (1H, t, J=8.2 Hz); 4.75 (2H, m); 3.80 (2H, m); 3.49 (2H, m); 3.41 (2H, m); 3.34 (2H, m), and N-aminoethylpropylamine-4-amino-1,8 naphthalimide 11b, each in about 98% purity.

### Example 12

### Synthesis of N-aminoproyvlethylamine-4-morpholino-1,8 naphthalimide 12a and N-aminoethylpropylamine-4-mompholino-1,8 naphthalimide 12b

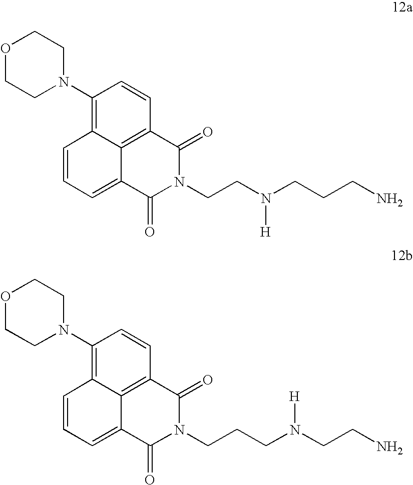

A mixture of 4-morpholino-1,8-naphthalic anhydride (0.317 gm, 1.06 mmole), 2-aminoethyl-1,3-propanediamine (0.44 ml, 3.38 mmole) and 2.5 ml ethanol was heated from 0° C. to 100° C. over 30 minutes then at 100° C. for 2 hours. LC/MS analysis showed the reaction was complete and the products were formed in a 3:1 ratio. The mixture was cooled, concentrated under vacuum, taken up in 4 ml 1.3 M TFA and 6 ml acetic acid and purified by prep. HPLC which separated the products N-aminopropylethylamine-(4-morpholino-1,8 naphthalimide 12a ¹H NMR (300 MHz, CF₃CO₂D): δ 8.80 (2H, m); 8.69 (1H, d, J=8.4 Hz); 8.19 (1H, d, J=8.2 Hz); 8.07 (1H, t, J=8.4 Hz); 4.74 (2H, m); 4.54 (2H, m); 4.21 (2H, m); 3.79 (2H, m); 3.44 (2H, m); 3.34 (m, 2H); 2.36 (2H, m), and N-aminoethylpropylamine-(4-morpholino-1,8 naphthalimide 12b each in about 93-99% purity.

### Example 13

### Synthesis of N-methanesulfonyloxyethyl-(3-nitro-1,8 naphthalimide 13

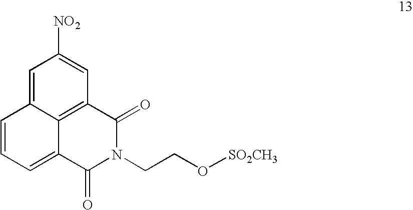

N-hydoxyethyl-3-nitro-1,8 naphthalimide was prepared from 3-nitro-1,8 naphthalimide and ethanolamine in ethanol by microwave heating at 150° C. for 5 minutes, and precipitation from boiling toluene. Methanesulfonyl chloride (1.05 ml, 13 mmole) was added to a solution of N-hydoxyethyl-3-nitro-1,8 naphthalimide (1.70 gm, 5.94 mmole) and 100 ml pyridine. After several hours stirring at room temperature under nitrogen, one liter of water was added, and the precipitate was filtered to give N-methanesulfonyloxyethyl-(3-nitro-1,8 naphthalimide 13(2.0 gm, 92% yield). ¹H NMR (300 MHz, DMSO-d₆): δ 9.51 (1H, d, J=2.0 Hz); 8.98 (1H, d, J=2.0 Hz); 8.81 (1H, d, J=8.2 Hz); 8.71 (1H, d, J=7.4 Hz); 8.07 (1H, t, J=7.5 Hz); 4.46 (4H, m); 3.15 (3H, s).

### Example 14

### Synthesis of N-iodoethyl-(3-nitro-1,8 naphthalimide 14

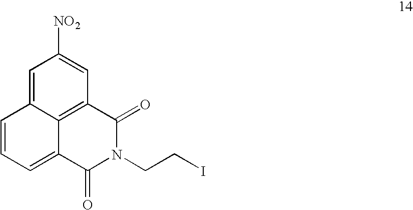

N-methanesulfonyloxyethyl-(3-nitro-1,8 naphthalimide 13 (2.0 gm, 5.49 mmole) was dissolved in 250 ml 2-butanone and treated with sodium iodide (5.15 gm, 33.9 mmole) and stirred overnight at room temperature under nitrogen. The precipitate was filtered and the eluate washed with sat. NaCl, dried over MgSO4, and concentrated under vacuum to give N-iodoethyl-(3-nitro-1,8 naphthalimide 14. ¹H NMR (300 MHz, DMSO-d₆): δ 9.51 (1H, d, J=2.1 Hz); 8.97 (1H, d, J=2.1 Hz); 8.80 (1H, d, J=8.3 Hz); 8.71 (1H, d, J=6.9 Hz); 8.07 (1H, t, J=7.4 Hz); 4.40 (2H, t, J=7.4 Hz); 3.41 (2H, t, J=7.4 Hz).

### Example 15

### Synthesis of N-(2,4-dinitrophenylaminoethylethoxy)-3-nitro-1,8 naphthalimide 15

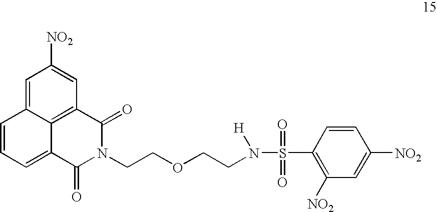

A solution of N-aminoethylethoxy-3-nitro-1,8 naphthalimide 7 (TFA salt, 0.190 gm, 0.043 mmole), triethylamine (0.18 ml, 1.29 mmole), and 5 ml DMF was cooled to 0° C. 2,4-Dinitrobenzenesulfonyl chloride (0.128 gm, 0.47 mmole) was added and the solution was allowed to warm to room temperature and stir under nitrogen for an hour. LC/MS analysis showed that sulfonation was virtually complete. A slight excess of sodium ethoxide in 1 ml ethanol was added to quench remaining 2,4-dinitrobenzenesulfonyl chloride. The mixture was filtered through celite, rinsed with 15 ml DMF and 20 ml ethanol. The filtrate was concentrated under vacuum, dissolved in 6 ml DMF and purified by prep. HPLC to give N-(2,4-dinitrophenylaminoethylethoxy)-3-nitro-1,8 naphthalimide 15 in 61% yield. ¹H NMR (300 MHz, CDCl₃): δ9.32 (1H, d, J=2.4 Hz); 9.16 (1H, d, J=2.1 Hz); 8.80(1H, d, J=7.8 Hz); 8.63 (1H, d, J=2.1 Hz); 8.48 (m, 2H); 8.33 (1H, d, J=8.4 Hz); 7.98 (1H, t, J=7.8 Hz); 5.92 (1H, m); 4.35 (2H, t, J=5.4 Hz); 3.70 (2H, t, J=5.4 Hz); 3.62 (2H, t, J=5.0 Hz); 3.32 (2H, m).

### Example 16a

### Synthesis of N, N′-(bis-aminoethyl-1,3-propanediamine)-bis-4-nitro-1,8 naphthalimide 16a

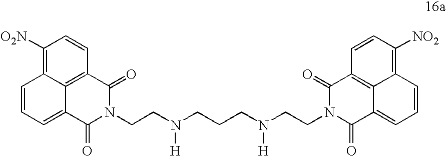

A solution of N,N-bis(aminoethyl)-1,3-propanediamine (0.91 gm, 5.29 mmole) in 5 ml N-methylmorpholine (NMM) was added to a solution of 4-nitro-1,8-naphthalic anhydride (2.54 gm, 9.92 mmole) in 10 ml NMM. The reaction was stirred at room temperature under nitrogen for 5 minutes, then heated at 38° C. for one hour, then at 120° C. (reflux) for 2 hours. The mixture was filtered hot, concentrated under vacuum, dissolved in a minimum of dichloromethane, and purified by silica gel chromatography to give N,N′-(bis-aminoethyl-1,3-propanediamine)-bis-4-nitro-1,8 naphthalimide 16a. MS m/z 611 (M+H)⁺.

### Example 16b

### Synthesis of N, N′-(bis-aminoethyl-1,3-propanediamine)-bis-4-nitro-1,8 naphthalimide 16b

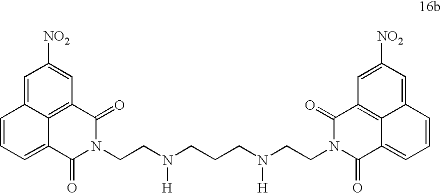

Following the same procedure as Example 16a, N,N′-(bis-aminoethyl-1,3-propanediamine)-bis-3-nitro-1,8 naphthalimide 16b was prepared from 3-nitro-1,8-naphthalic anhydride (1.00 gm, 3.91 mmole) and N,N-bis(aminoethyl)-1,3-propanediamine (11.7 mmole, 3 equiv) in 3 ml, at 100° C. for 5 minutes.

### Example 17

### Synthesis of N, N′-(bis-aminoethyl-1,3-pronanediamine)-bis-4-chloro-1,8 naphthalimide 17

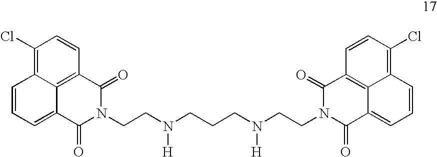

A solution of N,N-bis(aminoethyl)-1,3-propanediamine (also: 1,4,8,11-tetraazaundecane, 0.87 ml, 5.03 mmole) in 2 ml ethanol was slowly added to a solution of 4-chloro-1,8-naphthalic anhydride (2.35 gm, 10.10 mmole) in 12 ml NMM. The reaction was stirred at room temperature under nitrogen for 5 minutes, then heated at 38° C. for 45 minutes, the heat was increased slowly to 115° C. and held for 1.5 hours. The mixture was cooled, filtered, concentrated under vacuum, dissolved in a minimum of dichloromethane, and purified by silica gel chromatography to give the bis TFA salt of N,N′-(bis-aminoethyl-1,3-propanediamine)-bis-4-chloro-1,8 naphthalimide 17 as a bright yellow solid. MS m/z 589 (M⁺).

### Example 18

### Synthesis of N, N′-(bis-aminoethyl-1,3-propanediamine)-bis-3-bromo-1,8 naphthalimide 18

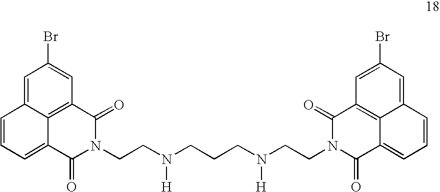

A solution of N,N-bis(aminoethyl)-1,3-propanediamine (also: 1,4,8,11-tetraazaundecane, 0.081 ml, 0.47 mmole) in 5 ml dioxane was slowly added to a solution of 3-bromo-1,8-naphthalic anhydride (0.267 gm, 0.93 mmole) in 1.2 ml NMM. The reaction was stirred at room temperature under nitrogen for 5 minutes, then heated at 38° C. for 45 minutes, the heat was increased slowly to 115° C. and held for 1.5 hours. The mixture was cooled, filtered, concentrated under vacuum, dissolved in a minimum of dichloromethane, and purified by silica gel chromatography to give the bis TFA salt of N,N′-(bis-aminoethyl-1,3-propanediamine)-bis-3-bromo-1,8 naphthalimide 18 as a white solid. MS m/z 679 (M+H)⁺.

### Example 19

### Synthesis of N, N′-(bis-aminoethyl-1,3-propanediamine)-bis-4-amino-1,8 naphthalimide 19

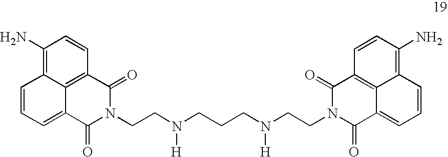

Palladium on carbon (10% Pd/C, 76 mg) was added to a solution of N,N′-(bis-aminoethyl-1,3-propanediamine)-bis-4-nitro-1,8 naphthalimide 16a (about 60% pure, 0.12 gm, 0.12 mmole) and 20 ml DMF. The flask was flushed with hydrogen gas and the reaction was stirred at room temperature overnight. The mixture was filtered though celite, rinsing with DMF, concentrated, and purified by prep. HPLC to give N N, N′-(bis-aminoethyl-1,3-propanediamine)-bis-4-amino-1,8 naphthalimide 19 as a red solid. MS m/z 551 (M+H)⁺.

### Example 20

### Synthesis of N, N′-(bis-aminoethyl-1,3-propanediamine)-bis-4-morpholino-1,8 naphthalimide 20

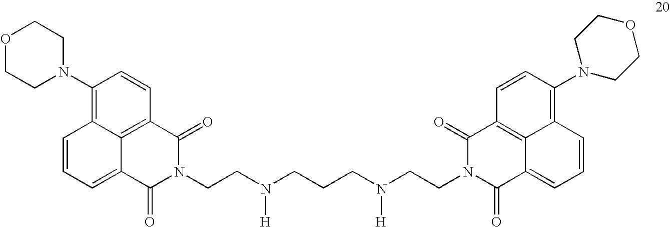

A solution of N,N′-(bis-aminoethyl-1,3-propanediamine)-bis-4-bromo-1,8 naphthalimide (19 mg, 0.021 mmole) and 1 ml NMM was heated in a sealed tube to 70° C. and held for 3 hours, then increased to 100° C. and held for 2 hours. The mixture was cooled, dissolved in 5 ml acetic acid and 1 ml 0.1% aqueous TFA and purified by prep. HPLC to give N,N′-(bis-aminoethyl-1,3-propanediamine)-bis-4-morpholino-1,8 naphthalimide 20 as the bis-TFA salt. MS m/z 691(M+H)⁺.

### Example 21

### Synthesis of N, N′-(bis-aminoethyl-1,3-propanediamine)-bis-4-dimethylamino-1,8 naphthalimide 21

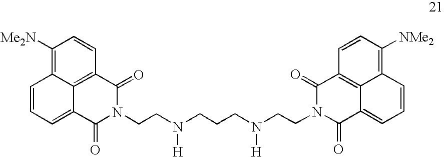

A solution of N,N′-(bis-aminoethyl-1,3-propanediamine)-bis-4-bromo-1,8 naphthalimide (16 mg, 0.017 mmole) and 1.5 ml 40% aqueous dimethylamine was placed in a sealed tube for one hour at room temperature, then heated to 60° C. and held for 1 hour, then increased to 70° C. and held for 30 minutes. Dimethylformamide (0.75 ml) was added and heating at 70° C. was continued for 1.5 hours, then let stand at room temperature for 48 hours. The mixture was cooled, dissolved in 5 ml acetic acid and 1 ml 0.1% aqueous TFA and purified by prep. HPLC to give the bright orange solid N,N′-(bis-aminoethyl-1,3-propanediamine)-bis-4-dimethylamino-1,8 naphthalimide 21 as the bis-TFA salt. MS m/z 607 (M+H)⁺.

### Example 22

### Synthesis of N, N′-(bis-aminoethyl-1,3-propanediamine)-bis-(4-N-methoxyethyl)-1,8 naphthalimide 22

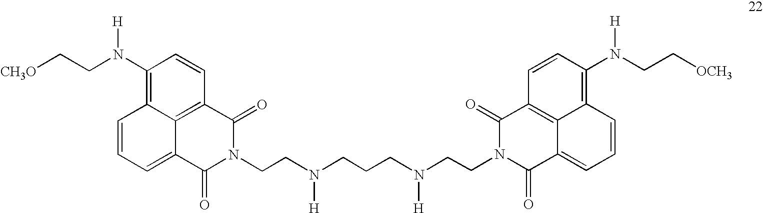

A solution of N,N′-(bis-aminoethyl-1,3-propanediamine)-bis-4-bromo-1,8 naphthalimide (26 mg, 0.028 mmole) and 4 ml of 2-methoxyethylamine was placed in a sealed tube and the temperature was raised to 80° C. over 15 minutes and held for 3.5 hours. The mixture was cooled, concentrated, dissolved in 2 ml DMF and 8 ml 0.1% aqueous TFA, and purified by prep. HPLC to give the orange solid N,N′-(bis-aminoethyl-1,3-propanediamine)-bis-(4-N-methoxyethyl)-1,8 naphthalimide 22 as the bis-TFA salt. MS m/z 667 (M+H)⁺.

### Example 23

### Synthesis of N, N′-(bis-aminoethyl-1,3-propanediamine)-bis-(4-N-hydroxyethyl)-1,8 naphthalimide 23

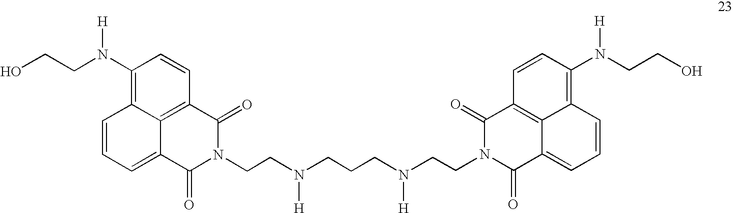

A solution of N,N′-(bis-aminoethyl-1,3-propanediamine)-bis-4-bromo-1,8 naphthalimide (36 mg, 0.040 mmole) and 2 ml of ethanolamine was placed in a sealed tube and the temperature was raised to 80° C. over 15 minutes and held overnight. The mixture was cooled, concentrated, dissolved in 2 ml DMF and 8 ml 0.1% aqueous TFA, and purified by prep. HPLC to give the bright red solid N,N′-(bis-aminoethyl-1,3-propanediamine)-bis-(4-N-hydroxyethyl)-1,8 naphthalimide 23 as the bis-TFA salt. MS m/z 639 (M+H)⁺.

### Example 24a

### Synthesis of N, N′-(bis-aminoethyl-1,3-propanediamine)-bis-(4-N-methylpiperidine)-1,8 naphthalimide 24a

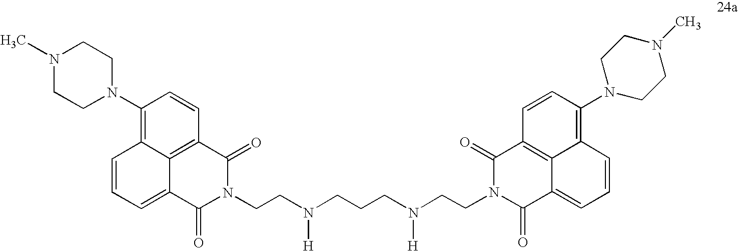

A solution of N,N′-(bis-aminoethyl-1,3-propanediamine)-bis-4-bromo-1,8 naphthalimide (0.112 gm, 0.12 mmole) and 7 ml of 1-methylpiperazine was placed in a 15 ml sealed tube and the temperature was raised to 80° C. over 15 minutes and held 15 hours. The mixture was cooled, concentrated, dissolved in 1 ml acetic acid and 2 ml 0.1% aqueous TFA, and purified by prep. HPLC to give N,N′-(bis-aminoethyl-1,3-propanediamine)-bis-(4-N-methylpiperidine)-1,8 naphthalimide 24a as the tetra-TFA salt. MS m/z 717 (M+H)⁺.

### Example 24b

### Synthesis of N, N′-(bis-aminoethyl-1,3-propanediamine)-bis-(4-N-imidazolyl)-1,8 naphthalimide 24b

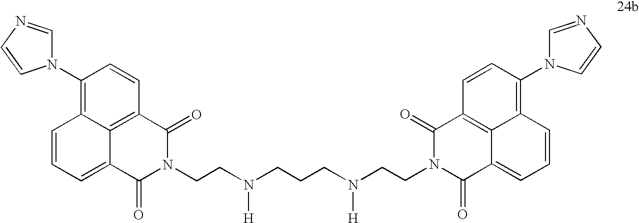

Following the same protocol as Example 24a, N,N′-(bis-aminoethyl-1,3-propanediamine)-bis-(4-N-imidazolyl)-1,8 naphthalimide 24b was prepared from N,N′-(bis-aminoethyl-1,3-propanediamine)-bis-4-bromo-1,8 naphthalimide, imidazole, potassium carbonate, in DMF with heating.

### Example 24c

### Synthesis of N¹-methyl, N, N′-(bis-aminoethyl-1,3-propanediamine)-bis-(4-N-imidazolyl)-1,8 naphthalimide 24c

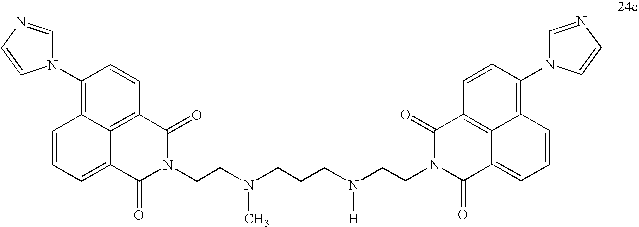

Also following the same protocol as Example 24a, N¹-methyl, N,N′-(bis-aminoethyl-1,3-propanediamine)-bis-(4-N-imidazolyl)-1,8 naphthalimide 24c was prepared from N¹-methyl, N,N′-(bis-aminoethyl-1,3-propanediamine)-bis-4-bromo-1,8 naphthalimide and imidazole.

### Example 24d

### Synthesis of N,N′-(bis-aminoethyl-1,3-propanediamine)-bis-(4-azido)-1,8 naphthalimide 24d

Also following the same protocol as Example 24a, N,N′-(bis-aminoethyl-1,3-propanediamine)-bis-(4-azido)-1,8 naphthalimide 24d was prepared from N,N′-(bis-aminoethyl-1,3-propanediamine)-bis-4-bromo-1,8 naphthalimide, sodium azide, potassium carbonate, in DMF with heating at 150° C. for 5 minutes.

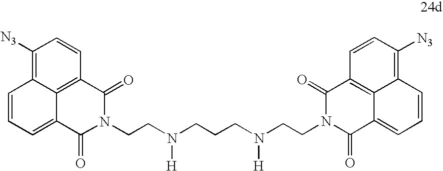

### Example 25

### Synthesis of N, N′-(bis-N-formyl-aminoethyl-1,3-propanediamine)-bis-3-nitro-1,8 naphthalimide 25a and enamminium 25b

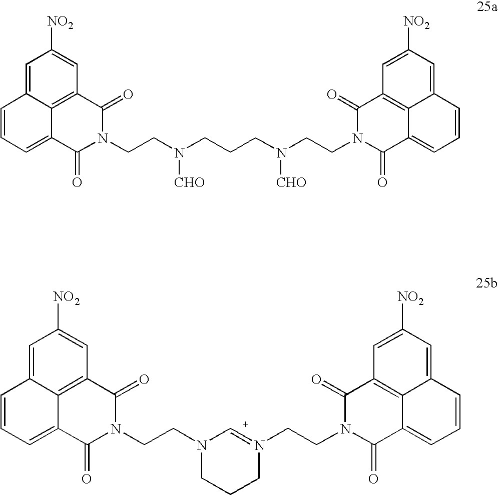

A solution of N,N′-(bis-aminoethyl-1,3-propanediamine)-bis-3-nitro-1,8 naphthalimide (0.029 gm, 0.047 mmole), 5 ml ethylformate, and 2 ml DMF was refluxed under nitrogen for 3.5 hours. The mixture was cooled, concentrated, dissolved in 1 ml acetic acid and 2 ml 0.1% aqueous TFA, and purified by prep. HPLC to separate N,N′-(bis-N-formyl-aminoethyl-1,3-propanediamine)-bis-3-nitro-1,8 naphthalimide 25a (minor product), and the cyclized enamminium salt 25b (major product) ¹H NMR (300 MHz, DMSO-d6): δ 9.77 (d, 2H, J=2.4 Hz); 8.89 (d, 2H, J=2.4 Hz); 8.75 (2H, d, J=7.5 Hz); 8.59 (2H, d, J=7.2 Hz); 8.30 (s, 1H); 8.00 (2H, t, J=7.2 Hz); 4.16 (m, 4H); 3.58 (m, 8H); 2.09 (m, 2H).

### Example 26

### Synthesis of N, N′-(N-benzyl-aminoethyl-1,3-propanediamine)-bis-3-nitro-1,8 naphthalimide 26a and N, N′-(bis-N-benzyl-aminoethyl-1,3-propanediamine)-bis-3-nitro-1.8 naphthalimide 26b

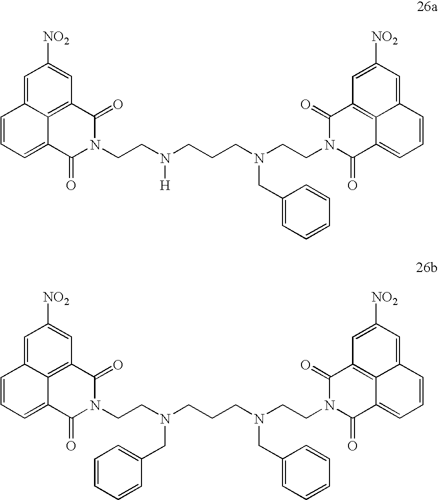

Benzylbromide (0.27 ml, 0.22 mmole), followed by 0.5 N NaOH (0.44 ml, 0.22 mmole) was added to a solution of N,N′-(bis-aminoethyl-1,3-propanediamine)-bis-3-nitro-1,8 naphthalimide (0.112 gm, 0.18 mmole) in 20 ml DMF under nitrogen and stirred overnight. The mixture was filtered and the filtrate was purified by prep. HPLC to separate N,N′-(N-benzyl-aminoethyl-1,3-propanediamine)-bis-3-nitro-1,8 naphthalimide 26a MS m/z 701 (M+H)⁺ and N,N′-(bis-N-benzyl-aminoethyl-1,3-propanediamine)-bis-3-nitro-1,8 naphthalimide 26b MS m/z 791 (M+H)⁺, each as the bis TFA salt.

### Example 27

### Synthesis of N, N′-(N-allyl-aminoethyl-1,3-propanediamine)-bis-3-nitro-1,8 naphthalimide 27a and N, N′-(bis-N-allyl-aminoethyl- 13-propanediamine)-bis-3-nitro-1.8 naphthalimide 27b

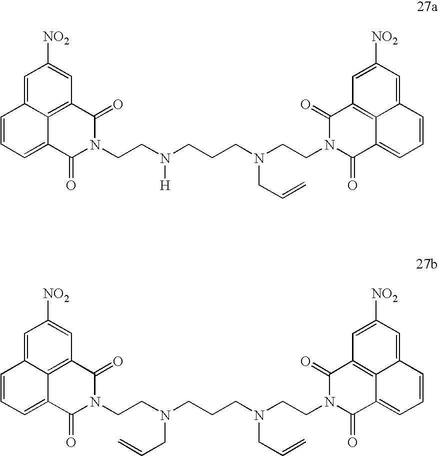

Allylbromide (0.12 ml, 0.14 mmole), followed by 0.5 N NaOH (0.27 ml, 0.14 mmole) was added to a solution of N,N′-(bis-aminoethyl-1,3-propanediamine)-bis-3-nitro-1,8 naphthalimide (0.040 gm, 0.065 mmole) in 15 ml DMF under nitrogen and stirred overnight. The mixture was filtered and the filtrate was purified by prep. HPLC to separate N,N′-(N-allyl-aminoethyl-1,3-propanediamine)-bis-3-nitro-1,8 naphthalimide 27a MS m/z 651 (M+H)⁺, and N,N′-(bis-N-allyl-aminoethyl-1,3-propanediamine)-bis-3-nitro-1,8 naphthalimide 27b MS m/z 691 (M+H)⁺, each as the bis TFA salt.

### Example 28

### Synthesis of N, N′-(bis-N-acetamidomethyl-aminoethyl-1,3-propanediamine)-bis-3-nitro-1,8 naphthalimide 28

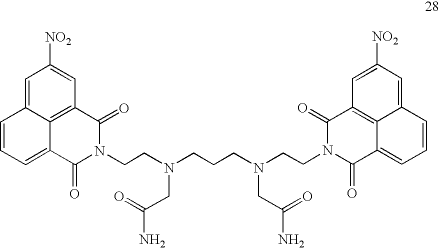

A mixture of N,N′-(bis-aminoethyl-1,3-propanediamine)-bis-3-nitro-1,8 naphthalimide (0.040 gm, 0.065 mmole), 2-bromoacetamide (0.045 gm, 0.32 mmole), cesium carbonate (CsCO₃, 0.022 gm, 0.067 mmole) and 3 ml DMF was stirred under nitrogen overnight at room temperature. LC/MS analysis showed starting N,N′-(bis-aminoethyl-1,3-propanediamine)-bis-3-nitro-1,8 naphthalimide and N,N′-(bis-N-acetamidomethyl-aminoethyl-1,3-propanediamine)-bis-3-nitro-1,8 naphthalimide 28. MS m/z 725 (M+H)⁺.

### Example 29

### Synthesis of N, N′-(N-acetyl-aminoethyl-1,3-propanediamine)-bis-3-nitro-1,8 naphthalimide

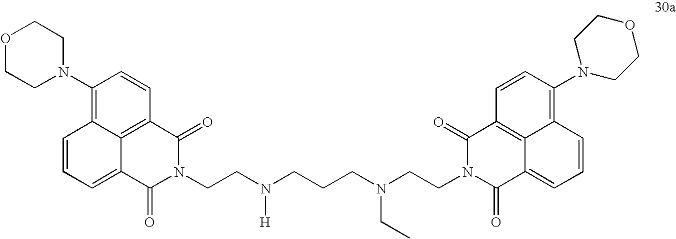

A mixture of N,N′-(N—F₁₇BOC-aminoethyl-1,3-propanediamine)-bis-3-nitro-1,8 naphthalimide (14 mg, 0.010 mmole), having the structure: 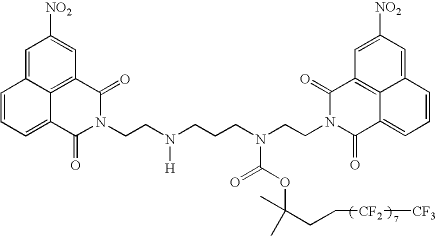
and 3 ml acetic anhydride was refluxed (110° C.) for one hour, then cooled. Several drops of water was added and the solution was concentrated to a white solid. One ml of TFA was added, mixed and let stand for an hour at room temperature. Concentration under vacuum gave the TFA salt as a yellow solid, N,N′-(N-acetyl-aminoethyl-1,3-propanediamine)-bis-3-nitro-1,8 naphthalimide 29. MS m/z 653 (M+H)⁺.

### Example 30a

### Synthesis of N, N′-(N-ethyl, bis-aminoethyl-1,3-propanediamine)-bis-4-morpholino-1,8 naphthalimide 30a

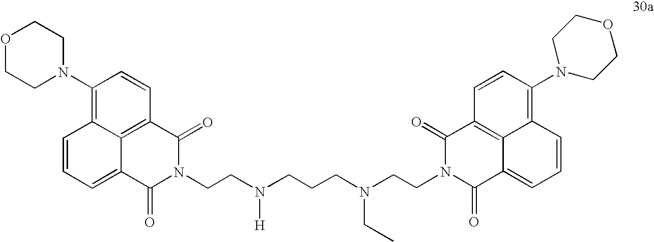

A mixture of the bis-TFA salt of N,N′-(bis-aminoethyl-1,3-propanediamine)-bis-4-morpholino-1,8 naphthalimide 20 (32 mg, 0.035 mmole), cesium carbonate (36 mg, 0.11 mmole), 5 ml acetonitrile, and 5 ml DMF was stirred at room temperature under nitrogen. Ethyl iodide (0.031 ml, 0.038 mmole) was added and stirred overnight. After 2 ml 10% TFA was added, the mixture was concentrated under vacuum, dissolved in 3.5 ml of 1.3 M aqueous TFA and 6 ml acetic acid, and injected onto a prep. HPLC column to give N,N′-(N-ethyl, bis-aminoethyl-1,3-propanediamine)-bis-4-morpholino-1,8 naphthalimide 30a MS m/z 719 (M+H)⁺, as well as a small amount of the bis-ethyl compound.

### Example 30b

### Synthesis of N, N′-(N-cyclopropylmethyl, bis-aminoethyl-1,3-propanediamine)-bis-4-N-imidazolyl-1,8 naphthalimide 30b

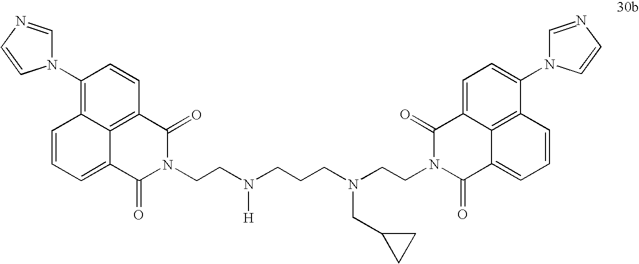

Following Example 30a, N,N′-(N-cyclopropylmethyl, bis-aminoethyl-1,3-propanediamine)-bis-4-N-imidazolyl-1,8 naphthalimide 30b was prepared from (bromomethyl)cyclopropane and N,N′-(bis-aminoethyl-1,3-propanediamine)-bis-4-N-imidazolyl-1,8 naphthalimide 24b.

### Example 30c

### Synthesis of N, N′-(N-cyclopropylmethyl, bis-aminoethyl-1,3-propanediamine)-bis-3-nitro-1,8 naphthalimide 30c

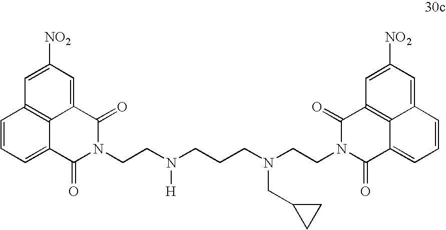

Following Example 30a, N,N′-(N-cyclopropylmethyl, bis-aminoethyl-1,3-propanediamine)-bis-3-nitro-1,8 naphthalimide 30c was prepared from (bromomethyl)cyclopropane and N,N′-(bis-aminoethyl-1,3-propanediamine)-bis-3-nitro-1,8 naphthalimide 16b.

### Example 31

### Synthesis of N, N′-(N-(4-acetylbenzamide), bis-aminoethyl-1,3-propanediamine)-bis-3-nitro-1,8 naphthalimide 31a and N, N′-(bis-N-(4-acetylbenzamide), bis-aminoethyl-1,3-proplanediamine)-bis-3-nitro-1,8 naphthalimide 31b

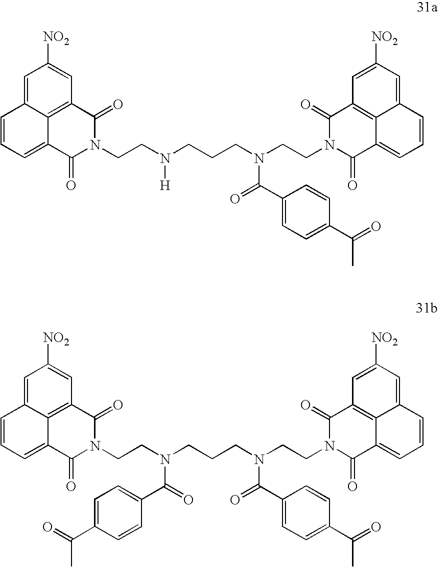

4-Acetylbenzoic acid (0.061 gm, 0.36 mmole), diisopropylethylamine (0.13 ml, 0.72 mmole), 2-(7-Aza-1H-benzotriazole-1-yl)-1,1,3,3-tetramethyluronium hexafluorophosphate (HATU, 0.133 gm, 0.35 mmole), and 3 ml DMF were stirred under nitrogen for 15 minutes at room temperature. A solution of N,N′-(bis-aminoethyl-1,3-propanediamine)-bis-3-nitro-1,8 naphthalimide (0.212 gm, 0.35 mmole) and 12 ml DMF was added. After 30 minutes, LC/MS analysis showed the presence of reactant, and products 31a and 31b. The reaction was stirred overnight at room temperature, quenched with aqueous TFA, concentrated, dissolved in acetic acid and DMF and purified by prep. HPLC to give separated and purified N,N′-(N-(4-acetylbenzamide), bis-aminoethyl-1,3-propanediamine)-bis-3-nitro-1,8 naphthalimide 31a MS m/z 757 (M+H)⁺, and N,N′-(bis-N-(4-acetylbenzamide), bis-aminoethyl-1,3-propanediamine)-bis-3-nitro-1,8 naphthalimide 31b.

### Example 32

### Synthesis of N, N′-(N-(3-benzoylpropionamide), bis-aminoethyl-1,3-propanediamine)-bis-3-nitro-1,8 naphthalimide 32

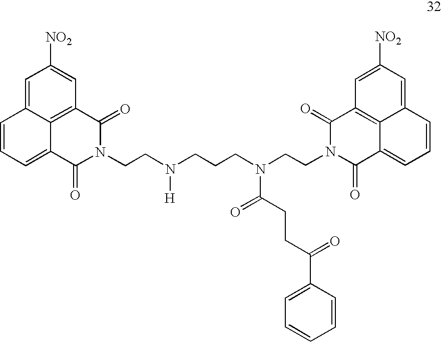

3-benzoylpropionic acid (0.037 gm, 0.20 mmole), diisopropylethylamine (0.07 ml, 0.40 mmole), HATU (0.077 gm, 0.20 mmole), and 2 ml DMF were stirred under nitrogen for 10 minutes at room temperature, then it was added to a solution of N,N′-(bis-aminoethyl-1,3-propanediamine)-bis-3-nitro-1,8 naphthalimide (0.162 gm, 0.35 mmole), diisopropylethylamine (0.17 ml, 0.90 mmole) and 6 ml DMF was added. After 30 minutes, LC/MS analysis showed the presence of reactant and product 32. The reaction was stirred overnight at room temperature, quenched with aqueous TFA, concentrated, dissolved in acetic acid and DMF and purified by prep. HPLC to give N,N′-(N-(3-benzoylpropionamide), bis-aminoethyl-1,3-propanediamine)-bis-3-nitro-1,8 naphthalimide 32. MS m/z 771 (M+H)⁺.

### Example 33a

### Synthesis of N, N′-(N-(levulinamide), bis-aminoethyl-1,3-propanediamine)-bis-3-nitro-1,8 naphthalimide 33a

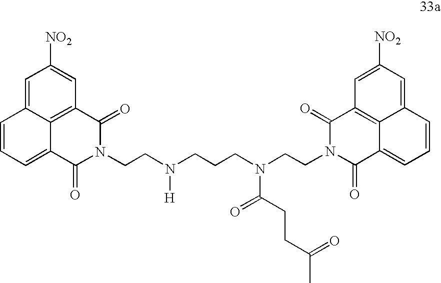

Levulinic acid (0.015 ml, 0.15 mmole), diisopropylethylamine (0.04 ml, 0.25 mmole), HATU (0.054 gm, 0.15 mmole), and 1 ml DMF were stirred under nitrogen for 15 minutes at room temperature, then it was added to a solution of N,N′-(N—F₁₇BOC-aminoethyl-1,3-propanediamine)-bis-3-nitro-1,8 naphthalimide (62 mg, 0.049 mmole), having the structure:

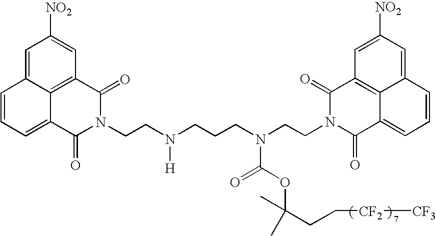

diisopropylethylamine (DIEA, 0.02 ml), and 1 ml DMF at room temperature. LC/MS analysis showed the presence of reactant and product 32. The reaction was stirred overnight at room temperature, quenched with aqueous TFA to hydrolyze the fluorocarbamate protecting group, concentrated, dissolved in acetic acid and DMF and purified by prep. HPLC to give N,N′-(N-(levulinamide), bis-aminoethyl-1,3-propanediamine)-bis-3-nitro-1,8 naphthalimide 33a. MS m/z 709 (M+H)⁺.

### Example 33b

### Synthesis of N, N′-(N-(tert-butylmalonamide), bis-aminoethyl-1,3-propanediamine)-bis-4-N-imidazolyl-1,8 naphthalimide 33b

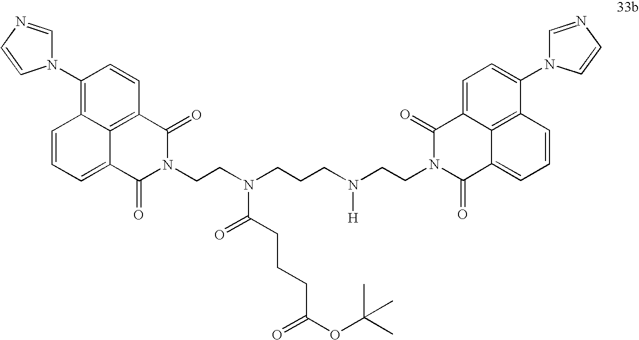

Following the same protocol as Example 33a, N-(tert-butylmalonamide), bis-aminoethyl-1,3-propanediamine)-bis-4-N-imidazolyl-1,8 naphthalimide 33b was prepared from mono-tert-butyl malonic acid and N,N′-(N—F₁₇BOC-aminoethyl-1,3-propanediamine)-bis-4-N-imidazolyl-1,8 naphthalimide, followed by hydrolysis of the N—F₁₇BOC group.

### Example 34

### Synthesis of N, N′-(bis-2-acetamido-1,3-propanediamine)-bis-4-morpholino- 1,8 naphthalimide

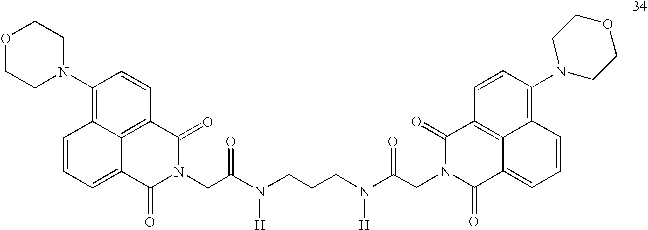

The bis TFA salt of 1,3-bisglycyl-1,3 diaminopropane 2 (22 mg, 0.53 mmole) and DIEA (0.046 ml, 0.26 mmole) were dissolved in 0.5 ml DMF at room temperature under nitrogen. 4-Morpholino-1,8-naphthalic anhydride 1 (31 mg, 0.11 mmole) was added. The mixture was microwave heated at 150° C. for 5 minutes, then heating was increased to 200° C. for 10 minutes. The reaction was quenched with 4 ml 0.3M aqueous TFA and purified by prep. HPLC to give N,N′-(bis-2-acetamido-1,3-propanediamine)-bis-4-morpholino-1,8 naphthalimide 34. MS m/z 719 (M+H)⁺.

### Example 35

### Synthesis of N, N′-(bis-ethyl, malondiamide)-bis-4-morpholino-1,8 naphthalimide 35

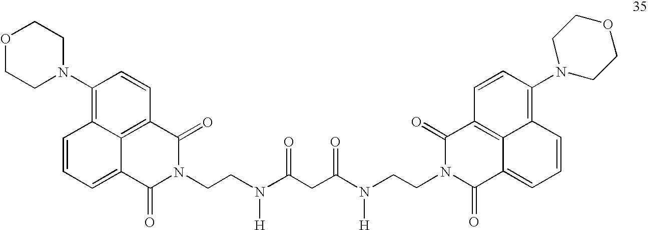

The TFA salt of N′,N³-bis(2-aminoethyl)malonamide 3 (0.041 mmole) and DIEA (0.058 ml, 0.33 mmole) were dissolved in 0.5 ml DMF at room temperature under nitrogen. 4-Morpholino-1,8-naphthalic anhydride 1 (23 mg, 0.082 mmole) was added. The mixture was microwave heated at 180° C. for 10 minutes, then heating was increased to 200° C. for 5 minutes. The reaction was quenched with 4 ml 1.3M aqueous TFA and purified by prep. HPLC to give N,N′-(bis-ethyl, malondiamide)-bis-4-morpholino-1,8 naphthalimide 35. MS m/z 719 (M+H)⁺.

### Example 36

### Synthesis of N, N′-(bis-ethyl, malondiamide)-bis-3-nitro-1,8 naphthalimide 36

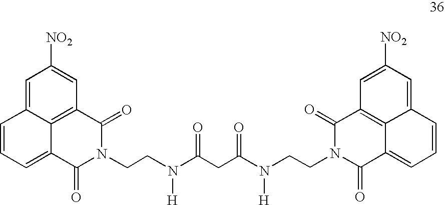

The TFA salt of N′,N³-bis(2-aminoethyl)malonamide 3 (0.074 mmole) and DIEA (0.10 ml, 0.59 mmole) were dissolved in 1 ml DMF at room temperature under nitrogen. 3-Nitro-1,8-naphthalic anhydride (36 mg, 0.148 mmole) was added. The mixture was microwave heated at 150° C. for 5 minutes. The reaction was quenched with 4 ml 1.3M aqueous TFA and purified by prep. HPLC to give N,N′-(bis-ethyl, malondiamide)-bis-3-nitro-1,8 naphthalimide 36. MS m/z 639 (M+H)⁺.

### Example 37

### Synthesis of N, N′-2-acetamido-1,3-propanediamine-ethyl)-bis-4-amino-1,8 naphthalimide 37

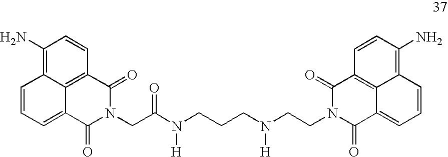

A solution of N-glycyl-4-amino-1,8 naphthalimide 5 (25 mg, 0.10 mmole), DIEA (0.043 ml, 0.25 mmole), Benzotriazole- 1-yl-oxy-tris-pyrrolidino-phosphonium hexafluorophosphate (PyBOP, 52 mg, 0.10 mmole), and 0.5 ml DMF was stirred under nitrogen at room temperature for 75 minutes. A solution of the TFA salt of N-aminopropylethylamine-4-amino-1,8 naphthalimide 11a (57 mg, 0.103 mmole) and DIEA (0.070 ml, 0.40 mmole), and 1 ml DMF was stirred under nitrogen at room temperature for 40 minutes. The two solutions were mixed and stirred overnight. The mixture was concentrated under vacuum, diluted with aqueous TFA, and purified by prep. HPLC to give N,N′-2-acetamido-1,3-propanediamine-ethyl)-bis-4-amino-1,8 naphthalimide 37. MS m/z 565 (M+H)⁺.

### Example 38

### Synthesis of N, N′-2-acetamido-1,2-ethanediamine-propyl)-bis-4-morpholino-1,8 naphthalimide 38

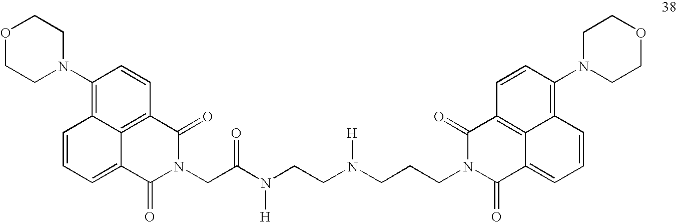

A solution of N-glycyl-4-morpholino-1,8 naphthalimide 6 (25 mg, 0.10 mmole), DIEA (0.043 ml, 0.25 mmole), benzotriazole-1-yl-oxy-tris-pyrrolidino-phosphonium hexafluorophosphate (PyBOP, 52 mg, 0.10 mmole), and 0.5 ml DMF was stirred under nitrogen at room temperature for 75 minutes. A solution of the TFA salt of N-aminoethylpropylamine-(4-morpholino-1,8 naphthalimide 12b (57 mg, 0.103 mmole) and DIEA (0.070 ml, 0.40 mmole), and 1 ml DMF was stirred under nitrogen at room temperature for 40 minutes. The two solutions were mixed and stirred overnight. The mixture was concentrated under vacuum, diluted with aqueous TFA, and purified by prep. HPLC to give the TFA salt of N,N′-2-acetamido-1,3-ethanediamine-propyl)-bis-4-morpholino-1,8 naphthalimide 38. MS m/z 705 (M+H)⁺.

### Example 39

### Synthesis of N, N′-2-acetamido- 1,2-ethanediamine-propyl)-bis-4-amino-1,8 naphthalimide 39

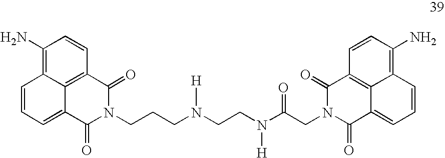

A first solution of N-glycyl-4-amino-1,8 naphthalimide 5 (31 mg, 0.114 mmole), DIEA (0.040 ml, 0.23 mmole), benzotriazole-1-yl-oxy-tris-pyrrolidino-phosphonium hexafluorophosphate (PyBOP, 61 mg, 0.12 mmole), and 1 ml DMF was stirred under nitrogen at room temperature for 75 minutes. A second solution of the TFA salt of N-aminoethylpropylamine-4-amino-1,8 naphthalimide 11b (63 mg, 0.115 mmole) and DIEA (0.070 ml, 0.40 mmole), and 1 ml DMF was stirred under nitrogen at room temperature for 40 minutes. The first solution was added slowly to the second solution and the resultant mixture was stirred overnight. The mixture was quenched with 10% aqueous TFA, concentrated under vacuum, and purified by prep. HPLC to give the TFA salt of N,N′-2-acetamido-1,3-ethanediamine-propyl)-bis-4-amino-1,8 naphthalimide 39. MS m/z 565 (M+H)⁺.

### Example 40

### Synthesis of N, N′-2-acetamido-1,2-ethanediamine-propyl)-bis-3-nitro-1,8 naphthalimide 40

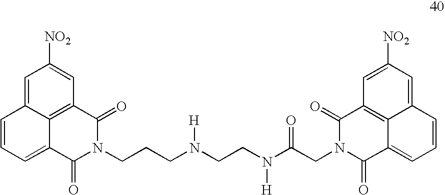

A first solution of N-glycyl-3-nitro-1,8 naphthalimide 4 (47 mg, 0.145 mmole), DIEA (0.038 ml, 0.21 mmole), benzotriazole-1-yl-oxy-tris-pyrrolidino-phosphonium hexafluorophosphate (PyBOP, 111 mg, 0.23 mmole), and 1 ml DMF was stirred under nitrogen at room temperature for 75 minutes. A second solution of the TFA salt of N-aminoethylpropylamine-3-nitro-1,8 naphthalimide 10b (106 mg, 0.177 mmole) and DIEA (0.092 ml, 0.53 mmole), and 1 ml DMF was stirred under nitrogen at room temperature for 40 minutes. The first solution was added slowly to the second solution and the resultant mixture was stirred overnight. The mixture was quenched with 10% aqueous TFA, concentrated under vacuum, and purified by prep. HPLC to give the TFA salt of N,N′-2-acetamido-1,2-ethanediamine-propyl)-bis-3-nitro-1,8 naphthalimide 40. MS m/z 625 (M+H)⁺.

### Example 41

### Synthesis of N, N′-2-acetamido-1,2-propanediamine-ethyl)-bis-4-morpholino-1,8 naphthalimide 41

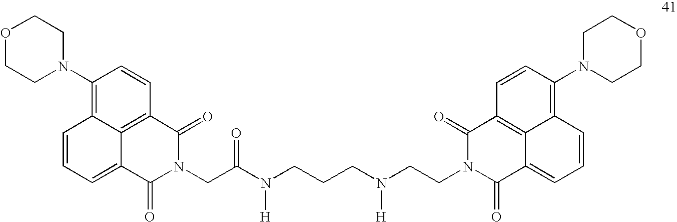

A first solution of N-glycyl-4-morpholino-1,8 naphthalimide 6 (31 mg, 0.092 mmole), DIEA (0.038 ml, 0.21 mmole), benzotriazole-1-yl-oxy-tris-pyrrolidino-phosphonium hexafluorophosphate (PyBOP, 57 mg, 0.11 mmole), and 1 ml DMF was stirred under nitrogen at room temperature for 75 minutes. A second solution of the TFA salt of N-aminopropylethylamine-(4-morpholino-1,8 naphthalimide 12a (57 mg, 0.092 mmole) and DIEA (0.050 ml, 0.53 mmole), and 1 ml DMF was stirred under nitrogen at room temperature for 40 minutes. The first solution was added slowly to the second solution and the resultant mixture was stirred overnight. The mixture was quenched with 10% aqueous TFA, concentrated under vacuum, and purified by prep. HPLC to give the TFA salt of N,N′-2-acetamido-1,2-propanediamine-ethyl)-bis-4-morpholino-1,8 naphthalimide 41. MS m/z 705 (M+H)⁺.

### Example 42

### Synthesis of N, N′-2-acetamido-1,2-pronanediamine-ethyl)-bis-3-nitro-1,8 naphthalimide 42

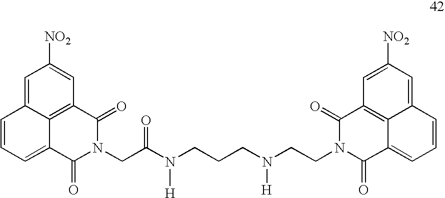

A first solution of N-glycyl-3-nitro-1,8 naphthalimide 4 (37 mg, 0.114 mmole), DIEA (0.040 ml, 0.23 mmole), benzotriazole- 1-yl-oxy-tris-pyrrolidino-phosphonium hexafluorophosphate (PyBOP, 78 mg, 0.16 mmole), and 1 ml DMF was stirred under nitrogen at room temperature for 75 minutes. A second solution of the TFA salt of N-aminopropylethylamine-3-nitro-1,8 naphthalimide 10a (66 mg, 0.105 mmole) and DIEA (0.055 ml, 0.31 mmole), and 1 ml DMF was stirred under nitrogen at room temperature for 40 minutes. The first solution was added slowly to the second solution and the resultant mixture was stirred overnight. The mixture was quenched with 10% aqueous TFA, concentrated under vacuum, and purified by prep. HPLC to give the TFA salt of N,N′-2-acetamido-1,2-propanediamine-ethyl)-bis-3-nitro-1,8 naphthalimide 42. MS m/z 625 (M+H)⁺.

### Example 43

### Synthesis of N, N′-2-acetamido-2-ethyleneoxyethyl)-bis-3-nitro-1,8 naphthalimide 43

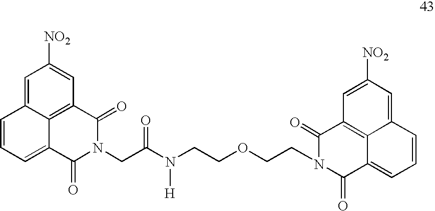

A first solution of N-glycyl-3-nitro-1,8 naphthalimide 4 (18 mg, 0.052 mmole), DIEA (0.030 ml, 0.17 mmole), benzotriazole-1-yl-oxy-tris-pyrrolidino-phosphonium hexafluorophosphate (PyBOP, 28 mg, 0.054 mmole), and 0.2 ml DMF was mixed under nitrogen at room temperature for 30 minutes. A second solution of the TFA salt of N-aminoethylethoxy-3-nitro-1,8 naphthalimide 7 (25 mg, 0.052 mmole) and DIEA (0.020 ml, 0.12 mmole), and 0.5 ml DMF was mixed under nitrogen at room temperature for 40 minutes. The first solution was added slowly to the second solution and the resultant mixture was stirred overnight. The mixture was quenched with 10% aqueous TFA, concentrated under vacuum, and purified by prep. HPLC to give the TFA salt of N,N′-2-acetamido-2-ethyleneoxyethyl)-bis-3-nitro-1,8 naphthalimide 43. MS m/z 612 (M+H)⁺.

### Example 44

### Synthesis of N, N′-(4-aza-octanyl)-bis-4-bromo-1,8 naphthalimide 44

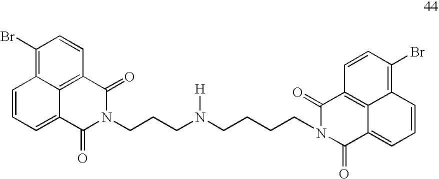

A solution of 3-bromo-1,8-naphthalic anhydride (0.409 gm, 1.40 mmole), N¹-(3-aminopropyl)butane-1,4-diamine (spermidine, 0.11 ml, 0.70 mmole), and 4 ml ethanol was microwave heated at 150° C. for 5 minutes. The mixture was cooled, filtered, concentrated under vacuum, dissolved in 1 ml acetic acid and 0.5 ml DMF, and purified by prep. HPLC to give the TFA salt of N,N′-(4-aza-octanyl)-bis-4-bromo-1,8 naphthalimide 44. MS m/z 664 (M+H)⁺.

### Example 45

### Synthesis of N, N′-(4-aza-octanyl)-bis-4-morpholino-1,8 naphthalimide 45

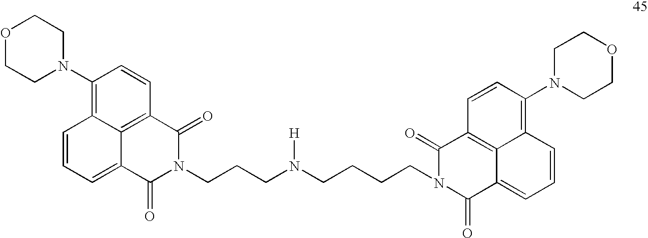

A solution of N,N′-(4-aza-octanyl)-bis-4-bromo-1,8 naphthalimide 44 (0.029 gm, 0.031 mmole) and 3 ml morpholine was heated at 80° C. for 16 hours. LC/MS analysis showed the reaction was complete. The mixture was cooled, concentrated, dissolved in 2.5 ml acetic acid and 0.5 ml 0.1% aqueous TFA, and purified by prep. HPLC to give N,N′-(4-aza-octanyl)-bis-4-morpholino-1,8 naphthalimide 45. MS m/z 676 (M+H)⁺.

### Example 46

### Synthesis of N, N′-(2-ethoxy-N-(2,4-dinitrobenzensulfonyl)-ethylethanamine)-bis-3-nitro-1,8 naphthalimide 46

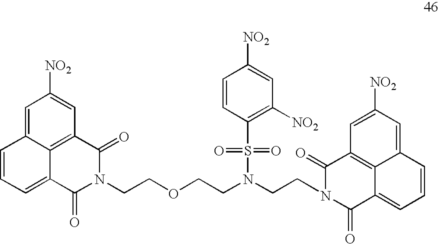

Cesium carbonate (0.144 gm, 0.442 mmole) was added to N-(2,4-dinitrophenylaminoethylethoxy)-3-nitro-1,8 naphthalimide 15 (79 mg, 0.14 mmole) in 1.5 ml DMF under nitrogen at room temperature. N-Iodoethyl-(3-nitro-1,8 naphthalimide 14 (0.11 gm, 0.28 mmole) in 7.5 ml was added, and the mixture was stirred overnight at 40° C. The mixture was concentrated, and purified by prep. HPLC to give N,N′-(2-ethoxy-N-(2,4-dinitrobenzensulfonyl)-ethylethanamine)-bis-3-nitro-1,8 naphthalimide 46. MS m/z 827 (M⁺).

### Example 47

### Synthesis of N, N′-(2-ethoxy-N-ethylethanamine)-bis-3-nitro-1,8 naphthalimide 47

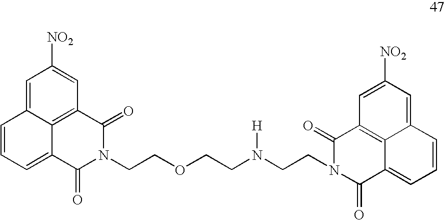

A solution of N,N′-(2-ethoxy-N-(2,4-dinitrobenzensulfonyl)-ethylethanamine)-bis-3-nitro-1,8 naphthalimide 46 (36 mg, 0.043 mmole), cesium carbonate (0.045 gm, 0.14 mmole). thiophenol (0.045 ml, 0.043 mmole) and 2 ml DMF was stirred under nitrogen at room temperature for 20 minutes. The mixture was concentrated, and purified by prep. HPLC to give N,N′-(2-ethoxy-N-ethylethanamine)-bis-3-nitro-1,8 naphthalimide 47. MS n7z 598 (M+H)⁺.

### Example 48

### Synthesis of N, N′-(bis-2-acetamido-1,3-propanediamine)-bis-4-amino-1,8 naphthalimide 48

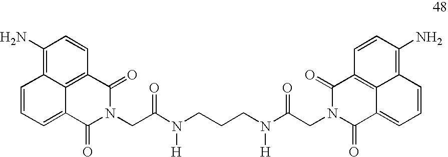

The bis HCl salt of 1,3-bis glycyl-1,3 diaminopropane 2 (37 mg, 0.141 mmole) and DIEA (0.058 ml, 0.33 mmole) were dissolved in 2 ml ethanol at room temperature under nitrogen. 4-Amino-1,8-naphthalic anhydride (63 mg, 0.28 mmole) and DIEA (0.045 ml, 0.26 mmole) in 1 ml ethanol was added. The mixture was microwave heated at 150° C. for 5 minutes. The reaction was quenched with 4 ml 1.3M aqueous TFA and purified by prep. HPLC to give N,N′-(bis-2-acetamido-1,3-propanediamine)-bis-4-amino-1,8 naphthalimide 48.

### Example 49

### Synthesis of N¹, N² bis methyl. N, N′-(bis-aminoethyl-1,3-propanediamine)-4-N-imidazolyl, 4′-(3-aminopropyl)amino)-1,8 naphthalimide 49

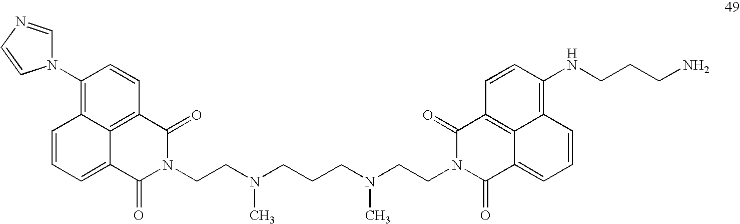

A solution of the trifluoroacetate salt of N′,N² bis methyl, N,N′-(bis-aminoethyl-1,3-propanediamine)-4-N-imidazolyl, 4′-bromo-1,8 naphthalimide (0.050 gm, 0.048 mmole), 1,3 propanediamine (0.18 ml, 2.4 mmole), ethanol, DMF, and N-methylmorpholine (NMM) was heated at 150° C. for 5 minutes. Preparatory HPLC gave N′,N² bis methyl, N,N′-(bis-aminoethyl-1,3-propanediamine)-4-N-imidazolyl, 4′-(3-aminopropyl)amino)-1,8 naphthalimide 49.

### Example 50

### Synthesis of N¹, N² bis methyl, N, N′-(bis-aminoethyl-1,3-propanediamine)-4-N-imidazolyl, 4′-(4-mercaptopropylpiperazinyl)-1,8 naphthalimide 50

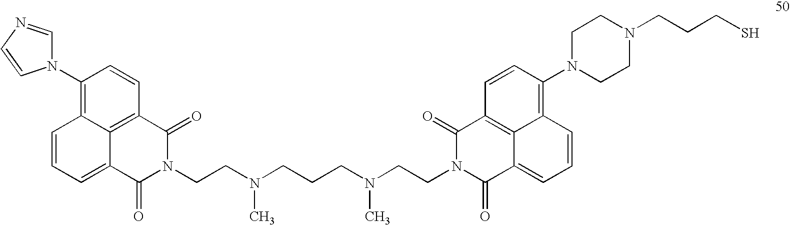

A solution of the trifluoroacetate salt of N′,N² bis methyl, N,N′-(bis-aminoethyl-1,3-propanediamine)-4-N-imidazolyl, 4′-bromo-1,8 naphthalimide, an excess of piperazine, ethanol, DMF, and N-methylmorpholine (NMM) was heated. The piperazinyl adduct was isolated and treated with an excess of ethylene sulfide. Preparatory HPLC gave N′,N bis methyl, N,N′-(bis-aminoethyl-1,3-propanediamine)-4-N-imidazolyl, 4′-(4-mercaptopropylpiperazinyl)-1,8 naphthalimide 50.

### Example 51

### Synthesis of N, N′-(bis-2-acetamido-1,3-propanediamine)-4-piperazinyl, 4′-(4N-(3-mercaptopropyl)-piperazinyl-1,8 naphthalimide 51

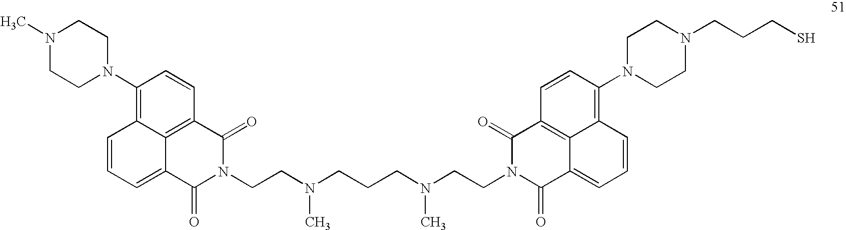

N,N′-(Bis-2-acetamido-1,3-propanediamine)-4-piperazinyl, 4′-(4N-(3-mercaptopropyl)-piperazinyl-1,8 naphthalimide 51 was prepared following the protocol of Example 50.

### Example 52

### Synthesis of N, N′-(N¹-ethyl, N²—(N-methyl, N—BOC glycyl), bis-aminoethyl-1,3-propanediamine)-bis-2-nitro-1,8 naphthalimide 52

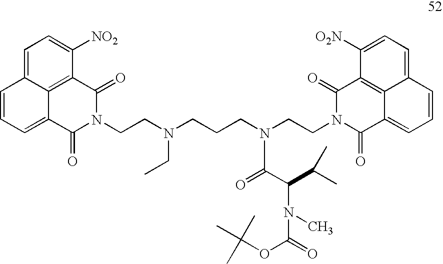

2-Fluoro-1-ethyl-pyridinium tetrafluoroborate (FEP, 0.079 gm), N-Methyl, N—BOC glycine (0.070 gm, 0.37 mmole), DIEA (0.071 ml), and 1 ml DMF were mixed and stirred under nitrogen at room temperature then added to a solution of N,N′-(N¹-ethyl, N²—H, bis-aminoethyl-1,3-propanediamine)-bis-2-nitro-1,8 naphthalimide (0.31 gm, 0.37 mmole), DIEA (0.81 mmole) in 3 ml DMF, to give N,N′-(N₁-ethyl, N 2-(N-methyl, N—BOC glycyl), bis-aminoethyl-1,3-propanediamine)-bis-2-nitro-1,8 naphthalimide 52: MS m/z 852.4 (M+H)⁺.

### Example 53

### Synthesis of N, N′-(N¹-ethyl, N²—(N-methyl glycyl), bis-aminoethyl-1,3-propanediamine)-bis-2-nitro-1,8 naphthalimide 53

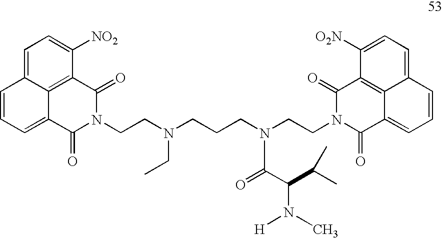

The BOC group was removed with acid to give N,N′-(N′-ethyl, N²—(N-methyl glycyl), bis-aminoethyl-1,3-propanediamine)-bis-2-nitro-1,8 naphthalimide 53. MS m/z 752.1 (M+H)⁺.

### Example 54

### Synthesis of MC-vc-PAB-(N, N′-2-acetamido-1,3-propanediamine-ethyl)-bis-4-amino-1,8 naphthalimide) 101

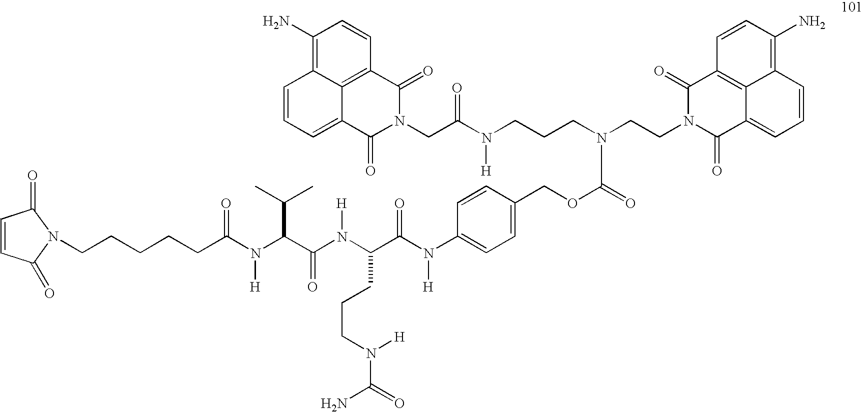

A mixture of N,N′-2-acetamido-1,3-propanediamine-ethyl)-bis-4-amino-1,8 naphthalimide 37 (18 mg, 0.025 mmole), DIEA (0.0044 ml, 0.05 mmole) and 1.2 ml DMF was stirred at room temperature under nitrogen for 10 minutes, then maleimido-caproyl-valine-citrulline-para-aminobenzyl-4-nitrophenylcarbonate (MC-vc-PAB-OPNP, 19 mg, 0.025 mmole) having the structure: 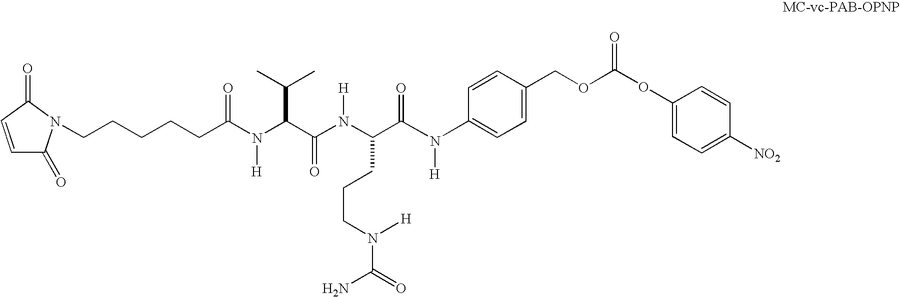
and DIEA (0.005 ml) were added, and the mixture was stirred at room temperature overnight. The mixture was quenched with 0.20 ml 1.3M aqueous TFA and 0.30 ml acetic acid, and purified by prep. HPLC to give MC-vc-PAB-(N,N′-2-acetamido-1,3-propanediamine-ethyl)-bis-4-amino-1,8 naphthalimide) 101. MS m/z 1164(M+H)⁺.

### Example 55

### Synthesis of MC-vc-PAB-(N, N′-2-acetamido-1,3-ethanediamine-propyl)-bis-4-morpholino-1,8 naphthalimide) 102

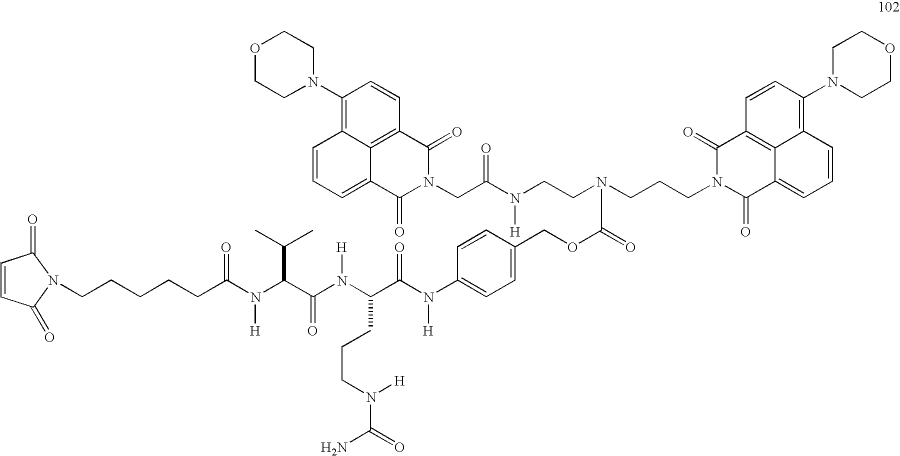

A mixture of N,N′-2-acetamido-1,3-ethanediamine-propyl)-bis-4-morpholino-1,8 naphthalimide 38 (9 mg, 0.011 mmole), DIEA (0.0044 ml, 0.05 mmole) and 1.2 ml DMF was stirred at room temperature under nitrogen for 10 minutes, then maleimido-caproyl-valine-citrulline-para-aminobenzyl-4-nitrophenylcarbonate (MC-vc-PAB-OPNP, 18 mg, 0.025 mmole) and DIEA (0.005 ml) were added, and the mixture was stirred at room temperature overnight. The mixture was quenched with 0.20 ml 1.3M aqueous TFA and 0.30 ml acetic acid, and purified by prep. HPLC to give MC-vc-PAB-(N,N′-2-acetamido-1,3-ethanediamine-propyl)-bis-4-morpholino-1,8 naphthalimide) 102. MS m/z 1304 (M+H)⁺.

### Example 56

### Synthesis of MC-vc-PAB-(N, N′-2-acetamido-1,2-propanediamine-ethyl)-bis-4-morpholino-1,8 naphthalimide) 103

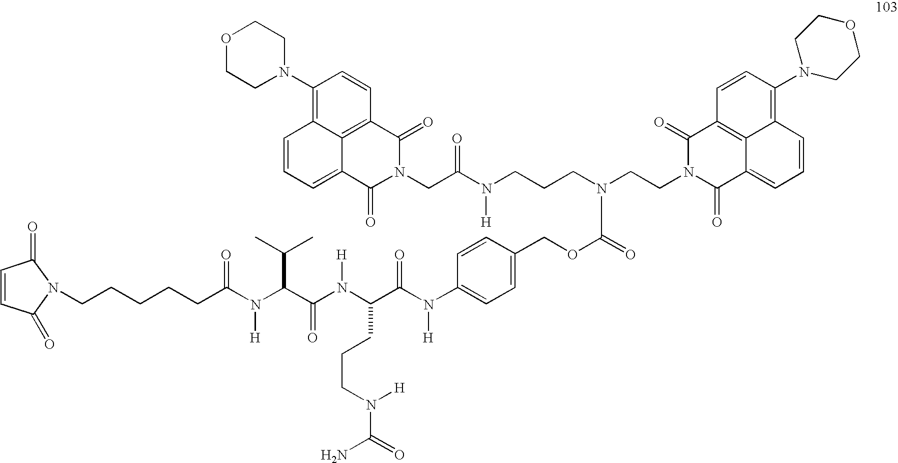

A mixture of N,N′-2-acetamido-1,2-propanediamine-ethyl)-bis-4-morpholino-1,8 naphthalimide 41 (9 mg, 0.011 mmole), DIEA (0.0044 ml, 0.05 mmole) and 1.2 ml DMF was stirred at room temperature under nitrogen for 10 minutes, then maleimido-caproyl-valine-citrulline-para-aminobenzyl-4-nitrophenylcarbonate (MC-vc-PAB-OPNP, 7 mg, 0.011 mmole) and DIEA (0.005 ml) were added, and the mixture was stirred at room temperature overnight. The mixture was quenched with 0.20 ml 1.3M aqueous TFA and 0.30 ml acetic acid, and purified by prep. HPLC to give MC-vc-PAB-(N,N′-2-acetamido-1,3-propanediamine-ethyl)-bis-4-morpholino-1,8 naphthalimide) 103. MS m/z 1304 (M+H)⁺.

### Example 57

### Synthesis of MC-vc-PAB-(N, N′-2-acetamido-1,3-ethanediamine-propyl)-bis-4-amino-1,8 naphthalimide) 104

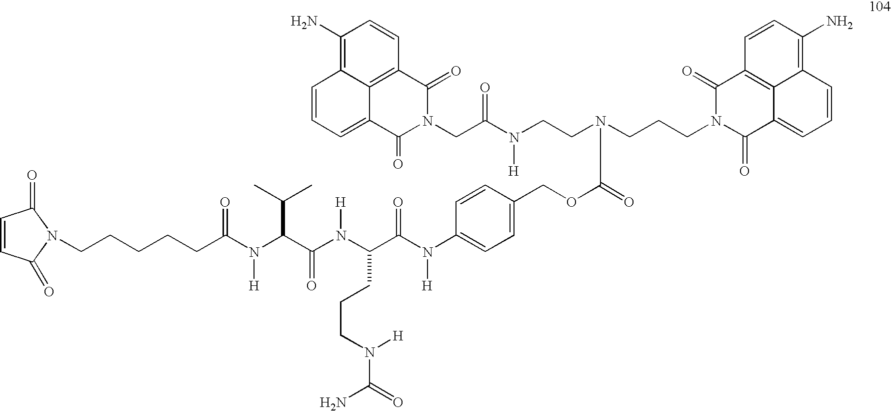

A mixture of N,N′-2-acetamido-1,3-ethanediamine-propyl)-bis-4-amino-1,8 naphthalimide 39 (10 mg, 0.013 mmole), DIEA (0.003 ml, 0.03 mmole) and 0.7 ml DMF was stirred at room temperature under nitrogen for 10 minutes, then maleimido-caproyl-valine-citrulline-para-aminobenzyl-4-nitrophenylcarbonate (MC-vc-PAB-OPNP, 10 mg, 0.013 mmole) and DIEA (0.005 ml) were added, and the mixture was stirred at room temperature overnight. The mixture was quenched with 0.10 ml 1.3M aqueous TFA and 0.30 ml acetic acid, and purified by prep. HPLC to give MC-vc-PAB-(N,N′-2-acetamido-1,3-ethanediamine-propyl)-bis-4-amino-1,8 naphthalimide) 104. MS m/z 1164 (M+H)⁺.

### Example 58

### Synthesis of MC-vc-PAB-(N,N′-2-acetamido-1,2-propanediamine-ethyl)-bis-3-nitro-1,8 naphthalimide) 105

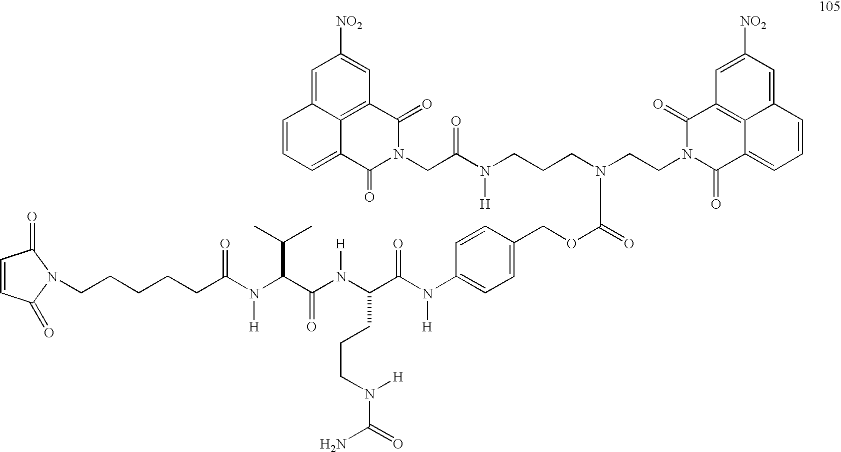

A mixture of N, N′-2-acetamido-1,2-propanediamine-ethyl)-bis-3-nitro-1,8 naphthalimide 42 (21 mg, 0.026 mmole), DIEA (0.016 ml, 0.092 mmole) and 0.2 ml DMF was stirred at room temperature under nitrogen for 10 minutes, then maleimido-caproyl-valine-citrulline-para-aminobenzyl-4-nitrophenylcarbonate (MC-vc-PAB-OPNP, 26 mg, 0.035 mmole) and DIEA (0.013 ml, 0.075 mmole) were added, and the mixture was stirred at room temperature overnight. The mixture was quenched with 0.50 ml 1.3M aqueous TFA and purified by prep. HPLC to give MC-vc-PAB-(N,N′-2-acetamido-1,3-propanediamine-ethyl)-bis-3-nitro-1,8 naphthalimide) 105. MS m/z 1223 (M⁺).

### Example 59

### Synthesis of MC-vc-PAB-(N,N′-2-acetamido-1,3-ethanediamine-propyl)-bis-3-nitro-1,8 naphthalimide) 106

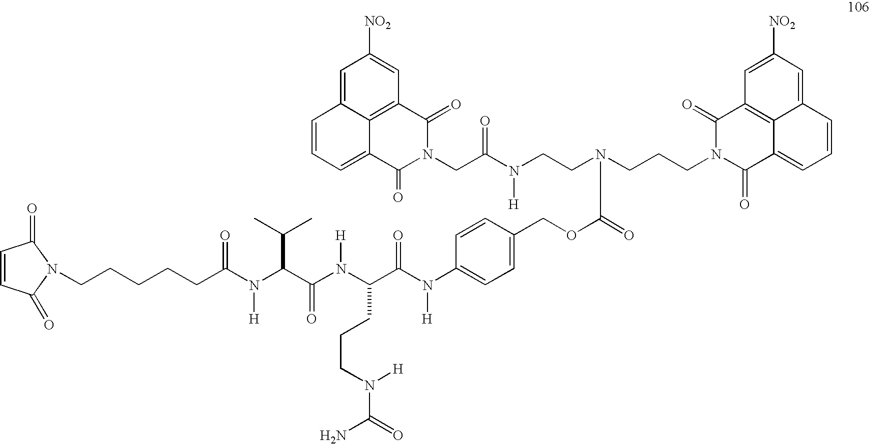

A mixture of N,N′-2-acetamido-1,2-ethanediamine-propyl)-bis-3-nitro-1,8 naphthalimide 40 (9 mg, 0.012 mmole), DIEA (0.004 ml, 0.023 mmole) and 0.7 ml DMF was stirred at room temperature under nitrogen for 10 minutes, then maleimido-caproyl-valine-citrulline-para-aminobenzyl-4-nitrophenylcarbonate (MC-vc-PAB-OPNP, 9 mg, 0.012 mmole) and DIEA (0.005 ml) were added, and the mixture was stirred at room temperature overnight. The mixture was quenched with 0.1 ml 1.3M aqueous TFA and 0.30 ml acetic acid, and purified by prep. HPLC to give MC-vc-PAB-(N,N′-2-acetamido-1,3-ethanediamine-propyl)-bis-3-nitro-1,8 naphthalimide) 106. MS m/z 1224 (M+H)⁺.

### Example 60

### Synthesis of MC-vc-PAB-(N. N′-(4-aza-octanyl)-bis-3-nitro-1,8 naphthalimide) 107

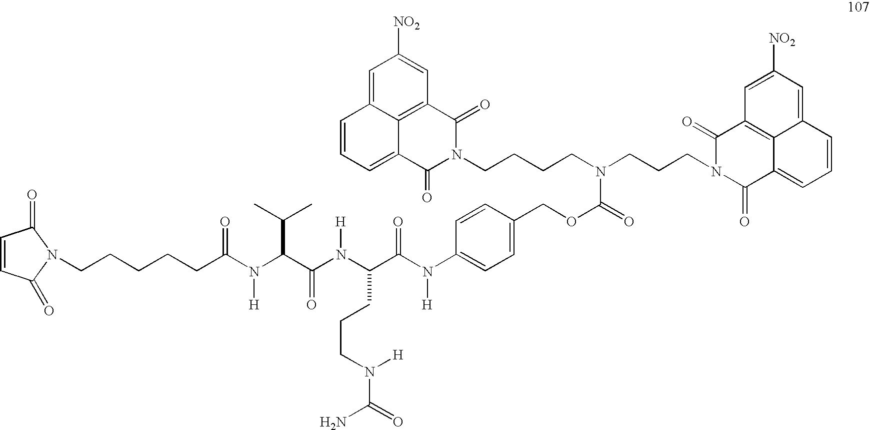

A mixture of N,N′-(4-aza-octanyl)-bis-3-nitro-1,8 naphthalimide (15 mg, 0.021 mmole), DIEA (0.010 ml, 0.057 mmole) and 0.2 ml DMF was stirred at room temperature under nitrogen for 10 minutes, then maleimido-caproyl-valine-citrulline-para-aminobenzyl-4-nitrophenylcarbonate (MC-vc-PAB-OPNP, 15 mg, 0.021 mmole) N-hydroxybenzotriazole (HOBt, (0.002 mmole) and DIEA (0.005 ml) in 0.8 ml DMF were added, and the mixture was stirred at 35° C. for 1.5 hours. The mixture was filtered and purified by prep. HPLC to give MC-vc-PAB-(N,N′-(4-aza-octanyl)-bis-3-nitro-1,8 naphthalimide) 107. MS m/z 1195 (M+H)⁺.

### Example 61

### Synthesis of MC-vc-PAB-(N, N′-(2-ethoxy-N-ethylethanamine)-bis-3-nitro-1,8 naphthalimide) 108

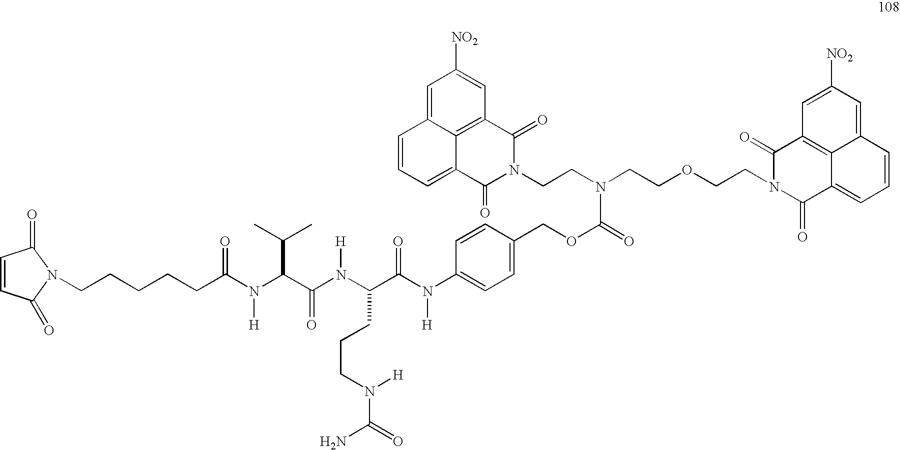

A mixture of N,N′-(2-ethoxy-N-ethylethanamine)-bis-3-nitro-1,8 naphthalimide 47 (7 mg, 0.009 mmole), DIEA (0.004 ml, 0.023 mmole) and 0.7 ml DMF was stirred at room temperature under nitrogen for 10 minutes, then maleimido-caproyi-valine-citruiline-para-aminobenzyl-4-nitrophenylcarbonate (MC-vc-PAB-OPNP, 7 mg, 0.009 mmole) and DIEA (0.005 ml) were added, and the mixture was stirred at room temperature overnight. The mixture was diluted with 1.6 ml DMF, and purified by prep. HPLC to give MC-vc-PAB-(N,N′-(2-ethoxy-N-ethylethanamine)-bis-3-nitro-1,8 naphthalimide) 108. MS m/z 1196 (M⁺).

### Example 62

### Synthesis of MC-hydrazone-(N,N′-(N-(levulinamide), bis-aminoethyl-1,3-propanediamine)-bis-3-nitro-1,8 naphthalimide) 109

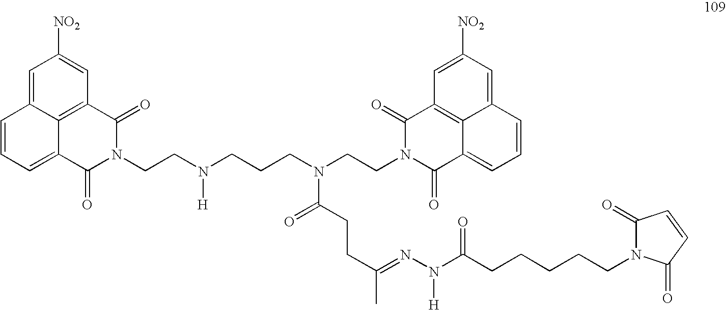

The TFA salt of N,N′-(N-(levulinamide), bis-aminoethyl-1,3-propanediamine)-bis-3-nitro-1,8 naphthalimide 33a (15 mg, 0.018 mmole), N-[6-maleimidocaproic acid]hydrazide (EMCH, Pierce Biotechnology, 20 mg, 0.088 mmole), acetic acid (0.010 ml, 0.002 mmole), and 3 ml DMF were stirred at room temperature for about 48 hours. The mixture was diluted with DMF and purified by prep. HPLC to give MC-hydrazone-(N,N′-(N-(levulinamide), bis-aminoethyl-1,3-propanediamine)-bis-3-nitro-1,8 naphthalimide) 109. MS m/z 916 (M+H)⁺.

### Example 63

### Synthesis of MC-hydrazone-(N, N′-(N-(levulinamide), bis-aminoethyl-1,3-propanediamine)-bis-3-nitro-1,8 naphthalimide) 110

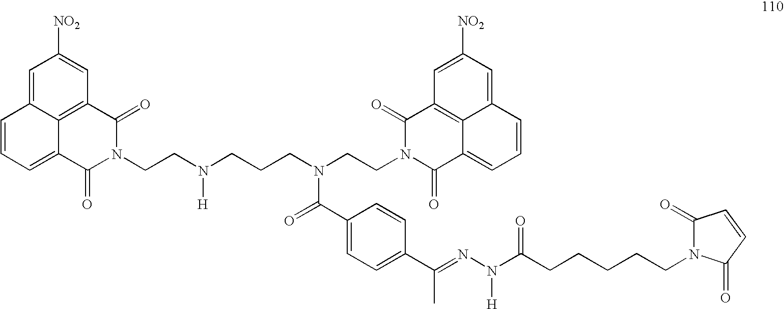

The TFA salt of N,N′-(N-(4-acetylbenzamide), bis-aminoethyl-1,3-propanediamine)-bis-3-nitro-1,8 naphthalimide 31a (34 mg, 0.039 mmole), EMCH (Pierce Biotechnology, 28 mg, 0.124 mmole), acetic acid (0.011 ml, 0.002 mmole), 5 ml ethanol, and 1 ml DMF were stirred at room temperature overnight. The mixture was diluted with DMF and purified by prep. HPLC to give MC-hydrazone-(N,N′-(N-(levulinamide), bis-aminoethyl-1,3-propanediamine)-bis-3-nitro-1,8 naphthalimide) 110. MS m/z 964 (M+H)⁺.

### Example 64

### Synthesis of MC-vc-PAB-(N,N′-(bis-aminoethyl-1,3-propanediamine)-bis-(4-N-methylpiperidine)-1,8 naphthalimide) 111

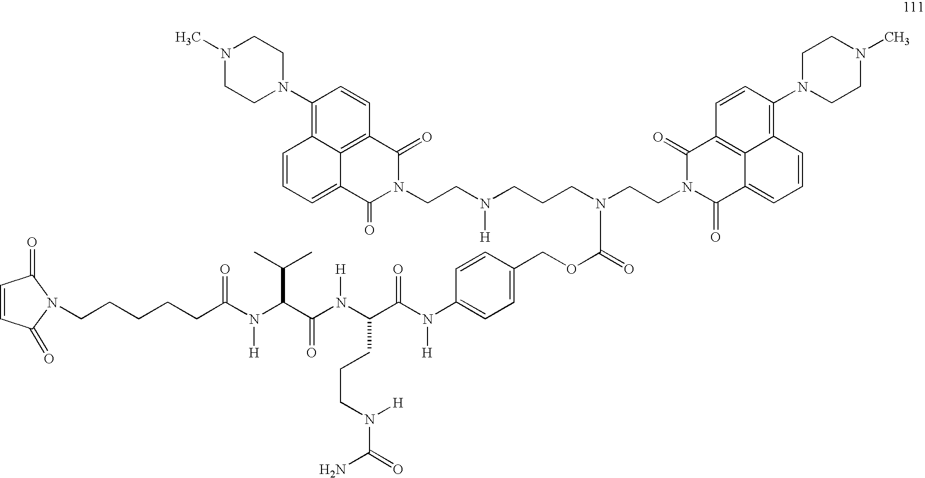

A mixture of the tetra-TFA salt of N,N′-(bis-aminoethyl-1,3-propanediamine)-bis-(4-N-methylpiperidine)-1,8 naphthalimide 24 (11 mg, 0.009 mmole), DIEA (0.008 ml, 0.092 mmole) and 0.2 ml DMF was stirred at room temperature under nitrogen for 10 minutes, then maleimido-caproyl-valine-citrulline-para-aminobenzyl-4-nitrophenylcarbonate (MC-vc-PAB-OPNP, 7 mg, 0.009 mmole), HOBt (0.002 mmole), DIEA (0.013 ml, 0.075 mmole) in 3 ml DMF were added, and the mixture was stirred at room temperature overnight. The mixture was quenched with 0.50 ml 1.3M aqueous TFA and purified by prep. HPLC to give MC-vc-PAB-(N,N′-(bis-aminoethyl-1,3-propanediamine)-bis-(4-N-methylpiperidine)-1,8 naphthalimide) 111. MS m/z 1315 (M)⁺.

### Example 64a

### Synthesis of MC-vc-PAB-(N, N′-(bis-aminoethyl-1,3-propanediamine)-bis-(4-N-imidazolyl)-1,8 naphthalimide) 111a

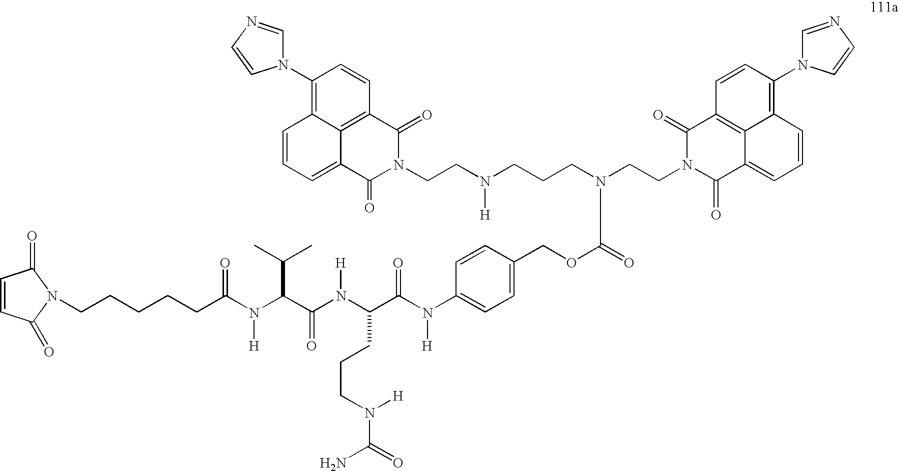

Following the protocol of Example 64, MC-val-cit-PAB-OPNP and N,N′-(bis-aminoethyl-1,3-propanediamine)-bis-(4-N-imidazolyl)-1,8 naphthalimide 24b were reacted to give MC-vc-PAB-(N,N′-(bis-aminoethyl-1,3-propanediamine)-bis-(4-N-imidazolyl)-1,8 naphthalimide) 111a.

### Example 64b

### Synthesis of MC-ala-phe-PAB-(N, N′-(bis-aminoethyl- 13-propanediamine)-bis-(4-N-imidazolyl)-1,8 naphthalimide) 111b

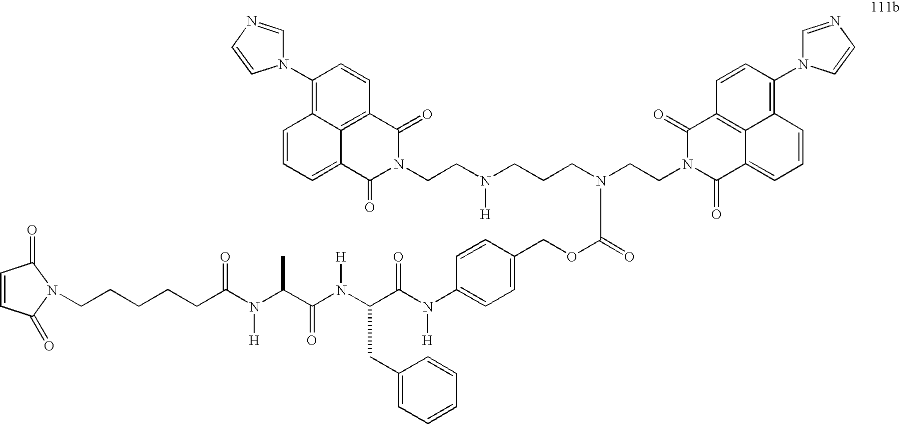

Following the protocol of Example 64, MC-ala-phe-PAB-OPNP and N,N′-(bis-aminoethyl-1,3-propanediamine)-bis-(4-N-imidazolyl)-1,8 naphthalimide 24b were reacted to give MC-ala-phe-PAB-(N,N′-(bis-aminoethyl-1,3-propanediamine)-bis-(4-N-imidazolyl)-1,8 naphthalimide) 11 lb.

### Example 65

### Synthesis of MP-vc-PAB-(N, N′-(bis-aminoethyl-1,3-propanediamine)-bis-(4-N-methylpiperidine)-1,8 naphthalimide) 112

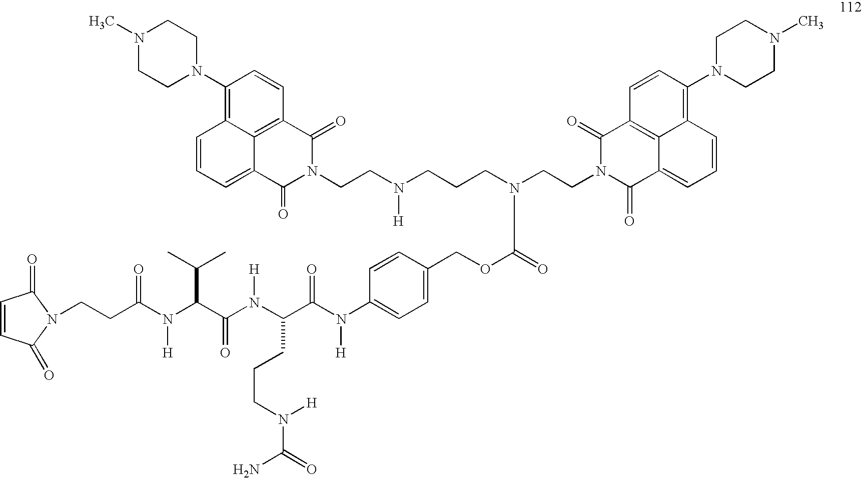

A mixture of the tetra-TFA salt of N,N′-(bis-aminoethyl-1,3-propanediamine)-bis-(4-N-methylpiperidine)-1,8 naphthalimide 24 (23 mg, 0.020 mmole), DIEA (0.017 ml, 0.098 mmole) and 0.2 ml DMF was stirred at room temperature under nitrogen for 10 minutes, then maleimido-propanoyl-valine-citrulline-para-aminobenzyl-4-nitrophenylcarbonate (MP-vc-PAB-OPNP, 9 mg, 0.012 mmole), DIEA (0.013 ml, 0.075 mmole) in 3 ml DMF were added, and the mixture was stirred at room temperature overnight. The mixture was quenched with 0.50 ml 1.3M aqueous TFA and purified by prep. HPLC to give MP-vc-PAB-(N, N′-(bis-aminoethyl-1,3-propanediamine)-bis-(4-N-methylpiperidine)-1,8 naphthalimide) 112. MS m/z 1331 (M+H)⁺.

### Example 67

### Synthesis of MC-(N, N′-(bis-aminoethyl-1,3-propanediamine)-bis-(4-N-methylpiperidine)-1,8 naphthalimide) 113

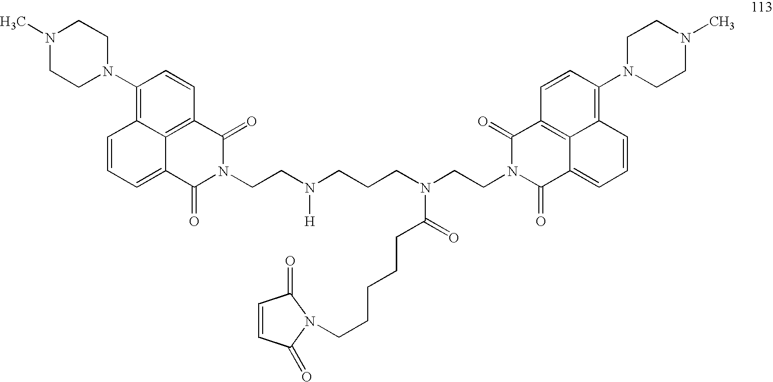

A mixture of 6-maleimidocaproic acid (3.3 mg, 0.015 mmole), PyBOP (7 mg, 0.014 mmole), DIEA (0.004 ml, 0.024 mmole), HOBt (2 mg, 0.013 mmole) and 0.2 ml DMF was stirred at room temperature under nitrogen for 5 minutes, then added to a solution of the tetra-TFA salt of N,N′-(bis-aminoethyl-1,3-propanediamine)-bis-(4-N-methylpiperidine)-1,8 naphthalimide 24 (14 mg, 0.012 mmole), DIEA (0.010 ml, 0.059 mmole) in 0.2 ml DMF. The mixture was stirred at room temperature for 2 hours, quenched with 7 ml 0.1% aqueous TFA and 2 ml acetic acid, and purified by prep. HPLC to give MC-(N,N′-(bis-aminoethyl-1,3-propanediamine)-bis-(4-N-methylpiperidine)-1,8 naphthalimide) 113. MS m/z 910 (M+).

### Example 68

### Synthesis of MC-(N, N′-(bis-aminoethyl-1,3-propanediamine)-bis-(4-N-imidazolyl)-1,8 naphthalimide) 113a

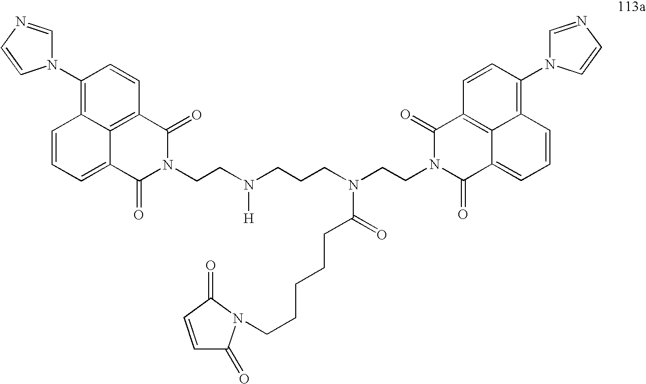

Following the procedure of Example 67, N,N′-(bis-aminoethyl-1,3-propanediamine)-bis-(4-N-imidazolyl)-1,8 naphthalimide 24b was converted to the maleimidocaproyl amide, MC-(N,N′-(bis-aminoethyl-1,3-propanediamine)-bis-(4-N-imidazolyl)-1,8 naphthalimide) 113a.

### Example 69

### Synthesis of tBu-Adip-vc-PAB-(N, N′-(bis-aminoethyl-1,3-propanediamine)-bis-(4-N-methylpiperidine)-1,8 naphthalimide) 114

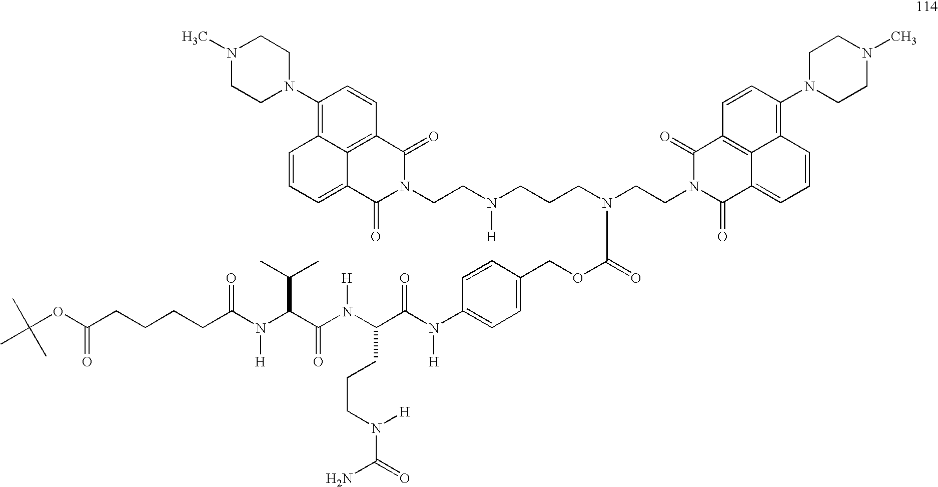

A mixture of the tetra-TFA salt of N,N′-(bis-aminoethyl-1,3-propanediamine)-bis-(4-N-methylpiperidine)-1,8 naphthalimide 24 (10 mg, 0.009 mmole), DIEA (0.012 ml, 0.070 mmole) and 0.2 ml DMF was stirred at room temperature under nitrogen for 10 minutes, then tert-butyladipate-valine-citrulline-para-aminobenzyl-4-nitrophenylcarbonate (tBuAdip-vc-PAB-OPNP, 6.4 mg, 0.009 mmole), DIEA (0.013 ml, 0.075 mmole) in 3 ml DMF were added, and the mixture was stirred at room temperature overnight. The mixture was quenched with 0.050 ml 1.3M aqueous TFA and purified by prep. HPLC to give tBu-Adip-vc-PAB-(N,N′-(bis-aminoethyl-1,3-propanediamine)-bis-(4-N-methylpiperidine)-1,8 naphthalimide) 114. MS m/z 1306 (M)⁺.

### Example 70

### Synthesis of N¹-acetyl. N²-Adip-vc-PAB-(N,N′-(bis-aminoethyl-1,3-propanediamine)-bis-(4-N-methylpiperidine)-1,8 naphthalimide) 115

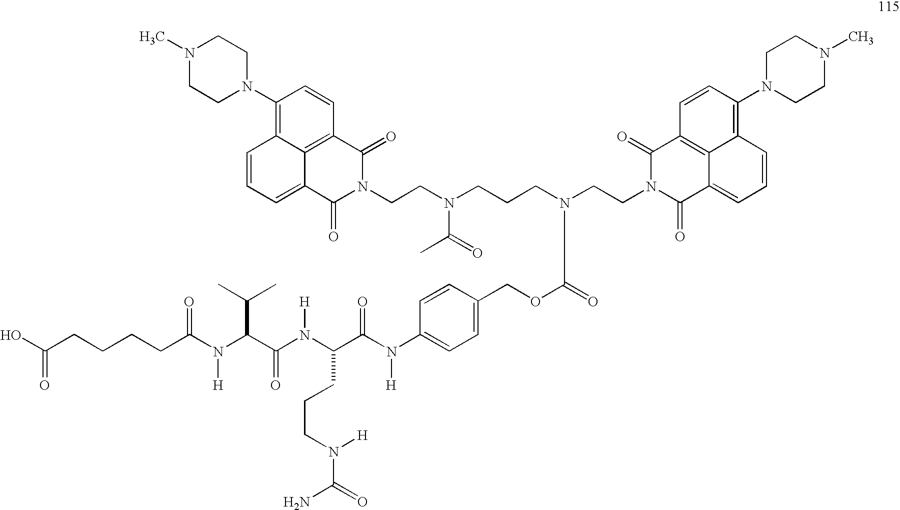

A mixture of the TFA salt of tBu-Adip-vc-PAB-(N,N′-(bis-aminoethyl-1,3-propanediamine)-bis-(4-N-methylpiperidine)-1,8 naphthalimide) 114 (2 mg, 0.001 mmole), acetic anhydride (0.013 mmole), triethylamine (0.013 mmole) and 0.5 ml dichloromethane were stirred at room temperature for about 25 minutes, then one drop of water was added and concentrated under vacuum to give N¹-acetyl, N²-t-BuAdip-vc-PAB-(N,N′-(bis-aminoethyl-1,3-propanediamine)-bis-(4-N-methylpiperidine)-1,8 naphthalimide). A solution of 10% TFA (2 ml) was added and stirred for 2.5 hours and concentrated to give N¹-acetyl, N²-Adip-vc-PAB-(N,N′-(bis-aminoethyl-1,3-propanediamine)-bis-(4-N-methylpiperidine)-1,8 naphthalimide) 115.

### Example 71

### Synthesis of N¹-acetyl. N²—NHS-Adip-vc-PAB-(N, N′-(bis-aminoethyl-1,3-propanediamine)-bis-(4-N-methylpiperidine)-1,8 naphthalimide) 116

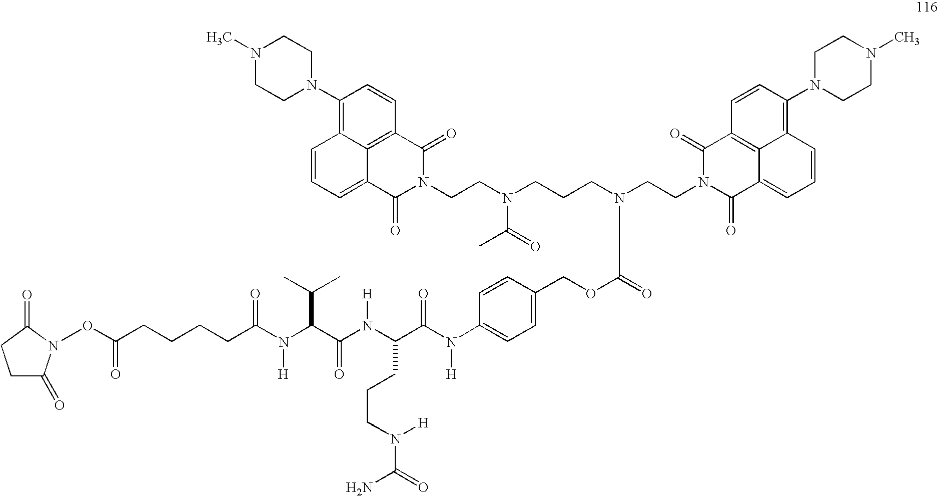

A mixture of N¹-acetyl, N²-Adip-vc-PAB-(N,N′-(bis-aminoethyl-1,3-propanediamine)-bis-(4-N-methylpiperidine)-1,8 naphthalimide) 115 (about 2 mg, 0.001 mmole), N,N′-disuccinimidyl carbonate (DSC, 10 mg, 0.037 mmole), 0.1 ml acetonitrile, and 0.1 ml DMF was stirred at room temperature for about 1.5 hours, then quenched with acetic acid and dilute aqueous TFA, concentrated under vacuum, and purified by prep. HPLC to give N′-acetyl, N²—NHS-Adip-vc-PAB-(N,N′-(bis-aminoethyl-1,3-propanediamine)-bis-(4-N-methylpiperidine)-1,8 naphthalimide) 116. MS m/z 1391 (M+H)⁺.

### Example 72

### Synthesis of N¹-methyl, N²-MC-af-PAB-(N, N′-(bis-aminoethyl-1,3-propanediamine)-bis-(4-N-imidazolyl)-1,8 naphthalimide) 117

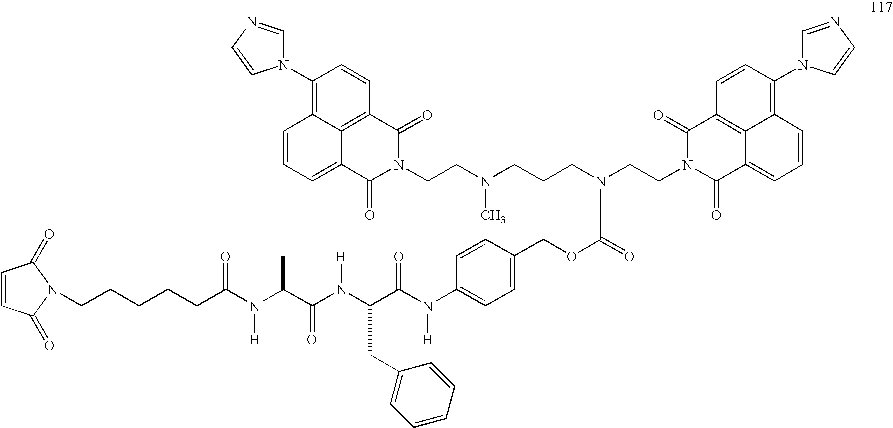

Following the protocols of the foregoing Examples, N¹-methyl, N²-MC-af-PAB-(N,N′-(bis-aminoethyl-1,3-propanediamine)-bis-(4-N-imidazolyl)-1,8 naphthalimide) 117 was prepared.

### Example 73

### Synthesis of N¹-methyl, N²-(MC-vc-PAB-N-methylglycyl)-(N, N′-(bis-aminoethyl-1,3-propanediamine)-bis-(4-N-imidazolyl)-1,8 naphthalimide) 118

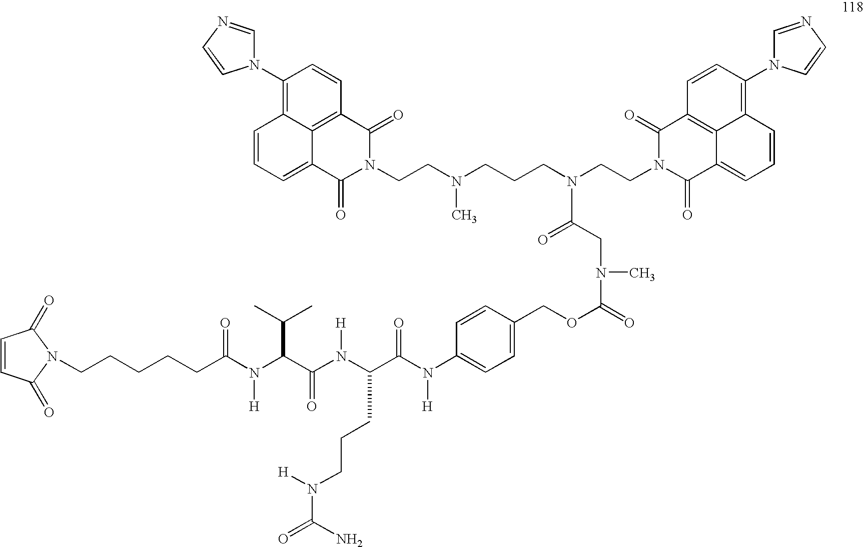

Following the protocols of the foregoing Examples, N¹-methyl, N²-(MC-vc-PAB-N-methylglycyl)-(N,N′-(bis-aminoethyl-1,3-propanediamine)-bis-(4-N-imidazolyl)-1,8 naphthalimide) 118 was prepared.

### Example 74

### Synthesis of N¹-methyl, N²-(MC-af-PAB-N-methylglycyl)-(N, N′-(bis-aminoethyl-1,3-propanediamine)-bis-(4-N-imidazolyl)-1,8 naphthalimide) 119

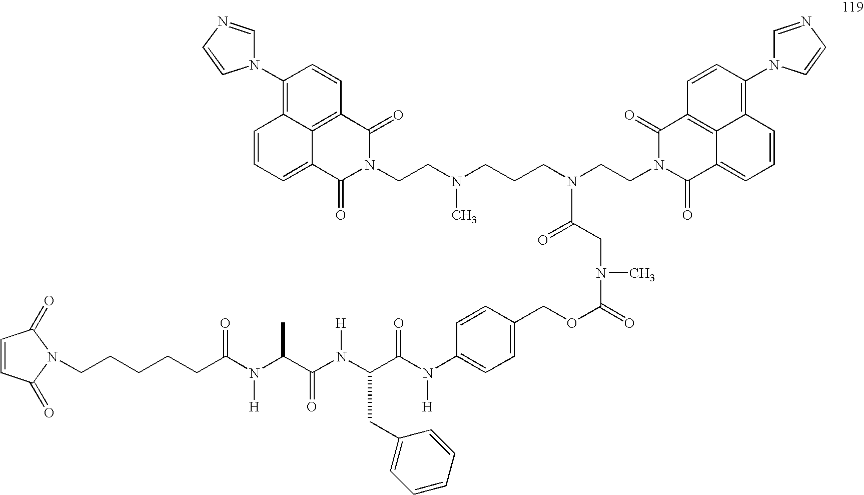

Following the protocols of the foregoing Examples, N¹-methyl, N²-(MC-af-PAB-N-methylglycyl)-(N,N′-(bis-aminoethyl-1,3-propanediamine)-bis-(4-N-imidazolyl)-1,8 naphthalimide) 119 was prepared.

### Example 75

### Synthesis of N¹-methyl, N²-(MC-vc-PAB-(3-N-methylpropanamide))-(N, N′-(bis-aminoethyl-1,3-propanediamine)-bis-(4-N-imidazolyl)-1,8 naphthalimide) 120

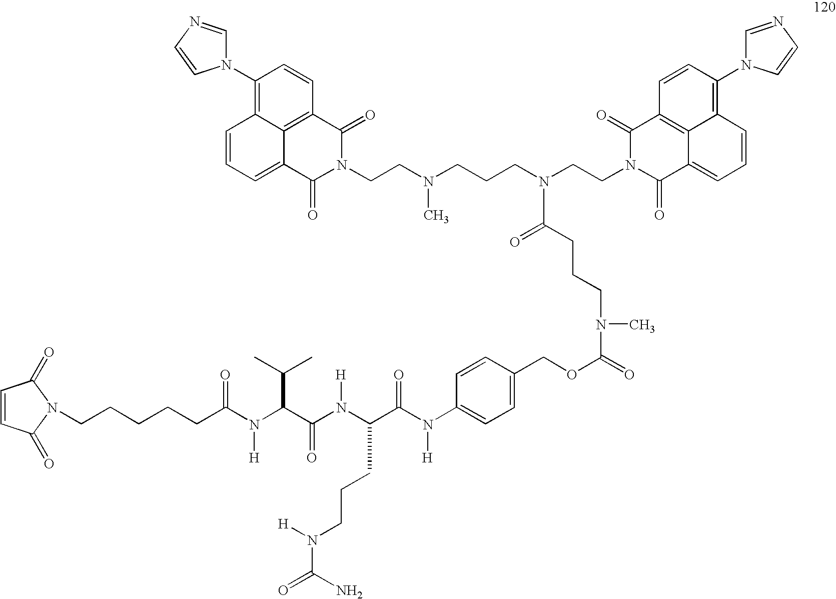

Following the protocols of the foregoing Examples, N¹-methyl, N²-(MC-vc-PAB-(3-N-methylpropanamide))—(N,N′-(bis-aminoethyl-1,3-propanediamine)-bis-(4-N-imidazolyl)-1,8 naphthalimide) 120 was prepared.

### Example 76

### Synthesis of N¹-methyl, N²-(MC-af-PAB-(3-N-methylpropanamide))-(N, N′-(bis-aminoethyl-1,3-propanediamine)-bis-(4-N-imidazolyl)-1,8 naphthalimide) 121

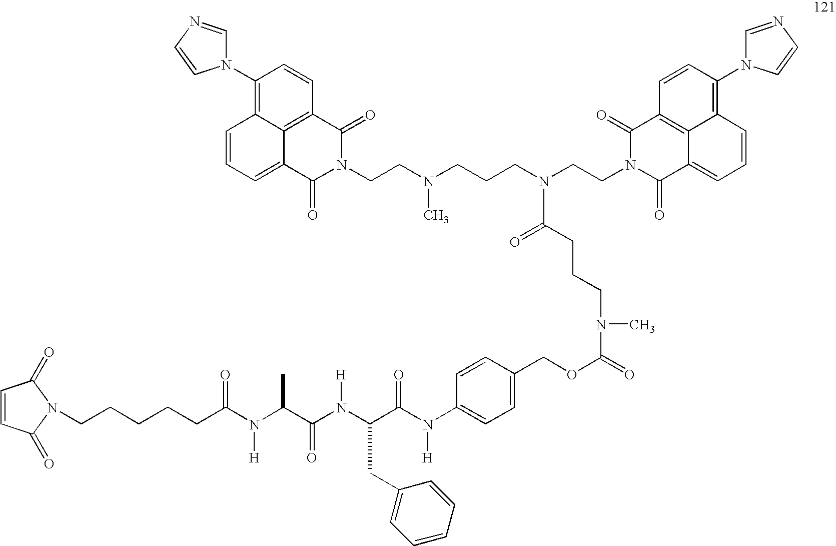

Following the protocols of the foregoing Examples, N¹-methyl, N²-(MC-af-PAB-(3-N-methylpropanamide))—(N,N′-(bis-aminoethyl-1,3-propanediamine)-bis-(4-N-imidazolyl)-1,8 naphthalimide) 121 was prepared.

### Example 77

### Synthesis of N, N′-(bis-aminoethyl-1,3-bis N-methyl-propanediamine)-(4-N-imidazolyl, 4′-N-(MC-vc-PAB)-1,8 naphthalimide) 122

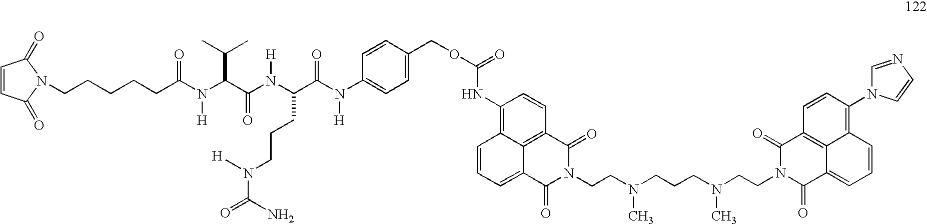

Following the protocols of the foregoing Examples, N,N′-(bis-aminoethyl-1,3-bis N-methyl-propanediamine)-(4-N-imidazolyl, 4′-N-(MC-vc-PAB)-1,8 naphthalimide) 122 was prepared.

### Example 78

### Synthesis of N, N′-(bis-aminoethyl-1,3-bis N-methyl-propanediamine)-(4-N-imidazolyl, 4′-N-(MC-af-PAB)-1,8 naphthalimide) 122a

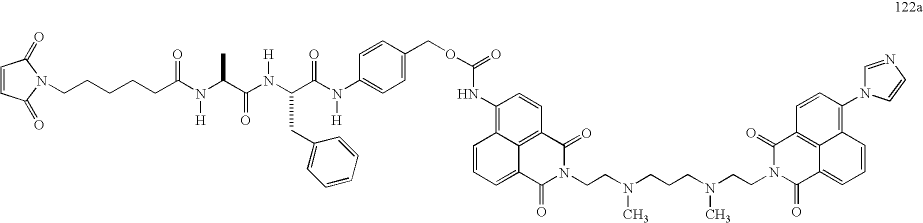

Following the protocols of the foregoing Examples, N,N′-(bis-aminoethyl-1,3-bis N-methyl-propanediamine)-(4-N-imidazolyl, 4′-N-(MC-af-PAB)-1,8 naphthalimide) 122a was prepared.

### Example 79

### Synthesis of N, N′-(bis-aminoethyl-1,3-bis N-methyl-propanediamine)-(4-N-imidazolyl, 4′-N-(MC-vc)-1,8 naphthalimide) 123

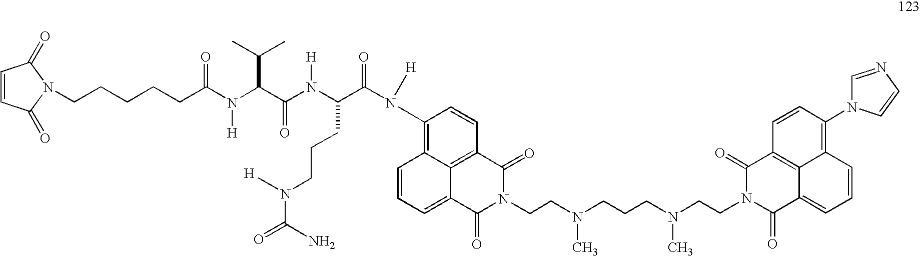

Following the protocols of the foregoing Examples, N,N′-(bis-aminoethyl-1,3-bis N-methyl-propanediamine)-(4-N-imidazolyl, 4′-N-(MC-vc)-1,8 naphthalimide) 123 was prepared.

### Example 80

### Synthesis of N, N′-(bis-aminoethyl-1,3-bis N-methyl-propanediamine)-(3-nitro, 4′-N-(MP-gly-val-cit)-1,8 naphthalimide) 123a

Following the protocols of the foregoing Examples, N,N′-(bis-aminoethyl-1,3-bis N-methyl-propanediamine)-(4-N-imidazolyl, 4′-N-(MC-vc)-1,8 naphthalimide) 123a was prepared.

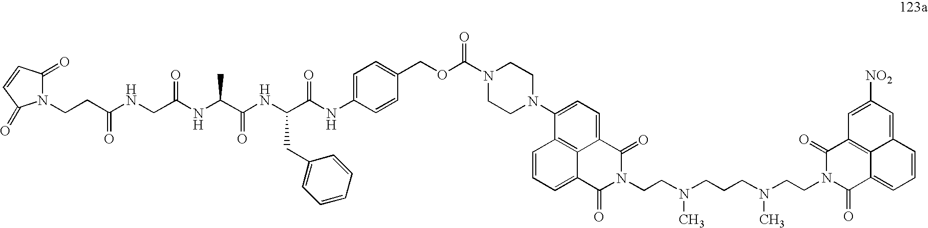

### Example 81

### Synthesis of N¹-ethyl, N²-MC-af-PAB-(N, N′-(bis-aminoethyl-1,3-propanediamine)-bis 3-nitro-1,8 naphthalimide) 124

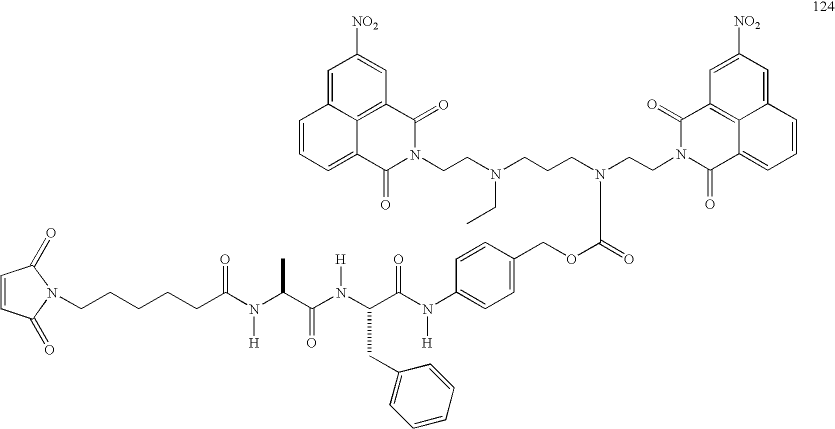

Following the protocols of the foregoing Examples, N¹-ethyl, N²-MC-af-PAB-(N,N′-(bis-aminoethyl-1,3-propanediamine)-bis 3-nitro-1,8 naphthalimide) 124 was prepared.

### Example 82

### Synthesis of N¹-ethyl, N²-MC-af-PAB-(N-methylvaline)-(N, N′-(bis-aminoethyl-1,3-propanediamine)-bis 3-nitro-1,8 naphthalimide) 125

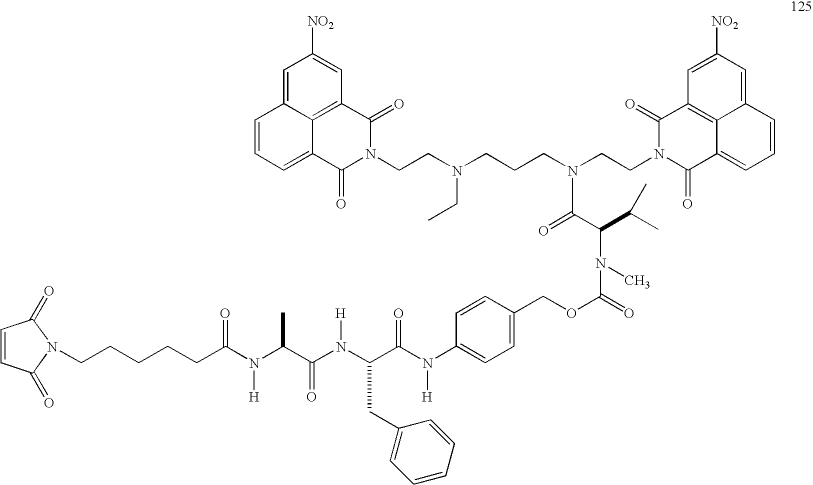

Following the protocols of the foregoing Examples, N′-ethyl, N²-MC-af-PAB-(N-methylvaline)-(N, N′-(bis-aminoethyl-1,3-propanediamine)-bis 3-nitro-1,8 naphthalimide) 125 was prepared.

### Example 83

### Synthesis of N¹—H, N²-MC-vc-PAB-(N,N′-(bis-aminoethyl-1,3-propanediamine)-bis 3-nitro-1,8 naphthalimide) 126

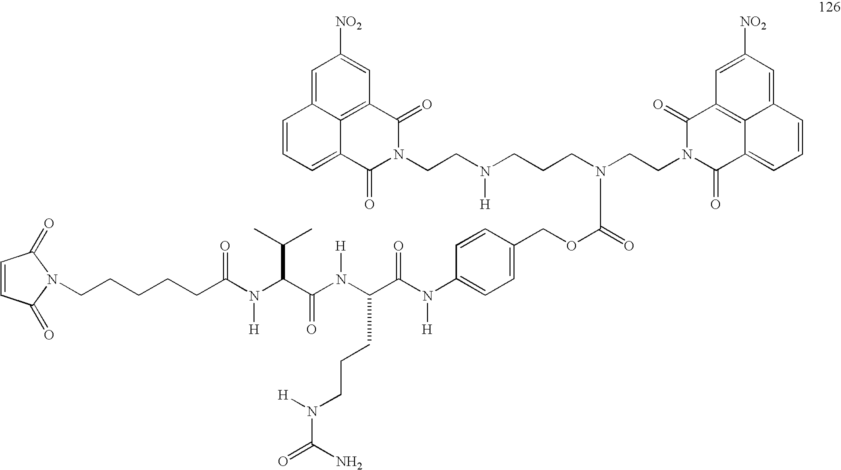

Following the protocols of the foregoing Examples, N′-H, N²-MC-vc-PAB-(N,N′-(bis-aminoethyl-1,3-propanediamine)-bis 3-nitro-1,8 naphthalimide) 126 was prepared.

### Example 84

### Synthesis of N¹—H, N²-MC-vc-PAB-(N, N′-(bis-aminoethyl-1,3-propanediamine)-3-nitro, 4-amino-1,8 naphthalimide) 126a

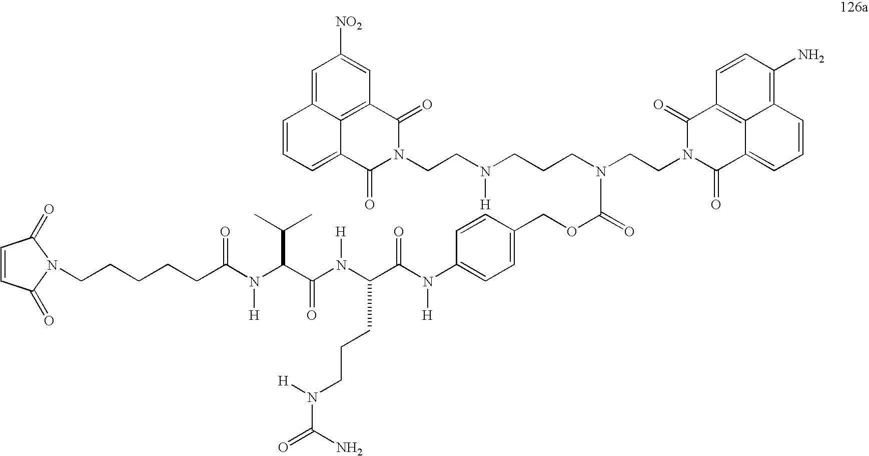

Following the protocols of the foregoing Examples, N′-H, N²-MC-vc-PAB-(N,N′-(bis-aminoethyl-1,3-propanediamine)-3-nitro, 4-amino-1,8 naphthalimide) 126a was prepared.

### Example 85

### Synthesis of N¹—H, N²-MC-af-PAB-(N,N′-(bis-aminoethyl-1,3-propanediamine)-bis 3-nitro-1,8 naphthalimide) 127

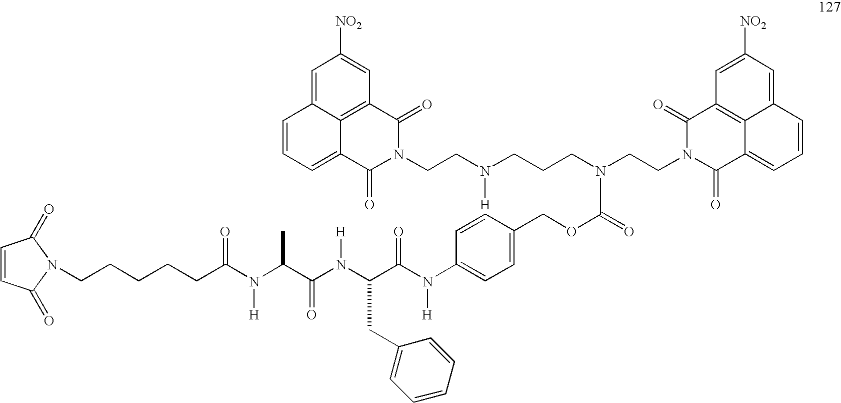

Following the protocols of the foregoing Examples, N¹—H, N²-MC-af-PAB-(N,N′-(bis-aminoethyl-1,3-propanediamine)-bis 3-nitro-1,8 naphthalimide) 127 was prepared.

### Example 86

### Synthesis of N¹—H, N²-(tert-butyladipate-jly-gly-gly-PAB)-(N, N′-(bis-aminoethyl-1,3-propanediamine)-bis 3-nitro-1,8 naphthalimide) 128

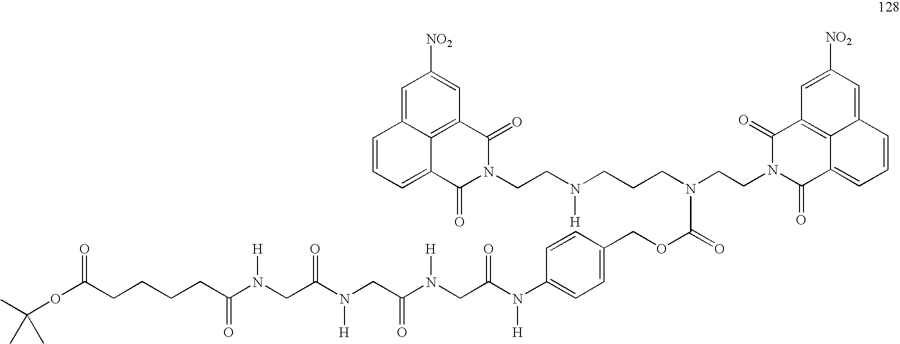

Following the protocols of the foregoing Examples, N′-H, N²-(tert-butyladipate-gly-gly-gly-PAB)-(N,N′-(bis-aminoethyl-1,3-propanediamine)-bis 3-nitro-1,8 naphthalimide) 128 was prepared.

### Example 87

### Synthesis of N¹—H, N²-(MC-val-cit)-(N, N′-(bis-aminoethyl-1,3-propanediamine)-bis 3-nitro-1,8 naphthalimide) 129

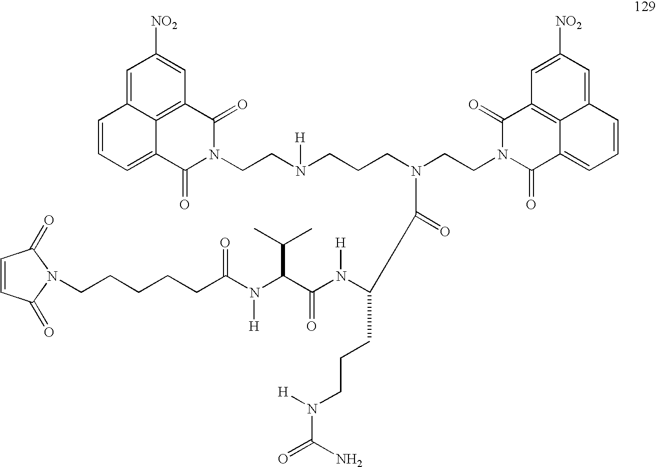

Following the protocols of the foregoing Examples, N′-H, N²-(MC-val-cit)-(N,N′-(bis-aminoethyl-1,3-propanediamine)-bis 3-nitro-1,8 naphthalimide) 129 was prepared.

### Example 88

### Synthesis of N¹—H, N²-(MC-vc-gly)-(N, N′-(bis-aminoethyl-1,3-propanediamine)-bis 3-nitro-1,8 naphthalimide) 130

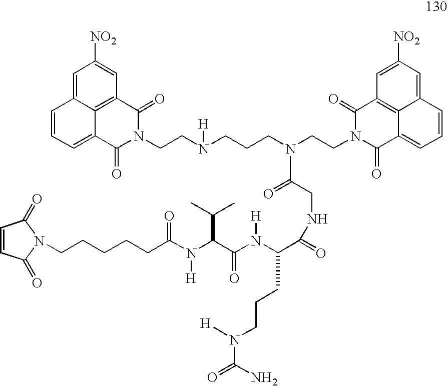

Following the protocols of the foregoing Examples, N′-H, N²-(MC-Vc-gly)-(N,N′-(bis-aminoethyl-1,3-propanediamine)-bis 3-nitro-1,8 naphthalimide) 130 was prepared.

### Example 89

### Synthesis of N¹—H, N²-(MC-af)-(N,N′-(bis-aminoethyl-1,3-propanediamine)-bis 3-nitro-1,8 naphthalimide) 131

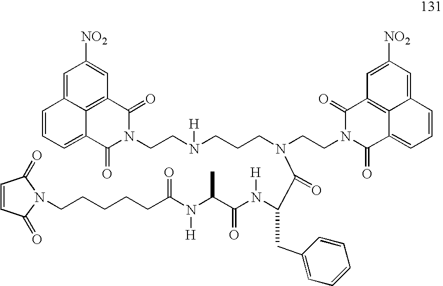

Following the protocols of the foregoing Examples, N′-H, N²-(MC-af)-(N,N′-(bis-aminoethyl-1,3-propanediamine)-bis 3-nitro-1,8 naphthalimide) 131 was prepared.

### Example 90

### Synthesis of N′-H, N²-(MC-ala-phe-gly)-(N,N′-(bis-aminoethyl-1,3-propanediamine)-bis 3-nitro-1,8 naphthalimide) 132

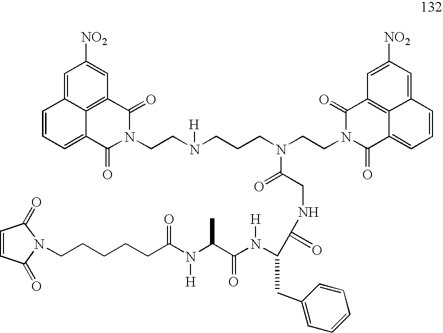

Following the protocols of the foregoing Examples, N′-H, N²-(MC-ala-phe-gly)-(N,N′-(bis-aminoethyl-1,3-propanediamine)-bis 3-nitro-1,8 naphthalimide) 132 was prepared.

### Example 91

### Synthesis of N¹—H, N²-(succinic-gly-val-cit)-(N, N′-(bis-aminoethyl-1,3-propanediamine)-bis 3-nitro-1,8 naphthalimide) 133

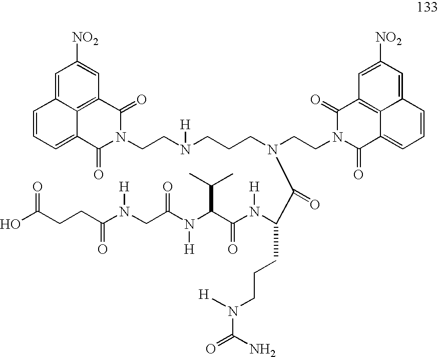

Following the protocols of the foregoing Examples, N¹—H, N²-(succinic-gly-val-cit)-(N,N′-(bis-aminoethyl-11,3-propanediamine)-bis 3-nitro-1,8 naphthalimide) 133 was prepared.

### Example 92

### Synthesis of N¹—H, N²-(succinic-gly-val-cit-gly)-(N, N′-(bis-aminoethyl-1,3-propanediamine)-bis 3-nitro-1,8 naphthalimide) 134

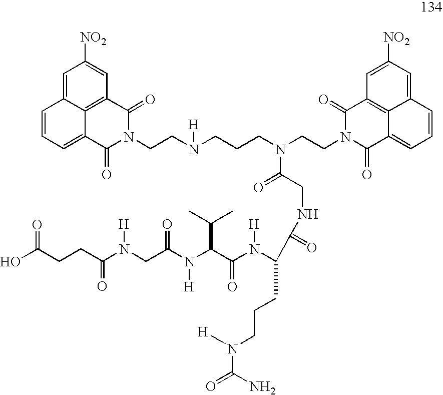

Following the protocols of the foregoing Examples, N′-H, N²-(succinic-gly-val-cit-gly)-(N,N′-(bis-aminoethyl-1,3-propanediamine)-bis 3-nitro-1,8 naphthalimide) 134 was prepared.

### Example 93

### Synthesis of N¹—H, N²-(succinic-gly-ala-phe)-(N,N′-(bis-aminoethyl-1,3-propanediamine)-bis 3-nitro-1,8 naphthalimide) 135

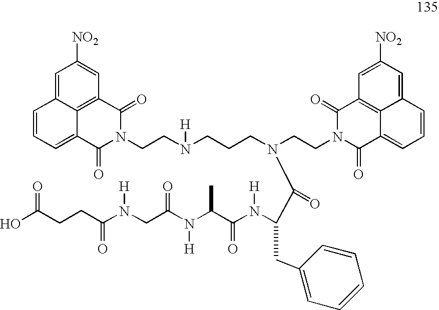

Following the protocols of the foregoing Examples, N′-H, N²-(succinic-gly-ala-phe)-(N,N′-(bis-aminoethyl-1,3-propanediamine)-bis 3-nitro-1,8 naphthalimide) 135 was prepared.

### Example 94

### Synthesis of N¹—H, N²-(N-hydroxysuccinimide-succinic-gly-ala-phe)-(N, N′-(bis-aminoethyl-1,3-propanediamine)-bis 3-nitro-1,8 naphthalimide) 135a

Following the protocol of Example 69, acid 135 from Example 89 was converted to the NHS ester, N′-H, N²—(N-hydroxysuccinimide-succinate-gly-ala-phe)-(N,N′-(bis-aminoethyl-1,3-propanediamine)-bis 3-nitro-1,8 naphthalimide) 135a.

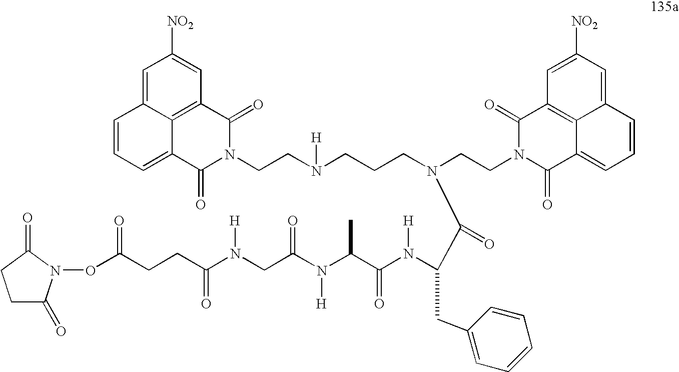

### Example 95

### Synthesis of N¹—H, N²-(succinic-gly-ala-phe-gly)-(N, N′-(bis-aminoethyl-1,3-propanediamine)-bis 3-nitro-1,8 naphthalimide) 136

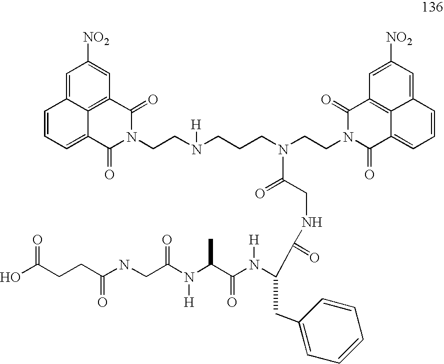

Following the protocols of the foregoing Examples, N′-H, N²-(succinic-gly-ala-phe-gly)-(N,N′-(bis-aminoethyl-1,3-propanediamine)-bis 3-nitro-1,8 naphthalimide) 136 was prepared.

### Example 96

### Synthesis of N¹-ethyl, N²-(MC-vc-PAB-N-methylvaline)-(N, N′-(bis-aminoethyl-1,3-propanediamine)-bis 2-nitro-1,8 naphthalimide) 137

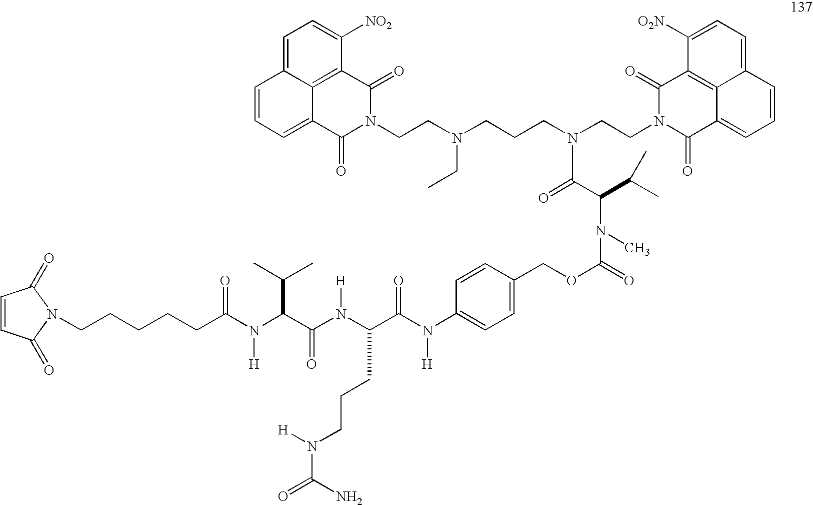

Following the protocols of the foregoing Examples, N¹-ethyl, N²-(MC-vc-PAB-N-methylvaline)-(N, N′-(bis-aminoethyl-1,3-propanediamine)-bis 2-nitro-1,8 naphthalimide) 137 was prepared.

### Example 97

### Synthesis of N¹-ethyl, N²-(maleimido-4-oxo-caproyl-vc-PAB-N-methylvaline)-(N, N′-(bis-aminoethyl-1,3-propanediamine)-bis 2-nitro-1,8 naphthalimide) 138

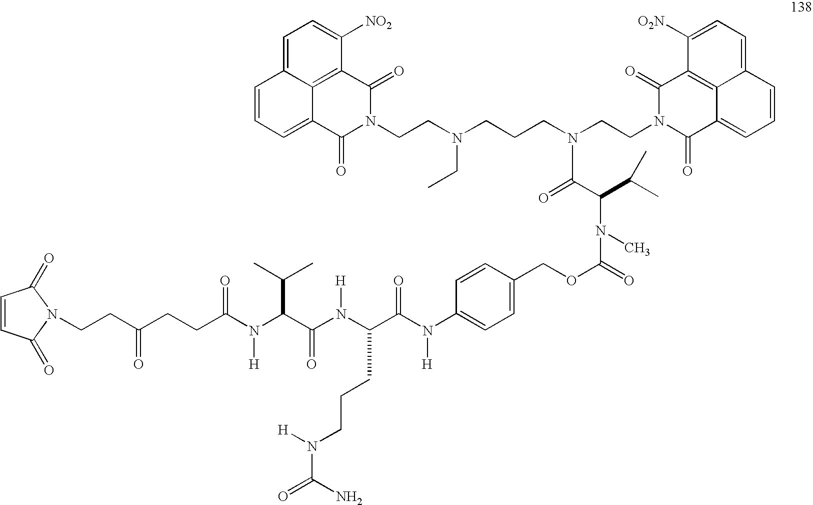

Following the protocols of the foregoing Examples, N¹-ethyl, N²-(maleimido-4-oxo-caproyl-vc-PAB-N-methylvaline)-(N,N′-(bis-aminoethyl-1,3-propanediamine)-bis 2-nitro-1,8 naphthalimide) 138 was prepared.

### Example 98

### Synthesis of N¹-methyl, N²—(N-methylglycyl)-(N, N′-(bis-aminoethyl-1,3-propanediamine)-bis 4-N-imidazolyl-1,8 naphthalimide) 139

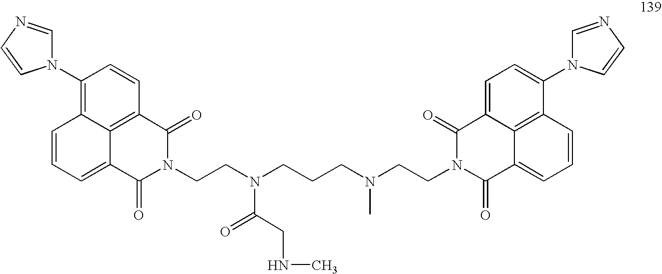

Following the protocols of the foregoing Examples, N¹-methyl, N²-(N-methylglycyl)-(N,N′-(bis-aminoethyl-1,3-propanediamine)-bis 4-N-imidazolyl-1,8 naphthalimide) 139 was prepared.

### Example 99

### Synthesis of N¹—H, N²-(methoxyethoxyethoxyacetamide)-(N, N′-(bis-aminoethyl-1,3-propanediamine)-bis 4-N-imidazolyl-1,8 naphthalimide) 140

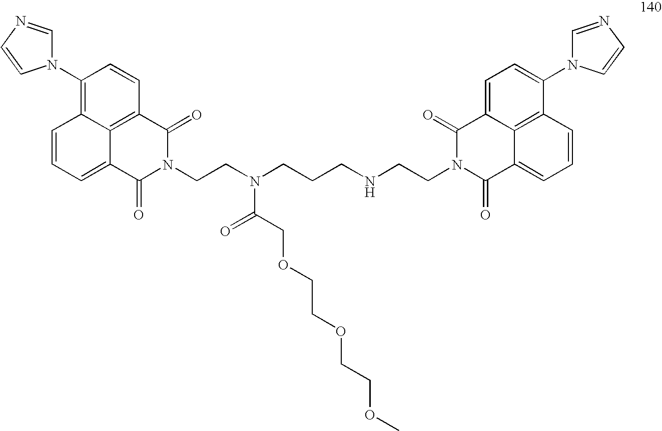

Following the protocols of the foregoing Examples, N′-H, N²-(methoxyethoxyethoxyacetamide)-(N, N′-(bis-aminoethyl-1,3-propanediamine)-bis 4-N-imidazolyl-1,8 naphthalimide) 140 was prepared.

### Example 100

### Synthesis of N¹-(MC-vc-PAB), N²-(methoxyethoxyethoxyacetamide)-(N, N′-(bis-aminoethyl-1,3-propanediamine)-bis 4-N-imidazolyl-1,8 naphthalimide) 141

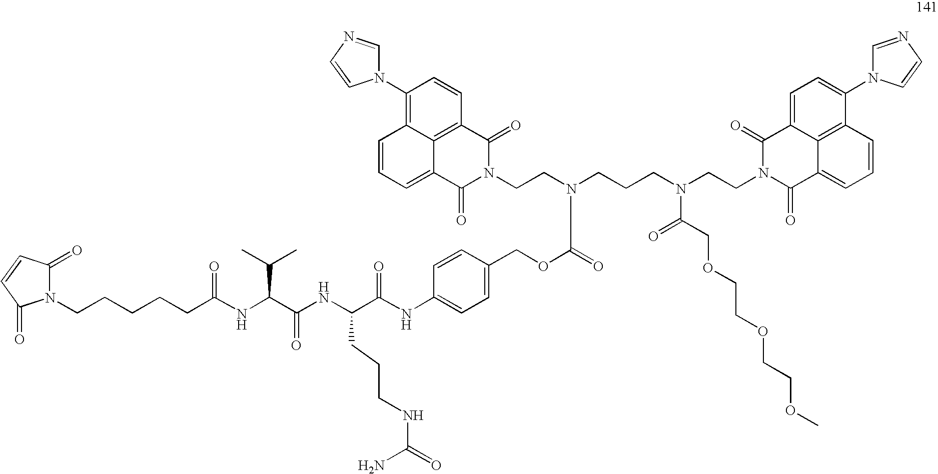

Following the protocols of the foregoing Examples, N¹-(MC-vc-PAB), N²-(methoxyethoxyethoxyacetamide)-(N,N′-(bis-aminoethyl-1,3-propanediamine)-bis 4-N-imidazolyl-1,8 naphthalimide) 141 was prepared.

### Example 101

### Synthesis of N¹-(MC-af-PAB), N²-(methoxyethoxyethoxyacetamide)-(N, N′-(bis-aminoethyl-1,3-propanediamine)-bis 4-N-imidazolyl-1,8 naphthalimide) 142

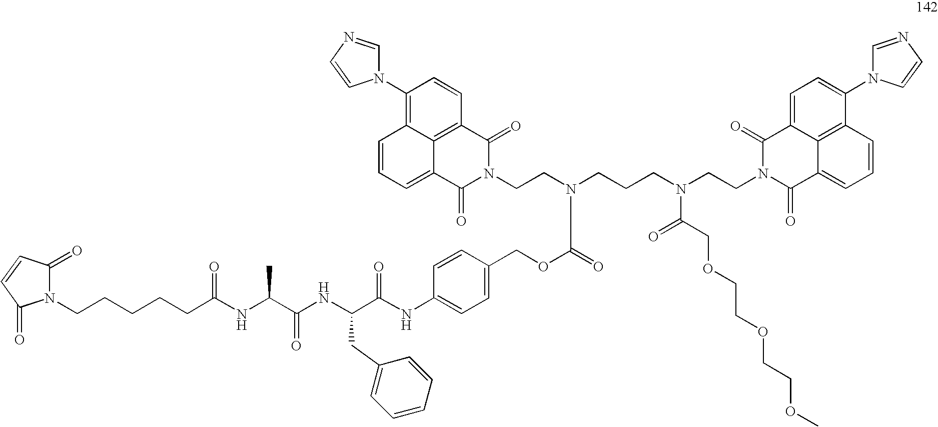

Following the protocols of the foregoing Examples, N¹-(MC-af-PAB), N²-(methoxyethoxyethoxyacetamide)-(N,N′-(bis-aminoethyl-1,3-propanediamine)-bis 4-N-imidazolyl-1,8 naphthalimide) 142 was prepared.

### Example 102

### Synthesis of N¹-cyclopropylmethyl, N²-MP-gly-val-cit-PAB-(N, N′-(bis-aminoethyl-1,3-propanediamine)-bis 3-nitro-1,8 naphthalimide) 143

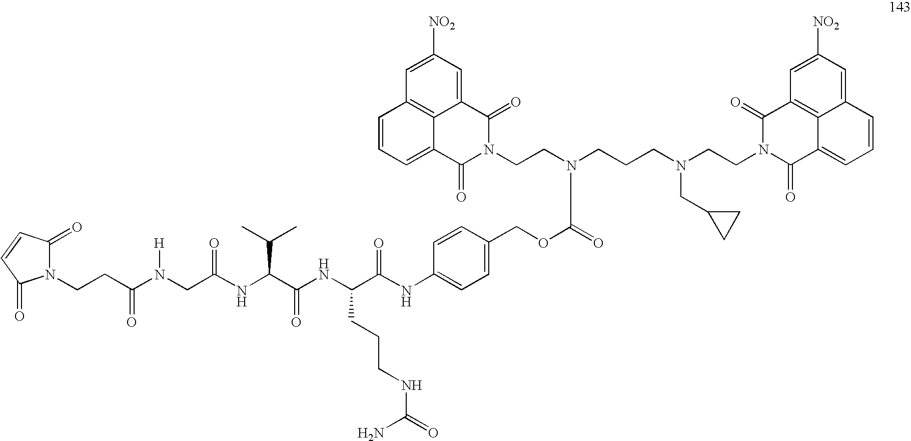

Following the protocols of the foregoing Examples, MP-gvc-PAB-OPNP and N,N′-(N-cyclopropylmethyl, bis-aminoethyl-1,3-propanediamine)-bis-3-nitro-1,8 naphthalimide 30c were reacted to give N¹-cyclopropylmethyl, N²-maleimidopropyl-gly-val-cit-PAB-(N,N′-(bis-aminoethyl-1,3-propanediamine)-bis 3-nitro-1,8 naphthalimide) 143.

### Example 103

### Preparation of trastuzumab- MC-vc-PAB-(N,N′-2-acetamido-1,3-ethanediamine-propyl)-bis-4-morpholino-1,8 naphthalimide) 201 by conjugation of trastuzumab and 102

One vial containing 440 mg HERCEPTIN® (huMAb4D5-8, rhuMAb HER2, U.S. Pat. No. 5,821,337) antibody) was dissolved in 50 mL MES buffer (25 mM MES, 50 mM NaCl, pH 5.6) and loaded on a cation exchange column (Sepharose S, 15 cm×1.7 cm) that had been equilibrated in the same buffer. The column was then washed with the same buffer (5 column volumes). Trastuzumab was eluted by raising the NaCl concentration of the buffer to 200 mM. Fractions containing the antibody were pooled, diluted to 10 mg/mL, and dialyzed into a buffer containing 50 mM potassium phosphate, 50 mM NaCl, 2 mM EDTA, pH 6.5.

Trastuzumab, dissolved in 500 mM sodium borate and 500 mM sodium chloride at pH 8.0 is treated with an excess of 100 mM dithiothreitol (DTT). After incubation at 37° C. for about 30 minutes, the buffer is exchanged by elution over Sephadex G25 resin and eluted with PBS with 1 mM DTPA. The thiol/Ab value is checked by determining the reduced antibody concentration from the absorbance at 280 nm of the solution and the thiol concentration by reaction with DTNB (Aldrich, Milwaukee, Wis.) and determination of the absorbance at 412 nm. The reduced antibody dissolved in PBS is chilled on ice.

The drug linker reagent, maleimidocaproyl-(valine-citrulline)-(para-aminobenzyloxycarbonyl)-(N, N′-2-acetamido-1,3-ethanediamine-propyl)-bis-4-morpholino-1,8 naphthalimide) 102, dissolved in DMSO, is diluted in acetonitrile and water at known concentration, and added to the chilled reduced antibody trastuzumab in PBS. After about one hour, an excess of maleimide is added to quench the reaction and cap any unreacted antibody thiol groups. The reaction mixture is concentrated by centrifugal ultrafiltration and 201 is purified and desalted by elution through G25 resin in PBS, filtered through 0.2 gm filters under sterile conditions, and frozen for storage.

### Example 104

### Preparation of Trastuzumab-MC-vc-PAB-(N, N′-(bis-aminoethyl-1,3-propanediamine)-bis-(4-N-imidazolyl)-1,8 naphthalimide) 202

Following the protocol of Example 103, antibody drug conjugate, trastuzumab- MC-vc-PAB-(N,N′-(bis-aminoethyl-1,3-propanediamine)-bis-(4-N-imidazolyl)-1,8 naphthalimide) 202, was prepared by conjugation of trastuzumab and MC-vc-PAB-(N,N′-(bis-aminoethyl-1,3-propanediamine)-bis-(4-N-imidazolyl)-1,8 naphthalimide) 111a.

### Example 105

### Preparation of Trastuzumab-MC-ala-phe-PAB-(N, N′-(bis-aminoethyl-1,3-pronanediamine)-bis-(4-N-imidazolyl)-1,8 naphthalimide) 203

Following the protocol of Example 103, antibody drug conjugate, trastuzumab-MC-ala-phe-PAB-(N, N′-(bis-aminoethyl-1,3-propanediamine)-bis-(4-N-imidazolyl)-1,8 naphthalimide) 203, was prepared by conjugation of trastuzumab and MC-ala-phe-PAB-(N,N′-(bis-aminoethyl-1,3-propanediamine)-bis-(4-N-imidazolyl)-1,8 naphthalimide) 111b.

### Example 106

### Preparation of Trastuzumab-(succinate-gly-ala-phe)-(N, N′-(bis-aminoethyl-1,3-propanediamine)-bis 3-nitro-1,8 naphthalimide) 204

Following the protocol of Example 103 for isolating trastuzumab, antibody drug conjugate, trastuzumab-(succinate-gly-ala-phe)-(N,N′-(bis-aminoethyl-1,3-propanediamine)-bis 3-nitro-1,8 naphthalimide) 204, was prepared by conjugation of trastuzumab and N′-H, N²—(N-hydroxysuccinimide-succinate-gly-ala-phe)-(N,N′-(bis-aminoethyl-1,3-propanediamine)-bis 3-nitro-1,8 naphthalimide) 135a.

### Example 107

### Preparation of Trastuzumab-MC-val-cit-PAB-(N, N′-(bis-aminoethyl-1,3-propanediamine)-3-nitro, 4-amino-1,8 naphthalimide) 205

Following the protocol of Example 103, antibody drug conjugate, trastuzumab-MC-val-cit-PAB-(N, N′-(bis-aminoethyl-1,3-propanediamine)-3-nitro, 4-amino-1,8 naphthalimide) 205, was prepared by conjugation of trastuzumab and (N′-H, N²-MC-vc-PAB-(N,N′-(bis-aminoethyl-1,3-propanediamine)-3-nitro, 4-amino-1,8 naphthalimide) 126a.

### Example 108

### Preparation of trastuzumab-MC-(N, N′-(bis-aminoethyl-1,3-propanediamine)-bis-(4-N-imidazolyl)-1,8 naphthalimide) 206

Following the protocol of Example 103, antibody drug conjugate, trastuzumab-MC-(N,N′-(bis-aminoethyl-1,3-propanediamine)-bis-(4-N-imidazolyl)-1,8 naphthalimide) 206, was prepared by conjugation of trastuzumab and MC-(N,N′-(bis-aminoethyl-1,3-propanediamine)-bis-(4-N-imidazolyl)-1,8 naphthalimide) 113a.

### Example 109

### Preparation of Trastuzumab-MC-(N, N′-(bis-aminoethyl-1,3-propanediamine)-bis-(4-N-imidazolyl)-1,8 naphthalimide) 207

Following the protocol of Example 103, antibody drug conjugate, trastuzumab-N¹-cyclopropylmethyl, N²-maleimidopropyl-gly-val-cit-PAB-(N,N′-(bis-aminoethyl-1,3-propanediamine)-bis 3-nitro-1,8 naphthalimide) 207, was prepared by conjugation of trastuzumab and N¹-cyclopropylmethyl, N²-maleimidopropyl-gly-val-cit-PAB-(N,N′-(bis-aminoethyl-1,3-propanediamine)-bis 3-nitro-1,8 naphthalimide) 143.

### Example 110

### In vitro Cell Proliferation Assay

Efficacy of ADC were measured by a cell proliferation assay employing the following protocol (Promega Corp. Technical Bulletin TB288; Mendoza et al (2002) Cancer Res. 62:5485-5488):
1. An aliquot of 100 μl of cell culture containing about 10⁴ cells (SKBR-3, BT474, MCF7 or MDA-MB-468) in medium was deposited in each well of a 96-well, opaque-walled plate.
2. Control wells were prepared containing medium and without cells.
3. ADC was added to the experimental wells and incubated for 3-5 days.
4. The plates were equilibrated to room temperature for approximately 30 minutes.
5. A volume of CellTiter-Glo Reagent equal to the volume of cell culture medium present in each well was added.
6. The contents were mixed for 2 minutes on an orbital shaker to induce cell lysis.
7. The plate was incubated at room temperature for 10 minutes to stabilize the luminescence signal.
8. Luminescence was recorded and reported in graphs as RLU=relative luminescence units.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All references cited herein are incorporated by reference in their entirety and for all purposes to the same extent as if each individual publication or patent or patent application was specifically and individually indicated to be incorporated by reference in its entirety for all purposes.

We claim:

1. An antibody-drug conjugate compound comprising an antibody covalently attached by a linker to one or more 1,8 bis-naphthalimide drug moieties, the compound having Formula I
Ab-(L-D)ₚ I
or a pharmaceutically acceptable salt or solvate thereof, wherein
Ab is an antibody;
L is a linker covalently attached to an Ab, and L is covalently attached to D;
D is a 1,8 bis-naphthalimide drug moiety selected from Formulas Ia and IIb: 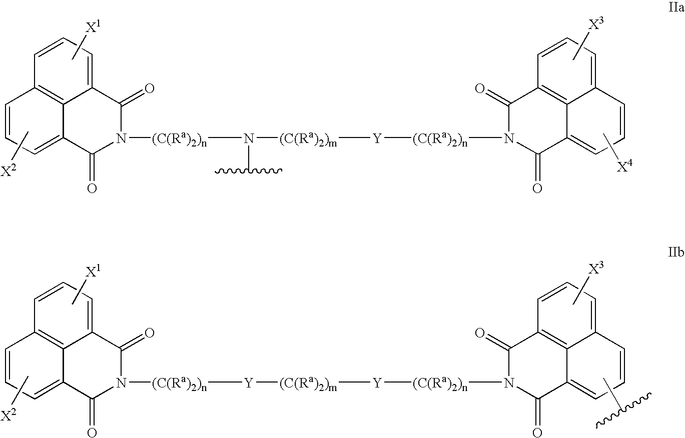
the wavy line indicates the covalent attachment to L;
Y is N(R^{b}), C(R^{a})₂, O, or S;
R^{a} is independently selected from H, F, Cl, Br, I, OH, —N(R^{b})₂, —N(R^{b})₃⁺, C₁-C₈ alkylhalide, carboxylate, sulfate, sulfamate, sulfonate, —SO₂R^{b}, —S(═O)R^{b}, —SR^{b}, —SO₂N(R^{b})₂, —C(═O)R^{b}, —CO₂R^{b}, —C(═O)N(R^{b})₂, —CN, —N₃, —NO₂, C₁-C₈ alkoxy, C₁-C₈ trifluoroalkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or when taken together, two Ra groups on the same carbon atom form a carbonyl (═O), or on different carbon atoms form a carbocyclic, heterocyclic, or aryl ring of 3 to 7 carbon atoms;
R^{b} is independently selected from H, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle;
where C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from F, Cl, Br, I, OH, —N(R^{b})₂, —N(R^{b})₃⁺, C₁-C₈ alkylhalide, carboxylate, sulfate, sulfamate, sulfonate, C₁-C₈ alkylsulfonate, C₁-C₈ alkylamino, 4-dialkylaminopyridinium, C₁-C₈ alkylhydroxyl, C₁-C₈ alkylthiol, —SO₂R^{b}, —S(═O)R^{b}, —SR^{b}, —SO₂N(R^{b})₂, C(═O)R^{b}, CO₂R^{b}, —C(═O)N(R^{b})₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate;
m is 1, 2, 3, 4, 5, or 6;
n is independently selected from 1, 2, and 3;
X¹, X², X³, and X⁴ are independently selected from F, Cl, Br, I, OH, —N(R^{b})₂, —N(R^{b})₃⁺, —N(R^{b})C(═O)R^{b}, —N(R^{b})C(═O)N(R^{b})₂, —N(R^{b})SO₂N(R^{b})₂, —N(R^{b})SO₂R^{b}, OR, OC(═O)R^{b}, OC(═O)N(R^{b})₂, C₁-C₈ alkylhalide, carboxylate, sulfate, sulfamate, sulfonate, —SO₂R^{b}, —SOAr, —SAr, —SO₂N(R^{b})₂, —SOR^{b}, —CO₂R^{b}, —C(═O)N(R^{b})₂, —CN, —N₃, —NO₂, C₁-C₈ alkoxy, C₁-C₈ trifluoroalkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀ aryl, C₆-C₂₀ substituted aryl, C₁-C₂₀ heterocycle, and C₁-C₂₀ substituted heterocycle; or
X¹ and X² together, and X³ and X⁴ together, independently form —CH₂CH₂— or —CH₂ CH₂CH₂—;
D may independently have more than one X¹, X², X³, or X⁴; and where D has more than one X¹, X², X³, or X⁴, then two X¹, X², X³, or X⁴ may form a fused C₆-C₂₀ aryl, C₆-C₂₀ substituted aryl, C₁-C₂₀ heterocycle, or C₁-C₂₀ substituted heterocycle; and
p is an integer from 1 to 20.

2. The compound of claim 1 wherein p is 2 to 8.

3. The compound of claim 1 where m is 3 and n is 2.

4. The compound of claim 1 where each R^{a} is H.

5. The compound of claim 1 wherein D is a 1,8 bis-naphthalimide drug moiety selected from Formula IIa:

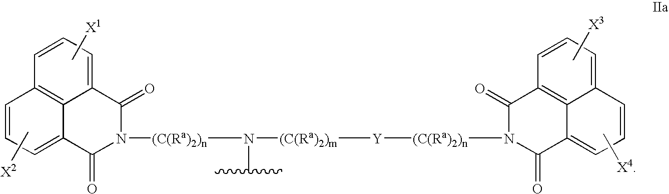

6. The compound of claim 1 wherein Y is N(R^{b}); n is 2; m is 3; and R^{a} and R^{b} are H

7. The compound of claim 6 wherein D is selected from the structures:

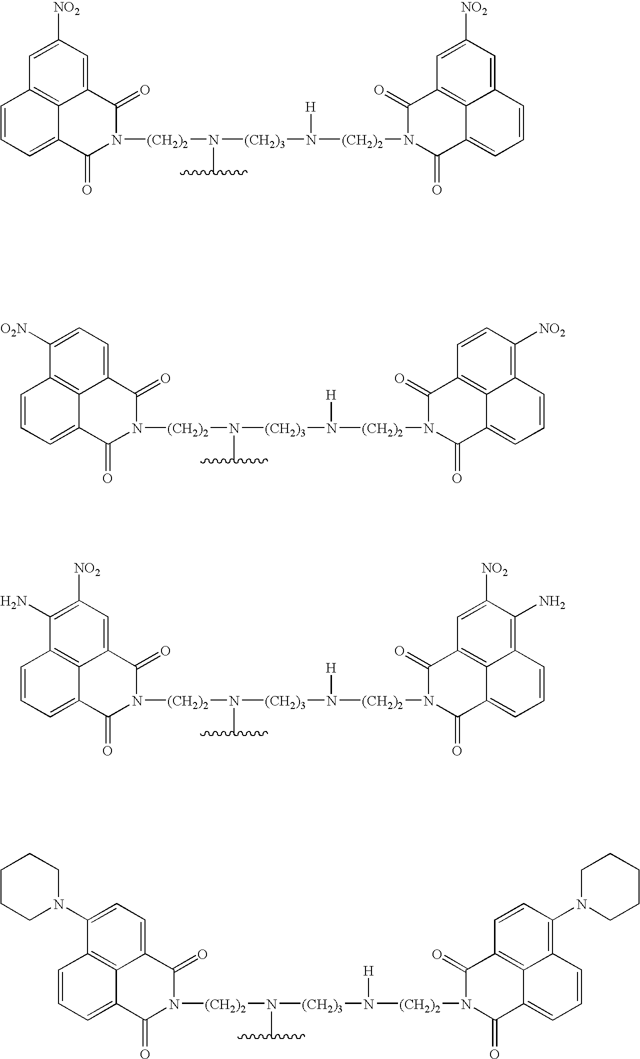

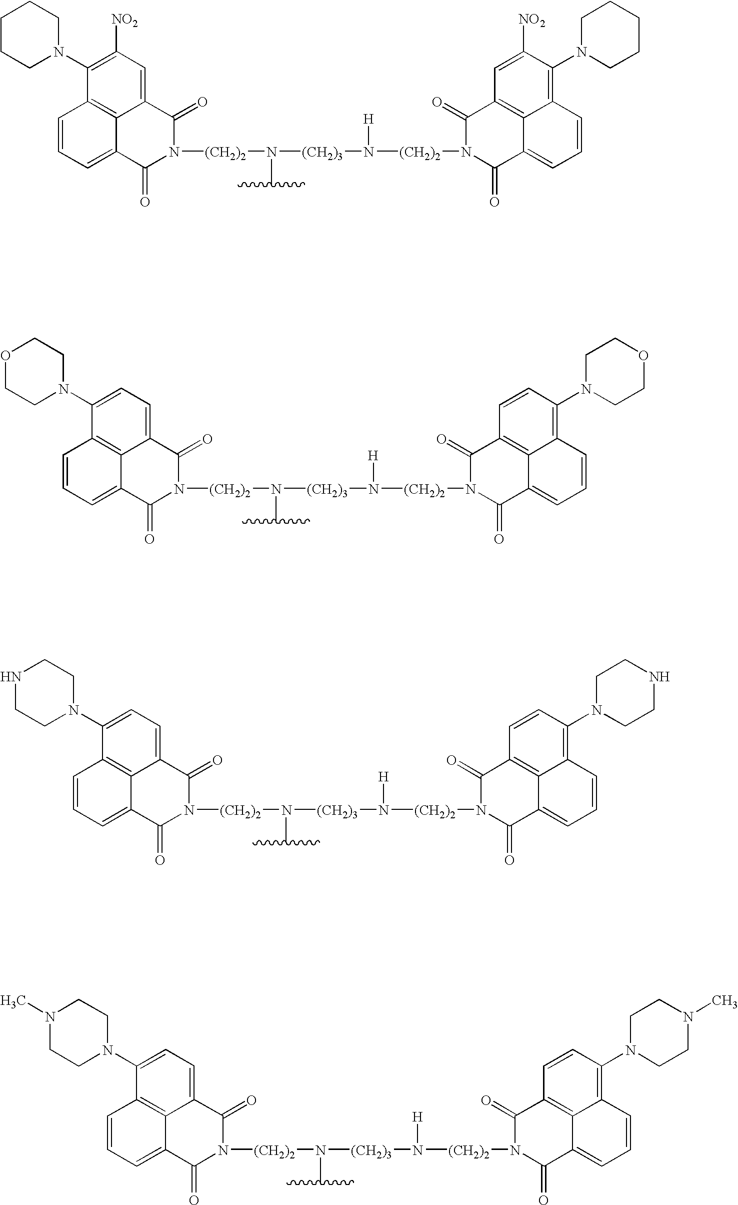

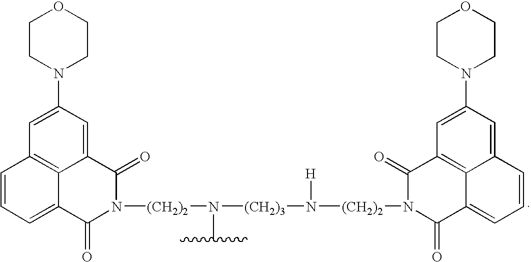

8. The compound of claim 6 wherein D is selected from the structures:

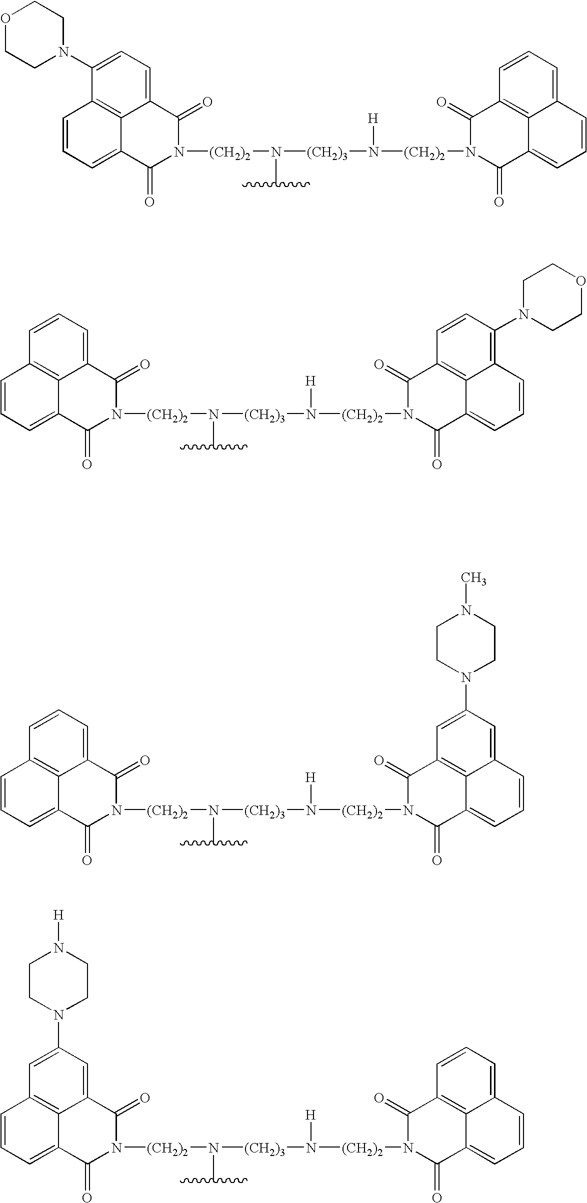

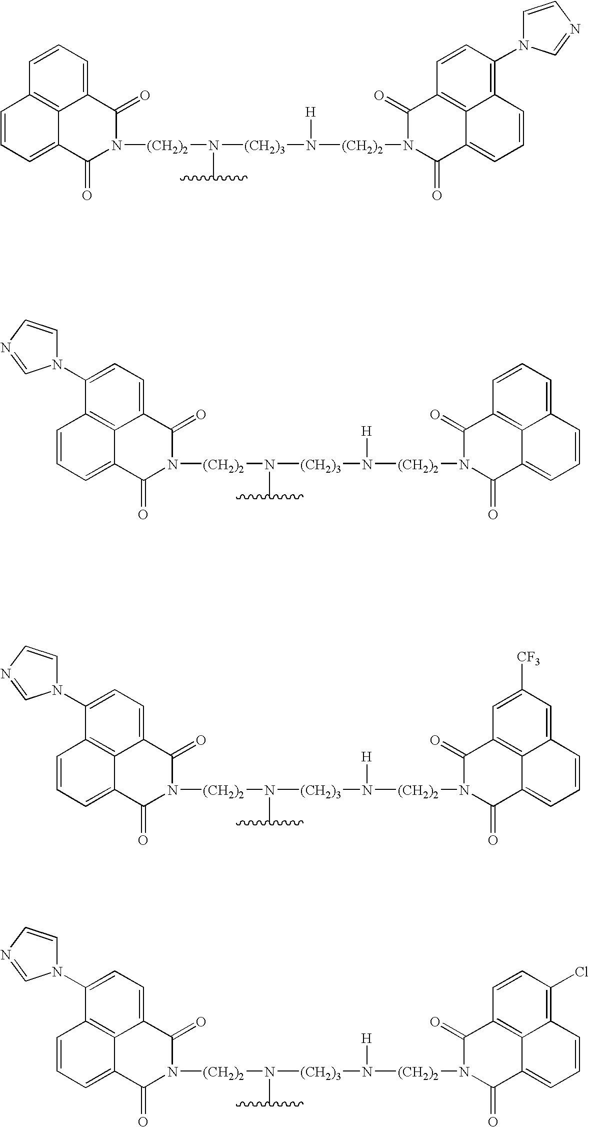

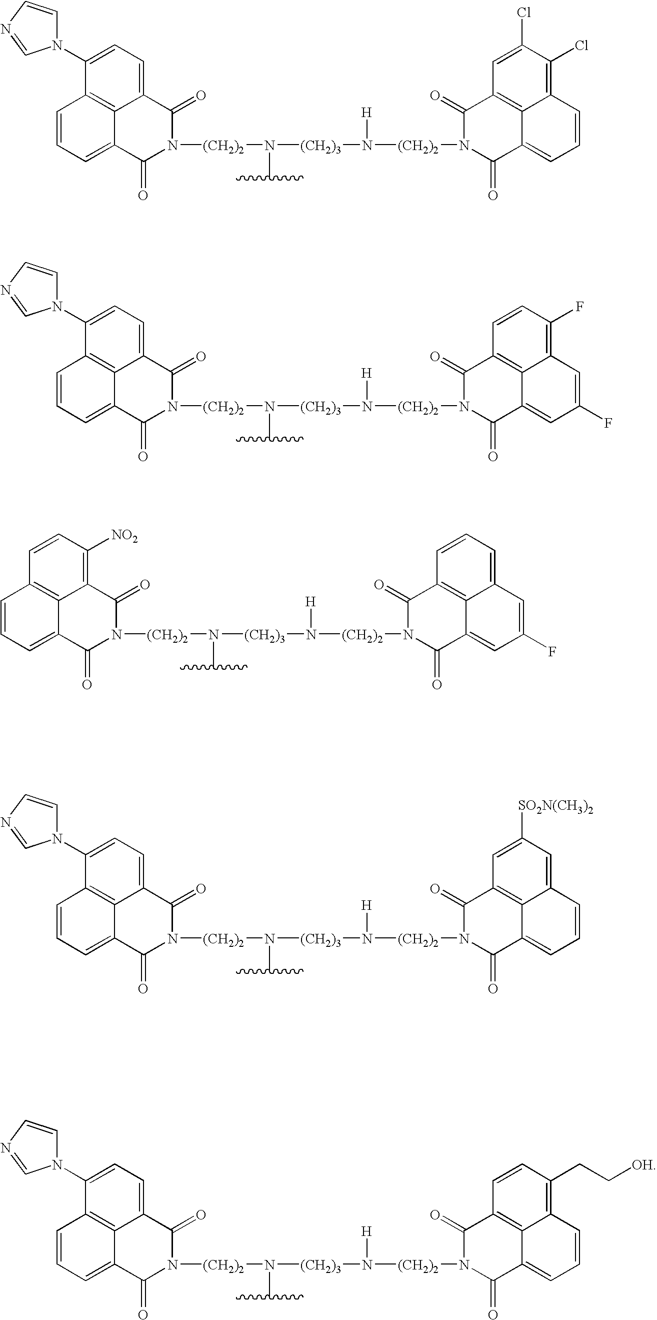

9. The compound of claim 5 wherein X¹ and X² together, or X³ and X⁴ together, independently form —CH₂CH₂— or —CH₂CH₂CH₂—.

10. The compound of claim 9 wherein D is selected from the structures:

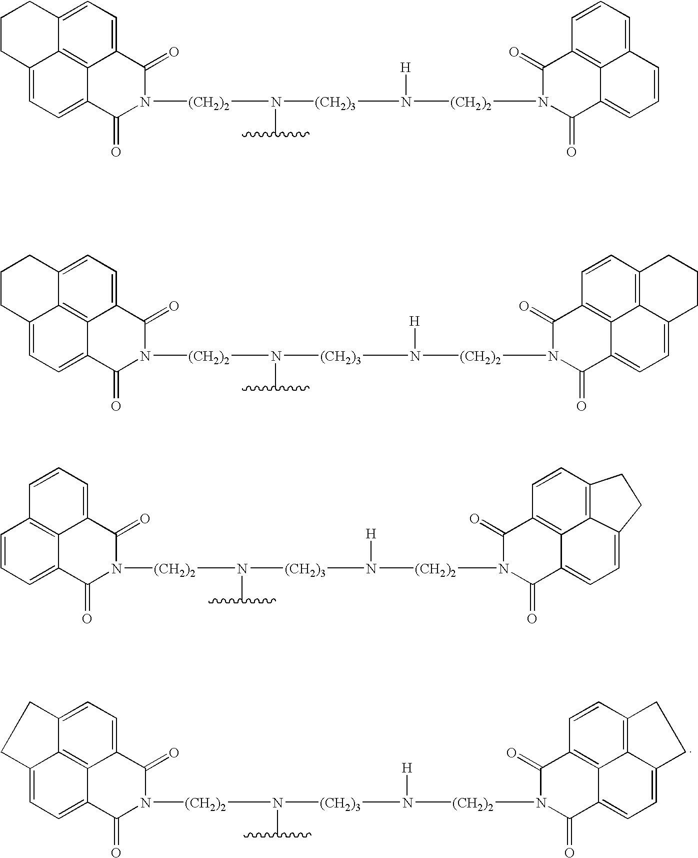

11. The compound of claim 5 wherein two X¹, X², X³, or X⁴ on adjacent carbon atoms form a fused C₆-C₂₀ aryl, C₆-C₂₀ substituted aryl, C₁-C₂₀ heterocycle, or C₁-C₂₀ substituted heterocycle.

12. The compound of claim 11 wherein D is selected from the structures:

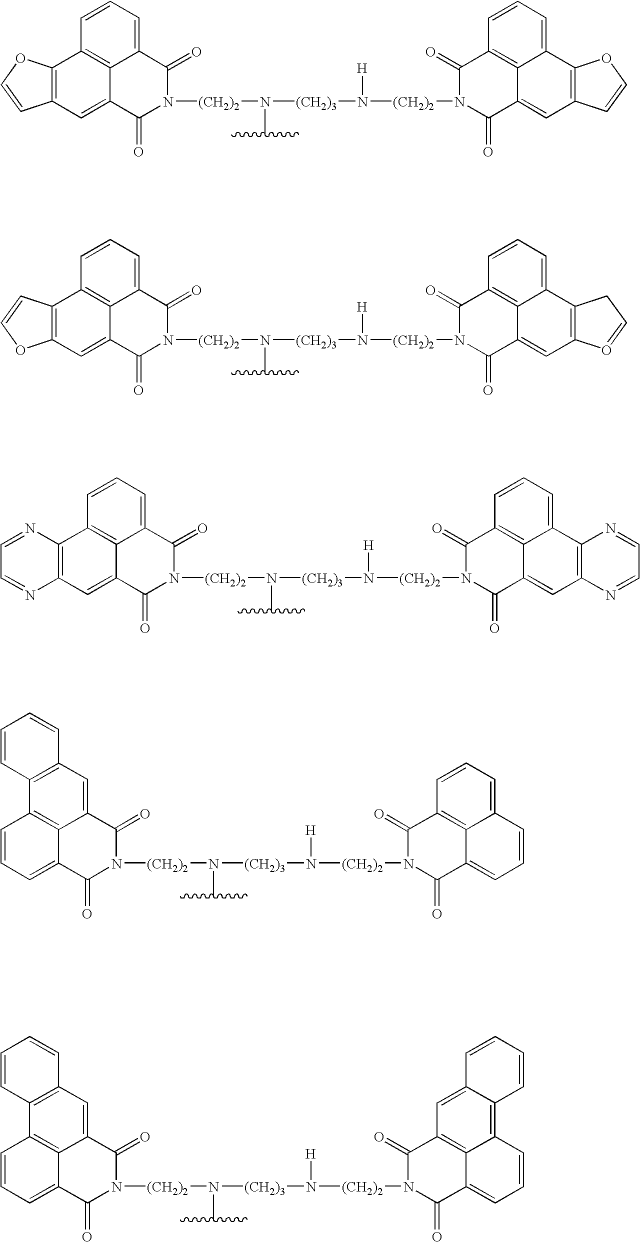

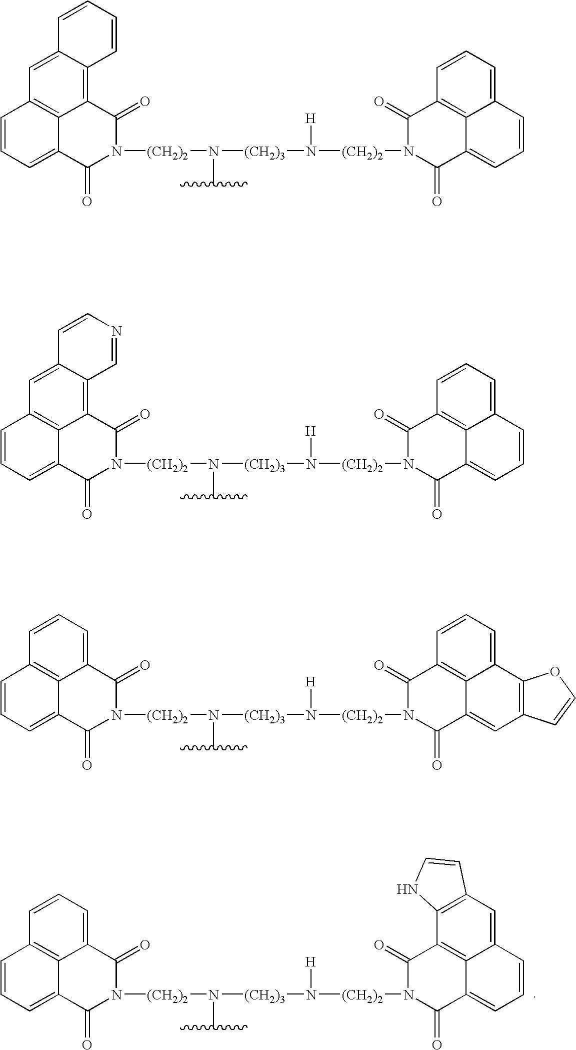

13. The compound of claim 5 wherein Y is N(R^{b}); m is 3; and n is 2.

14. The compound of claim 13 wherein D is selected from the structures:

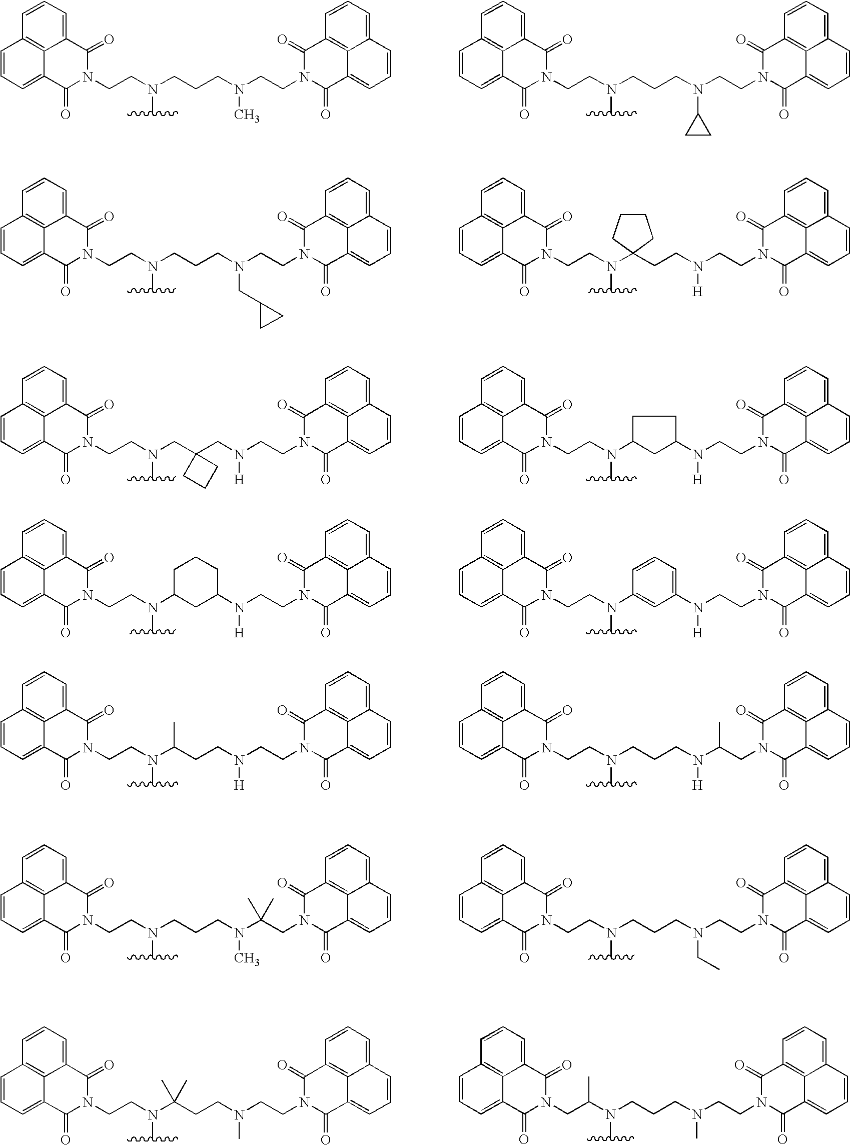

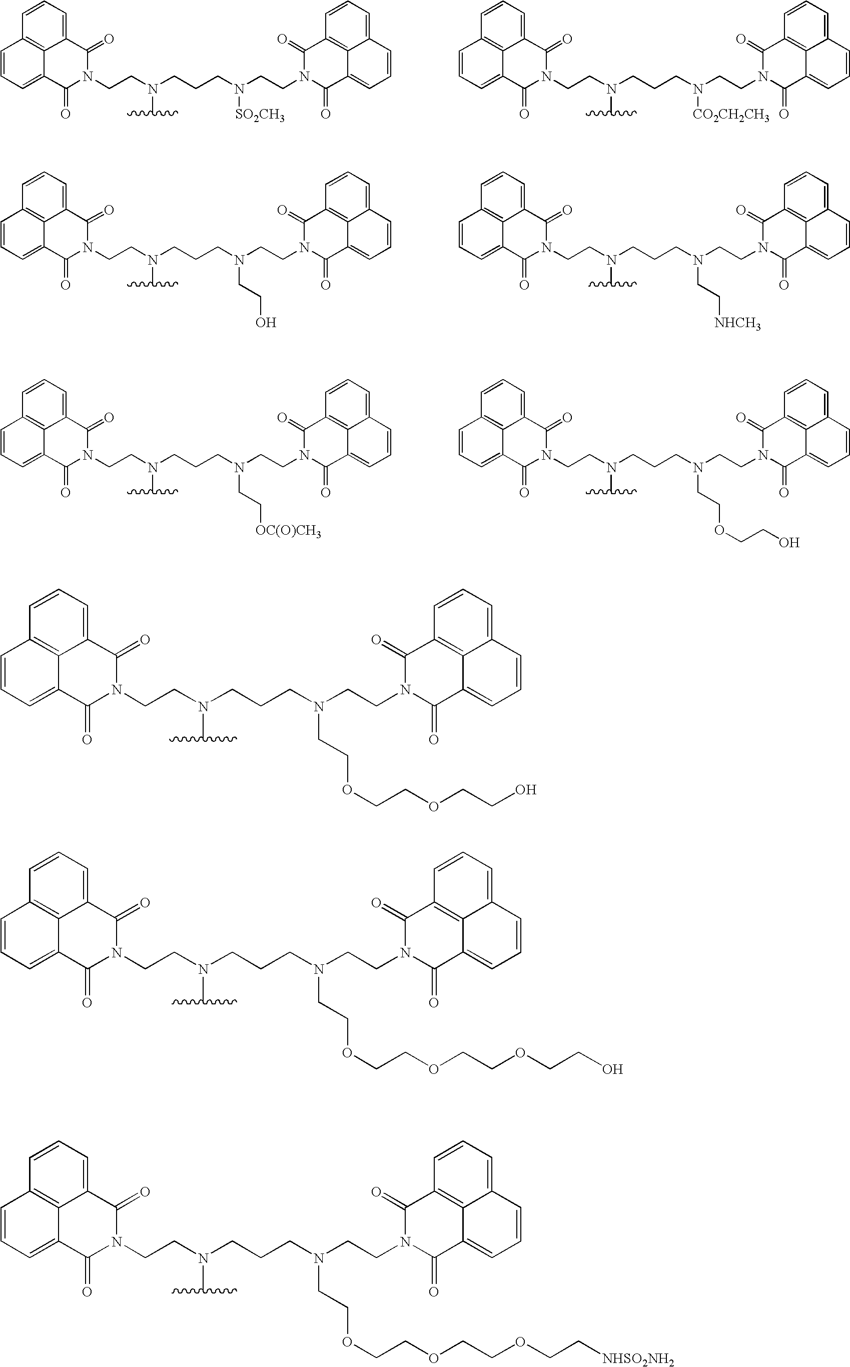

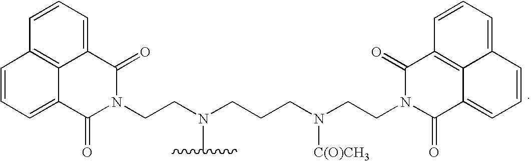

15. The compound of claim 5 wherein Y is N, and R^{a} and R^{b} are H, and D is selected from the structures:

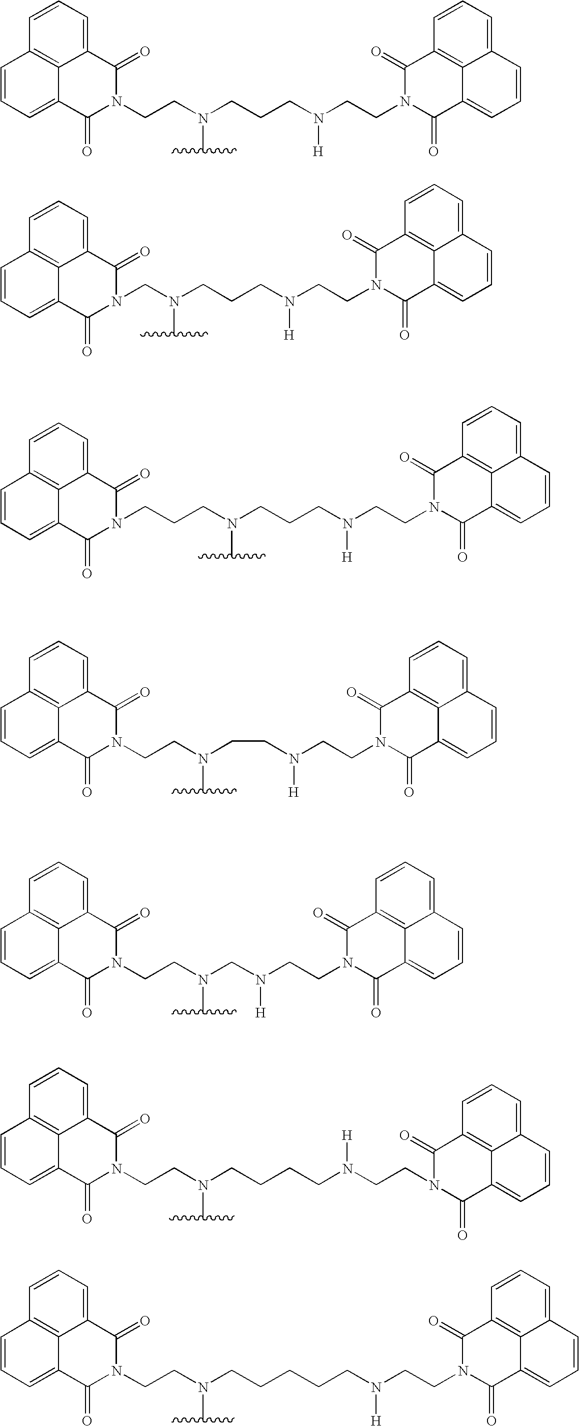

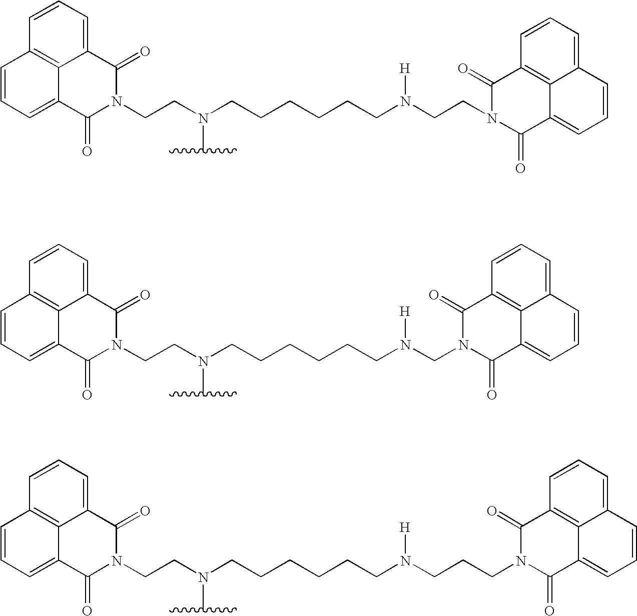

16. The compound of claim 5 where R^{b} is H.

17. The compound of claim 5 where R^{b} is CH₃.

18. The compound of claim 5 wherein Y is O or S.

19. The compound of claim 18 wherein D is selected from the structures:

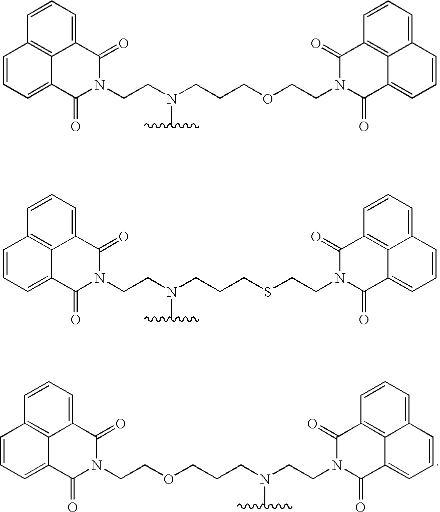

20. The compound of claim 5 wherein X¹ and X² are C₁-C₂₀ heterocycle.

21. The compound of claim 20 wherein one or both of X¹ and X² are 1-imidazolyl.

22. The compound of claim 21 wherein X¹ and X² are 1-imidazolyl at the 4 position of the 1,8 naphthalimide groups.

23. The compound of claim 22 wherein D has the structure:

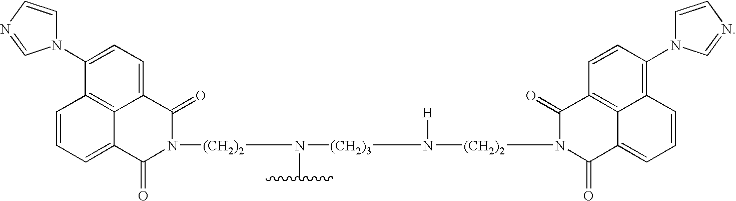

24. The compound of claim 10 wherein the antibody-drug conjugate is H-MC-af-PAB-(bis 4-imidazolyl E) 203.

25. The compound of claim 1 wherein D is a 1,8 bis-naphthalimide drug moiety selected from Formula IIb:

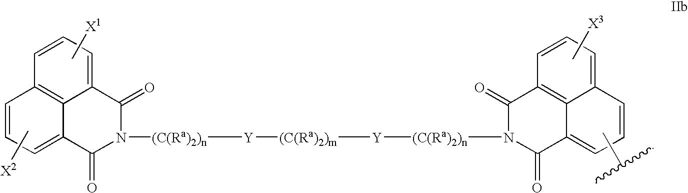

26. The compound of claim 25 wherein Y is N(R^{b}), and D is selected from the structures:

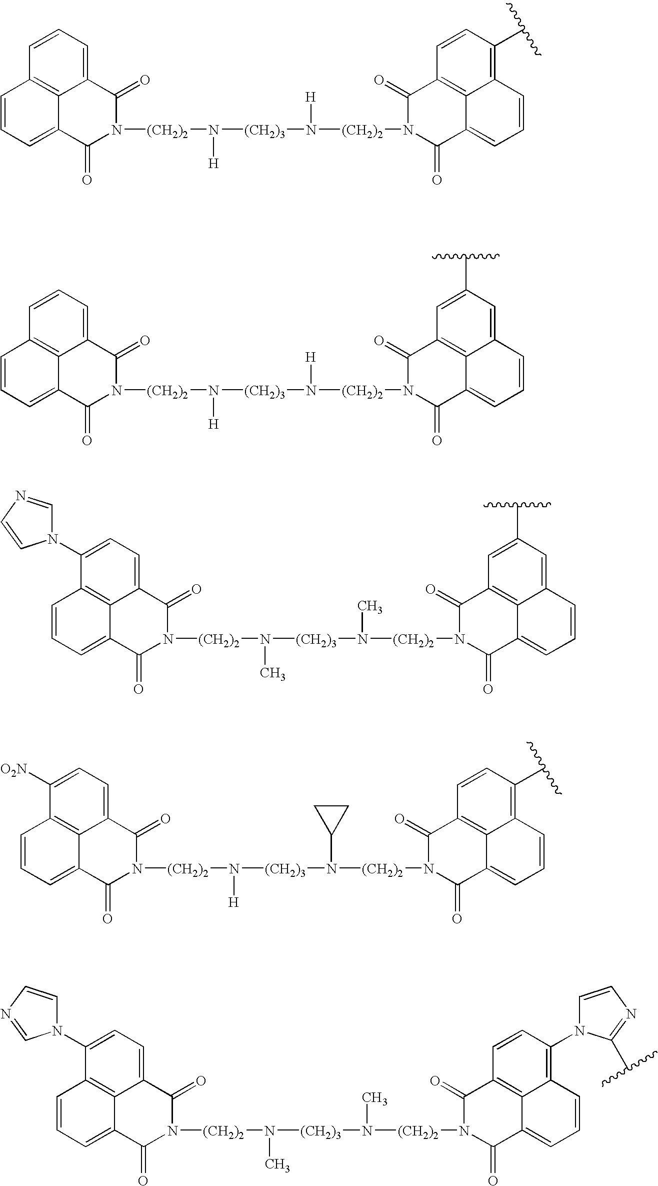

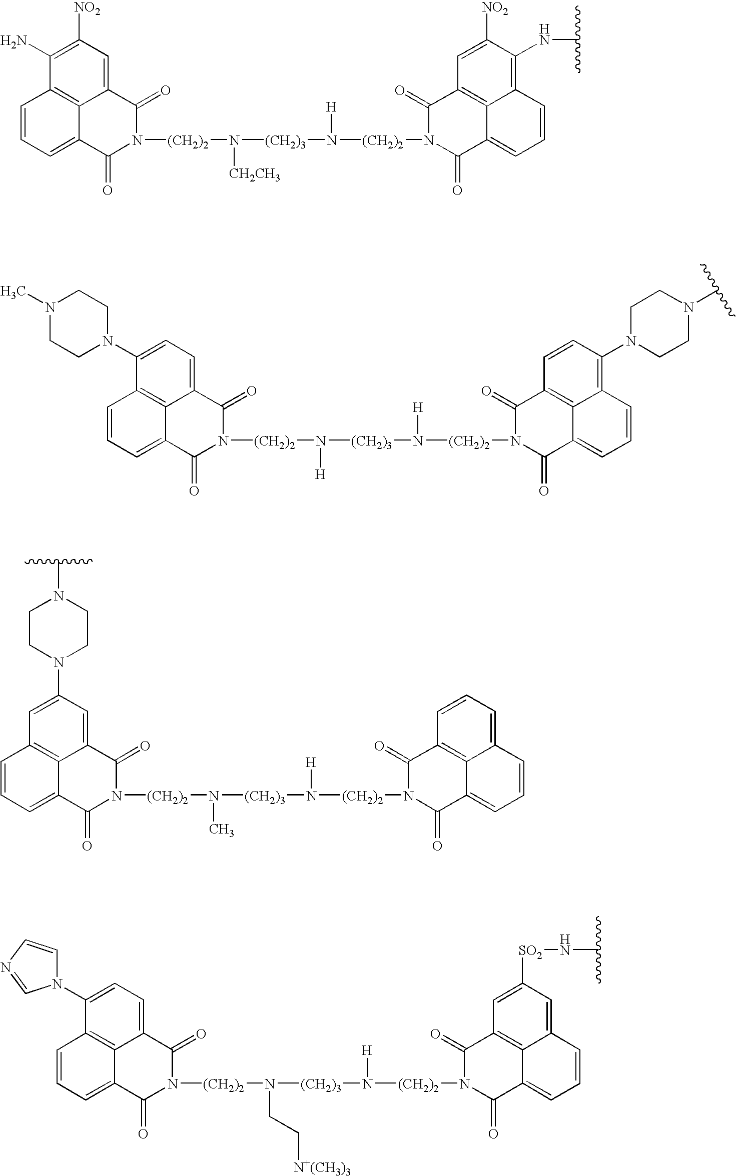

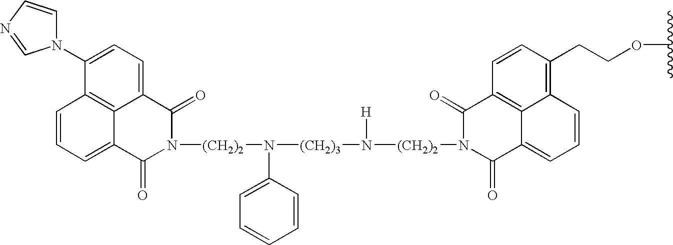

27. The compound of claim 25 wherein one or two of Y is O or S.

28. The compound of claim 27 wherein D is selected from the structures:

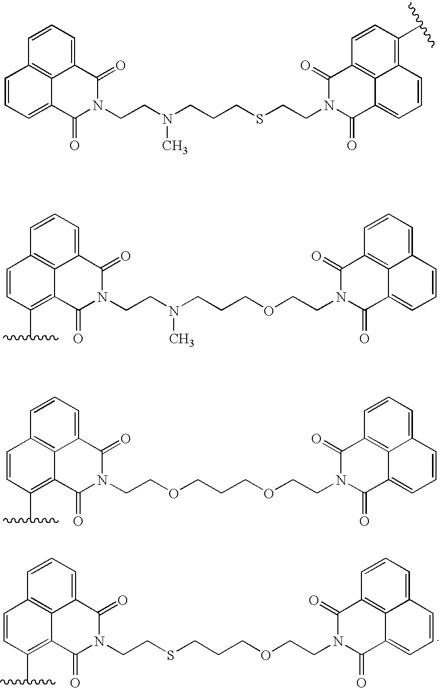

29. The compound of claim 25 where R^{b} is H.

30. The compound of claim 25 where R^{b} is CH₃.

31. The compound of claim 1 having Formula Ia:
Ab [symbol] Aₐ-W_{w}-SP_{y}-D)ₚ Ia
wherein A is a Stretcher unit,
a is 0 or 1,
each W is independently an Amino Acid unit,
w is an integer ranging from 0 to 12,
SP is a Spacer unit, and
y is 0, 1 or 2.

32. The compound of claim 31 selected from the formulas: 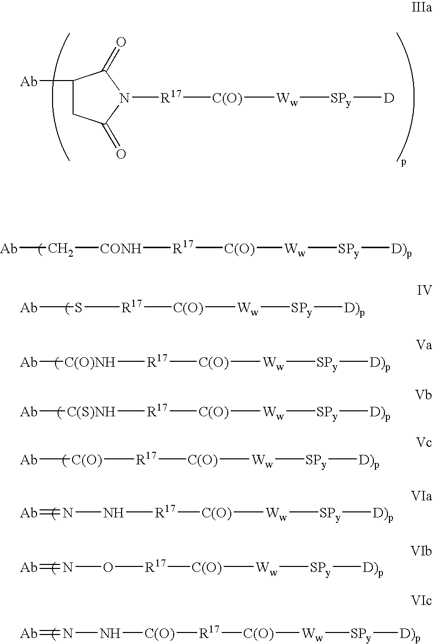
wherein R¹⁷ is selected from (CH₂)ᵣ, C₃-C₈ carbocyclyl, O—(CH₂)ᵣ, arylene, (CH₂)ᵣ-arylene, -arylene-(CH₂)ᵣ—, (CH₂)—(C₃-C₈ carbocyclyl), (C₃-C₈ carbocyclyl)-(CH₂)ᵣ, C₃-C₈ heterocyclyl, (CH₂)ᵣ—(C₃-C₈ heterocyclyl), -(C₃-C₈ heterocyclyl)-(CH₂)ᵣ—, -(CH₂)ᵣC(O)NR^{b}(CH₂)ᵣ—, -(CH₂CH₂O)ᵣ(CH₂)ᵣ—, -(CH₂)ᵣO(CH₂CH₂O)ᵣ(CH₂)ᵣ—, —(CH₂)ᵣC(O)NR^{b}(CH₂CH₂O)ᵣ(CH₂)ᵣ—, -(CH₂)ᵣC(O)NR^{b}(CH₂CH₂O)ᵣ—CH₂—, -(CH₂CH₂O)ᵣC(O)NR^{b}(CH₂CH₂O)ᵣ(CH₂)ᵣ—, -(CH₂CH₂O)C(O)NR^{b}(CH₂CH₂O), —CH₂—, and —(CH₂CH₂O)ᵣC(O)NR^{b}(CH₂)ᵣ—;
where r is independently an integer ranging from 1-10.

33. The compound of claim 32 having the formula:

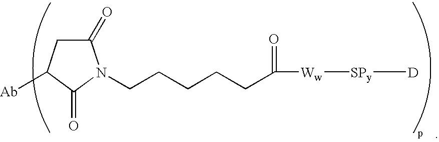

34. The compound of claim 33 having the formula: 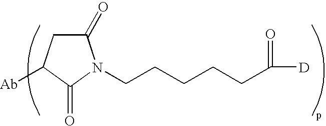
wherein w and y are each 0.

35. The compound of claim 31 having the formula:

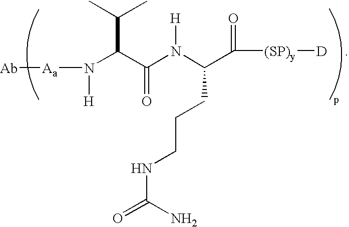

36. The compound of claim 35 having the formula:

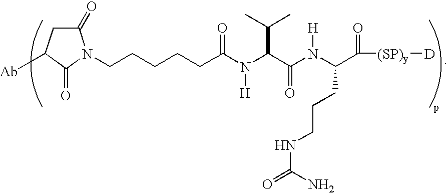

37. The compound of claim 36 having the formula:

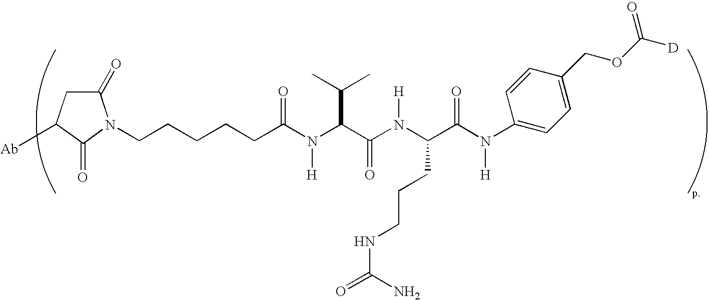

38. The compound of claim 31 wherein w is an integer ranging from 2 to 12.

39. The compound of claim 38 wherein w is 2.

40. The compound of claim 39 wherein W_{w} is -valine-citrulline-.

41. The compound of claim 40 wherein W_{w} is -alanine-phenylalanine-.

42. The compound of claim 1 selected from the structures: 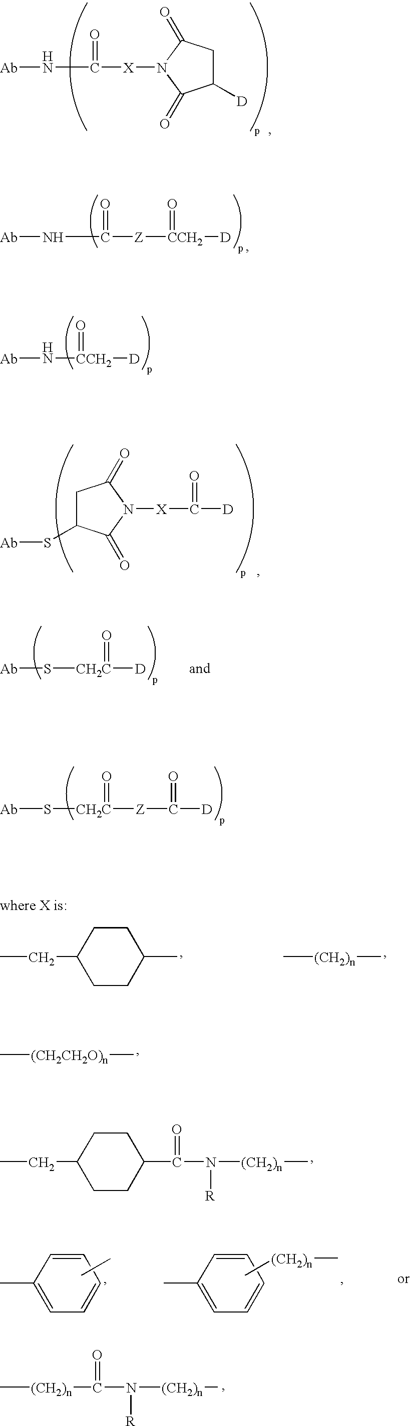 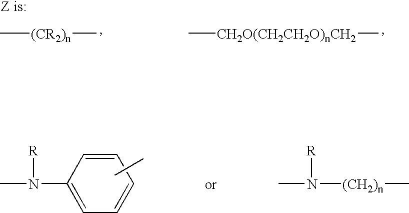
R is independently H or C₁-C₆ alkyl; and n is 1 to 12.

43. The compound of claim 42 having the structure:

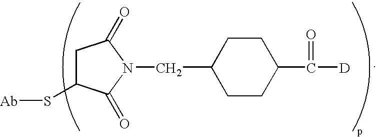

44. The compound of claim 42 having the structure:

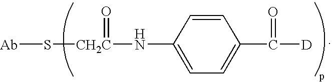

45. The compound of claim 1 wherein the antibody binds to one or more tumor-associated antigens or cell-surface receptors selected from (1)-(36):
(1) BMPR1B (bone morphogenetic protein receptor-type IB, Genbank accession no. NM_{—}001203);
(2) E16 (LAT1, SLC7A5, Genbank accession no. NM_{—}003486);
(3) STEAP1 (six transmembrane epithelial antigen of prostate, Genbank accession no. NM_{—}012449);
(4) 0772P (CA125, MUC16, Genbank accession no. AF361486);
(5) MPF (MPF, MSLN, SMR, megakaryocyte potentiating factor, mesothelin, Genbank accession no. NM_{—}005823);
(6) Napi3b (NAPI-3B, NPTIIb, SLC34A2, solute carrier family 34 (sodium phosphate), member 2, type II sodium-dependent phosphate transporter 3b, Genbank accession no. NM_{—}006424);
(7) Sema 5b (FLJ10372, KIAA1445, Mm.42015, SEMA5B, SEMAG, Semaphorin 5b Hlog, sema domain, seven thrombospondin repeats (type I and type 1-like), transmembrane domain (TM) and short cytoplasmic domain, (semaphorin) 5B, Genbank accession no. AB040878);
(8) PSCA hlg (2700050C12Rik, C530008016Rik, RIKEN cDNA 2700050C12, RIKEN cDNA 2700050C12 gene, Genbank accession no. AY358628);
(9) ETBR (Endothelin type B receptor, Genbank accession no. AY275463);
(10) MSG783 (RNF124, hypothetical protein FLJ20315, Genbank accession no. NM_{—}017763);
(11) STEAP2 (HGNC_{—}8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138);
(12) TrpM4 (BR22450, FLJ20041, TRPM4, TRPM4B, transient receptor potential cation channel, subfamily M, member 4, Genbank accession no. NM_{—}017636);
(13) CRIPTO (CR, CR1, CRGF, CRIPTO, TDGF1, teratocarcinoma-derived growth factor, Genbank accession no. NP_{—}003203 or NM_{—}003212);
(14) CD21 (CR2 (Complement receptor 2) or C3DR (C3d/Epstein Barr virus receptor) or Hs.73792 Genbank accession no. M26004);
(15) CD79b (CD79B, CD79p, IGb (immunoglobulin-associated beta), B29, Genbank accession no. NM_{—}000626);
(16) FcRH2 (IFGP4, IRTA4, SPAP1A (SH2 domain containing phosphatase anchor protein 1a), SPAP1B, SPAP1C, Genbank accession no. NM_{—}030764);
(17) HER2 (Genbank accession no. M11730);
(18) NCA (Genbank accession no. M18728);
(19) MDP (Genbank accession no. BC017023);
(20) IL20Rα (Genbank accession no. AF184971);
(21) Brevican (Genbank accession no. AF229053);
(22) EphB2R (Genbank accession no. NM_{—}004442);
(23) ASLG659 (Genbank accession no. AX092328);
(24) PSCA (Genbank accession no. AJ297436);
(25) GEDA (Genbank accession no. AY260763;
(26) BAFF-R (B cell—activating factor receptor, BLyS receptor 3, BR3, NP_{—}443177.1); and
(27) CD22 (B-cell receptor CD22-β isoform, NP-001762.1);
(28) CD79a (CD79α, CD79a, immunoglobulin-associated alpha, a B cell-specific protein that covalently interacts with Ig beta (CD79B) and forms a complex on the surface with Ig M molecules, transduces a signal involved in B-cell differentiation, Genbank accession No. NP_{—}001774.1);
(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia, Genbank accession No. NP_{—}001707.1);
(30) HLA-DOB (Beta subunit of MHC class II molecule (Ia antigen) that binds peptides and presents them to CD4+ T lymphocytes, Genbank accession No. NP_{—}002111.1);
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability, Genbank accession No. NP_{—}002552.2);
(32) CD72 (B-cell differentiation antigen CD72, Lyb-2, Genbank accession No. NP_{—}001773.1);
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis, Genbank accession No. NP_{—}005573.1);
(34) FCRH1 (Fc receptor-like protein 1, a putative receptor for the immunoglobulin Fc domain that contains C2 type Ig-like and ITAM domains, may have a role in B-lymphocyte differentiation, Genbank accession No. NP_{—}443170.1);
(35) IRTA2 (Immunoglobulin superfamily receptor translocation associated 2, a putative immunoreceptor with possible roles in B cell development and lymphomagenesis; deregulation of the gene by translocation occurs in some B cell malignancies, Genbank accession No. NP_{—}112571.1); and
(36) TENB2 (putative transmembrane proteoglycan, related to the EGF/heregulin family of growth factors and follistatin, Genbank accession No. AF179274.

46. The antibody-drug conjugate compound of claim 1 wherein the antibody specifically binds to a receptor encoded by an ErbB gene.

47. The antibody-drug conjugate compound of claim 46 wherein the antibody specifically binds to an ErbB receptor selected from EGFR, HER2, HER3 and HER4.

48. The antibody-drug conjugate compound of claim 47 which specifically binds to the extracellular domain of the HER2 receptor and inhibits growth of tumor cells which overexpress HER2 receptor.

49. The antibody-drug conjugate compound of claim 1 wherein the antibody is a monoclonal antibody.

50. The antibody-drug conjugate compound of claim 1 wherein the antibody is a bispecific antibody.

51. The antibody-drug conjugate compound of claim 1 wherein the antibody is a chimeric antibody.

52. The antibody-drug conjugate compound of claim 1 wherein the antibody is a humanized antibody.

53. The antibody-drug conjugate compound of claim 52 wherein the humanized antibody is selected from huMAb4D5-1, huMAb4D5-2, huMAb4D5-3, huMAb4D5-4, huMAb4D5-5, huMAb4D5-6, huMAb4D5-7 and huMAb4D5-8 (trastuzumab).

54. The antibody-drug conjugate compound of claim 53 wherein the antibody is huMAb4D5-8 (trastuzumab).

55. The conjugate of claim 1 wherein the antibody is an antibody fragment.

56. The conjugate of claim 55 wherein the antibody fragment is a Fab fragment.

57. A pharmaceutical composition comprising an effective amount of the antibody-drug conjugate compound of claim 1, or a pharmaceutically acceptable salt thereof, and a pharmaceutically acceptable diluent, carrier or excipient.

58. The pharmaceutical composition of claim 57 further comprising a therapeutically effective amount of chemotherapeutic agent selected from a tubulin-forming modulator, a topoisomerase inhibitor, and a DNA binder.

59. A method for killing or inhibiting the proliferation of tumor cells or cancer cells comprising treating tumor cells or cancer cells in a cell culture medium with an amount of the compound of claim 1, or a pharmaceutically acceptable salt or solvate thereof, being effective to kill or inhibit the proliferation of the tumor cells or cancer cells.

60. The use of an antibody-drug conjugate compound of claim 1 in the preparation of a medicament for the treatment of cancer.

61. An assay for detecting cancer cells comprising exposing cells to an antibody-drug conjugate compound of claim 1; and determining the extent of binding of the antibody-drug conjugate compound to the cells.

62. The assay of claim 61 wherein the cells are breast tumor cells.

63. The assay of claim 61 wherein the extent of binding is determined by measuring levels of ErbB-encoding nucleic acid by fluorescent in situ hybridization (FISH).

64. The assay of claim 61 wherein the extent of binding is determined by immunohistochemistry (1HC).

65. An article of manufacture comprising
an antibody-drug conjugate compound of claim 1;
a container; and
a package insert or label indicating that the compound can be used to treat cancer.

66. The article of manufacture of claim 65 wherein said package insert of label indicates that the compound can be used to treat cancer characterized by the overexpression of an ErbB2 receptor.

67. The article of manufacture of claim 66 wherein the cancer is breast cancer.

68. A method of making an antibody drug conjugate compound comprising conjugating a 1,8 bis naphthalimide drug moiety and an antibody.
